SEQUENCE LISTING (55 sequences)

SEQ ID NO: 1 - protein, 303 aa, Cyberlindnera jadinii
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 2 - protein, 301 aa, Aspergillus flavus
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 3 - protein, 302 aa, Arthrobacter globiformis
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 4 - protein, 304 aa, Artificial Sequence
  Description of Artificial Sequence Synthetic polypeptide
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 5 - protein, 494 aa, Bacillus subtilis
MFTMDDLNQMDTQTLTDTLGSIFEHSSWIAERSAALRPFSSLSDLHRKMTGIVKAADRETQLDLIKKHPRLGTKKTMSDDSVREQQNAGLGKLEQQEYEEFLMLNEHYYDRFGFPFILAVKGKTKQDIHQALLARLESERETEFQQALIEIYRIARFRLADIITEKGETQMKRTMSYGKGNVFAYRTYLKPLTGVKQIPESSFAGRDNTVVGVDVTCEIGGEAFLPSFTDGDNTLVVATDSMKNFIQRHLASYEGTTTEGFLHYVAHRFLDTYSHMDTITLTGEDIPFEAMPAYEEKELSTSRLVFRRSRNERSRSVLKAERSGNTITITEQYSEIMDLQLVKVSGNSFVGFIRDEYTTLPEDGNRPLFVYLNISWQYENTNDSYASDPARYVAAEQVRDLASTVFHELETPSIQNLIYHIGCRILARFPQLTDVSFQSQNHTWDTVVEEIPGSKGKVYTEPRPPYGFQHFTVTREDAEKEKQKAAEKCRSLKA

SEQ ID NO: 6 - protein, 300 aa, Cellulomonas flavigena
MAVGDVVLGANQYGKAEVRLVRVTRDTPVHEIEDLTVTTQLRGDFAACHTTGDNAHVVATDTQKNTVYAFARTHGVGSPEAFLLRLARHFVDGFDQVTGGRFAADVHAWDRIAVDGKPHDHAFVRTGAGTRRAVVLVDGDDVHVVSGFTGATVLKSTGSEFWGFPRDRYTTLAETKDRILATSVTAWWRWTTPDVDVEARYPVVKDLLLSTFAQVHSLALQHTIFEMGRAVLEACDDVAEVRLSCPNKHHLLVDLEPFGLENPGEVFHAADRPYGLIEAAVHREGDPPVPHVWASVPGFV

SEQ ID NO: 7 - protein, 303 aa, Mus musculus
MAHYHDNYGKNDEVEFVRTGYGKDMVKVLHIQRDGKYHSIKEVATSVQLTLRSKKDYLHGDNSDIIPTDTIKNTVHVLAKLRGIRNIETFAMNICEHFLSSFNHVTRAHVYVEEVPWKRFEKNGIKHVHAFIHTPTGTHFCEVEQMRNGPPVIHSGIKDLKVLKTTQSGFEGFLKDQFTTLPEVKDRCFATQVYCKWRYQRRDVDFEAIWGAVRDIVLQKFAGPYDKGEYSPSVQKTLYDIQVLSLSQLPEIEDMEISLPNIHYFNIDMSKMGLINKEEVLLPLDNPYGKITGTVKRKLPSRL

SEQ ID NO: 8 - protein, 309 aa, Glycine max
MAQQEVVEGFKFEQRHGKERVRVARVWKTRQGQHFIVEWRVGITLFSDCVNSYLRDDNSDIVATDTMKNTVYAKAKECSDILSAEEFAILLAKHFVSFYQKVTGAIVNIVEKPWERVTVDGQPHEHGFKLGSEKHTTEAIVQKSGSLQLTSGIEGLSVLKTTQSGFVNFIRDKYTALPDTRERMVATEVTALWRYSYESLYSLPQKPLYFTEKYQEVKKVLADTFFGPPKGGVYSPSVQNTLYLMAKATLNRFPDIAYVSLKLPNLHFIPVNISNQDGPIVKFEDDVYLPTDEPHGSIQASLSRLWSKL

SEQ ID NO: 9 - protein, 298 aa, Danio rerio
MATTSNQNVEFVRTGYGKNMVKVLHIRREGNHHHIIELIANVQLTLKTRKDYLTGDNSDIIPTDTVKNTVHALAKLKGIKSIESFALDICEHFLTAFNHVTRVKVNIDEVPWKRLEKNGVEHNHAFIHCPEALRFCEAEQYLSKTPVVHSGLKDMKVLKTTQTGFEGFLRDRFTTLTDAKDRFFCTSVYARWRYNTINVAFDAAWKAVKDTVIQKFAGPYDRGEYSPSVQKTLYDTQLLVLDRIPEVEEIEIIMPNQHYFVIDMTKIGLSNKDEVYLPLDNPSGNITGTVCRKPRARM

SEQ ID NO: 10 - protein, 296 aa, Schizosaccharomyces pombe
MSETTYVKQCAYGKTLVRFMKKDICPKTKTHTVYEMDVQSLLTGELEESYTKADNSIVVPTDTQKNTIYVFAKNNDVSVPEVFAAKLAKHFVDKYKHIHGAALDITITPWTRMEVQGKPHSHSFIRNPGETRKTHVVFSEGKGFDVVSSLKDVLVLKSTGSGFTNFHKCEFTTLPEVTDRIFSTSIDCNYTFKHFDTFEELAGFDFNSIYEKVKEITLETFALDDSESVQATMYKMADTIINTYPAINEVYYALPNKHYFEINLAPFNIDNLGSNCSLYQPQAYPSGYITCTVARK

SEQ ID NO: 11 - protein, 304 aa, Macaca mulatta
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVIHSGTKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 12 - protein, 307 aa, Oryza sativa
MADRLELQGRHGKSRVRVSRVWRRPAAAGGHVIVEWNVAVSVVSDCLPSYTSDDNSAIVATDSIKNTVYVKAKECTEIVSMEEFAVILGRHFTSLYPQVSEATVTIAERPWERVVVDGKPHSHGFKLGVEKHVTEVIVKKSGNLLINSGIQGYSLLKTTQSGFEKFVRDRYTLLPDTRERIVATEVTAWWRYPFEHVSQIPSKSFCFTQRYQDVKKVLADTFFGPPDVGVYSPSVQNTLYLMAKEVLNRFPDIASVQLRMPNLHFIPVNLGNKENPGLVKFADDVYLPTDEPHGTIEATVSRPKSKL

SEQ ID NO: 13 - protein, 350 aa, Penicillium freii
MILRSANSLHSRSNKYSFKLLDNTIFLPFSILYFFFSSIFQRYHSKFKMSALAAARYGKDNVRVCKVHRDEKTGIQTVVEMTVCVLLEGDIETSYTKADNSVVVATDSIKNTIFITAKQNPVTPPELFGSILGTHFIEKYSHIHAAHVNIVTHRWVRLDIDGKPHPHSFIKPGSETRNVQVDVIEGKGIDINSSIKGLTVLKSTGSQFWGFVRDEYTTLKETWDRLLSTDVAANWQWRRFTGLADVKTHSEKFNAAWEAARAITLKTFADDNSASVQATMYKMGEQILAAVPLLDTVEYALPNIHFFEVDLSWHKGIKNTGKDAEVYAPQSNPNGLIKCTVGRAGQKAKL

SEQ ID NO: 14 - protein, 302 aa, Aspergillus niger
MSSPVTAARYGKDNVRVYKVHRDEKTGVQTVVEMTVCVLLEGDIDTSYTKADNSVIVATDSIKNTIYITAKQNPVTPPELFGSILGSHFITKYNHIHAAHVNIITHRWTRMTIDGKPHPHSFLRDGEETRNVQVDVVEGKGVDITSSLAKLTVLKSTNSQFWGFLRDEYTTLPETWDRILSTDVDASWQWRRFSGLDEVRATVPHFDATWAAAREITLKTFAEDNSASVQNTMYKMAEQILARQSLLETVEYSLPNKHYFEVDLSWHKGLKNTGKDAEVYAPQTNPNGLIKCTVGRNTKAKL

SEQ ID NO: 15 - protein, 352 aa, Drosophila melanogaster
MFATPLRQPAAANHQTPKNSAGMDEHGKPYQYEITDHGYGKDAVKVLHVSRNGPVHAIQEFEVGTHLKLYSKKDYYQGNNSDIVATDSQKNTVYLLAKKHGIESPEKFALLLARHFINKYSHVEEAHVHVEAYPWQRVCQEETRTNVNGKCENGVQGNCDFSSIDNRSLHNHAFIFTPTALHYCDVVIRRTDPKQTVITGIKGLRVLKTTQSSFVNFVNDEFRSLPDQYDRIFSTVVDCSWEYSDTENLDFLRAWQTVKNIIIRNFAGDPQVGVSSPSVQHTLYLSERQVLDVLPQVSVISMTMPNKHYFNFDTKPFQKIAPGDNNEVFIPVDKPHGTIYAQLARKNINSHL

SEQ ID NO: 16 - protein, 553 aa, Homo sapiens
MAFSELLDLVGGLGRFQVLQTMALMVSIMWLCTQSMLENFSAAVPSHRCWAPLLDNSTAQASILGSLSPEALLAISIPPGPNQRPHQCRRFRQPQWQLLDPNATATSWSEADTEPCVDGWVYDRSIFTSTIVAKWNLVCDSHALKPMAQSIYLAGILVGAAACGPASDRFGRRLVLTWSYLQMAVMGTAAAFAPAFPVYCLFRFLLAFAVAGVMMNTGTLLMEWTAARARPLVMTLNSLGFSFGHGLTAAVAYGVRDWTLLQLVVSVPFFLCFLYSWWLAESARWLLTTGRLDWGLQELWRVAAINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQF

SEQ ID NO: 17 - protein, 540 aa, Homo sapiens
MARKQNRNSKELGLVPLTDDTSHAGPPGPGRALLECDHLRSGVPGGRRRKDWSCSLLVASLAGAFGSSFLYGYNLSVVNAPTPYIKAFYNESWERRHGRPIDPDTLTLLWSVTVSIFAIGGLVGTLIVKMIGKVLGRKHTLLANNGFAISAALLMACSLQAGAFEMLIVGRFIMGIDGGVALSVLPMYLSEISPKEIRGSLGQVTAIFICIGVFTGQLLGLPELLGKESTWPYLFGVIVVPAVVQLLSLPFLPDSPRYLLLEKHNEARAVKAFQTFLGKADVSQEVEEVLAESRVQRSIRLVSVLELLRAPYVRWQVVTVIVTMACYQLCGLNAIWFYTNSIFGKAGIPPAKIPYVTLSTGGIETLAAVFSGLVIEHLGRRPLLIGGFGLMGLFFGTLTITLTLQDHAPWVPYLSIVGILAIIASFCSGPGGIPFILTGEFFQQSQRPAAFIIAGTVNWLSNFAVGLLFPFIQKSLDTYCFLVFATICITGAIYLYFVLPETKNRTYAEISQAFSKRNKAYPPEEKIDSAVTDGKINGRP

SEQ ID NO: 18 - protein, 550 aa, Homo sapiens
MAFSKLLEQAGGVGLFQTLQVLTFILPCLMIPSQMLLENFSAAIPGHRCWTHMLDNGSAVSTNMTPKALLTISIPPGPNQGPHQCRRFRQPQWQLLDPNATATSWSEADTEPCVDGWVYDRSVFTSTIVAKWDLVCSSQGLKPLSQSIFMSGILVGSFIWGLLSYRFGRKPMLSWCCLQLAVAGTSTIFAPTFVIYCGLRFVAAFGMAGIFLSSLTLMVEWTTTSRRAVTMTVVGCAFSAGQAALGGLAFALRDWRTLQLAASVPFFAISLISWWLPESARWLIIKGKPDQALQELRKVARINGHKEAKNLTIEVLMSSVKEEVASAKEPRSVLDLFCVPVLRWRSCAMLVVNFSLLISYYGLVFDLQSLGRDIFLLQALFGAVDFLGRATTALLLSFLGRRTIQAGSQAMAGLAILANMLVPQDLQTLRVVFAVLGKGCFGISLTCLTIYKAELFPTPVRMTADGILHTVGRLGAMMGPLILMSRQALPLLPPLLYGVISIASSLVVLFFLPETQGLPLPDTIQDLESQKSTAAQGNRQEAVTVESTSL

SEQ ID NO: 19 - protein, 563 aa, Homo sapiens
MAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRWAPTQKEAGIYPRKGKQTRQQQEHQKYMVPLQASAQEKNGL

SEQ ID NO: 20 - protein, 542 aa, Homo sapiens
MTFSEILDRVGSMGHFQFLHVAILGLPILNMANHNLLQIFTAATPVHHCRPPHNASTGPWVLPMGPNGKPERCLRFVHPPNASLPNDTQRAMEPCLDGWVYNSTKDSIVTEWDLVCNSNKLKEMAQSIFMAGILIGGLVLGDLSDRFGRRPILTCSYLLLAASGSGAAFSPTFPIYMVFRFLCGFGISGITLSTVILNVEWVPTRMRAIMSTALGYCYTFGQFILPGLAYAIPQWRWLQLTVSIPFFVFFLSSWWTPESIRWLVLSGKSSKALKILRRVAVFNGKKEEGERLSLEELKLNLQKEISLAKAKYTASDLFRIPMLRRMTFCLSLAWFATGFAYYSLAMGVEEFGVNLYILQIIFGGVDVPAKFITILSLSYLGRHTTQAAALLLAGGAILALTFVPLDLQTVRTVLAVFGKGCLSSSFSCLFLYTSELYPTVIRQTGMGVSNLWTRVGSMVSPLVKITGEVQPFIPNIIYGITALLGGSAALFLPETLNQPLPETIEDLENWSLRAKKPKQEPEVEKASQRIPLQPHGPGLGSS

SEQ ID NO: 21 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 22 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 23 - protein, 467 aa, Homo sapiens
MQMDNRLPPKKVPGFCSFRYGLSFLVHCCNVIITAQRACLNLTMVVMVNSTDPHGLPNTSTKKLLDNIKNPMYNWSPDIQGIILSSTSYGVIIIQVPVGYFSGIYSTKKMIGFALCLSSVLSLLIPPAAGIGVAWVVVCRAVQGAAQGIVATAQFEIYVKWAPPLERGRLTSMSTSGFLLGPFIVLLVTGVICESLGWPMVFYIFGACGCAVCLLWFVLFYDDPKDHPCISISEKEYITSSLVQQVSSSRQSLPIKAILKSLPVWAISTGSFTFFWSHNIMTLYTPMFINSMLHVNIKENGFLSSLPYLFAWICGNLAGQLSDFFLTRNILSVIAVRKLFTAAGFLLPAIFGVCLPYLSSTFYSIVIFLILAGATGSFCLGGVFINGLDIAPRYFGFIKACSTLTGMIGGLIASTLTGLILKQDPESAWFKTFILMAAINVTGLIFYLIVATAEIQDWAKEKQHTRL

SEQ ID NO: 24 - protein, 1325 aa, Homo sapiens
MLPVYQEVKPNPLQDANLCSRVFFWWLNPLFKIGHKRRLEEDDMYSVLPEDRSQHLGEELQGFWDKEVLRAENDAQKPSLTRAIIKCYWKSYLVLGIFTLIEESAKVIQPIFLGKIINYFENYDPMDSVALNTAYAYATVLTFCTLILAILHHLYFYHVQCAGMRLRVAMCHMIYRKALRLSNMAMGKTTTGQIVNLLSNDVNKFDQVTVFLHFLWAGPLQAIAVTALLWMEIGISCLAGMAVLIILLPLQSCFGKLFSSLRSKTATFTDARIRTMNEVITGIRIIKMYAWEKSFSNLITNLRKKEISKILRSSCLRGMNLASFFSASKIIVFVTFTTYVLLGSVITASRVFVAVTLYGAVRLTVTLFFPSAIERVSEAIVSIRRIQTFLLLDEISQRNRQLPSDGKKMVHVQDFTAFWDKASETPTLQGLSFTVRPGELLAVVGPVGAGKSSLLSAVLGELAPSHGLVSVHGRIAYVSQQPWVFSGTLRSNILFGKKYEKERYEKVIKACALKKDLQLLEDGDLTVIGDRGTTLSGGQKARVNLARAVYQDADIYLLDDPLSAVDAEVSRHLFELCICQILHEKITILVTHQLQYLKAASQILILKDGKMVQKGTYTEFLKSGIDFGSLLKKDNEESEQPPVPGTPTLRNRTFSESSVWSQQSSRPSLKDGALESQDTENVPVTLSEENRSEGKVGFQAYKNYFRAGAHWIVFIFLILLNTAAQVAYVLQDWWLSYWANKQSMLNVTVNGGGNVTEKLDLNWYLGIYSGLTVATVLFGIARSLLVFYVLVNSSQTLHNKMFESILKAPVLFFDRNPIGRILNRFSKDIGHLDDLLPLTFLDFIQTLLQVVGVVSVAVAVIPWIAIPLVPLGIIFIFLRRYFLETSRDVKRLESTTRSPVFSHLSSSLQGLWTIRAYKAEERCQELFDAHQDLHSEAWFLFLTTSRWFAVRLDAICAMFVIIVAFGSLILAKTLDAGQVGLALSYALTLMGMFQWCVRQSAEVENMMISVERVIEYTDLEKEAPWEYQKRPPPAWPHEGVIIFDNVNFMYSPGGPLVLKHLTALIKSQEKVGIVGRTGAGKSSLISALFRLSEPEGKIWIDKILTTEIGLHDLRKKMSIIPQEPVLFTGTMRKNLDPFNEHTDEELWNALQEVQLKETIEDLPGKMDTELAESGSNFSVGQRQLVCLARAILRKNQILIIDEATANVDPRTDELIQKKIREKFAHCTVLTIAHRLNTIIDSDKIMVLDSGRLKEYDEPYVLLQNKESLFYKMVQQLGKAEAAALTETAKQVYFKRNYPHIGHTDHMVTNTSNGQPSTLTIFETAL

SEQ ID NO: 25 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 26 - protein, 15 aa, Artificial Sequence
  Description of Artificial Sequence Synthetic peptide
GGGGSGGGGSGGGGS

SEQ ID NO: 27 - protein, 9 aa, Artificial Sequence
  Description of Artificial Sequence Synthetic peptide
YPYDVPDYA

SEQ ID NO: 28 - protein, 10 aa, Artificial Sequence
  Description of Artificial Sequence Synthetic peptide
EQKLISEEDL

SEQ ID NO: 29 - protein, 8 aa, Artificial Sequence
  Description of Artificial Sequence Synthetic peptide
DYKDDDDK

SEQ ID NO: 30 - protein, 5 aa, Artificial Sequence
  Description of Artificial Sequence Synthetic peptide
  MOD_RES (3)..(3): Any amino acid
LPXTG

SEQ ID NO: 31 - protein, 5 aa, Artificial Sequence
  Description of Artificial Sequence Synthetic peptide
  MOD_RES (3)..(3): Any amino acid
LPXTA

SEQ ID NO: 32 - protein, 9 aa, Artificial Sequence
  Description of Artificial Sequence Synthetic peptide
GGGGSGGGG

SEQ ID NO: 33 - protein, 24 aa, Artificial Sequence
  Description of Artificial Sequence Synthetic peptide
GGGGSGGGGSGGGGSGGGGSGGGG

SEQ ID NO: 34 - protein, 299 aa, Cyberlindnera jadinii
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGSVYDIAKAADKGIFDNVYNQAREITLTTFALENSPSVQATMFNMATQILEKACSVYSVSYALPNKHYFLIDLKWKGLENDNELFYPSPHPNGLIKCTVVRKE

SEQ ID NO: 35 - protein, 297 aa, Aspergillus flavus
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSL

SEQ ID NO: 36 - protein, 300 aa, Artificial Sequence
  Description of Artificial Sequence Synthetic polypeptide
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKL

SEQ ID NO: 37 - protein, 490 aa, Bacillus subtilis
MFTMDDLNQMDTQTLTDTLGSIFEHSSWIAERSAALRPFSSLSDLHRKMTGIVKAADRETQLDLIKKHPRLGTKKTMSDDSVREQQNAGLGKLEQQEYEEFLMLNEHYYDRFGFPFILAVKGKTKQDIHQALLARLESERETEFQQALIEIYRIARFRLADIITEKGETQMKRTMSYGKGNVFAYRTYLKPLTGVKQIPESSFAGRDNTVVGVDVTCEIGGEAFLPSFTDGDNTLVVATDSMKNFIQRHLASYEGTTTEGFLHYVAHRFLDTYSHMDTITLTGEDIPFEAMPAYEEKELSTSRLVFRRSRNERSRSVLKAERSGNTITITEQYSEIMDLQLVKVSGNSFVGFIRDEYTTLPEDGNRPLFVYLNISWQYENTNDSYASDPARYVAAEQVRDLASTVFHELETPSIQNLIYHIGCRILARFPQLTDVSFQSQNHTWDTVVEEIPGSKGKVYTEPRPPYGFQHFTVTREDAEKEKQKAAEKCR

SEQ ID NO: 38 - protein, 299 aa, Mus musculus
MAHYHDNYGKNDEVEFVRTGYGKDMVKVLHIQRDGKYHSIKEVATSVQLTLRSKKDYLHGDNSDIIPTDTIKNTVHVLAKLRGIRNIETFAMNICEHFLSSFNHVTRAHVYVEEVPWKRFEKNGIKHVHAFIHTPTGTHFCEVEQMRNGPPVIHSGIKDLKVLKTTQSGFEGFLKDQFTTLPEVKDRCFATQVYCKWRYQRRDVDFEAIWGAVRDIVLQKFAGPYDKGEYSPSVQKTLYDIQVLSLSQLPEIEDMEISLPNIHYFNIDMSKMGLINKEEVLLPLDNPYGKITGTVKRKL

SEQ ID NO: 39 - protein, 305 aa, Glycine max
MAQQEVVEGFKFEQRHGKERVRVARVWKTRQGQHFIVEWRVGITLFSDCVNSYLRDDNSDIVATDTMKNTVYAKAKECSDILSAEEFAILLAKHFVSFYQKVTGAIVNIVEKPWERVTVDGQPHEHGFKLGSEKHTTEAIVQKSGSLQLTSGIEGLSVLKTTQSGFVNFIRDKYTALPDTRERMVATEVTALWRYSYESLYSLPQKPLYFTEKYQEVKKVLADTFFGPPKGGVYSPSVQNTLYLMAKATLNRFPDIAYVSLKLPNLHFIPVNISNQDGPIVKFEDDVYLPTDEPHGSIQASLSRL

SEQ ID NO: 40 - protein, 300 aa, Macaca mulatta
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVIHSGTKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKL

SEQ ID NO: 41 - protein, 303 aa, Oryza sativa
MADRLELQGRHGKSRVRVSRVWRRPAAAGGHVIVEWNVAVSVVSDCLPSYTSDDNSAIVATDSIKNTVYVKAKECTEIVSMEEFAVILGRHFTSLYPQVSEATVTIAERPWERVVVDGKPHSHGFKLGVEKHVTEVIVKKSGNLLINSGIQGYSLLKTTQSGFEKFVRDRYTLLPDTRERIVATEVTAWWRYPFEHVSQIPSKSFCFTQRYQDVKKVLADTFFGPPDVGVYSPSVQNTLYLMAKEVLNRFPDIASVQLRMPNLHFIPVNLGNKENPGLVKFADDVYLPTDEPHGTIEATVSRP

SEQ ID NO: 42 - protein, 346 aa, Penicillium freii
MILRSANSLHSRSNKYSFKLLDNTIFLPFSILYFFFSSIFQRYHSKFKMSALAAARYGKDNVRVCKVHRDEKTGIQTVVEMTVCVLLEGDIETSYTKADNSVVVATDSIKNTIFITAKQNPVTPPELFGSILGTHFIEKYSHIHAAHVNIVTHRWVRLDIDGKPHPHSFIKPGSETRNVQVDVIEGKGIDINSSIKGLTVLKSTGSQFWGFVRDEYTTLKETWDRLLSTDVAANWQWRRFTGLADVKTHSEKFNAAWEAARAITLKTFADDNSASVQATMYKMGEQILAAVPLLDTVEYALPNIHFFEVDLSWHKGIKNTGKDAEVYAPQSNPNGLIKCTVGRAGQ

SEQ ID NO: 43 - protein, 523 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAARE

SEQ ID NO: 44 - protein, 548 aa, Homo sapiens
MGFEELLEQVGGFGPFQLRNVALLALPRVLLPLHFLLPIFLAAVPAHRCALPGAPANFSHQDVWLEAHLPREPDGTLSSCLRFAYPQALPNTTLGEERQSRGELEDEPATVPCSQGWEYDHSEFSSTIATESQWDLVCEQKGLNRAASTFFFAGVLVGAVAFGYLSDRFGRRRLLLVAYVSTLVLGLASAASVSYVMFAITRTLTGSALAGFTIIVMPLELEWLDVEHRTVAGVLSSTFWTGGVMLLALVGYLIRDWRWLLLAVTLPCAPGILSLWWVPESARWLLTQGHVKEAHRYLLHCARLNGRPVCEDSFSQEAVSKVAAGERVVRRPSYLDLFRTPRLRHISLCCVVVWFGVNFSYYGLSLDVSGLGLNVYQTQLLFGAVELPSKLLVYLSVRYAGRRLTQAGTLLGTALAFGTRLLVSSDMKSWSTVLAVMGKAFSEAAFTTAYLFTSELYPTVLRQTGMGLTALVGRLGGSLAPLAALLDGVWLSLPKLTYGGIALLAAGTALLLPETRQAQLPETIQDVERKSAPTSLQEEEMPMKQVQN

SEQ ID NO: 45 - protein, 420 aa, Homo sapiens
MATKTELSPTARESKNAQDMQVDETLIPRKVPSLCSARYGIALVLHFCNFTTIAQNVIMNITMVAMVNSTSPQSQLNDSSEVLPVDSFGGLSKAPKSLPAKSSILGGQFAIWEKWGPPQERSRLCSIALSGMLLGCFTAILIGGFISETLGWPFVFYIFGGVGCVCCLLWFVVIYDDPVSYPWISTSEKEYIISSLKQQVGSSKQPLPIKAMLRSLPIWSICLGCFSHQWLVSTMVVYIPTYISSVYHVNIRDNGLLSALPFIVAWVIGMVGGYLADFLLTKKFRLITVRKIATILGSLPSSALIVSLPYLNSGYITATALLTLSCGLSTLCQSGIYINVLDIAPRYSSFLMGASRGFSSIAPVIVPTVSGFLLSQDPEFGWRNVFFLLFAVNLLGLLFYLIFGEADVQEWAKERKLTRL

SEQ ID NO: 46 - protein, 467 aa, Homo sapiens; identical to SEQ ID NO: 23

SEQ ID NO: 47 - protein, 509 aa, Homo sapiens
MELKKSPDGGWGWVIVFVSFLTQFLCYGSPLAVGVLYIEWLDAFGEGKGKTAWVGSLASGVGLLASPVCSLCVSSFGARPVTIFSGFMVAGGLMLSSFAPNIYFLFFSYGIVVGLGCGLLYTATVTITCQYFDDRRGLALGLISTGSSVGLFIYAALQRMLVEFYGLDGCLLIVGALALNILACGSLMRPLQSSDCPLPKKIAPEDLPDKYSIYNEKGKNLEENINILDKSYSSEEKCRITLANGDWKQDSLLHKNPTVTHTKEPETYKKKVAEQTYFCKQLAKRKWQLYKNYCGETVALFKNKVFSALFIAILLFDIGGFPPSLLMEDVARSSNVKEEEFIMPLISIIGIMTAVGKLLLGILADFKWINTLYLYVATLIIMGLALCAIPFAKSYVTLALLSGILGFLTGNWSIFPYVTTKTVGIEKLAHAYGILMFFAGLGNSLGPPIVGWFYDWTQTYDIAFYFSGFCVLLGGFILLLAALPSWDTCNKQLPKPAPTTFLYKVASNV

SEQ ID NO: 48 - protein, 10 aa, Artificial Sequence
  Description of Artificial Sequence Synthetic peptide
  MOD_RES (4)..(5): Any amino acid
GGGXXGGGGS

SEQ ID NO: 49 - protein, 10 aa, Artificial Sequence
  Description of Artificial Sequence Synthetic peptide
GGGGSGGGGS

SEQ ID NO: 50 - protein, 10 aa, Artificial Sequence
  Description of Artificial Sequence Synthetic peptide
GSGSGSGSGS

SEQ ID NO: 51 - protein, 7 aa, Artificial Sequence
  Description of Artificial Sequence Synthetic peptide
PSTSTST

SEQ ID NO: 52 - protein, 7 aa, Artificial Sequence
  Description of Artificial Sequence Synthetic peptide
EIDKPSQ

SEQ ID NO: 53 - protein, 19 aa, Artificial Sequence
  Description of Artificial Sequence Synthetic peptide
MYGKIIFVLLLSEIVSISA

SEQ ID NO: 54 - protein, 131 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 55 - DNA, 54 bp, Thosea asigna virus
gagggcagaggaagtcttctaacatgcggtgacgtggaggsgsstcccggccct

(19) United States
(12) Patent Application Publication
(10) Pub. No.: US 2019/0309269 A1
(43) Pub. Date: Oct. 10, 2019

(54) THERAPEUTIC CELL SYSTEMS AND METHODS FOR TREATING HYPERURICEMIA AND GOUT

(71) Applicant: Rubius Therapeutics, Inc., Cambridge, MA (US)

(72) Inventors: Lenka Hoffman, Watertown, MA (US); Thomas Joseph Wickham, Groton, MA (US)

(21) Appl. No.: 16/359,979

(22) Filed: Mar. 20, 2019

Related U.S. Application Data
(60) Provisional application No. 62/737,066, filed on Sep. 26, 2018.
(60) Provisional application No. 62/692,150, filed on Jun. 29, 2018.
(60) Provisional application No. 62/680,498, filed on Jun. 4, 2018.
(60) Provisional application No. 62/645,791, filed on Mar. 20, 2018.

(51) Int. Cl. C12N 9/06; C12N 9/08; A61P 3/00; A61K 35/18; A61K 35/19; C12N 5/078

(52) U.S. Cl. CPC C12N 9/0048; C12Y 107/03003; C12N 9/0065; C12Y 111/01006; C12N 5/0644; A61K 35/18; A61K 35/19; C12N 5/0641; A61P 3/00

(57) ABSTRACT

The present disclosure relates to erythroid cells that have been engineered to comprise a uricase, a uric acid transporter, or both a uricase and a uric acid transporter. The engineered erythroid cells of the present disclosure are useful in degrading uric acid inside the engineered erythroid cell. The engineered erythroid cells of the present disclosure are useful in methods of treating hyperuricemia. The engineered erythroid cells of the present disclosure are also useful in methods of treating gout, and in particular chronic refractory gout.

### RELATED APPLICATIONS

This application claims priority to U.S. Provisional Patent Application No. 62/645,791, filed on Mar. 20, 2018, U.S. Provisional Patent Application No. 62/680,498, filed on Jun. 4, 2018, U.S. Provisional Patent Application No. 62/692,150, filed on Jun. 29, 2018, and U.S. Provisional Patent Application No. 62/737,066, filed on Sep. 26, 2018, the entire contents of each of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Mar. 18, 2019, is named 129267-00705_SL.txt and is 147,397 bytes in size.

### BACKGROUND

Uric acid has been identified as a marker for a number of metabolic and hemodynamic abnormalities (Stack et al. (2015) Curr. Med. Res. Opin. Suppl 2: 21-26).

Gout is a painful, debilitating and progressive metabolic and inflammatory disease caused by abnormally elevated levels of uric acid in the blood stream. Gout is marked by recurrent attacks of red, tender, hot, and/or swollen joints. This leads to the deposition of painful, needle-like uric acid crystals in and around the connective tissue of the joints and in the kidneys, resulting in inflammation, the formation of disfiguring nodules, intermittent attacks of severe pain and kidney damage. In addition, evidence suggests that the chronic elevation of uric acid associated with gout, known as hyperuricemia, may also have systemic consequences, including an increased risk for kidney dysfunction and cardiovascular disease. After years of repetitive attacks, patients suffer the development of a chronic arthritis associated with the deposition of urate crystals and the build up to tophi in the joints (as well as in the tissues of the kidney and heart). The involved joints tend to become chronically deformed and painful and the risk for developing kidney stones, chronic renal insufficiency, and cardiovascular disease increases. This later stage, more involved clinical presentation is generally referred to a chronic tophaceaous gout. Once patients reach this stage they generally suffer multiple attacks every year.

Gout most often affects middle-aged to elderly men and postmenopausal women. The disease is typically associated with hyperuricemia (serum urate level >=6.8 mg per deciliter, which is the upper limit of solubility) and painful episodic acute flares which generally resolve in one to two weeks. Disease prevalence is increasing, and the number of patients suffering could be as high as 6-8 million in the United States, although the relapsing and remitting nature of the disease makes it difficult to estimate a precise number. Prevalence increases with age and risk factors include insulin resistance and obesity, as well as a purine rich diet (meat and seafood). Gout is the most common form of inflammatory arthritis in men over the age of 40 and represents a significant unmet medical need with limited treatment options.

While many patients are well controlled with diet and lifestyle changes, as well as generic medications for acute attacks, many patients still experience poor urate control and progressive symptoms. Patients who experience two attacks per year, or who present with tophi are considered candidates for urate lowering therapy. Three classes of drugs are approved for lowering urate levels: xanthine oxidase inhibitors, uricosuric agents, and uricase agents.

The first line standard of care for chronic disease remains xanthine oxidase inhibition (e.g., febuxostat, allopurinol) which blocks uric acid synthesis, and for many this is an effective treatment option. For patients who require yet more control or are contraindicated, the uricosuric agent Lesinurad may be prescribed. Lesinurad is an oral inhibitor of the urate/anion exchanger URAT1, which is the transporter that mediates reuptake of uric acid from the proximal tubules of the kidney, and which thus drives renal elimination of urate. Lesinurad was approved in December 2015, but carries a renal toxicity associated black box warning and is contraindicated in patients with renal impairment.

Approximately 50,000-60,000 people fail all therapy and are thus considered chronic refractory. These patients are candidates for uricase agents which break down uric acid directly. KRYSTEXXA® (pegloticase) is a porcine-derived, pegylated, recombinant urate oxidase that was approved by the FDA for the treatment of chronic refractory gout in September 2010. Uricase converts uric acid into allatonin which is then excreted. While physicians describe Krystexxa as effective, in the pivotal program the primary endpoint was achieved in only 42% of biweekly cohort and 35% of monthly cohort (v. 0% for placebo). Infusion reactions occurred in 26% of patients receiving q2 week dosing and 40% of patients receiving q4 dosing. Infusion reaction related discontinuation of therapy occurred in 11 and 13% and the label includes a boxed warning for anaphylaxis. Perhaps most concerning, there were eight serious cardiovascular events in patients in the Phase III trials versus one in the placebo arm. Krystexxa administration is inconvenient with patients having to undergo a 4-hour premedication/infusion process once every two weeks.

Overall, treatment options for the majority of the chronic refractory gout population are limited. There remains a need in the art for improved ways to treat gout, and in particular, to treat chronic refractory gout.

### SUMMARY OF THE INVENTION

The present invention provides erythroid cells that have been engineered to comprise a uric acid degrading polypeptide. The engineered erythroid cells of the invention are expected to overcome the limitations of existing therapies, and bring relief from debilitating and crippling pain to tens of thousands of patients suffering from gout, and in particular from refractory gout.

In particular, the present disclosure relates to erythroid cells that are engineered to comprise a uric acid degrading polypeptide (e.g., uricase), a uric acid transporter, or both a uric acid degrading polypeptide and a uric acid transporter. The engineered erythroid cells can be nucleated, e.g., erythrocyte precursor cells, or can be enucleated, e.g., reticulocytes or erythrocytes. These engineered erythroid cells are useful in the treatment of diseases and conditions associated with hyperuricemia, and in particular, gout. The present disclosure is based, at least in part, on the discovery that an engineered erythroid cell having at least about 1×10-¹⁰ units of uricolytic activity per cell is considered to be clinically useful. Thus, the present disclosure is based in part on the selection of a uric acid degrading polypeptide (e.g., uricase) and optionally together with a uric acid transporter that, when expressed in an erythroid cell and at a sufficient copy number, achieve the target activity of at least about 1e-10 units of uricolytic activity per cell.

In some embodiments, pharmaceutical compositions comprising the engineered erythroid cells described herein are administered to a subject (e.g., a human subject) for treatment of a disease or disorder. For example, the pharmaceutical composition may be administered intravenously to the subject. In some embodiments, the engineered erythroid cells circulate for up to 120 days while shielding the uric acid degrading polypeptide and/or uric acid transporter from the subject's immune system. These engineered erythroid cells may act as circulating metabolic factories to effectively replace the subject's mutated or missing enzymes, and reduce uric acid levels in the subject.

In one aspect, the disclosure features an erythroid cell engineered to degrade uric acid, comprising a first exogenous polypeptide comprising a uric acid degrading polypeptide, or a variant thereof, wherein the erythroid cell comprises at least 50,000 copies of the first exogenous polypeptide. In one embodiment, the engineered erythroid cell comprises at least about 100,000 copies of the first exogenous polypeptide. In one embodiment, the engineered erythroid cell comprises at least about 150,000 copies of the first exogenous polypeptide. In one embodiment, the engineered erythroid cell comprises at least about 170,000 copies of the first exogenous polypeptide. In one embodiment, the engineered erythroid cell comprises about 50,000-300,000 copies of the first exogenous polypeptide. In one embodiment, the uric acid degrading polypeptide is a uricase, or a variant thereof. In a further embodiment, the uric acid degrading polypeptide is selected from the group consisting of a HIU hydrolase, or a variant thereof, an OHCU decarboxylase, or a variant thereof, an allantoinase, or variant thereof, an allantoicase, or a variant thereof, a myeloperoxidase, or variant thereof, a FAD-dependent urate hydroxylase, or variant thereof, an xanthine dehydrogenase, or variant thereof, a nucleoside deoxyribosyltransferase, or variant thereof, a dioxotetrahydropyrimidine phosphoribosyltransferase, or variant thereof, a dihydropyrimidinase, or variant thereof, and a guanine deaminase, or a variant thereof. In one embodiment, the uricase has a specific activity that is greater than between about 5 μmol/min/mg to about 50 μmol/min/mg at a neutral pH. In one embodiment, the uricase has a specific activity that is greater than about 50 μmol/min/mg at a neutral pH. In one embodiment, the uricase has a specific activity that is greater than about 100 μmol/min/mg at a neutral pH. In one embodiment, the engineered erythroid cell is an enucleated cell, e.g. an erythrocyte or a reticulocyte.

In another aspect, the disclosure features an erythroid cell engineered to degrade uric acid, comprising a first exogenous polypeptide comprising a uric acid degrading polypeptide, wherein the engineered erythroid cell has uricolytic activity of at least about 1-2e-10 units/cell. In one embodiment, the engineered erythroid cell has uricolytic activity of at least about 3e-10 to about 6e-10 units/cell. In one embodiment, the engineered erythroid cell comprises at least 100,000 copies of the first exogenous polypeptide. In one embodiment, the engineered erythroid cell comprises at least 150,000 copies of the first exogenous polypeptide. In one embodiment, the engineered erythroid cell comprises at least about 170,000 copies of the first exogenous polypeptide. In one embodiment, the engineered erythroid cell comprises about 50,000-300,000 copies of the first exogenous polypeptide. In one embodiment, the uric acid degrading polypeptide is a uricase, or a variant thereof. In some embodiments, the uric acid degrading polypeptide is selected from the group consisting of a HIU hydrolase, or a variant thereof, an OHCU decarboxylase, or a variant thereof, an allantoinase, or variant thereof, an allantoicase, or a variant thereof, a myeloperoxidase, or variant thereof, a FAD-dependent urate hydroxylase, or variant thereof, an xanthine dehydrogenase, or variant thereof, a nucleoside deoxyribosyltransferase, or variant thereof, a dioxotetrahydropyrimidine phosphoribosyltransferase, or variant thereof, a dihydropyrimidinase, or variant thereof, and a guanine deaminase, or variant thereof. In one embodiment, the uricase has a specific activity that is greater than about 50 μmol/min/mg at a neutral pH. In one embodiment, the uricase has a specific activity that is greater than about 100 μmol/min/mg at a neutral pH. In one embodiment, the engineered erythroid cell is an enucleated cell, e.g. an erythrocyte or a reticulocyte.

In another aspect, the disclosure features an erythroid cell engineered to degrade uric acid, comprising a first exogenous polypeptide comprising a uric acid degrading polypeptide, or a variant thereof, wherein the specific activity of the uric acid degrading polypeptide is greater than between about 5 μmol/min/mg to about 50 μmol/min/mg at a neutral pH. In one embodiment, the specific activity of the uric acid degrading polypeptide is greater than about 50 μmol/min/mg at a neutral pH. In one embodiment, the specific activity of the uric acid degrading polypeptide is greater than about 100 μmol/min/mg at a neutral pH. In one embodiment, the engineered erythroid cell comprises at least about 50,000 copies of the first exogenous polypeptide. In one embodiment, the engineered erythroid cell comprises at least about 100,000 copies of the first exogenous polypeptide. In one embodiment, the engineered erythroid cell comprises at least 150,000 copies of the first exogenous polypeptide. In one embodiment, the engineered erythroid cell comprises at least about 170,000 copies of the first exogenous polypeptide. In one embodiment, the engineered erythroid cell comprises about 50,000-300,000 copies of the first exogenous polypeptide. In one embodiment, the uric acid degrading polypeptide is a uricase, or a variant thereof. In some embodiments, the uric acid degrading polypeptide is selected from the group consisting of: a HIU hydrolase, or a variant thereof, an OHCU decarboxylase, or a variant thereof, an allantoinase, or variant thereof, an allantoicase, or a variant thereof, a myeloperoxidase, or variant thereof, a FAD-dependent urate hydroxylase, or variant thereof, an xanthine dehydrogenase, or variant thereof, a nucleoside deoxyribosyltransferase, or variant thereof, a dioxotetrahydropyrimidine phosphoribosyltransferase, or variant thereof, a dihydropyrimidinase, or variant thereof, and a guanine deaminase, or variant thereof, or any combination thereof. In one embodiment of the above aspects and embodiments, the erythroid cell has uricolytic activity of at least about 1-2e-10 units/cell. In one embodiment, the erythroid cell has uricolytic activity of at least about 3e-10 to about 6e-10 units/cell. In one embodiment of the above aspects and embodiments, the uricase is a fungal uricase. In one embodiment, the fungal uricase is derived from Candida utilis. In one embodiment, the uricase derived from Candida utilis comprises an amino acid sequence that is at least 95% identical (e.g., 96%, 97%, 98%, 99% or 100% identical) to the amino acid sequence set forth in SEQ ID NO: 1. In one embodiment, the fungal uricase is derived from Aspergillus flavus. In one embodiment, the uricase derived from Aspergillus flavus comprises an amino acid sequence that is at least 95% identical (e.g., 96%, 97%, 98%, 99% or 100% identical) to the amino acid sequence set forth in SEQ ID NO: 2. In one embodiment, the fungal uricase is derived from Penicillium freii. In one embodiment, the uricase derived from Penicillium freii comprises an amino acid sequence that is at least 95% identical (e.g., 96%, 97%, 98%, 99% or 100% identical) to the amino acid sequence set forth in SEQ ID NO: 13. In one embodiment, the fungal uricase is derived from Aspergillus niger. In one embodiment, the uricase derived from Aspergillus niger comprises an amino acid sequence that is at least 95% identical (e.g., 96%, 97%, 98%, 99% or 100% identical) to the amino acid sequence set forth in SEQ ID NO: 14. In one embodiment of any one of the above aspects and embodiments, the uricase is a yeast uricase. In one embodiment, the yeast uricase is derived from Schizosaccharomyces pombe (Fission yeast). In one embodiment, the uricase derived from Schizosaccharomyces pombe (Fission yeast) comprises an amino acid sequence that is at least 95% identical (e.g., 96%, 97%, 98%, 99% or 100% identical) to the amino acid sequence set forth in SEQ ID NO: 10. In one embodiment of the above aspects and embodiments, the uricase is a bacterial uricase. In one embodiment, the bacterial uricase is derived from Arthrobacter globiformi. In one embodiment, the uricase derived from Arthrobacter globiformi comprises an amino acid sequence that is at least 95% identical (e.g., 96%, 97%, 98%, 99% or 100% identical) to the amino acid sequence set forth in SEQ ID NO: 3. In one embodiment, the bacterial uricase is derived from Bacillus subtilis. In one embodiment, the uricase derived from Bacillus subtilis comprises an amino acid sequence that is at least 95% identical (e.g., 96%, 97%, 98%, 99% or 100% identical) to the amino acid sequence set forth in SEQ ID NO: 5. In one embodiment, the bacterial uricase is derived from Cellulomonas flavigena. In one embodiment, the uricase derived from Cellulomortas flavigena comprises an amino acid sequence that is at least 95% identical (e.g., 96%, 97%, 98%, 99% or 100% identical) to the amino acid sequence set forth in SEQ ID NO: 6. In one embodiment of the above aspects and embodiments, the uricase is a mammalian uricase. In one embodiment, the mammalian uricase is derived from Mus musculus. In one embodiment, the uricase derived from Mus musculus comprises an amino acid sequence that is at least 95% identical (e.g., 96%, 97%, 98%, 99% or 100% identical) to the amino acid sequence set forth in SEQ ID NO: 7. In one embodiment, the mammalian uricase is derived from Danio rerio (Zebrafish). In one embodiment, the uricase derived from Danio rerio (Zebrafish) comprises an amino acid sequence that is at least 95% identical (e.g., 96%, 97%, 98%, 99% or 100% identical) to the amino acid sequence set forth in SEQ ID NO: 9. In one embodiment, the mammalian uricase is derived from Macaca mulatta (Rhesus macaque). In one embodiment, the uricase derived from Macaca mulatta (Rhesus macaque) comprises an amino acid sequence that is at least 95% identical (e.g., 96%, 97%, 98%, 99% or 100% identical) to the amino acid sequence set forth in SEQ ID NO: 11. In one embodiment, the mammalian uricase is a chimeric mammalian uricase. In one embodiment, the chimeric mammalian uricase comprises an amino acid sequence that is at least 95% identical (e.g., 96%, 97%, 98%, 99% or 100% identical) to the amino acid sequence set forth in SEQ ID NO: 4. In one embodiment of the above aspects and embodiments, the uricase is a plant uricase. In one embodiment, the plant uricase is derived from Glycine max (Soybean). In one embodiment, the uricase derived from Glycine max (Soybean) comprises an amino acid sequence that is at least 95% identical (e.g., 96%, 97%, 98%, 99% or 100% identical) to the amino acid sequence set forth in SEQ ID NO: 8. In one embodiment, the plant uricase is derived from Oryza sativa subsp. japonica (Rice). In one embodiment, the uricase derived from Oryza sativa subsp. japonica (Rice) comprises an amino acid sequence that is at least 95% identical (e.g., 96%, 97%, 98%, 99% or 100% identical) to the amino acid sequence set forth in SEQ ID NO: 12. In one embodiment, the uricase derived from Drosophila melanogaster comprises an amino acid sequence that is at least 95% identical (e.g., 96%, 97%, 98%, 99% or 100% identical) to the amino acid sequence set forth in SEQ ID NO: 15. In one embodiment of the above aspects and embodiments, the neutral pH is from about 7.0 to about 7.5. In one embodiment of the above aspects and embodiments, the neutral pH is about 7.2 to about 7.4. In one embodiment of the above aspects and embodiments, the neutral pH is about 7.2. In one embodiment of the above aspects and embodiments, the uricase is a mutant uricase that retains the uricolytic activity of the wild type uricase. In one embodiment of the above aspects and embodiments, the first exogenous polypeptide is expressed inside the erythroid cell. In one embodiment of the above aspects and embodiments, the engineered erythroid cell further comprises a second exogenous polypeptide comprising a uric acid transporter, or a variant thereof. In one embodiment, the uric acid transporter is selected from the group consisting of: URAT1, GLUT9, OAT4, OAT1, OAT3, Gal-9, ABCG2, SLC34A2, MRP4, OAT2, NPT4, NPT1, and MCT9. In one embodiment, URAT1 comprises an amino acid sequence that is at least 95% identical (e.g., 96%, 97%, 98%, 99% or 100% identical) to the amino acid sequence set forth in SEQ ID NO: 16 ; wherein GLUT9 comprises an amino acid sequence that is at least 95% identical (e.g., 96%, 97%, 98%, 99% or 100% identical) to the amino acid sequence set forth in SEQ ID NO: 17; wherein OAT4 comprises an amino acid sequence that is at least 95% (e.g., 96%, 97%, 98%, 99% or 100% identical) identical to the amino acid sequence set forth in SEQ ID NO: 18; wherein OAT1 comprises an amino acid sequence that is at least 95% identical (e.g., 96%, 97%, 98%, 99% or 100% identical) to the amino acid sequence set forth in SEQ ID NO: 19; wherein OAT3 comprises an amino acid sequence that is at least 95% identical (e.g., 96%, 97%, 98%, 99% or 100% identical) to the amino acid sequence set forth in SEQ ID NO: 20; wherein Gal-9 comprises an amino acid sequence that is at least 95% identical (e.g., 96%, 97%, 98%, 99% or 100% identical) to the amino acid sequence set forth in SEQ ID NO: 21; wherein ABCG2 comprises an amino acid sequence that is at least 95% identical (e.g., 96%, 97%, 98%, 99% or 100% identical) to the amino acid sequence set forth in SEQ ID NO: 22; wherein SLC34A2 comprises an amino acid sequence that is at least 95% identical (e.g., 96%, 97%, 98%, 99% or 100% identical) to the amino acid sequence set forth in SEQ ID NO: 23 wherein MRP4 comprises an amino acid sequence that is at least 95% identical (e.g., 96%, 97%, 98%, 99% or 100% identical) to the amino acid sequence set forth in SEQ ID NO: 24; wherein OAT2 comprises an amino acid sequence that is at least 95% identical (e.g., 96%, 97%, 98%, 99% or 100% identical) to the amino acid sequence set forth in SEQ ID NO:44; wherein NPT4 comprises an amino acid sequence that is at least 95% identical (e.g., 96%, 97%, 98%, 99% or 100% identical) to the amino acid sequence set forth in SEQ ID NO:45; wherein NPT1 comprises an amino acid sequence that is at least 95% identical (e.g., 96%, 97%, 98%, 99% or 100% identical) to the amino acid sequence set forth in SEQ ID NO:46; and wherein MCT9 comprises an amino acid sequence that is at least 95% identical (e.g., 96%, 97%, 98%, 99% or 100% identical) to the amino acid sequence set forth in SEQ ID NO:47. In one embodiment, the second exogenous polypeptide is presented at the surface of the erythroid cell. In one embodiment, the erythroid cell comprises at least about 10,000 copies of the second exogenous polypeptide. In one embodiment, the erythroid cell comprises at least about 20,000 copies of the second exogenous polypeptide. In one embodiment, the erythroid cell comprises at least about 30,000 copies of the second exogenous polypeptide. In one embodiment, the erythroid cell comprises about 10,000-100,000 copies of the second exogenous polypeptide. In one embodiment, the first exogenous polypeptide is present at a copy number of no more than 10%, 20%, 30%, 40%, 50%, 60%, 70%, 80%, or 90% greater, or no more than 2, 3, 4, 5, 6, 7, 8, 9, 10, 20, 50, 100, 200, 500, or 1000 times greater than the copy number of the second exogenous polypeptide. In some embodiments, the uric acid transporter transports uric acid from outside the erythroid cell to the inside of the erythroid cell at a rate of between about 1.0e-10 and about 1.5e-10 μmol uric acid per minute per cell (μmol/min/cell). In some embodiments, the uric acid transporter transports uric acid from outside the erythroid cell to the inside of the erythroid cell at a rate of at least about 1.3e-10 μmol uric acid per minute per cell (μmol/min/cell). In some embodiments of the above aspects and embodiments, the erythroid cell further comprises a third exogenous polypeptide comprising a catalase, or a variant thereof. In some embodiments of the above aspects and embodiments, the erythroid cell when administered to a subject is capable of reducing serum uric acid level in the subject. In some embodiments, the level of serum uric acid is reduced to about 6.8 mg/dl or less. In some embodiments, the level of serum uric acid is reduced to about 6.0 mg/dl or less.

In some embodiments of the above aspects and embodiments, the engineered erythroid cell is an enucleated cell, e.g. an erythrocyte or a reticulocyte. In some embodiments of the above aspects and embodiments, the engineered erythroid cell is a reticulocyte. In some embodiments of all aspects of the invention, the uric acid degrading polypeptide is not uricase. In some embodiments of all aspects of the invention, the uric acid degrading polypeptide is not Aspergillus flavus Uricase.

In another aspect, the disclosure provides an engineered erythroid cell comprising a first exogenous polypeptide comprising a uric acid transporter, or a variant thereof. In some embodiments, the uric acid transporter is selected from the group consisting of URAT1, GLUT9, OAT4, OAT1, OAT3, Gal-9, ABCG2, SLC34A2, MRP4, OAT2, NPT4, NPT1, and MCT9. In some embodiments, URAT1 comprises an amino acid sequence that is at least 95% identical (e.g., 96%, 97%, 98%, 99% or 100% identical) to the amino acid sequence set forth in SEQ ID NO: 16; wherein GLUT9 comprises an amino acid sequence that is at least 95% identical (e.g., 96%, 97%, 98%, 99% or 100% identical) to the amino acid sequence set forth in SEQ ID NO: 17; wherein OAT4 comprises an amino acid sequence that is at least 95% identical (e.g., 96%, 97%, 98%, 99% or 100% identical) to the amino acid sequence set forth in SEQ ID NO: 18; wherein OAT1 comprises an amino acid sequence that is at least 95% identical (e.g., 96%, 97%, 98%, 99% or 100% identical) to the amino acid sequence set forth in SEQ ID NO: 19; wherein OAT3 comprises an amino acid sequence that is at least 95% identical (e.g., 96%, 97%, 98%, 99% or 100% identical) to the amino acid sequence set forth in SEQ ID NO: 20; wherein Gal-9 comprises an amino acid sequence that is at least 95% identical (e.g., 96%, 97%, 98%, 99% or 100% identical) to the amino acid sequence set forth in SEQ ID NO: 21; wherein ABCG2 comprises an amino acid sequence that is at least 95% identical (e.g., 96%, 97%, 98%, 99% or 100% identical) to the amino acid sequence set forth in SEQ ID NO: 22; wherein SLC34A2 comprises an amino acid sequence that is at least 95% identical (e.g., 96%, 97%, 98%, 99% or 100% identical) to the amino acid sequence set forth in SEQ ID NO: 23; wherein MRP4 comprises an amino acid sequence that is at least 95% identical (e.g., 96%, 97%, 98%, 99% or 100% identical) to the amino acid sequence set forth in SEQ ID NO: 24; wherein OAT2 comprises an amino acid sequence that is at least 95% identical (e.g., 96%, 97%, 98%, 99% or 100% identical) to the amino acid sequence set forth in SEQ ID NO:44; wherein NPT4 comprises an amino acid sequence that is at least 95% identical (e.g., 96%, 97%, 98%, 99% or 100% identical) to the amino acid sequence set forth in SEQ ID NO:45; wherein NPT1 comprises an amino acid sequence that is at least 95% identical (e.g., 96%, 97%, 98%, 99% or 100% identical) to the amino acid sequence set forth in SEQ ID NO:46; and wherein MCT9 comprises an amino acid sequence that is at least 95% identical (e.g., 96%, 97%, 98%, 99% or 100% identical) to the amino acid sequence set forth in SEQ ID NO:47. In some embodiments, the first exogenous polypeptide is presented at the surface of the engineered erythroid cell. In some embodiments, the erythroid cell comprises at least 10,000 copies of the first exogenous polypeptide. In some embodiments, the erythroid cell comprises at least 20,000 copies of the first exogenous polypeptide. In some embodiments, the erythroid cell comprises at least 30,000 copies of the second exogenous polypeptide. In some embodiments, the erythroid cell comprises about 10,000-100,000 copies of the first exogenous polypeptide. In some embodiments, the uric acid transporter transports uric acid from outside the erythroid cell to the inside of the erythroid cell at a rate of at least about 1.0e-10, or at least about 1.3e-10 μmol uric acid per minute per cell (μmol/min/cell). In some embodiments of the above aspects and embodiments, the engineered erythroid cell is an enucleated cell, e.g. an erythrocyte or a reticulocyte. In some embodiments of the above aspects and embodiments, the engineered erythroid cell is a reticulocyte.

In another aspect, the disclosure provides an engineered erythroid cell comprising a first exogenous polypeptide comprising a uricase, or a variant thereof, and a second exogenous polypeptide comprising a uric acid transporter, or a variant thereof.

In another aspect, the disclosure provides an engineered erythroid cell (e.g., enucleated erythroid cell) comprising a first exogenous polypeptide comprising a uricase, or a variant thereof, a second exogenous polypeptide comprising a uric acid transporter, or a variant thereof, and a third exogenous polypeptide comprising a catalase, or a variant thereof.

In another aspect, the disclosure provides an engineered erythroid cell comprising a first exogenous polypeptide comprising Candida utilis uricase, or a variant thereof, and a second exogenous polypeptide comprising human URAT1, or a variant thereof.

In another aspect, the disclosures provides an engineered erythroid cell comprising a first exogenous polypeptide comprising Candida utilis uricase, or a variant thereof, and a second exogenous polypeptide comprising a uric acid transporter selected from the group consisting of GLUT9, OAT4, OAT1, OAT3, Gal-9, ABCG2, SLC34A2, MRP4, OAT2, NPT4, NPT1, and MCT9.

In some embodiments of the above aspects, the engineered erythroid cell is an enucleated cell, e.g. an erythrocyte or a reticulocyte.

In another aspect, the disclosure provides a pharmaceutical composition comprising a plurality of the engineered erythroid cells of any one of the above aspects and embodiments, and a pharmaceutically acceptable carrier. In some embodiments, the pharmaceutical composition comprises a therapeutically effective dose of the engineered erythroid cells. In some embodiments, the pharmaceutical composition comprises between 1e10 and 1e12 engineered erythroid cells. In some embodiments, the pharmaceutical composition comprises at least 1e10, 2e10, 3e10, 4e10, 5e10, 6 e10, 7e10, 8e10, 9e10, or 1e11 engineered erythroid cells. In some embodiments, the pharmaceutical composition comprises at least 3e10 engineered erythroid cells. In some embodiments, the pharmaceutical composition comprises at least 1e10 engineered erythroid cells. In some embodiments, the pharmaceutical composition comprises about 1e10 engineered erythroid cells. In some embodiments, the engineered erythroid cells have about 1-6×10⁻¹⁰ units of uricolytic activity per cell. In some embodiments, the engineered erythroid cells have about 1-2×10⁻¹⁰ units of uricolytic activity per cell. In some embodiments, the engineered erythroid cells have about 3-6×10⁻¹⁰ units of uricolytic activity per cell. In some embodiments, the engineered erythroid cells have at least about 3×10⁻¹⁰ to about 6×−10⁻¹⁰ units of uricolytic activity per cell. In some embodiments, the engineered erythroid cells comprise about 10-20 units of uricolytic activity per dose. In some embodiments, the engineered erythroid cells comprise about 9-18 units of uricolytic activity per dose.

In another aspect, the disclosure provides a method of treating or preventing hyperuricemia in a subject, comprising administering to the subject the engineered erythroid cell of any of the foregoing aspects and embodiments, in an amount effective to treat or prevent hyperuricemia in the subject. In some embodiments, the subject has a serum urate level greater than about 6.8 mg/dl prior to administering the engineered erythroid cell. In some embodiments, the subject has a serum urate level greater than about 8.0 mg/dl prior to administering the engineered erythroid cell. In some embodiments, the subject has a serum urate level less than about 6.8 mg/dl after administering the engineered erythroid cell. In some embodiments, the subject has a serum urate level of about 6.0 mg/dl after administering the engineered erythroid cell. In some embodiments, the subject has been diagnosed with a disease selected from the group consisting of: gout, rheumatoid arthritis, osteoarthritis, cerebral stroke, ischemic heart disease, arrhythmia, and chronic renal disease. In a further embodiment, the gout is chronic refractory gout. In some embodiments, the subject has one or more risk factors for hyperuricemia selected from the group consisting of insulin resistance, obesity, a purine rich diet and advanced age. In some embodiments, the subject has been diagnosed with symptomatic gout with at least 3 gout flares in the previous 18 months. In some embodiments, the subject has been diagnosed with at least 1 gout tophus or gouty arthritis. In some embodiments, the subject has previously been treated with a urate lowering therapy, and failed to normalize level of serum uric acid to about 6.8 mg/dl or less. In some embodiments, the subject has a contraindication to allopurinol. In some embodiments, the subject has a failure to normalize uric acid to less than 6 mg/dL after at least 3 months of allopurinol treatment. In some embodiments, the engineered erythroid cell has about 1-6×10⁻¹⁰ units of uricolytic activity per cell. In some embodiments, the engineered erythroid cell has about 1-2×10⁻¹⁰ units of uricolytic activity per cell. In some embodiments, the engineered erythroid cell has about 3×10⁻¹⁰ to about 6×10⁻¹⁰ units of uricolytic activity per cell. In some embodiments, the effective amount of engineered erythroid cells comprises 10-20 units of uricolytic activity per dose. In some embodiments, the effective amount of engineered erythroid cells comprises 9-18 units of uricolytic activity per dose. In some embodiments, the subject is administered about 1×10¹⁰-1×10¹² engineered erythroid cells. In some embodiments, the subject is administered at least about 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ engineered erythroid cells. In some embodiments, the subject is administered about 7×10¹⁰-1.3×10¹¹ engineered erythroid cells. In some embodiments, the subject is administered about 1×10¹¹ engineered erythroid cells. In some embodiments, the subject is administered about 1×10¹⁰-5×10¹⁰ engineered erythroid cells. In some embodiments, the subject is administered about 3×10¹⁰ engineered erythroid cells. In some embodiments, the subject is administered about 3×10¹⁰ engineered erythroid cells. In some embodiments, the engineered erythroid cell is administered to the subject about once every four weeks. In some embodiments, the method further comprises administration of a second agent. In some embodiments, the engineered erythroid cell remains in the circulatory system of the subject up to 120 days.

In another aspect, the disclosure provides an engineered enucleated cell (e.g., engineered enucleated erythroid cell), comprising at a first exogenous polypeptide comprising a first exogenous polypeptide comprising a uric acid degrading polypeptide, or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure provides an engineered enucleated cell (e.g., engineered enucleated erythroid cell), comprising at a first exogenous polypeptide comprising a uric acid transporter, or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure provides an engineered enucleated cell (e.g., engineered enucleated erythroid cell), comprising at a first exogenous polypeptide comprising a uric acid degrading polypeptide, or a variant thereof, and a second exogenous polypeptide comprising a uric acid transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide and the second exogenous polypeptide.

In another aspect, the disclosure provides an engineered enucleated cell (e.g., engineered enucleated erythroid cell), comprising at a first exogenous polypeptide comprising a uric acid degrading polypeptide, or a variant thereof, a second exogenous polypeptide comprising a uric acid transporter, or a variant thereof, and a third exogenous polypeptide comprising a catalase, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); introducing an exogenous nucleic acid encoding the third exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide, the second exogenous polypeptide and the third exogenous polypeptide. In some embodiments, the uric acid degrading polypeptide is a uricase, or a variant thereof. In some embodiments, the uric acid degrading polypeptide is an allantoinase, or variant thereof. In some embodiments of the foregoing aspects, the exogenous nucleic acid comprises DNA or RNA. In some embodiments of the foregoing aspects, the introducing step comprises viral transduction. In some embodiments, the introducing step comprises electroporation. In some embodiments of the foregoing aspects, the introducing step comprises utilizing one or more of: liposome mediated transfer, adenovirus, adeno-associated virus, herpes virus, a retroviral based vector, lipofection, and a lentiviral vector. In some embodiments of the foregoing aspects, the introducing step comprises introducing the first exogenous nucleic acid encoding the first exogenous polypeptide by transfection of a lentiviral vector. In some embodiments of the foregoing aspects, the introducing step comprises introducing the first exogenous nucleic acid encoding the first exogenous polypeptide and the second exogenous nucleic acid encoding the second exogenous polypeptide by transfection of a lentiviral vector, wherein the first exogenous nucleic acid and the second exogenous nucleic acid are contained in the same lentiviral vector. In some embodiments of the foregoing aspects, the introducing step comprises introducing the first exogenous nucleic acid encoding the first exogenous polypeptide by transfection of a first lentiviral vector, and introducing the second exogenous nucleic acid encoding the second exogenous polypeptide by transfection of a second lentiviral vector. In some embodiments of the foregoing aspects, the lentiviral vector comprises a promoter selected from the group consisting of beta-globin promoter, murine stem cell virus (MSCV) promoter, Gibbon ape leukemia virus (GALV) promoter, human elongation factor lalpha (EFlalpha) promoter, CAG CMV immediate early enhancer and the chicken beta-actin (CAG), and human phosphoglycerate kinase 1 (PGK) promoter. In some embodiments of the foregoing aspects, the engineered erythroid cell comprises at least 100,000 copies of the first exogenous polypeptide. In some embodiments of the foregoing aspects, the engineered erythroid cell comprises at least 150,000 copies of the first exogenous polypeptide. In some embodiments of the foregoing aspects, the engineered erythroid cell comprises about 50,000-300,000 copies of the first exogenous polypeptide. In some embodiments of the foregoing aspects, the engineered erythroid cell comprises at least about 170,000 copies of the first exogenous polypeptide. In some embodiments of the foregoing aspects, the engineered erythroid cell comprises at least 10,000 copies of the first exogenous polypeptide. In some embodiments of the foregoing aspects, the engineered erythroid cell comprises at least 20,000 copies of the first exogenous polypeptide. In some embodiments of the foregoing aspects, the engineered erythroid cell comprises at least 30,000 copies of the second exogenous polypeptide. In some embodiments of the foregoing aspects, the engineered erythroid cell comprises at least about 10,000-100,000 copies of the first exogenous polypeptide. In some embodiments of the foregoing aspects, the engineered erythroid cell comprises at least 10,000 copies of the second exogenous polypeptide. In some embodiments of the foregoing aspects, the engineered erythroid cell comprises at least 20,000 copies of the second exogenous polypeptide. In some embodiments of the foregoing aspects, the engineered erythroid cell comprises at least 30,000 copies of the second exogenous polypeptide. In some embodiments of the foregoing aspects, the engineered erythroid cell comprises at least about 10,000-100,000 copies of the second exogenous polypeptide. In some embodiments of the foregoing aspects, the engineered enucleated cell comprises at least 50,000 copies of the third exogenous polypeptide. In some embodiments of the foregoing aspects, the engineered enucleated cell comprises about 10,000-100,000 copies of the third exogenous polypeptide.

In some embodiments, the first exogenous polypeptide, the second exogenous polypeptide, or both the first and second exogenous polypeptides (e.g., the uric acid degrading polypeptide, uric acid transporter, or both the uric acid degrading polypeptide and the uric acid transporter) expressed in an engineered erythroid cell have a prolonged in vivo half-life. In some embodiments, the first exogenous polypeptide, the second exogenous polypeptide, or both the first and second exogenous polypeptides have an in vivo half-life that is longer than the half-life of the first exogenous polypeptide, the second exogenous polypeptide, or both the first and second exogenous polypeptides, or a pegylated version of the first exogenous polypeptide, the second exogenous polypeptide, or both the first and second exogenous polypeptides, which are not comprised in an engineered erythroid cell.

In some embodiments, the first exogenous polypeptide, the second exogenous polypeptide, or both the first and second exogenous polypeptides have an in vivo half-life of between about 24 hours and 60 days. In some embodiments, the first exogenous polypeptide, the second exogenous polypeptide, or both the first and second exogenous polypeptides have an in vivo half-life of at least 24 hours. In some embodiments, the first exogenous polypeptide, the second exogenous polypeptide, or both the first and second exogenous polypeptides have an in vivo half-life of greater than 36 hours. In some embodiments, the first exogenous polypeptide, the second exogenous polypeptide, or both the first and second exogenous polypeptides have an in vivo half-life of greater than 48 hours. In some embodiments, the first exogenous polypeptide, the second exogenous polypeptide, or both the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first exogenous polypeptide, the second exogenous polypeptide, or both the first and second exogenous polypeptides have an in vivo half-life of 1 week, 2 weeks, 3 weeks, 1 month, 2 months, 3 months, 4 months or longer.

In some embodiments, the engineered erythroid cells of the invention do not cause an immune reaction when administered to a subject. In some embodiments, the engineered erythroid cells of the invention produce a reduced immune reaction when administered to a subject as compared to the exogenous polypeptides administered without the cell.

### BRIEF DESCRIPTION OF THE DRAWINGS

FIG. 1 is a graph that shows the specific activity (μmol/min/mg) of uricases present in HEK-293T cells. GFP-fused uricases from Candida utilis, Aspergillus flavus, and Arthrobacter globiformis were expressed in HEK-293T cells. The presence of uricase was quantified by determining the mean fluorescence intensity of GFP fluorescence in uricase expressing cells and comparing the fluorescence with that of GFP-conjugated beads. From the three uricases tested, uricase from Candida utilis yielded the highest specific activity at neutral pH.

FIG. 2A is a schematic that shows one method of producing uricase and uric acid transporter in engineered erythroid cells.

FIG. 2B is a schematic that shows one method of producing uricase and uric acid transporter in engineered erythroid cells. In this method, the T2A cleavage sequence in inserted between the uricase and uric acid transporter proteins.

FIG. 2C is a schematic that shows one method of producing uricase and uric acid transporter in engineered erythroid cells. In this method, an internal ribosome entry site (IRES) is inserted between the uricase and uric acid transporter genes.

FIG. 3 is a schematic that shows one method of producing uricase and uric acid transporter in engineered erythroid cells. In this method, uricase and uric acid transporter are produced as direct peptide fusions separated by a linker.

FIG. 4A is a schematic that shows one method of producing uricase and uric acid transporter and additionally catalase in engineered erythroid cells. In this method, uricase and uric acid transporter are exoressed by a first lentiviral vector and catalase is exoressed by a second lentiviral vector.

FIG. 4B is a schematic that shows one method of producing uricase and uric acid transporter and additionally catalase in engineered erythroid cells. In this method, catalase is fused to uricase and expressed by a first lentiviral vector, and the uric acid transporter is expressed by a second lentiviral vector.

FIG. 5 shows the production of eGFP-CuUricase in a mixture of nucleated and enucleated engineered erythroid cells on differentiation day 26. The amount of eGFP-CuUricase in the cells was quantified using eGFP-conjugated bead standards. It was calculated that engineered erythroid cells expressing eGFP-CuUricase contained about 170,000 eGFP-CuUricase molecules per cell.

FIG. 6 shows the pesence of HA-GPA-URAT1 in a mixture of nucleated and enucleated engineered erythroid cells on differentiation day 21. The amount of HA-GPA-URAT1 on the cells was quantified using fluorescently labeled anti-HA epitope tag antibody bound to QUANTUM Simply Cellular beads (Bangs Laboratories, Inc.). It was calculated that engineered erythroid cells comprising HA-GPA-URAT1 contained about 30,000 HA-GPA-URAT1 molecules per cell.

FIG. 7 is a graph that shows the results of a uric acid transport assay in order to determine the activity of erythroid cells engineered to comprise HA-GPA-URAT1. Cells were incubated with heavy uric acid at 37° C. At various time points, cells were quickly washed with ice-cold buffer, lysed, and the amount of heavy uric acid in the cells was measured by mass spectrometry. The transport rate of uric acid was measured to be 1.8e-11 μmole/minute/positive cell for control red cells comprising HA-GPA. For red cells comprising HA-GPA-URAT, uric acid transport rate was measured to be 1.3e-10 umole/minute/positive cell, an at least 10-fold increase over the HA-GPA control.

FIG. 8 is a graph that shows dose estimation (in units (U)) of erythroid cells engineered to comprise uricase and uric acid transporter.

FIG. 9A shows assessment of eGFP-AgUricase via eGFP fluorescence by flow cytometry and HA-GPA-Glut9 via anti-HA antibody cell surface staining and analysis via flow cytometry.

FIG. 9B is a graph that shows uric acid depletion (mg/dL) over time (min) in erythroid cells comprising eGFP-AgUricase and HA-GPA-Glut9 (RTX-Uricase+UA Transporter). Untransfected erythroid cells were used as control (RTX-CTRL).

### DETAILED DESCRIPTION

The present disclosure is based on the development of cells, e.g., erythroid cells or enucleated cells that have been engineered to include a uric acid degrading polypeptide, a uric acid transporter, or both a uric acid degrading polypeptide and a uric acid transporter. According to embodiments of the present disclosure, the engineered erythroid cells or enucleated cells are nucleated cells, or are enucleated cells. According to embodiments of the present disclosure, the uric acid degrading polypeptide is located inside the cell (e.g., expressed in an erythroid precursor cell) and the uric acid transporter is located at the surface of the erythroid cell (e.g., is present on the plasma membrane of the cell), such that the uric acid transporter ensures optimal uptake of uric acid into the cell. The engineered erythroid cells or enucleated cells of the present invention provide advantages to, for example, hypotonically loaded cells. In contrast to the erythroid cells of the present invention, which are engineered to comprise a uric acid degrading polypeptide inside the cell and at a high copy number, a hypotonically loaded erythroid cell is limited with respect to the levels of polypeptide that may be loaded into the cell, and in addition sometimes displays aberrant physical characteristics such as increased osmotic fragility, altered cell size, reduced hemoglobin concentration, or increased phosphatidylserine levels on the outer leaflet of the cell membrane. According to embodiments of the present disclosure, the engineered erythroid cells are nucleated erythroid cells, or are enucleated erythroid cells (e.g., reticuloyctes or erythrocytes).

The present disclosure is based, at least in part, on an experimental determination of the target uricase activity of an engineered erythroid cell or enucleated cell required to achieve clinical efficacy, and to establish clinical dosing feasibility. Taking into account the target uric acid degradation activity needed to achieve clinical efficacy and the uric acid uptake activity by a uric acid transporter comprised in the engineered erythroid cell or enucleated cell comprising uricase, levels of the uric acid degrading polypeptide present in the engineered erythroid cell or enucleated cell were optimized to meet the target activity. Moreover, the engineered erythroid cells or enucleated cells of the invention confer a prolonged in vivo half-life to the uric acid degrading polypeptide, uric acid transporter, or both the uric acid degrading polypeptide and the uric acid transporter comprised in the cells, as compared to the in vivo half-life of the uric acid degrading polypeptide and the uric acid transporter administered to a subject alone (i.e., not comprised in an erythroid cell). In addition, the engineered erythroid cells or enucleated cells of the invention may not cause an immune reaction when administered to a subject, or may have a reduced immune reaction when administered to a subject as compared to the immune reaction caused by the same exogenous polypeptides when administered to a subject without the cell). Without wishing to be bound by any particular theory, it is believed that a reduced immune reaction may result from the shielding or protection that erythroid cells or enucleated cells confer to the exogenous polypeptides against antibodies within a subject, thereby allowing the activity (e.g., enzymatic activity) of the one or more exogenous polypeptides to be preserved in vivo.

The engineered erythroid cells of the present invention provide advantages to, for example, hypotonically loaded cells. In contrast to the erythroid cells of the present invention, which are engineered to include a uric acid degrading polypeptide in the cell and at a high copy number, a hypotonically loaded erythroid cell is limited with respect to the levels of polypeptide that may be loaded into the cell, and in addition sometimes displays aberrant physical characteristics such as increased osmotic fragility, altered cell size, reduced hemoglobin concentration, or increased phosphatidylserine levels on the outer leaflet of the cell membrane.

Moreover, the engineered erythroid cells of the invention confer a prolonged in vivo half-life to the uric acid degrading polypeptide, and/or the uric acid transporter, as compared to the in vivo half-life of the uric acid degrading polypeptide, and/or the uric acid transporter when either of these polypeptides are administered to a subject alone (i.e., not present in or on an erythroid cell).

In addition, the engineered erythroid cells of the invention may not cause an immune reaction when administered to a subject, or may produce a reduced immune reaction when administered to a subject as compared to the immune reaction caused by the same exogenous polypeptides when administered to a subject without the cell. Without wishing to be bound by any particular theory, it is believed that a reduced immune reaction may result from the shielding or protection that the erythroid cells confer to the exogenous polypeptide against antibodies within a subject, thereby allowing the activity (e.g., enzymatic activity) of the one or more exogenous polypeptides to be preserved in vivo.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, encompasses variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein, an “additional therapeutic” refers to any therapeutic that is used in addition to another treatment. For example, when the method is one directed to treatment with the engineered erythroid cells described herein, and the method comprises the use of an additional therapeutic, the additional therapeutic is in addition to the engineered erythroid cells described herein. Generally, the additional therapeutic will be a different therapeutic. The additional therapeutic may be administered at the same time or at a different time and/or via the same mode of administration or via a different mode of administration, as that of the other therapeutic. In preferred embodiments, the additional therapeutic will be given at a time and in a way that will provide a benefit to the subject during the effective treatment window of the other therapeutic. When two compositions are administered with a specific time period, generally the time period is measured from the start of the first composition to the start of the second composition. As used herein, when two compositions are given within an hour, for example, the time before the start of the administration of the first composition is about an hour before the start of the administration of the second composition. In some embodiments, the additional therapeutic is another therapeutic for the treatment of gout or a condition associated with gout. As used herein, a “gout therapeutic” is any therapeutic that can be administered and from which a subject with gout may derive a benefit because of its administration. In some embodiments, the gout therapeutic is an oral gout therapeutic (i.e., a gout therapeutic that can be taken or given orally).

As used herein, “catalase” is meant to refer to an enzyme that breaks down hydrogen peroxide into oxygen and water (2H₂O₂→2H₂O+O₂). In some embodiments, a catalase refers to any one or more enzymes set forth in ExPasy ENZYME entry: EC 1.11.1.6.

As used herein, “dose” refers to a specific quantity of a pharmacologically active material for administration to a subject for a given time. Unless otherwise specified, the doses recited refer to an engineered erythroid cell comprising a uric acid degrading polypeptide as described herein, an engineered erythroid cell comprising a uric acid transporter as described herein, or an engineered erythroid cell comprising a uric acid degrading polypeptide and a uric acid transporter as described herein. In some embodiments, a dose of engineered erythroid cells refers to an effective amount of engineered erythroid cells. For example, In some embodiments a dose or effective amount of engineered erythroid cells comprises at least 10 units, or about 10-20 units, of uricolytic activity per dose. In some embodiments, a dose or effective amount of engineered erythroid cells refers to about 1×10¹⁰-1×10¹² engineered erythroid cells, or about 5×10¹⁰-5×10¹¹engineered erythroid cells per dose. In some embodiments, a dose or effective amount of engineered erythroid cells refers to about 1×10¹¹ engineered erythroid cells per dose. In some embodiments, a dose or effective amount of engineered erythroid cells refers to about 1×10¹⁰-5×10⁻¹⁰ engineered erythroid cells per dose. In some embodiments, a dose or effective amount of engineered erythroid cells refers to about 3×10¹⁰ engineered erythroid cells per dose. In some embodiments, the engineered erythroid cells have at least about 1×10⁻¹⁰ units, or at least about 2×10⁻¹⁰ units, or between about 1-2×10⁻¹⁰ units of uricolytic activity per cell. In some embodiments, the engineered erythroid cells have at least 3-6×10⁻¹⁰ units of uricoloytic activity per cell, e.g., the engineered erythroid cells have at least about 3×10⁻¹⁰ units, at least about 4×10⁻¹⁰ units, at least about 5×10⁻¹⁰ units or at least about 6×10⁻¹⁰ units of uricolytic activity per cell. In some embodiments, the engineered erythroid cells have between about 3-6×10⁻¹⁰ units of uricolytic activity per cell. In some embodiments, the engineered erythroid cells have about 3×10⁻¹⁰ units, about 4×10⁻¹⁰ units, about 5×10⁻¹⁰ units or about 6×10⁻¹⁰ units of uricolytic activity per cell. When referring to a dose for administration, in an embodiment of any one of the methods, compositions or kits provided herein, any one of the doses provided herein is the dose as it appears on a label/label dose.

As used herein, a “drug-induced” gout flare refers to an occurrence of or increased incidence of a gout flare associated with initiation of therapy to treat gout and/or administration of a therapeutic agent for the treatment of gout, for example, initiation of therapy with a xanthine oxidase inhibitor, urate oxidase, or a uricosuric agent. A gout flare is “associated” with initiation of gout therapy when the flare occurs contemporaneously or following at least a first administration of a therapeutic agent for the treatment of gout.

As used herein, an “elevated serum uric acid level” refers to any level of uric acid in a subject's serum that may lead to an undesirable result or would be deemed by a clinician to be elevated. In an embodiment, an elevated serum uric acid level refers to a level of uric acid considered to be above normal by the American Medical Association. In an embodiment, the subject of any one of the methods provided herein can have a serum uric acid level of >5 mg/dL, >6 mg/dL, or >7 mg/dL. Such a subject may be a hyperuremic subject. Whether or not a subject has elevated blood uric acid levels can be determined by a clinician, and in some embodiments, the subject is one in which a clinician has identified or would identify as having elevated serum uric acid levels.

As used herein, the term “endogenous” is meant to refer to a native form of compound (e.g., a small molecule) or process. For example, in some embodiments, the term “endogenous” refers to the native form of a nucleic acid or polypeptide in its natural location in the organism or in the genome of an organism.

As used herein, the term “an engineered cell” is meant to refer to a genetically-modified cell or progeny thereof. In some embodiments, an engineered cell (e.g. an engineered enucleated cell) can be produced using coupling reagents to link an exogenous polypeptide to the surface of the cell (e.g., using click chemistry).

As used herein, the term “enucleated” refers to a cell, e.g., a reticulocyte or mature red blood cell (erythrocyte) that lacks a nucleus. In an embodiment an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In an embodiment an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell. In an embodiment an enucleated cell lacks DNA. In an embodiment an enucleated cell is incapable of expressing a polypeptide, e.g., incapable of transcribing and/or translating DNA into protein, e.g., lacks the cellular machinery necessary to transcribe and/or translate DNA into protein. In some embodiments, an enucleated cell is an erythrocyte, a reticulocyte, or a platelet.

In some embodiments, the enucleated cells are not platelets, and therefore are “platelet free enucleated” cells (“PFE” cells). It should be understood that platelets do not have nuclei, and in this particular embodiment, platelets are not intended to be encompassed.

As used herein, “erythroid cell” includes a nucleated red blood cell, a red blood cell precursor, an enucleated mature red blood cell, and a reticulocyte. As used herein, an erythroid cell includes an erythroid precursor cell, a cell capable of differentiating into a reticulocyte or erythrocyte. For example, erythroid precursor cells include any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid precursor cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al., (2014) Mol. Ther. 22(2): 451-63, the entire contents of which are incorporated by reference herein). In addition, the cells may be intended for autologous use or provide a source for allogeneic transfusion. In some embodiments, erythroid cells are cultured. In an embodiment an erythroid cell is an enucleated red blood cell.

As used herein, the term “exogenous,” when used in the context of nucleic acid, includes a transgene and recombinant nucleic acids.

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell, or a progenitor of the cell, which nucleic acid is optionally not retained by the cell. In some embodiments, an exogenous polypeptide is a polypeptide conjugated to the surface of the cell by chemical or enzymatic means.

As used herein, the term “express” or “expression” refers to the process to produce a polypeptide, including transcription and translation. Expression may be, e.g., increased by a number of approaches, including: increasing the number of genes encoding the polypeptide, increasing the transcription of the gene (such as by placing the gene under the control of a constitutive promoter), increasing the translation of the gene, knocking out of a competitive gene, or a combination of these and/or other approaches.

As used herein, the terms “first”, “second” and “third”, etc. with respect to exogenous polypeptides are used for convenience of distinguishing when there is more than one type of exogenous polypeptide. Use of these terms is not intended to confer a specific order or orientation of the exogenous polypeptides unless explicitly so stated.

As used herein, the term “fragment” refers to sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence. Fragments may be derived from any contiguous portion of a nucleic acid or amino acid sequence of choice.

As used herein, the term “gene” is used broadly to refer to any segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein, the term “gout” generally refers to a disorder or condition associated with the buildup of uric acid, such as deposition of uric crystals in tissues and joints, and/or a clinically relevant elevated serum uric acid level. Accumulation of uric acid may be due to overproduction of uric acid or reduced excretion of uric acid. Gout may range from asymptomatic to severe and painful inflammatory conditions. A “disease, condition or disorder associated with gout” refers to any condition in a subject where the subject experiences local and/or systemic effects of gout, including inflammation and immune responses, and in which the condition is caused or exacerbated by, or the condition can result in or exacerbate, gout. A gout flare is an attack or exacerbation of gout symptoms, which can happen at any time. Gout flares can include gout flares that occur after the administration of a uric acid lowering therapy. As used herein, the term “chronic refractory gout” refers to symptomatic gout in which conventional urate-lowering therapies are contraindicated or ineffective to control gout and/or hyperuricemia. Chronic refractory gout is often characterized by recurrent gout flares, chronic gout arthropathy with or without bony erosions, visible progressive tophi, physical disability, and/or poor health-related quality of life.

As used herein, the term “hyperuricemia” refers to the presence of high levels of uric acid in the blood. Hyperuricemia may occur because of decreased excretion. Hyperuricemia may also occur from increased production, or a combination of the two mechanisms.

As used herein the term “nucleic acid molecule” refers to a single or double-stranded polymer of deoxyribonucleotide or ribonucleotide bases. It includes chromosomal DNA and self-replicating plasmids, vectors, mRNA, tRNA, siRNA, etc. which may be recombinant and from which exogenous polypeptides may be expressed when the nucleic acid is introduced into a cell.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.” (a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence. (b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences, 8:155-65 (1992), and Pearson, et al., Methods in Molecular Biology, 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995). Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination. (c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA). (d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. (e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid. Mutations may also be made to the nucleotide sequences of the present proteins by reference to the genetic code, including taking into account codon degeneracy.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered agent.

As used herein, the terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. According to some embodiments, the peptide is of any length or size.

As used herein, polypeptides referred to herein as “recombinant” refers to polypeptides which have been produced by recombinant DNA methodology, including those that are generated by procedures which rely upon a method of artificial recombination, such as the polymerase chain reaction (PCR) and/or cloning into a vector using restriction enzymes.

“Recombinant” polypeptides are also polypeptides having altered expression, such as a naturally occurring polypeptide with recombinantly modified expression in a cell, such as a host cell.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in particular embodiments the subject is a human.

As used herein, the phrase “subject in need” refers to a subject that (i) will be administered an engineered erythroid cell (or pharmaceutical composition comprising an engineered erythroid cell) according to the described invention, (ii) is receiving an engineered erythroid cell (or pharmaceutical composition comprising an engineered erythroid cell) according to the described invention; or (iii) has received an engineered erythroid cell (or pharmaceutical composition comprising an engineered erythroid cell) according to the described invention; or (iv) is in need of and/or would benefit from administration of an engineered erythroid cell (or pharmaceutical composition comprising an engineered erythroid cell) according to the described invention, unless the context and usage of the phrase indicates otherwise

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “therapeutic amount”, “therapeutically effective amount”, an “amount effective”, or “pharmaceutically effective amount” of an active agent (e.g. an engineered erythroid cell or enucleated cell as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, a “disease or disorder associated with hyperuricemia” refers to a disease or disorder typically associated with elevated levels of uric acid, including, but not limited to a metabolic disorder, e.g., metabolic syndrome, hyperuricemia, gout (e.g., chronic refractory gout, gout tophus and gouty arthritis), tumor-lysis syndrome, Lesch-Nyhan syndrome, cardiovascular disease, diabetes, hypertension, renal disease, or uric acid nephrolithiasis. Such disorders may optionally be acute or chronic. Elevated levels refer to levels that are higher than levels that are considered normal by the American Medical Association, although significantly lower levels are common in vegetarians due to a decreased intake of purine-rich meat.

As used herein, “uric acid”, also known as urate (the two terms are used interchangeably herein), refers to an end product of purine metabolism. Humans produce large quantities of uric acid. In human blood, uric acid concentrations between 3.6 mg/dL (˜214 μmol/L) and 8.3 mg/dL (˜494 μmol/L) (1 mg/dL=59.48 μmol/L) are considered normal by the American Medical Association. Uric acid concentrations can be measured in samples from a subject, e.g., blood or urine samples, using known methods.

As used herein, a “uric acid degrading polypeptide” or “uric acid degrading enzyme” refers to any polypeptide (enzyme) that is involved in catabolizing or degrading uric acid. Examples of uric acid degrading polypeptides include urate oxidase (also known as uricase), allantoinase and allantoicase. Other examples of uric acid degrading polypeptides are described herein and are not intended to be limiting. In an embodiment, a uric acid degrading polypeptide has uric acid as its substrate. In an embodiment, a uric acid degrading polypeptide catalyzes the hydrolysis of uric acid.

As used herein, “uricolytic activity” refers to the activity of degradation of uric acid. Uricolytic activity is measured in units, where one unit of activity is defined as degradation of 1 umol of uric acid per minute. In an embodiment, a uric acid degrading polypeptide alone has uricolytic activity. In an embodiment, two or more uric acid degrading polypeptides contribute to uricolytic activity.

As used herein, a “hydrogen peroxide degrading polypeptide” refers to any polypeptide (enzyme) that is involved in the breakdown of hydrogen peroxide. In an embodiment, a hydrogen peroxide degrading polypeptide breaks down hydrogen peroxide to water and oxygen.

As used herein, the term “variant” refers to a polypeptide which differs from the original protein from which it was derived (e.g., a wild-type protein) by one or more amino acid substitutions, deletions, insertions (i.e., additions), or other modifications. In some embodiments, these modifications do not significantly change the biological activity of the original protein. In many cases, a variant retains at least 10%, 20%, 30%, 40%, 50%, 60%, 70%, 80%, 90%, 95%, or 100% of the biological activity of original protein. The biological activity of a variant can also be higher than that of the original protein. A variant can be naturally-occurring, such as by allelic variation or polymorphism, or be deliberately engineered. For example, a variant may comprise a substitution at one or more amino acid residue positions to replace a naturally-occurring amino acid residue for a structurally similar amino acid residue. Structurally similar amino acids include: (I, L and V); (F and Y); (K and R); (Q and N); (D and E); and (G and A). In some embodiments, variants include (i) polymorphic variants and natural or artificial mutants, (ii) modified polypeptides in which one or more residues is modified, and (iii) mutants comprising one or more modified residues. Variants may differ from the reference sequence (e.g., by truncation, deletion, substitution, or addition) by no more than 1, 2, 3, 4, 5, 8, 10, 20, or 50 residues, and retains (or encodes a polypeptide that retains) a function of the wild-type protein from which they were derived.

The amino acid sequence of a variant is substantially identical to that of the original protein. In many embodiments, a variant shares at least 50%, 60%, 70%, 75%, 80%, 85%, 90%, 91%, 92%, 93%, 94%, 95%, 96%, 97%, 98%, or 99%, or more global sequence identity or similarity with the original protein. Sequence identity or similarity can be determined using various methods known in the art, such as Basic Local Alignment Tool (BLAST), dot matrix analysis, or the dynamic programming method. In one example, the sequence identity or similarity is determined by using the Genetics Computer Group (GCG) programs GAP (Needleman-Wunsch algorithm) The amino acid sequences of a variant and the original protein can be substantially identical in one or more regions, but divergent in other regions. A variant may include a fragment (e.g., a biologically active fragment of a polypeptide). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide.

### II. Engineered Cells

The present disclosure features erythroid cells and enucleated cells that are engineered to include at least one exogenous polypeptide comprising a uric acid degrading polypeptide, a uric acid transporter, or both. In some embodiments an enucleated cell is a erythroid cell, for example, that has lost its nucleus through differentiation from an erythroid precursor cell. It will be understood, however, that not all enucleated cells are erythroid cells and, accordingly, enucleated cells encompassed herein can also include, e.g., platelets. In some embodiments, populations of enucleated cells that do not include platelets are provided, and are therefore platelet free populations of enucleated cells. In certain aspects of the disclosure, the erythroid cell is a reticulocyte, or an erythrocyte (e.g., red blood cell (RBC)). Erythrocytes offer a number of advantages over other cells, including being non-autologous due to lack of major histocompatibility complex (MHC), having long circulation time, and being amenable to production in large numbers. In certain embodiments of the disclosure, the engineered erythroid cells are nucleated.

### Engineered Erythroid Cells and Enucleated Cells

The present disclosure provides erythroid cells and enucleated cells that are engineered to degrade uric acid by expression of a uric acid degrading polypeptide, a uric acid transporter, or both a uric acid degrading polypeptide and a uric acid transporter. In some embodiments an enucleated cell is a erythroid cell, for example, that has lost its nucleus through differentiation from an erythroid precursor cell. It will be understood, however, that not all enucleated cells are erythroid cells and, accordingly, enucleated cells encompassed herein can also include, e.g., platelets. In some embodiments, enucleated cells are not platelets and are therefore platelet free enucleated cells. In certain aspects of the disclosure, the erythroid cell is a reticulocyte or an erythrocyte (red blood cell (RBC)). Erythrocytes offer a number of advantages over other cells, including being non-autologous due to lack of major histocompatibility complex (MHC), having longer circulation time, and being amenable to production in large numbers. In certain aspects of the disclosure, the engineered erythroid cells are nucleated.

The engineered cells may be advantageously used to reduce uric acid concentration in the milieu surrounding the cell (e.g., in vitro or in vivo). For example, the engineered cells provided herein may be administered to a subject (e.g., a human subject) to reduce the concentration of uric acid in the subject (e.g., in the blood, plasma, or serum of the subject). In some embodiments, the disclosure provides an engineered erythroid cell comprising at least one (e.g., one, two, three, four, or more) exogenous polypeptide, wherein each exogenous polypeptide may comprise either at least one uric acid degrading polypeptide, at least one uric acid transporter, or both a uric acid degrading polypeptide and a uric acid transporter.

Any condition, disease or disorder in which a reduction of uric acid levels is desired may be treated by administering the engineered cells provided herein.

In some embodiments of any of the aspects herein, the engineered erythroid cell is a reticulocyte. In some embodiments of any of the aspects herein, the engineered erythroid cell is an erythrocyte.

### Uric Acid (Urate)

Uric acid is an end product of purine metabolism. Xanthine oxidase oxidizes oxypurines such as xanthine and hypoxanthine to uric acid. In humans and higher primates, uric acid is the final oxidation product of purine catabolism. In most other mammals, uricase (uricase) further oxidizes uric acid to allantoin.

In contrast to other mammals, humans lack the capacity to metabolize urate by hepatic uricase, due to mutational silencing of the enzyme. The loss of uricase in higher primates parallels the similar loss of the ability to synthesize ascorbic acid. This may be because in higher primates, uric acid partially replaces ascorbic acid. Both uric acid and ascorbate are strong reducing agents and potent antioxidants. In humans, about half the antioxidant capacity of plasma comes from urate. Urate body pool is about 1-1.2 g, daily turnover being 0.6-0.7 g. Two-thirds of the newly produced uric acid is excreted in urine, while the remaining one third has a biliary or intestinal elimination or undergoes bacterial uricolysis. It emerges, therefore, that the kidney is the main regulator of uric acid balance.

Humans produce large quantities of uric acid. In human blood, uric acid concentrations between 3.6 mg/dL (˜214 μmol/L) and 8.3 mg/dL (˜494 μmol/L) (1 mg/dL=59.48 μmol/L) are considered normal by the American Medical Association, although significantly lower levels are common in vegetarians due to a decreased intake of purine-rich meat. Uric acid is a weak organic acid of molecular weight 168 Daltons, with dissociation constants pKa1=5.75 and pKa2=10.3 [I]. Therefore, at physiological blood pH, almost all the urate species are in the form of monovalent-anion. The solubility of urate in blood is about 7.0 mg/dL, above which it may deposit in tissues as monosodium-urate-monohydrate. Only about 4-5% of urate is bound to plasma proteins. Relative to other mammals, humans have high urate levels in plasma, ranging between 3.5 and 7.5 mg/dL (200-450 μmol/L), males having 1.2 times greater urate levels than healthy females.

Disorders associated with high uric acid levels (hyperuricemia) include metabolic syndrome, hyperuricemia, gout (e.g., chronic refractory gout, gout tophus and gouty arthritis), tumor lysis syndrome, Lesch-Nyhan syndrome, cardiovascular disease, diabetes, hypertension, renal disease, metabolic syndrome, or uric acid nephrolithiasis. Such disorders can be treated with an engineered erythroid cell of the invention comprising a uric acid degrading polypeptide, a uric acid transporter polypeptide, or an engineered erythroid cell comprising a uric acid degrading polypeptide and a uric acid transporter polypeptide, e.g., a composition (e.g., a pharmaceutical composition) comprising said engineered erythroid cells, as described herein. In addition, such disorders can be treated by a combination of the engineered erythroid cells described herein, and another agent (e.g., a xanthine-oxidase inhibitor and/or an uricosuric and/or an antacid and/or a proton pump inhibitor). The treatment of various diseases and disorders associated with hyperuricemia is described herein.

Uric acid, a weak organic acid, has very low pH-dependent solubility in aqueous solutions. About 70% of urate elimination occurs in urine, the kidney standing as a major determinant of plasma levels. The complex renal handling results in a fractional clearance of less than 10%. Recently identified urate-specific transporter/channels are involved in tubular handling and extracellular transport. Extracellular fluid, rather than urine output, is the main regulator of urate excretion. A number of interfering agents, including widely used drugs such as aspirin, losartan, diuretics, may decrease or increase urate elimination. Hyperuricemia induced by hypouricosuria often accompanies the metabolic syndrome, and insulin resistance has been hypothesized as the common underlying defect. Hyperuricosuria, associated with dehydration or exercise, results in acute uric acid nephropathy, and causes an obstructive acute renal failure (ARF).

### Uric Acid Degrading Enzymes

In one aspect, the present disclosure provides an erythroid cell engineered to degrade uric acid, comprising an exogenous polypeptide comprising at least one uric acid degrading polypeptide, or a variant thereof. In some embodiments, the erythroid cell comprises more than one (e.g., two, three, four, five, or more) exogenous polypeptides, each comprising at least one uric acid degrading polypeptide, or a variant thereof. In some embodiments, the engineered cells described herein comprise more than one type of exogenous polypeptide, wherein each exogenous polypeptide comprises a uric acid degrading polypeptide, and wherein the uric acid degrading polypeptides are not the same (e.g., the uric acid degrading polypeptides may comprise different types of uric acid degrading polypeptides, or variants of the same type of uric acid degrading polypeptide). For example, in some embodiments, the engineered cell may comprise a first exogenous polypeptide comprising a uricase, or a variant thereof, and a second exogenous polypeptide comprising a uric acid degrading polypeptide that is not a uricase. In addition, an exogenous polypeptide may comprise more than one (e.g., one, two, three, four, five, or more) uric acid degrading polypeptide (e.g., two different uricases).

Many uric acid degrading polypeptides are known in the art and may be used as described herein. For example, the uric acid catabolism pathway includes several uric acid degrading enzymes. Urate oxidase is the first of three enzymes to convert uric acid to S-(+)-allantoin (allantoin). After uric acid is converted to 5-hydroxyisourate by urate oxidase, 5-hydroxyisourate (HIU) is converted to 2-oxo-4-hydroxy-4-carboxy-5-ureidoimidazoline (OHCU) by HIU hydrolase, and then to S-(+)-allantoin (allantoin) by 2-oxo-4-hydroxy-4-carboxy-5-ureidoimidazoline decarboxylase (OHCU decarboxylase). Allantoin is converted to allantoate by allantoinase. Allantoate is converted to urea by allantoicase. (Lee et al. 2013 PloS ONE 8(5):e64292). Any one or more of the enzymes involved in uric acid catabolism (i.e., uric acid degrading polypeptides) can be included in the erythroid cells described herein.

In some embodiments, the at least one uric acid degrading polypeptide is any enzyme that is capable of degrading uric acid (e.g., a uricase). In some embodiments, the at least one uric acid degrading polypeptide is any enzyme having uric acid as a substrate. In some embodiments, the at least one uric acid degrading polypeptide is any enzyme that is involved in uric acid catabolism, for example, an enzyme that degrades HIU (e.g., an HIU hydrolase), an enzyme that degrades OHCU (e.g., an OHCU decarboxylase), an enzyme that degrades allantoin (e.g., an allantoinase), or an enzyme that degrades allantoate (e.g., an allantoicase).

In some embodiments, the at least one uric acid degrading polypeptide, or variant thereof, can be derived from any source or species, e.g., mammalian, fungal, plant or bacterial sources, or can be recombinantly engineered. In some embodiments, the uric acid degrading polypeptide can be a chimeric uric acid degrading polypeptide, e.g., derived from two different species.

The exogenous polypeptides included in the engineered cells provided herein may comprise an exogenous polypeptide comprising any uric acid degrading polypeptide. In some embodiments, the uric acid degrading polypeptide comprises a uricase, or a variant thereof. In some embodiments, the uric acid degrading polypeptide comprises an HIU hydrolase, or a variant thereof. In some embodiments, the uric acid degrading polypeptide comprises an OHCU decarboxylase, or a variant thereof. In some embodiments, the uric acid degrading polypeptide comprises an allantoinase, or variant thereof. In some embodiments, the uric acid degrading polypeptide comprises an allantoicase, or a variant thereof. In some embodiments, the uric acid degrading polypeptide comprises a myeloperoxidase, or variant thereof. In some embodiments, the uric acid degrading polypeptide comprises a FAD-dependent urate hydroxylase, or variant thereof. In some embodiments, the uric acid degrading polypeptide comprises an xanthine dehydrogenase, or variant thereof. In some embodiments, the uric acid degrading polypeptide comprises a nucleoside deoxyribosyltransferase, or variant thereof. In some embodiments, the uric acid degrading polypeptide comprises a dioxotetrahydropyrimidine phosphoribosyltransferase, or variant thereof. In some embodiments, the uric acid degrading polypeptide comprises a dihydropyrimidinase, or variant thereof. In some embodiments, the uric acid degrading polypeptide comprises an guanine deaminase, or variant thereof. More than one uric acid degradation polypeptide, or variant thereof, may be combined in one or more erythroid cells, as described herein.

Uricases (also referred to as urate oxidase), and variants thereof, are described in detail below.

Allantoinase (E.C. 3.5.2.5), is a uric acid degrading polypeptide that catalyzes the reaction of allantoin to allantoate.

Urate is a substrate of myeloperoxidase (MPO; E.C. 1.11.2.2). Increased concentrations of urate in gout lead to the release of MPO from neutrophils and the oxidation of urate. Products of MPO and reactive metabolites of urate may contribute to the pathology of gout and hyperuricaemia. (Stamp et al. Rheumatology (Oxford). 2014 53(11):1958-65).

Flavin adenine dinucleotide (FAD)-dependent urate hydroxylase (E.C. 1.14.13.113) is a flavoprotein that catalyzes the reaction urate+NADH+H⁺+O₂=5-hydroxyisourate+NAD⁺+H₂O. The product 5-hydroxyisourate is spontaneously converted to allantoin.

Xanthine dehydrogenase (XO; E.C. 1.17.1.4), catalyzes the oxidation of hypoxanthine to xanthine and can further catalyze the oxidation of xanthine to uric acid. Xanthine dehydrogenases play an important role in the catabolism of purines in some species, including humans.

Nucleoside deoxyribosyltransferase (E.C. 2.4.2.6) belongs to the family of glycosyltransferases, specifically the pentosyltransferases, and catalyzes the chemical reaction 2-deoxy-D-ribosyl-base₁+base₂ 2-deoxy-D-ribosyl-base₂+base₁.

Dioxotetrahydropyrimidine phosphoribosyltransferase (E.C. 2.4.2.20) catalyzes the reaction pyrophosphate+a 2,4-dioxotetrahydropyrirnidine D-ribonucleotide=PRPP+a 2,4-dioxotetrahydropyrimidine.

Dihydropyrimidinase (DPYS; E.C. 3.5.2.2) catalyzes the conversion of 5,6-dihydrouracil to 3-ureidopropionate in pyrimidine metabolism.

Guanine deaminase (guanase; GAH; E.C. 3.5.4.3): in certain organisms, during the degradation of purines, guanine deaminase converts guanine to xanthine. In the aerobic degradative pathway, xanthine is oxidized in three steps to allantoic acid.

In some embodiments, the uric acid degrading polypeptide comprises or consists of a variant of the wild-type uric acid degrading polypeptide having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type uric acid degrading polypeptide (e.g., a uricase, a 5-hydroxyisourate (HIU) hydrolase, an 2-oxo-4-hydroxy-4-carboxy-5-ureidoimidazoline (OHCU) decarboxylase, an allantoinase, an allantoicase, a myeloperoxidase, a flavin adenine dinucleotide (FAD)-dependent urate hydroxylase, an xanthine dehydrogenase, a nucleoside deoxyribosyltransferase, or variant thereof, an dioxotetrahydropyrimidine phosphoribosyltransferase, or variant thereof, an dihydropyrimidinase, or variant thereof, and a guanine deaminase, or a variant thereof).

In some embodiments, an engineered erythroid cell or an enucleated cell comprises an exogenous polypeptide comprising a uric acid degrading polypeptide that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP)). The polypeptide of interest may be disposed in any configuration of the exogenous polypeptide (e.g., the polypeptide of interest may be fused to the N-terminus or C-terminus of the uric acid degrading polypeptide).

In some embodiments, the exogenous polypeptide may include a linker disposed between the uric acid degrading polypeptide and the at least one polypeptide of interest. In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO: 48), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Exemplary linkers include, but are not limited to, GGGGSGGGG (SEQ ID NO: 33), GGGGSGGGGS (SEQ ID NO: 49), GSGSGSGSGS (SEQ ID NO: 50), PSTSTST (SEQ ID NO: 51), and EIDKPSQ (SEQ ID NO: 52), and multimers thereof.

In some embodiments, the exogenous polypeptide comprises a transmembrane domain or a transmembrane polypeptide (e.g., SMIM1, GPA, or Kell) and a uric acid degrading polypeptide. In some embodiments, the transmembrane domain is derived from SMIM1. In some embodiments, the transmembrane domain is derived from GPA. For example, in some embodiments, the transmembrane domain is derived from GPA and comprises or consists of the amino acid sequence:

(SEQ ID NO: 54)
LSTTEVAMHTSTSSSVTKSYISSQTNDTHKRDTYAATPRAHEVSEISVRT
VYPPEEETGERVQLAHHFSEPEITLIIFGVMAGVIGTILLISYGIRRLIK
KSPSDVKPLPSPDTDVPLSSVEIENPETSDQ

Without wishing to be bound by any particular theory, fusion of a uric acid degrading polypeptide (e.g., uricase) to a transmembrane domain or a transmembrane polypeptide (e.g., GPA or a transmembrane domain of GPA) advantageously reduces the formation of aggregates by the uric acid degrading polypeptide. For example, overexpression of a uricase in mammalian cells has been reported to result in the formation of large aggregates that form crystalloid structures (see, e.g., Yokota et al. (1999) J. Histochem. Cytochem. 47(9): 1133-40). In contrast, surprisingly, when uricase is expressed in mammalian cells (e.g., erythroid cells) as a fusion with a transmembrane domain or a transmembrane polypeptide (e.g., GPA or a transmembrane domain of GPA), aggregation is minimized. Moreover, although uricase is generally a tetramer, the expression of uricase as a fusion with a transmembrane domain or a transmembrane polypeptide (e.g., GPA or a transmembrane domain of GPA) surprisingly does not inhibit the catalytic activity of uricase.

In some embodiments, the transmembrane domain or transmembrane polypeptide is disposed in the exogenous polypeptide such that the uric acid degrading polypeptide present in the exogenous polypeptide locates to the cytosol of the cell (e.g., proximate to the inner leaflet of the plasma membrane). In some embodiments, the transmembrane domain or transmembrane polypeptide is disposed in the exogenous polypeptide such that the uric acid degrading polypeptide present in the exogenous polypeptide locates in the outer surface of the cell (e.g., facing the extracellular milieu of the cell). In some embodiments, the exogenous polypeptide does not include a transmembrane domain or a transmembrane polypeptide. In some embodiments, the exogenous polypeptide does not include a polypeptide that is endogenous to the cell. In some embodiments, a linker (e.g., any linker provided herein) is disposed between the transmembrane domain or transmembrane polypeptide and the uric acid degrading polypeptide.

In some embodiments the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA. For example, in some embodiments, the leader or signal sequence is derived from GPA and comprises or consists of the amino acid sequence MYGKIIFVLLLSEIVSISA (SEQ ID NO: 53).

### Uricases

The disclosure provides, in one aspect, an erythroid cell engineered to degrade uric acid, comprising a first exogenous polypeptide comprising a uricase, or a variant thereof. In some embodiments, the erythroid cell comprises more than one (e.g., two, three, four, or five) exogenous polypeptide comprising a uricase.

Uricase (also referred to as UO, urate oxidase, urate:oxygen oxidoreductase (E.C. 1.7.3.3)) is an enzyme in the purine degradation pathway that catalyzes the oxidation of uric acid to 5-hydroxyisourate. Uricase is the first in a pathway of three enzymes to convert uric acid to S-(+)-allantoin. After uric acid is converted to 5-hydroxyisourate by urate oxidase, 5-hydroxyisourate (HIU) is converted to 2-oxo-4-hydroxy-4-carboxy-5-ureidoimidazoline (OHCU) by HIU hydrolase, and then to S-(+)-allantoin by 2-oxo-4-hydroxy-4-carboxy-5-ureidoimidazoline decarboxylase (OHCU decarboxylase). Without HIU hydrolase and OHCU decarboxylase, HIU will spontaneously decompose into racemic allantoin.

Uricase is an enzyme endogenous to most mammals, with the exception of humans and certain other primates, and is also found in plants, fungi, yeast, and bacteria. Humans do not produce enzymatically active uricase as a result of several mutations in the gene for uricase acquired during the evolution of higher primates. Wu, X, et al., (1992) J Mol Evol 34:78-84. As a consequence, in susceptible individuals, excessive concentrations of uric acid in the blood (hyperuricemia) and in the urine (hyperuricosuria) can lead to gout, disfiguring urate deposits (tophi), renal failure, and other related disorders, as described herein.

An engineered erythroid cell of the disclosure may comprise an exogenous polypeptide comprising a uricase, or variant thereof, wherein the uricase is derived from any source(s) known in the art, including mammalian, plant or microbial sources, as well as by recombinant technologies.

In some embodiments, the uricase, or uricase variant, is obtained from a fungal (including yeast) source. In some embodiments, the uricase is derived from Candida utilis (e.g., as described in Koyama et al., J. Biochem., 20:969-973, 1996 and U.S. Pat. No. 6,913,915, the contents of which are hereby incorporated herein by reference, and contained in pegsiticase (3Sbio/Selecta Biosciences, Inc.)). In some embodiments, the uricase is derived from the fungus Aspergillus flavus. In some embodiments, the uricase is the Aspergillus flavus uricase contained in rasburicase (ELITEK®; FASTURTEC®, Sanofi Genzyme).

Other fungal sources of uricases can include, for example, Candida (e.g., Candida tropicalis), Saccharomyces, Schizosaccaromyces, Emericella, Aspergillus (e.g., Aspergillus terreus, Aspergillus nidulas, Aspergillus niger, Aspergillus oryzae, Aspergillus tamarii, Aspergillus terricola, Aspergillus luchuensis, Aspergillus sydowi, Aspergillus wentii, Aspergillus fonsecaeus, Aspergillus clavatus, Aspergillus ustus, Aspergillus terreus and Aspergillus ochraceus), Neurospora (e.g., Neurospora crassa), Mucor (e.g., Mucor mucedo, Mucor hiemalis and Mucor racemosus), Rhizopus (e.g., Rhizopus oryzae and Rhizopus arrhizus), Absidia (e.g., Absidia glauca), Fusarium (e.g., Fusarium solani, Fusarium moniliforme, Fusarium coeruleum, Fusarium oxysporum, and Fusarium orthoceras), Alternaria (e.g., Alternaria tenuis), Penicillium (e.g., Penicillium freii, Penicillium frequentans, Penicillium granulatum, Penicillium griseum, Penicillium canescens, Penicillium spinulosum, Penicillium thomii, Penicillium waksmani, Penicillium raistrickii, Penicillium expansum, Penicillium purpurescens, Penicillium funiculosum, Penicillium spiculisporum, Penicillium velutinum, Penicillium purpurogenum, Penicillium lilacinum, and Penicillium rubrum), Cephalosporium, Stemphylium, Macrosporum (e.g., Macrosporium apiospermum), Stemphylium macrosporoideum, and Geotrichum candidum.

In some embodiments, the uricase, or uricase variant, is derived from a bacterium, such as bacterium belonging to Anthrobacter (e.g., Anthrobacter globiformis). In other embodiments, the uricase or uricase variant is derived from Streptomyces (e.g., Streptomyces cyanogenus, Streptomyces cellulosae and Streptomyces sulfureus), Bacillus (e.g., Bacillus subtilis, Bacillus megatherium, Bacillus thermocatenulatus, Bacillus fastidiosus, and Bacillus cereus), Pseudomonas aeruginosa, Cellumonas flavigena, or E. coli. Additional bacterial uricase sources include, but are not limited to, Deinococcus geothermalis uricase (NCBI Accession number WP_011525965), Deinococcus radiodurans uricase (NCBI Accession number WP_010887803), Granulicella tundricola uricase (NCBI Reference Sequence: WP_013581210.1), Solibacter usitatus uricase (NCBI Accession number WP_011682147), Terriglobus saanensis uricase (NCBI Accession number WP_013569963) and Kyrpidia tusciae uricase (NCBI Accession number ADG06709).

In some embodiments, the uricase, or uricase variant, is derived from a mammal, for example a pig, bovine, sheep, goat, baboon, rhesus macaque (Macaca mulatta), mouse (e.g., Mus musculus), rabbit, zebra fish (Danio rerio), or domestic animal. In one aspect of this embodiment, the uricase may comprise a porcine uricase, a bovine uricase, a porcine liver uricase, or a bovine liver uricase.

Alternatively, in some embodiments, the uricase, or uricase variant, is derived from an invertebrate, such as, for example, Drosophila, e.g., Drosophila melanogaster or Drosophila pseudoobscura or C. elegans.

The uricase, or uricase variant, may also comprise a plant uricase, for example, a uricase derived from soybean root nodule (Glycine max), leaves of chickpea (Cicer arietimum L.), broad bean (Vicia faba major L.), wheat (Triticum aestivum L.), Glycine max (soybean), rice (e.g., Oryza sativa subsp. japonica), Vigna unguiculata, or rust Puccinia recondite.

In some embodiments, the uricase is a chimeric uricase, in which portions of the uricase are derived from different sources. For example, a portion of the chimeric uricase may be obtained (e.g., derived) from one organism and one or more other portions of the chimeric uricase may be obtained (e.g., derived) from another organism. In some embodiments, a portion of the chimeric uricase is obtained from a pig and another portion of the chimeric uricase is obtained from a baboon. In some embodiments, the chimeric uricase may contain portions of porcine liver and/or baboon liver uricase. For example, the chimeric uricase may comprise all or a portion of a porcine uricase (Sus scrofa NP_999435) sequence, wherein the sequence contains the mutations R291K and T301S (PKS uricase). Alternatively, the uricase may comprise all or a portion of a baboon liver uricase (Papio hamadryas A36227) sequence in which tyrosine at amino acid residue 97 has been replaced by histidine, whereby the specific activity of the uricase may be increased by at least about 60% as compared to the wild-type uricase from which it was derived (i.e., lacking the amino acid substitution). In some embodiments, the chimeric uricase comprises a chimeric uricase described in U.S. Pat. No. 6,783,965 (the contents of which are hereby incorporated by reference herein), or comprises pegloticase (KRYSTEXXA®) (Horizon Rheumatology, Inc.). In some embodiments, the chimeric uricase comprises or consists of the amino acid sequence set forth in SEQ ID NO: 36.

In some preferred embodiments of the disclosure, the uricase (or variant thereof) comprises or consists of a uricase selected from those set forth in Table 1, below, including a uricase derived from Candida utilis, Aspergillus flavus, Arthrobacter globiformis, Baboon/porcine (chimera), Bacillus subtilis. Celluiomonas flavigena, Mus musculus, Glycine max (Soybean), Danio rerio (Zebrafish), Schizosaccharomyces pombe (Fission yeast), Macaca mulatta (Rhesus macaque), Oryza sativa subsp. japonica (Rice), Penicillium freii, Aspergillus niger, Drosophila melanogaster, or a baboon/porcine chimeric uricase. In some embodiments, the uricase comprises an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42.

TABLE 1
Exemplary Uricases

| SEQ ID NO | Uricase | Amino acid sequence |
|---|---|---|
| 1 | Candida utilis | MSTTLSSSTYGKDNVKFLKVKKDPQNPKKQEVMEA |
| | uricase | TVTCLLEGGFDTSYTEADNSSIVPTDTVKNTILVLAKT |
| | | TEIWPIERFAAKLATHFVEKYSHVSGVSVKIVQDRWVK |
| | | YAVDGKPHDHSFIHEGGEKRITDLYYKRSGDYKLSSAI |
| | | KDLTVLKSTGSMFYGYNKCDFTTLQPTTDRILSTDVDA |
| | | TWVWDNKKIGSVYDIAKAADKGIFDNVYNQAREITLTT |
| | | FALENSPSVQATMFNMATQILEKACSVYSVSYALPNKH |
| | | YFLIDLKWKGLENDNELFYPSPHPNGLIKCTVVRKEKT |
| | | KL |
| 2 | Aspergillus | SAVKAARYGKDNVRVYKVHKDEKTGVQTVYEMTVC |
| | flavus uricase | VLLEGEIETSYTKADNSVIVATDSIKNTIYITAKQNPV |
| | | TPPELFGSILGTHFIEKYNHIHAAHVNIVCHRWTRMDI |
| | | DGKPHPHSFIRDSEEKRNVQVDVVEGKGIDIKSSLSGL |
| | | TVLKSTNSQFWGFLRDEYTTLKETWDRILSTDVDATWQ |
| | | WKNFSGLQEVRSHVPKFDATWATAREVTLKTFAEDNSA |
| | | SVQATMYKMAEQILARQQLIETVEYSLPNKHYFEIDLS |
| | | WHKGLQNTGKNAEVFAPQSDPNGLIKCTVGRSSLKSKL |
| 3 | Arthrobacter | MTATAETSTGTKVVLGQNQYGKAEVRLVKVTRNTARHE |
| | globiformis | IQDLNVTSQLRGDFEAAHTAGDNAHVVATDTQKNTVYA |
| | uricase | FARDGFATTEEFLLRLGKHFTEGFDWVTGGRWAAQQFF |
| | | WDRINDHDHAFSRNKSEVRTAVLEISGSEQAIVAGIEG |
| | | LTVLKSTGSEFHGFPRDKYTTLQETTDRILATDVSARW |
| | | RYNTVEVDFDAVYASVRGLLLKAFAETHSLALQQTMYE |
| | | MGRAVIETHPEIDEIKMSLPNKHHFLVDLQPFGQDN |
| | | PNEVFYAADRPYGLIEATIQREGSRADHPIWSNIAGFC |
| 4 | Baboon/porcine | MAHYRNDYKKNDEVEFVRTGYGKDMIKVLHIQRDGKYH |
| | chimeric | SIKEVATSVQLTLSSKKDYLHGDNSDVIPTDTIKNTVN |
| | uricase | VLAKFKGIKSIETFAVTICEHFLSSFKHVIRAQVYVEE |
| | | VPWKRFEKNGVKHVHAFIYTPTGTHFCEVEQIRNGPPV |
| | | IHSGIKDLKVLKTTQSGFEGFIKDQFTTLPEVKDRCFA |
| | | TQVYCKWRYHQGRDVDFEATWDTVRSIVLQKFAGPYDK |
| | | GEYSPSVQKTLYDIQVLSLSRVPEIEDMEISLPNIHYF |
| | | NIDMSKMGLINKEEVLLPLDNPYGKITGTVKRKLSSRL |
| 5 | Bacillus | MFTMDDLNQM DTQTLTDTLG SIFEHSSWIA |
| | subtilis | ERSAALRPFS SLSDLHRKMT GIVKAADRET |
| | uricase | QLDLIKKHPR LGTKKTMSDD SVREQQNAGL |
| | | GKLEQQEYEE FLMLNEHYYD RFGFPFILAV |
| | | KGKTKQDIHQ ALLARLESER ETEFQQALIE |
| | | IYRIARFRLA DIITEKGETQ MKRTMSYGKG |
| | | NVFAYRTYLK PLTGVKQIPE SSFAGRDNTV |
| | | VGVDVTCEIG GEAFLPSFTD GDNTLVVATD |
| | | SMKNFIQRHL ASYEGTTTEG FLHYVAHRFL |
| | | DTYSHMDTIT LTGEDIPFEA MPAYEEKELS |
| | | TSRLVFRRSR NERSRSVLKA ERSGNTITIT |
| | | EQYSEIMDLQ LVKVSGNSFV GFIRDEYTTL |
| | | PEDGNRPLFV YLNISWQYEN TNDSYASDPA |
| | | RYVAAEQVRD LASTVFHELE TPSIQNLIYH |
| | | IGCRILARFP QLTDVSFQSQ NHTWDTVVEE |
| | | IPGSKGKVYT EPRPPYGFQH FTVTREDAEK |
| | | EKQKAAEKCR SLKA |
| 6 | Cellulomonas | MAVGDVVLGA NQYGKAEVRL VRVTRDTPVH |
| | flavigena | EIEDLTVTTQ LRGDFAACHT TGDNAHVVAT |
| | uricase | DTQKNTVYAF ARTHGVGSPE AFLLRLARHF |
| | | VDGFDQVTGG RFAADVHAWD RIAVDGKPHD |
| | | HAFVRTGAGT RRAVVLVDGD DVHVVSGFTG |
| | | ATVLKSTGSE FWGFPRDRYT TLAETKDRIL |
| | | ATSVTAWWRW TTPDVDVEAR YPVVKDLLLS |
| | | TFAQVHSLAL QHTIFEMGRA VLEACDDVAE |
| | | VRLSCPNKHH LLVDLEPFGL ENPGEVFHAA |
| | | DRPYGLIEAA VHREGDPPVP HVWASVPGFV |
| 7 | Mus musculus | MAHYHDNYGK NDEVEFVRTG YGKDMVKVLH |
| | uricase | IQRDGKYHSI KEVATSVQLT LRSKKDYLHG |
| | | DNSDIIPTDT IKNTVHVLAK LRGIRNIETF |
| | | AMNICEHFLS SFNHVTRAHV YVEEVPWKRF |
| | | EKNGIKHVHA FIHTPTGTHF CEVEQMRNGP |
| | | PVIHSGIKDL KVLKTTQSGF EGFLKDQFTT |
| | | LPEVKDRCFA TQVYCKWRYQ RRDVDFEAIW |
| | | GAVRDIVLQK FAGPYDKGEY SPSVQKTLYD |
| | | IQVLSLSQLP EIEDMEISLP NIHYFNIDMS |
| | | KMGLINKEEV LLPLDNPYGK ITGTVKRKLP |
| | | SRL |
| 8 | Glycine max | MAQQEVVEGF KFEQRHGKER VRVARVWKTR |
| | (Soybean) | QGQHFIVEWR VGITLFSDCV NSYLRDDNSD |
| | uricase | IVATDTMKNT VYAKAKECSD ILSAEEFAIL |
| | | LAKHFVSFYQ KVTGAIVNIV EKPWERVTVD |
| | | GQPHEHGFKL GSEKHTTEAI VQKSGSLQLT |
| | | SGIEGLSVLK TTQSGFVNFI RDKYTALPDT |
| | | RERMVATEVT ALWRYSYESL YSLPQKPLYF |
| | | TEKYQEVKKV LADTFFGPPK GGVYSPSVQN |
| | | TLYLMAKATL NRFPDIAYVS LKLPNLHFIP |
| | | VNISNQDGPI VKFEDDVYLP TDEPHGSIQA |
| | | SLSRLWSKL |
| 9 | Danio rerio | MATTSNQNVE FVRTGYGKNM VKVLHIRREG |
| | (Zebra fish) | NHHHIIELIA NVQLTLKTRK DYLTGDNSDI |
| | uricase | IPTDTVKNTV HALAKLKGIK SIESFALDIC |
| | | EHFLTAFNHV TRVKVNIDEV PWKRLEKNGV |
| | | EHNHAFIHCP EALRFCEAEQ YLSKTPVVHS |
| | | GLKDMKVLKT TQTGFEGFLR DRFTTLTDAK |
| | | DRFFCTSVYA RWRYNTINVA FDAAWKAVKD |
| | | TVIQKFAGPY DRGEYSPSVQ KTLYDTQLLV |
| | | LDRIPEVEEI EIIMPNQHYF VIDMTKIGLS |
| | | NKDEVYLPLD NPSGNITGTV CRKPRARM |
| 10 | Schizo- | MSETTYVKQC AYGKTLVRFM KKDICPKTKT |
| | saccharomyces | HTVYEMDVQS LLTGELEESY TKADNSIVVP |
| | pombe | TDTQKNTIYV FAKNNDVSVP EVFAAKLAKH |
| | (Fission yeast) | FVDKYKHIHG AALDITITPW TRMEVQGKPH |
| | uricase | SHSFIRNPGE TRKTHVVFSE GKGFDVVSSL |
| | | KDVLVLKSTG SGFTNFHKCE FTTLPEVTDR |
| | | IFSTSIDCNY TFKHFDTFEE LAGFDFNSIY |
| | | EKVKEITLET FALDDSESVQ ATMYKMADTI |
| | | INTYPAINEV YYALPNKHYF EINLAPFNID |
| | | NLGSNCSLYQ PQAYPSGYIT CTVARK |
| 11 | Macaca mulatta | MADYHNNYKK NDELEFVRTG YGKDMVKVLH |
| | (Rhesus | IQRDGKYHSI KEVATSVQLT LSSKKDYLHG |
| | macaque) | DNSDIIPTDT IKNTVHVLAK FKGIKSIEAF |
| | uricase | GVNICEYFLS SFNHVIRAQV YVEEIPWKRL |
| | | EKNGVKHVHA FIHTPTGTHF CEVEQLRSGP |
| | | PVIHSGTKDL KVLKTTQSGF EGFIKDQFTT |
| | | LPEVKDRCFA TQVYCKWRYH QCRDVDFEAT |
| | | WGTIRDLVLE KFAGPYDKGE YSPSVQKTLY |
| | | DIQVLSLSRV PEIEDMEISL PNIHYFNIDM |
| | | SKMGLINKEE VLLPLDNPYG KITGTVKRKL |
| | | SSRL |
| 12 | Oryza sativa | MADRLELQGR HGKSRVRVSR VWRRPAAAGG |
| | subsp. japonica | HVIVEWNVAV SVVSDCLPSY TSDDNSAIVA |
| | (Rice) uricase | TDSIKNTVYV KAKECTEIVS MEEFAVILGR |
| | | HFTSLYPQVS EATVTIAERP WERVVVDGKP |
| | | HSHGFKLGVE KHVTEVIVKK SGNLLINSGI |
| | | QGYSLLKTTQ SGFEKFVRDR YTLLPDTRER |
| | | IVATEVTAWW RYPFEHVSQI PSKSFCFTQR |
| | | YQDVKKVLAD TFFGPPDVGV YSPSVQNTLY |
| | | LMAKEVLNRF PDIASVQLRM PNLHFIPVNL |
| | | GNKENPGLVK FADDVYLPTD EPHGTIEATV |
| | | SRPKSKL |
| 13 | Penicillium | MILRSANSLH SRSNKYSFKL LDNTIFLPFS |
| | freii uricase | ILYFFFSSIF QRYHSKFKMS ALAAARYGKD |
| | | NVRVCKVHRD EKTGIQTVVE MTVCVLLEGD |
| | | IETSYTKADN SVVVATDSIK NTIFITAKQN |
| | | PVTPPELFGS ILGTHFIEKY SHIHAAHVNI |
| | | VTHRWVRLDI DGKPHPHSFI KPGSETRNVQ |
| | | VDVIEGKGID INSSIKGLTV LKSTGSQFWG |
| | | FVRDEYTTLK ETWDRLLSTD VAANWQWRRF |
| | | TGLADVKTHS EKFNAAWEAA RAITLKTFAD |
| | | DNSASVQATM YKMGEQILAA VPLLDTVEYA |
| | | LPNIHFFEVD LSWHKGIKNT GKDAEVYAPQ |
| | | SNPNGLIKCT VGRAGQKAKL |
| 14 | Aspergillus | MSSPVTAARY GKDNVRVYKV HRDEKTGVQT |
| | niger uricase | VVEMTVCVLL EGDIDTSYTK ADNSVIVATD |
| | | SIKNTIYITA KQNPVTPPEL FGSILGSHFI |
| | | TKYNHIHAAH VNIITHRWTR MTIDGKPHPH |
| | | SFLRDGEETR NVQVDVVEGK GVDITSSLAK |
| | | LTVLKSTNSQ FWGFLRDEYT TLPETWDRIL |
| | | STDVDASWQW RRFSGLDEVR ATVPHFDATW |
| | | AAAREITLKT FAEDNSASVQ NTMYKMAEQI |
| | | LARQSLLETV EYSLPNKHYF EVDLSWHKGL |
| | | KNTGKDAEVY APQTNPNGLI KCTVGRNTKA |
| | | KL |
| 15 | Drosophila | MFATPLRQPA AANHQTPKNS AGMDEHGKPY |
| | melanogaster | QYEITDHGYG KDAVKVLHVS RNGPVHAIQE |
| | uricase | FEVGTHLKLY SKKDYYQGNN SDIVATDSQK |
| | | NTVYLLAKKH GIESPEKFAL LLARHFINKY |
| | | SHVEEAHVHV EAYPWQRVCQ EETRTNVNGK |
| | | CENGVQGNCD FSSIDNRSLH NHAFIFTPTA |
| | | LHYCDVVIRR TDPKQTVITG IKGLRVLKTT |
| | | QSSFVNFVND EFRSLPDQYD RIFSTVVDCS |
| | | WEYSDTENLD FLRAWQTVKN IIIRNFAGDP |
| | | QVGVSSPSVQ HTLYLSERQV LDVLPQVSVI |
| | | SMTMPNKHYF NFDTKPFQKI APGDNNEVFI |
| | | PVDKPHGTIY AQLARKNINS HL |
| 34 | Candida utilis | MSTTLSSSTYGKDNVKFLKVKKDPQNPKKQEVMEATVT |
| | uricase | CLLEGGFDTSYTEADNSSIVPTDTVKNTILVLAKTTEI |
| | | WPIERFAAKLATHFVEKYSHVSGVSVKIVQDRWVKYAV |
| | | DGKPHDHSFIHEGGEKRITDLYYKRSGDYKLSSAIKDL |
| | | TVLKSTGSMFYGYNKCDFTTLQPTTDRILSTDVDATWV |
| | | WDNKKIGSVYDIAKAADKGIFDNVYNQAREITLTTFAL |
| | | ENSPSVQATMFNMATQILEKACSVYSVSYALPNKHYFL |
| | | IDLKWKGLENDNELFYPSPHPNGLIKCTVVRKE |
| 35 | Aspergillus | SAVKAARYGKDNVRVYKVHKDEKTGVQTVYEMTVCVLL |
| | flavus uricase | EGEIETSYTKADNSVIVATDSIKNTIYITAKQNPVTPP |
| | | ELFGSILGTHFIEKYNHIHAAHVNIVCHRWTRMDIDGK |
| | | PHPHSFIRDSEEKRNVQVDVVEGKGIDIKSSLSGLTVL |
| | | KSTNSQFWGFLRDEYTTLKETWDRILSTDVDATWQWKN |
| | | FSGLQEVRSHVPKFDATWATAREVTLKTFAEDNSASVQ |
| | | ATMYKMAEQILARQQLIETVEYSLPNKHYFEIDLSWHK |
| | | GLQNTGKNAEVFAPQSDPNGLIKCTVGRSSL |
| 36 | Baboon/porcine | MAHYRNDYKKNDEVEFVRTGYGKDMIKVLHIQRDGKYH |
| | chimeric | SIKEVATSVQLTLSSKKDYLHGDNSDVIPTDTIKNTVN |
| | uricase | VLAKFKGIKSIETFAVTICEHFLSSFKHVIRAQVYVEE |
| | | VPWKRFEKNGVKHVHAFIYTPTGTHFCEVEQIRNGPPV |
| | | IHSGIKDLKVLKTTQSGFEGFIKDQFTTLPEVKDRCFA |
| | | TQVYCKWRYHQGRDVDFEATWDTVRSIVLQKFAGPYDK |
| | | GEYSPSVQKTLYDIQVLSLSRVPEIEDMEISLPNIHYF |
| | | NIDMSKMGLINKEEVLLPLDNPYGKITGTVKRKL |
| 37 | Bacillus | MFTMDDLNQMDTQTLTDTLGSIFEHSSWIAERSAALRP |
| | subtilis | FSSLSDLHRKMTGIVKAADRETQLDLIKKHPRLGTKKT |
| | uricase | MSDDSVREQQNAGLGKLEQQEYEEFLMLNEHYYDRFGF |
| | | PFILAVKGKTKQDIHQALLARLESERETEFQQALIEIY |
| | | RIARFRLADIITEKGETQMKRTMSYGKGNVFAYRTYLK |
| | | PLTGVKQIPESSFAGRDNTVVGVDVTCEIGGEAFLPSF |
| | | TDGDNTLVVATDSMKNFIQRHLASYEGTTTEGFLHYVA |
| | | HRFLDTYSHMDTITLTGEDIPFEAMPAYEEKELSTSRL |
| | | VFRRSRNERSRSVLKAERSGNTITITEQYSEIMDLQLV |
| | | KVSGNSFVGFIRDEYTTLPEDGNRPLFVYLNISWQYEN |
| | | TNDSYASDPARYVAAEQVRDLASTVFHELETPSIQNLI |
| | | YHIGCRILARFPQLTDVSFQSQNHTWDTVVEEIPGSKG |
| | | KVYTEPRPPYGFQHFTVTREDAEKEKQKAAEKCR |
| 38 | Mus musculus | MAHYHDNYGKNDEVEFVRTGYGKDMVKVLHIQRDGKYH |
| | uricase | SIKEVATSVQLTLRSKKDYLHGDNSDIIPTDTIKNTVH |
| | | VLAKLRGIRNIETFAMNICEHFLSSFNHVTRAHVYVEE |
| | | VPWKRFEKNGIKHVHAFIHTPTGTHFCEVEQMRNGPPV |
| | | IHSGIKDLKVLKTTQSGFEGFLKDQFTTLPEVKDRCFA |
| | | TQVYCKWRYQRRDVDFEAIWGAVRDIVLQKFAGPYDKG |
| | | EYSPSVQKTLYDIQVLSLSQLPEIEDMEISLPNIHYFN |
| | | IDMSKMGLINKEEVLLPLDNPYGKITGTVKRKL |
| 39 | Glycine max | MAQQEVVEGFKFEQRHGKERVRVARVWKTRQGQHFIVE |
| | (Soybean) | WRVGITLFSDCVNSYLRDDNSDIVATDTMKNTVYAKAK |
| | uricase | ECSDILSAEEFAILLAKHFVSFYQKVTGAIVNIVEKPW |
| | | ERVTVDGQPHEHGFKLGSEKHTTEAIVQKSGSLQLTSG |
| | | IEGLSVLKTTQSGFVNFIRDKYTALPDTRERMVATEVT |
| | | ALWRYSYESLYSLPQKPLYFTEKYQEVKKVLADTFFGP |
| | | PKGGVYSPSVQNTLYLMAKATLNRFPDIAYVSLKLPNL |
| | | HFIPVNISNQDGPIVKFEDDVYLPTDEPHGSIQASLSR |
| | | L |
| 40 | Macaca mulatta | MADYHNNYKKNDELEFVRTGYGKDMVKVLHIQRDGKYH |
| | (Rhesus | SIKEVATSVQLTLSSKKDYLHGDNSDIIPTDTIKNTVH |
| | macaque) | VLAKFKGIKSIEAFGVNICEYFLSSFNHVIRAQVYVEE |
| | uricase | IPWKRLEKNGVKHVHAFIHTPTGTHFCEVEQLRSGPPV |
| | | IHSGTKDLKVLKTTQSGFEGFIKDQFTTLPEVKDRCFA |
| | | TQVYCKWRYHQCRDVDFEATWGTIRDLVLEKFAGPYDK |
| | | GEYSPSVQKTLYDIQVLSLSRVPEIEDMEISLPNIHYF |
| | | NIDMSKMGLINKEEVLLPLDNPYGKITGTVKRKL |
| 41 | Oryza sativa | MADRLELQGRHGKSRVRVSRVWRRPAAAGGHVIVEWNV |
| | subsp. japonica | AVSVVSDCLPSYTSDDNSAIVATDSIKNTVYVKAKECT |
| | (Rice) uricase | EIVSMEEFAVILGRHFTSLYPQVSEATVTIAERPWERV |
| | | VVDGKPHSHGFKLGVEKHVTEVIVKKSGNLLINSGIQG |
| | | YSLLKTTQSGFEKFVRDRYTLLPDTRERIVATEVTAWW |
| | | RYPFEHVSQIPSKSFCFTQRYQDVKKVLADTFFGPPDV |
| | | GVYSPSVQNTLYLMAKEVLNRFPDIASVQLRMPNLHFI |
| | | PVNLGNKENPGLVKFADDVYLPTDEPHGTIEATVSRP |
| 42 | Penicillium | MILRSANSLHSRSNKYSFKLLDNTIFLPFSILYFFFSS |
| | i uricase | IFQRYHSKFKMSALAAARYGKDNVRVCKVHRDEKTGIQ |
| | | TVVEMTVCVLLEGDIETSYTKADNSVVVATDSIKNTIF |
| | | ITAKQNPVTPPELFGSILGTHFIEKYSHIHAAHVNIVT |
| | | HRWVRLDIDGKPHPHSFIKPGSETRNVQVDVIEGKGID |
| | | INSSIKGLTVLKSTGSQFWGFVRDEYTTLKETWDRLLS |
| | | TDVAANWQWRRFTGLADVKTHSEKFNAAWEAARAITLK |
| | | TFADDNSASVQATMYKMGEQILAAVPLLDTVEYALPNI |
| | | HFFEVDLSWHKGIKNTGKDAEVYAPQSNPNGLIKCTVG |
| | | RAGQ |

In some embodiments, the uricase comprises the Candida utilis uricase comprising the amino acid sequence set forth in SEQ ID NO:1. In some embodiments, the uricase comprises the Aspergillus flavus uricase comprising the amino acid sequence set forth in SEQ ID NO:2. In some embodiments, the uricase comprises the Arthrobacter globiformis uricase comprising the amino acid sequence set forth in SEQ ID NO:3. In some embodiments, the uricase comprises the baboon/porcine chimeric uricase comprising the amino acid sequence set forth in SEQ ID NO:4. In some embodiments, the uricase comprises the Bacillus subtilis uricase comprising the amino acid sequence set forth in SEQ ID NO:5. In some embodiments, the uricase comprises the Cellulomortas flavigena uricase comprising the amino acid sequence set forth in SEQ ID NO:6. In some embodiments, the uricase comprises the Mus musculus uricase comprising the amino acid sequence set forth in SEQ ID NO:7. In some embodiments, the uricase comprises the Glycine max (Soybean) uricase comprising the amino acid sequence set forth in SEQ ID NO:8. In some embodiments, the uricase comprises the Danio rerio (Zebrafish) uricase comprising the amino acid sequence set forth in SEQ ID NO:9. In some embodiments, the uricase comprises the Schizosaccharomyces pombe (Fission yeast) uricase comprising the amino acid sequence set forth in SEQ ID NO:10. In some embodiments, the uricase comprises the Macaca mulatta (Rhesus macaque) uricase comprising the amino acid sequence set forth in SEQ ID NO:11. In some embodiments, the uricase comprises the Oryza sativa subsp. japonica (Rice) uricase comprising the amino acid sequence set forth in SEQ ID NO:12. In some embodiments, the uricase comprises the Penicillium freii uricase comprising the amino acid sequence set forth in SEQ ID NO:13. In some embodiments, the uricase comprises the Aspergillus niger uricase comprising the amino acid sequence set forth in SEQ ID NO:14. In some embodiments, the uricase comprises the Drosophila melanogaster uricase comprising the amino acid sequence set forth in SEQ ID NO:15. In some embodiments, the uricase comprises the Candida utilis uricase comprising the amino acid sequence set forth in SEQ ID NO: 34. In some embodiments, the uricase comprises the Aspergillus flavus uricase comprising the amino acid sequence set forth in SEQ ID NO: 35. In some embodiments, the uricase comprises the baboon/porcine chimeric uricase comprising the amino acid sequence set forth in SEQ ID NO: 36. In some embodiments, the uricase comprises the Bacillus subtilis uricase comprising the amino acid sequence set forth in SEQ ID NO: 37. In some embodiments, the uricase comprises the Mus musculus uricase comprising the amino acid sequence set forth in SEQ ID NO: 38. In some embodiments, the uricase comprises the Glycine max uricase comprising the amino acid sequence set forth in SEQ ID NO: 39. In some embodiments, the uricase comprises the Macaca mulatta uricase comprising the amino acid sequence set forth in SEQ ID NO: 40. In some embodiments, the uricase comprises the Oryza sativa uricase comprising the amino acid sequence set forth in SEQ ID NO: 41. In some embodiments, the uricase comprises the Penicillium freii uricase comprising the amino acid sequence set forth in SEQ ID NO: 42.

In some embodiments, the uricase comprises a variant of a wild-type uricase having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42. In some embodiments, the uricase variant possesses a function of the uricase from which it was derived (e.g., the ability to catalyze the oxidation of uric acid (urate) to 5-hydroxyisourate).

In a particular embodiment, the uricase comprises an amino acid sequence that is at least about 95% identical (e.g., 96%, 97%, 98%, 99% or 100% identical) to an amino acid sequence set forth in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 34, SEQ ID NO: 35X, SEQ ID NO: 36, SEQ ID NO: 37, SEQ ID NO: 38, SEQ ID NO: 39, SEQ ID NO: 40, SEQ ID NO: 41, or SEQ ID NO: 42.

In a particular embodiment, the uricase consists of the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42.

In some embodiments, the uricase, or variant thereof, is engineered to delete a peroxisome targeting signal (e.g., a native peroxisome targeting signal). In some embodiments, the uricase, or variant thereof, lacks a peroxisome targeting signal.

In some embodiments, the uricase comprises a fragment of a wild-type uricase. In some embodiments, the fragment of the uricase comprises at least 10, at least 20, at least 30, at least 40, at least 50, at least 60, at least 70, at least 80, at least 90, at least 100, at least 110, at least 120, at least 130, at least 140, at least 150 or at least 160 amino acid residues (e.g., contiguous amino acid residues) of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42, or a variant thereof. In some embodiments, the fragment of the uricase comprises fewer than 20, fewer than 30, fewer than 40, fewer than 50, fewer than 60, fewer than 70, fewer than 80, fewer than 90, fewer than 100, fewer than 110, fewer than 120, fewer than 130, fewer than 140, fewer than 150 or fewer than 160 amino acid residues (e.g. contiguous amino acid residues) of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42, or a variant thereof. In some embodiments, fragments or variants of the uricase retain at least 50%, at least 55%, at least 60%, at least 65%, at least 70%, at least 75%, at least 80%, at least 85%, at least 90%, at least 95%, at least 98% or at least 99% of the function (e.g., the ability to catalyze the oxidation of uric acid (urate) to 5-hydroxyisourate) as compared to the uricase from which it was derived.

In general, a variant uricase, from any origin, may be produced, for example, to enhance production of the protein in an engineered cell, to improve turnover/half-life of the protein or mRNA encoding the protein, and/or to modulate (enhance or reduce) the enzymatic activity of the uricase. The uricase, whatever the source, may also be in a form that is truncated, either at the amino terminal, or at the carboxyl terminal, or at both terminals.

In some embodiments, the invention provides an engineered erythroid cell (e.g. an engineered erythroid precursor cell) comprising a nucleic acid sequence encoding a uric acid degrading polypeptide as described herein. In some embodiments, the invention provides an engineered erythroid cell prepared by using a nucleic acid sequence encoding a uric acid degrading polypeptide (e.g. a uricase) as described herein. In some embodiments, the nucleic acid sequence encodes a uricase as described herein.

In some embodiments, the uricase is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the corresponding wild-type uricase nucleic acid (from any source) that encodes a protein that possesses a function of a uricase described herein, e.g., the encoded protein can catalyze the oxidation of uric acid (urate) to 5-hydroxyisourate.

In some embodiments, the nucleic acid sequence encoding the uric acid degrading polypeptide (e.g., uricase) or a component thereof, is codon optimized (e.g., codon optimized for expression in a human cell). For example, in some embodiments, the nucleic acid sequence encoding the uricase is codon optimized. In other embodiments, the nucleic acid sequence encoding the uric acid degrading polypeptide (e.g., uricase) or a component thereof is not codon optimized. For example, in some embodiments, the nucleic acid sequence encoding the uricase is not codon optimized.

In some embodiments, the uric acid degrading polypeptide is not uricase. In some embodiments, the uric acid degrading polypeptide is not an Aspergillus flavus uricase. In some embodiments, the uric acid degrading polypeptide is not an Aspergillus flavus uricase comprising an amino acid sequence at least 80% identical (e.g., at least 80%, 85%, 90%, 95%, 96%, 97%, 98%, or 99%) to SEQ ID NO: 2 or SEQ ID NO: 35. In some embodiments, the uric acid degrading polypeptide is not an Aspergillus flavus uricase consisting of SEQ ID NO: 2 or SEQ ID NO: 35.

In some embodiments, the exogenous polypeptide is a fusion polypeptide comprising a uricase, or a variant thereof, linked to a heterologous protein sequence (e.g., via a linker).

### Uric Acid Transporters

In one aspect, the disclosure provides an engineered erythroid cell comprising a first exogenous polypeptide comprising a uric acid transporter, or a variant thereof. In some embodiments, the disclosure provides an engineered erythroid cell comprising at least one (e.g., one, two, three, four, or more) exogenous polypeptide comprising a uric acid transporter. In some embodiments, the disclosure provides an engineered erythroid cell comprising more than one exogenous polypeptide, each comprising a uric acid transporter.

In another aspect, the disclosure provides an erythroid cell engineered to degrade uric acid, wherein the cell comprises a first exogenous polypeptide comprising a uric acid degrading polypeptide, e.g., uricase, or a variant thereof, and further comprises a second exogenous polypeptide comprising a uric acid transporter, or a variant thereof.

In yet another aspect, the disclosure provides an engineered cell comprising at least one (e.g., one two, three, four, or more) exogenous polypeptide, wherein the exogenous polypeptide comprises both a uric acid degrading polypeptide (or a variant thereof) and a uric acid transporter (or a variant thereof). Without wishing to be bound by any particular theory, engineered cells comprising an exogenous polypeptide that comprises both a uric acid degrading polypeptide and a uric acid transporter may improve turnover of uric acid (e.g., the catalysis of uric acid) by facilitating the transfer of uric acid from the uric acid transporter to the uric acid degrading polypeptide, thereby microcompartmentalizing the channeling and catalysis of uric acid.

Uric acid transporters regulate uric acid transport in the kidney and thereby regulate plasma uric acid levels (see, So and Thorens, 2010, J. of Clin. Investigation, 120(6):1791, the contents of which are hereby incorporated herein by reference).

In some embodiments, an erythroid cell of the disclosure comprises an exogenous polypeptide comprising a uric acid transporter selected from the group consisting of URAT1, GLUT9, OAT4, OAT1, OAT3, Gal-9, ABCG2, SLC34A2, MRP4, OAT2, NPT4, NPT1, and MCT9. In some embodiments, the uric acid transporter is a human uric acid transporter.

URAT1 (also referred to as uric acid transporter 1; SLC22A12; solute carrier family 22 member 12), is a member of the organic anion transport (OAT) family. It is a 12-trans-membrane domain-containing protein found in the apical membrane of proximal tubule epithelial cells and transports uric acid in exchange for CL⁻ or organic anions (So and Thorens, 2010, J. of Chin. Investigation, 120(6):1791). It is a high-affinity uric acid/anion exchanger that is responsible for the reabsorption of filtered uric acids from urine. The loss-of-function mutation of the SLC22A12 gene (G774A) results in renal hypouricemia and is a suppressing factor for the development of gout and hyperurecemia (Taniguchi et al., 2005, Arthritis and Rheumatology, 52(8)).

GLUT9 (also referred to as SLC2A9; Solute Carrier Family 2 Member 9), is a protein having 12-transmembrane domains, a large extracellular loop between the first and second transmembrane domains, and both amino- and carboxyterminal ends on the cytoplasmic side. GLUT9 exists as two alternatively spliced variants, GLUT9a and GLUT9b, that encode different amino terminal cytoplasmic tails. GLUT9 is responsible for the voltage-driven efflux of uric acid from cells to the blood. Human GLUT9a has 540 amino acids and is encoded by 12 exons, and human GLUT9b has 512 amino acids and is encoded by 13 exons. In both humans and mice, GLUT9b expression is restricted to liver and kidney and GLUT9a is broadly distributed in liver, kidney, intestine, leukocytes, and chondrocytes (Mobasheri et al. 2005 Cell Biol. Int. 29(4); 249; Shikhman et al. 2001 J. Immunol. 167(12):7001).

OAT4 (also referred to as organic anion transporter 4; SLC22A9; Solute Carrier Family 22 Member 11), is a multi-specific anion transporter present in the apical membrane of epithelial cells from the proximal tubule, and is involved in luminal uric acid reabsorption (Cha et al. 2000 J. Biol. Chem; 275(6):4507-4512).

OAT1 (also referred to as organic anion transporter 1; St.C22A6; Solute Carrier Family 22 Member 6), is an organic anion and uric acid transporter that functions as a uric acid/dicarboxylate exchanger and is found in the basolateral side of epithelial cells from the proximal tubule.

OAT2 (also referred to as organic anion transporter 2, organic acid transporter 11, Solute Carrier Family 22, and SLC22A7), is an organic anion and uric acid transporter which appears to be localized to the basolateral membrane of the kidney.

OAT3 (also referred to as organic anion transporter 3; SLC22A8: Solute Carrier Family 22 Member 8), is also an organic anion and uric acid transporter that functions as a uric acid/dicarboxylate exchanger. Like OAT1, OAT3 is found in the basolateral side of epithelial cells from the proximal tubule.

Gal-9 (also referred to as galectin-9; UAT; Lectin, Galactoside-Binding, Soluble, 9), is a multifunctional protein that can function as a uric acid channel/transporter, a regulator of thymocyte-epithelial cell interactions, a tumor antigen, an eosinophil chemotactic factor, and a mediator of apoptosis. Cial-9 is expressed in a wide variety of tissues and is present in at least three isoforms (Lipkowitz et al., 2002 Glycoconj J.,19(7-9):491-8).

ABCG2 (also referred to as ATP-binding cassette sub-family G member 2), is a multidrug resistance protein expressed in the apical membrane of proximal collecting duct cells and functions as a uric acid efflux transporter.

SLC34A2 (also referred to as sodium-dependent phosphate transport protein 1; Solute Carrier Family 34 Member 2)

MRP4 (also referred to as multidrug resistance-associated protein 4; ABCC4), is a multidrug resistance protein found in the apical membrane of proximal tubule epithelial cells. It is believed to control ATP-dependent uric acid extrusion from cells into the tubule lumen and contribute to uric acid excretion.

NPT1 (also referred to Na(+)/PI cotransporter 1, Solute Carrier Family 17 Member 1, SLC17A1, and NAPI-1) is a membrane protein that transports various substrates, including uric acid located in the renal proximal tubule in humans.

NPT4 (also referred to as Na(+)/PI cotransporter 4, Solute Carrier Family 17 Member 3, SLC17A3, and GOUT4) is a voltage-driven protein transporter that excretes intracellular uric acid and organic anions from the blood into renal tubule cells.

MCT9 (also referred to as monocarboxylate transporter 9, Solute Carrier Family 16 Member 9, SLC16A9, and) is a proton-linked monocarboxylate transporter that transports uric acid.

In some embodiments, the engineered erythroid cell provided herein comprises at least one exogenous polypeptide comprising a uric acid transporter selected from the group consisting of URAT1, GLUT9, OAT4, OAT1, OAT3, Gal-9, ABCG2, SLC34A2, MRP4, OAT2, NPT4, NPT1, and MCT9, or a variant thereof. In some embodiments, the uric acid transporter is derived from or is a human uric acid transporter.

In some preferred embodiments, the erythroid cell of the disclosure comprises an exogenous polypeptide comprising a uric acid transporter selected from those set forth in Table 2, below, comprising or consisting of the amino acid sequence of any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 44, SEQ ID NO: 45, SEQ ID NO: 46, or SEQ ID NO: 47, or a variant thereof.

TABLE 2
Exemplary Uric Acid Transporters

| | Uric acid | |
|---|---|---|
| SEQ ID NO: | transporter | Amino acid sequence |
| 16 | Homo | MAFSELLDLVGGLGRFQVLQTMALMVSIMWLCTQSMLENFS |
| | sapiens | AAVPSHRCWAPLLDNSTAQASILGSLSPEALLAISIPPGPN |
| | URAT1 | QRPHQCRRFRQPQWQLLDPNATATSWSEADTEPCVDGWVYD |
| | | RSIFTSTIVAKWNLVCDSHALKPMAQSIYLAGILVGAAACG |
| | | PASDRFGRRLVLTWSYLQMAVMGTAAAFAPAFPVYCLFRFL |
| | | LAFAVAGVMMNTGTLLMEWTAARARPLVMTLNSLGFSFGHG |
| | | LTAAVAYGVRDWTLLQLVVSVPFFLCFLYSWWLAESARWLL |
| | | TTGRLDWGLQELWRVAAINGKGAVQDTLTPEVLLSAMREEL |
| | | SMGQPPASLGTLLRMPGLRFRTCISTLCWFAFGFTFFGLAL |
| | | DLQALGSNIFLLQMFIGVVDIPAKMGALLLLSHLGRRPTLA |
| | | ASLLLAGLCILANTLVPHEMGALRSALAVLGLGGVGAAFTC |
| | | ITIYSSELFPTVLRMTAVGLGQMAARGGAILGPLVRLLGVH |
| | | GPWLPLLVYGTVPVLSGLAALLLPETQSLPLPDTIQDVQNQ |
| | | AVKKATHGTLGNSVLKSTQF |
| 17 | Homo | MARKQNRNSKELGLVPLTDDTSHAGPPGPGRALLECDHLRS |
| | sapiens | GVPGGRRRKDWSCSLLVASLAGAFGSSFLYGYNLSVVNAPT |
| | Glut9 | PYIKAFYNESWERRHGRPIDPDTLTLLWSVTVSIFAIGGLV |
| | | GTLIVKMIGKVLGRKHTLLANNGFAISAALLMACSLQAGAF |
| | | EMLIVGRFIMGIDGGVALSVLPMYLSEISPKEIRGSLGQVT |
| | | AIFICIGVFTGQLLGLPELLGKESTWPYLFGVIVVPAVVQL |
| | | LSLPFLPDSPRYLLLEKHNEARAVKAFQTFLGKADVSQEVE |
| | | EVLAESRVQRSIRLVSVLELLRAPYVRWQVVTVIVTMACYQ |
| | | LCGLNAIWFYTNSIFGKAGIPPAKIPYVTLSTGGIETLAAV |
| | | FSGLVIEHLGRRPLLIGGFGLMGLFFGTLTITLTLQDHAPW |
| | | VPYLSIVGILAIIASFCSGPGGIPFILTGEFFQQSQRPAAF |
| | | IIAGTVNWLSNFAVGLLFPFIQKSLDTYCFLVFATICITGA |
| | | IYLYFVLPETKNRTYAEISQAFSKRNKAYPPEEKIDSAVTD |
| | | GKINGRP |
| 18 | Homo | MAFSKLLEQAGGVGLFQTLQVLTFILPCLMIPSQMLLENFS |
| | sapiens | AAIPGHRCWTHMLDNGSAVSTNMTPKALLTISIPPGPNQGP |
| | OAT4 | HQCRRFRQPQWQLLDPNATATSWSEADTEPCVDGWVYDRSV |
| | | FTSTIVAKWDLVCSSQGLKPLSQSIFMSGILVGSFIWGLLS |
| | | YRFGRKPMLSWCCLQLAVAGTSTIFAPTFVIYCGLRFVAAF |
| | | GMAGIFLSSLTLMVEWTTTSRRAVTMTVVGCAFSAGQAALG |
| | | GLAFALRDWRTLQLAASVPFFAISLISWWLPESARWLIIKG |
| | | KPDQALQELRKVARINGHKEAKNLTIEVLMSSVKEEVASAK |
| | | EPRSVLDLFCVPVLRWRSCAMLVVNFSLLISYYGLVFDLQS |
| | | LGRDIFLLQALFGAVDFLGRATTALLLSFLGRRTIQAGSQA |
| | | MAGLAILANMLVPQDLQTLRVVFAVLGKGCFGISLTCLTIY |
| | | KAELFPTPVRMTADGILHTVGRLGAMMGPLILMSRQALPLL |
| | | PPLLYGVISIASSLVVLFFLPETQGLPLPDTIQDLESQKST |
| | | AAQGNRQEAVTVESTSL |
| 19 | Homo | MAFNDLLQQVGGVGRFQQIQVTLVVLPLLLMASHNTLQNFT |
| | sapiens | AAIPTHHCRPPADANLSKNGGLEVWLPRDRQGQPESCLRFT |
| | OAT1 | SPQWGLPFLNGTEANGTGATEPCTDGWIYDNSTFPSTIVTE |
| | | WDLVCSHRALRQLAQSLYMVGVLLGAMVFGYLADRLGRRKV |
| | | LILNYLQTAVSGTCAAFAPNFPIYCAFRLLSGMALAGISLN |
| | | CMTLNVEWMPIHTRACVGTLIGYVYSLGQFLLAGVAYAVPH |
| | | WRHLQLLVSAPFFAFFIYSWFFIESARWHSSSGRLDLTLRA |
| | | LQRVARINGKREEGAKLSMEVLRASLQKELTMGKGQASAME |
| | | LLRCPTLRHLFLCLSMLWFATSFAYYGLVMDLQGFGVSIYL |
| | | IQVIFGAVDLPAKLVGFLVINSLGRRPAQMAALLLAGICIL |
| | | LNGVIPQDQSIVRTSLAVLGKGCLAASFNCIFLYTGELYPT |
| | | MIRQTGMGMGSTMARVGSIVSPLVSMTAELYPSMPLFIYGA |
| | | VPVAASAVTVLLPETLGQPLPDTVQDLESRWAPTQKEAGIY |
| | | PRKGKQTRQQQEHQKYMVPLQASAQEKNGL |
| 20 | Homo | MTFSEILDRVGSMGHFQFLHVAILGLPILNMANHNLLQIFT |
| | sapiens | AATPVHHCRPPHNASTGPWVLPMGPNGKPERCLRFVHPPNA |
| | OAT3 | SLPNDTQRAMEPCLDGWVYNSTKDSIVTEWDLVCNSNKLKE |
| | | MAQSIFMAGILIGGLVLGDLSDRFGRRPILTCSYLLLAASG |
| | | SGAAFSPTFPIYMVFRFLCGFGISGITLSTVILNVEWVPTR |
| | | MRAIMSTALGYCYTFGQFILPGLAYAIPQWRWLQLTVSIPF |
| | | FVFFLSSWWTPESIRWLVLSGKSSKALKILRRVAVFNGKKE |
| | | EGERLSLEELKLNLQKEISLAKAKYTASDLFRIPMLRRMTF |
| | | CLSLAWFATGFAYYSLAMGVEEFGVNLYILQIIFGGVDVPA |
| | | KFITILSLSYLGRHTTQAAALLLAGGAILALTFVPLDLQTV |
| | | RTVLAVFGKGCLSSSFSCLFLYTSELYPTVIRQTGMGVSNL |
| | | WTRVGSMVSPLVKITGEVQPFIPNIIYGITALLGGSAALFL |
| | | PETLNQPLPETIEDLENWSLRAKKPKQEPEVEKASQRIPLQ |
| | | PHGPGLGSS |
| 21 | Homo | MAFSGSQAPYLSPAVPFSGTIQGGLQDGLQITVNGTVLSSS |
| | sapiens | GTRFAVNFQTGFSGNDIAFHFNPRFEDGGYVVCNTRQNGSW |
| | Gal-9 | GPEERKTHMPFQKGMPFDLCFLVQSSDFKVMVNGILFVQYF |
| | | HRVPFHRVDTISVNGSVQLSYISFQNPRTVPVQPAFSTVPF |
| | | SQPVCFPPRPRGRRQKPPGVWPANPAPITQTVIHTVQSAPG |
| | | QMFSTPAIPPMMYPHPAYPMPFITTILGGLYPSKSILLSGT |
| | | VLPSAQRFHINLCSGNHIAFHLNPRFDENAVVRNTQIDNSW |
| | | GSEERSLPRKMPFVRGQSFSVWILCEAHCLKVAVDGQHLFE |
| | | YYHRLRNLPTINRLEVGGDIQLTHVQT |
| 22 | Homo | MSSSNVEVFIPVSQGNTNGFPATASNDLKAFTEGAVLSFHN |
| | sapiens | ICYRVKLKSGFLPCRKPVEKEILSNINGIMKPGLNAILGPT |
| | ABCG2 | GGGKSSLLDVLAARKDPSGLSGDVLINGAPRPANFKCNSGY |
| | | VVQDDVVMGTLTVRENLQFSAALRLATTMTNHEKNERINRV |
| | | IQELGLDKVADSKVGTQFIRGVSGGERKRTSIGMELITDPS |
| | | ILFLDEPTTGLDSSTANAVLLLLKRMSKQGRTIIFSIHQPR |
| | | YSIFKLFDSLTLLASGRLMFHGPAQEALGYFESAGYHCEAY |
| | | NNPADFFLDIINGDSTAVALNREEDFKATEIIEPSKQDKPL |
| | | IEKLAEIYVNSSFYKETKAELHQLSGGEKKKKITVFKEISY |
| | | TTSFCHQLRWVSKRSFKNLLGNPQASIAQIIVTVVLGLVIG |
| | | AIYFGLKNDSTGIQNRAGVLFFLTTNQCFSSVSAVELFVVE |
| | | KKLFIHEYISGYYRVSSYFLGKLLSDLLPMRMLPSIIFTCI |
| | | VYFMLGLKPKADAFFVMMFTLMMVAYSASSMALAIAAGQSV |
| | | VSVATLLMTICFVFMMIFSGLLVNLTTIASWLSWLQYFSIP |
| | | RYGFTALQHNEFLGQNFCPGLNATGNNPCNYATCTGEEYLV |
| | | KQGIDLSPWGLWKNHVALACMIVIFLTIAYLKLLFLKKYS |
| 23 | Homo | MQMDNRLPPKKVPGFCSFRYGLSFLVHCCNVIITAQRACLN |
| | sapiens | LTMVVMVNSTDPHGLPNTSTKKLLDNIKNPMYNWSPDIQGI |
| | SLC34A2 | ILSSTSYGVIIIQVPVGYFSGIYSTKKMIGFALCLSSVLSL |
| | | LIPPAAGIGVAWVVVCRAVQGAAQGIVATAQFEIYVKWAPP |
| | | LERGRLTSMSTSGFLLGPFIVLLVTGVICESLGWPMVFYIF |
| | | GACGCAVCLLWFVLFYDDPKDHPCISISEKEYITSSLVQQV |
| | | SSSRQSLPIKAILKSLPVWAISTGSFTFFWSHNIMTLYTPM |
| | | FINSMLHVNIKENGFLSSLPYLFAWICGNLAGQLSDFFLTR |
| | | NILSVIAVRKLFTAAGFLLPAIFGVCLPYLSSTFYSIVIFL |
| | | ILAGATGSFCLGGVFINGLDIAPRYFGFIKACSTLTGMIGG |
| | | LIASTLTGLILKQDPESAWFKTFILMAAINVTGLIFYLIVA |
| | | TAEIQDWAKEKQHTRL |
| 24 | Homo | MLPVYQEVKPNPLQDANLCSRVFFWWLNPLFKIGHKRRLEE |
| | sapiens | DDMYSVLPEDRSQHLGEELQGFWDKEVLRAENDAQKPSLTR |
| | MRP4 | AIIKCYWKSYLVLGIFTLIEESAKVIQPIFLGKIINYFENY |
| | | DPMDSVALNTAYAYATVLTFCTLILAILHHLYFYHVQCAGM |
| | | RLRVAMCHMIYRKALRLSNMAMGKTTTGQIVNLLSNDVNKF |
| | | DQVTVFLHFLWAGPLQAIAVTALLWMEIGISCLAGMAVLII |
| | | LLPLQSCFGKLFSSLRSKTATFTDARIRTMNEVITGIRIIK |
| | | MYAWEKSFSNLITNLRKKEISKILRSSCLRGMNLASFFSAS |
| | | KIIVFVTFTTYVLLGSVITASRVFVAVTLYGAVRLTVTLFF |
| | | PSAIERVSEAIVSIRRIQTFLLLDEISQRNRQLPSDGKKMV |
| | | HVQDFTAFWDKASETPTLQGLSFTVRPGELLAVVGPVGAGK |
| | | SSLLSAVLGELAPSHGLVSVHGRIAYVSQQPWVFSGTLRSN |
| | | ILFGKKYEKERYEKVIKACALKKDLQLLEDGDLTVIGDRGT |
| | | TLSGGQKARVNLARAVYQDADIYLLDDPLSAVDAEVSRHLF |
| | | ELCICQILHEKITILVTHQLQYLKAASQILILKDGKMVQKG |
| | | TYTEFLKSGIDFGSLLKKDNEESEQPPVPGTPTLRNRTFSE |
| | | SSVWSQQSSRPSLKDGALESQDTENVPVTLSEENRSEGKVG |
| | | FQAYKNYFRAGAHWIVFIFLILLNTAAQVAYVLQDWWLSYW |
| | | ANKQSMLNVTVNGGGNVTEKLDLNWYLGIYSGLTVATVLFG |
| | | IARSLLVFYVLVNSSQTLHNKMFESILKAPVLFFDRNPIGR |
| | | ILNRFSKDIGHLDDLLPLTFLDFIQTLLQVVGVVSVAVAVI |
| | | PWIAIPLVPLGIIFIFLRRYFLETSRDVKRLESTTRSPVFS |
| | | HLSSSLQGLWTIRAYKAEERCQELFDAHQDLHSEAWFLFLT |
| | | TSRWFAVRLDAICAMFVIIVAFGSLILAKTLDAGQVGLALS |
| | | YALTLMGMFQWCVRQSAEVENMMISVERVIEYTDLEKEAPW |
| | | EYQKRPPPAWPHEGVIIFDNVNFMYSPGGPLVLKHLTALIK |
| | | SQEKVGIVGRTGAGKSSLISALFRLSEPEGKIWIDKILTTE |
| | | IGLHDLRKKMSIIPQEPVLFTGTMRKNLDPFNEHTDEELWN |
| | | ALQEVQLKETIEDLPGKMDTELAESGSNFSVGQRQLVCLAR |
| | | AILRKNQILIIDEATANVDPRTDELIQKKIREKFAHCTVLT |
| | | IAHRLNTIIDSDKIMVLDSGRLKEYDEPYVLLQNKESLFYK |
| | | MVQQLGKAEAAALTETAKQVYFKRNYPHIGHTDHMVTNTSN |
| | | GQPSTLTIFETAL |
| 44 | Homo | MGFEELLEQVGGFGPFQLRNVALLALPRVLLPLHFLLPIFL |
| | sapiens | AAVPAHRCALPGAPANFSHQDVWLEAHLPREPDGTLSSCLR |
| | OAT2/ | FAYPQALPNTTLGEERQSRGELEDEPATVPCSQGWEYDHSE |
| | SLC22A7 | FSSTIATESQWDLVCEQKGLNRAASTFFFAGVLVGAVAFGY |
| | | LSDRFGRRRLLLVAYVSTLVLGLASAASVSYVMFAITRTLT |
| | | GSALAGFTIIVMPLELEWLDVEHRTVAGVLSSTFWTGGVML |
| | | LALVGYLIRDWRWLLLAVTLPCAPGILSLWWVPESARWLLT |
| | | QGHVKEAHRYLLHCARLNGRPVCEDSFSQEAVSKVAAGERV |
| | | VRRPSYLDLFRTPRLRHISLCCVVVWFGVNFSYYGLSLDVS |
| | | GLGLNVYQTQLLFGAVELPSKLLVYLSVRYAGRRLTQAGTL |
| | | LGTALAFGTRLLVSSDMKSWSTVLAVMGKAFSEAAFTTAYL |
| | | FTSELYPTVLRQTGMGLTALVGRLGGSLAPLAALLDGVWLS |
| | | LPKLTYGGIALLAAGTALLLPETRQAQLPETIQDVERKSAP |
| | | TSLQEEEMPMKQVQN |
| 45 | Homo | MATKTELSPTARESKNAQDMQVDETLIPRKVPSLCSARYGI |
| | sapiens | ALVLHFCNFTTIAQNVIMNITMVAMVNSTSPQSQLNDSSEV |
| | NPT4/ | LPVDSFGGLSKAPKSLPAKSSILGGQFAIWEKWGPPQERSR |
| | SLC17A3 | LCSIALSGMLLGCFTAILIGGFISETLGWPFVFYIFGGVGC |
| | | VCCLLWFVVIYDDPVSYPWISTSEKEYIISSLKQQVGSSKQ |
| | | PLPIKAMLRSLPIWSICLGCFSHQWLVSTMVVYIPTYISSV |
| | | YHVNIRDNGLLSALPFIVAWVIGMVGGYLADFLLTKKFRLI |
| | | TVRKIATILGSLPSSALIVSLPYLNSGYITATALLTLSCGL |
| | | STLCQSGIYINVLDIAPRYSSFLMGASRGFSSIAPVIVPTV |
| | | SGFLLSQDPEFGWRNVFFLLFAVNLLGLLFYLIFGEADVQE |
| | | WAKERKLTRL |
| 46 | Homo | MQMDNRLPPKKVPGFCSFRYGLSFLVHCCNVIITAQRACLN |
| | sapiens | LTMVVMVNSTDPHGLPNTSTKKLLDNIKNPMYNWSPDIQGI |
| | NPT1/ | ILSSTSYGVIIIQVPVGYFSGIYSTKKMIGFALCLSSVLSL |
| | SLC17A1 | LIPPAAGIGVAWVVVCRAVQGAAQGIVATAQFEIYVKWAPP |
| | | LERGRLTSMSTSGFLLGPFIVLLVTGVICESLGWPMVFYIF |
| | | GACGCAVCLLWFVLFYDDPKDHPCISISEKEYITSSLVQQV |
| | | SSSRQSLPIKAILKSLPVWAISTGSFTFFWSHNIMTLYTPM |
| | | FINSMLHVNIKENGFLSSLPYLFAWICGNLAGQLSDFFLTR |
| | | NILSVIAVRKLFTAAGFLLPAIFGVCLPYLSSTFYSIVIFL |
| | | ILAGATGSFCLGGVFINGLDIAPRYFGFIKACSTLTGMIGG |
| | | LIASTLTGLILKQDPESAWFKTFILMAAINVTGLIFYLIVA |
| | | TAEIQDWAKEKQHTRL |
| 47 | Homo | MELKKSPDGGWGWVIVFVSFLTQFLCYGSPLAVGVLYIEWL |
| | sapiens | DAFGEGKGKTAWVGSLASGVGLLASPVCSLCVSSFGARPVT |
| | mono- | IFSGFMVAGGLMLSSFAPNIYFLFFSYGIVVGLGCGLLYTA |
| | carboxylate | TVTITCQYFDDRRGLALGLISTGSSVGLFIYAALQRMLVEF |
| | transporter | YGLDGCLLIVGALALNILACGSLMRPLQSSDCPLPKKIAPE |
| | 9/MCT9/ | DLPDKYSIYNEKGKNLEENINILDKSYSSEEKCRITLANGD |
| | SLC16A9 | WKQDSLLHKNPTVTHTKEPETYKKKVAEQTYFCKQLAKRKW |
| | | QLYKNYCGETVALFKNKVFSALFIAILLFDIGGFPPSLLME |
| | | DVARSSNVKEEEFIMPLISIIGIMTAVGKLLLGILADFKWI |
| | | NTLYLYVATLIIMGLALCAIPFAKSYVTLALLSGILGFLTG |
| | | NWSIFPYVTTKTVGIEKLAHAYGILMFFAGLGNSLGPPIVG |
| | | WFYDWTQTYDIAFYFSGFCVLLGGFILLLAALPSWDTCNKQ |
| | | LPKPAPTTFLYKVASNV |

In some embodiments, the uric acid transporter comprises a URAT1 comprising the amino acid sequence set forth in SEQ ID NO:16. In some embodiments, the uric acid transporter comprises a GLUTS comprising the amino acid sequence set forth in SEQ ID NO:17. In some embodiments, the uric acid transporter comprises a OAT4 comprising the amino acid sequence set forth in SEQ ID NO:18. In some embodiments, the uric acid transporter comprises a OAT1 comprising the amino acid sequence set forth in SEQ ID NO:19. In some embodiments, the uric acid transporter comprises a OATS comprising the amino acid sequence set forth in SEQ ID NO:20. In some embodiments, the uric acid transporter comprises a Gal-9 comprising the amino acid sequence set forth in SEQ ID NO:21. In some embodiments, the uric acid transporter comprises a ABCG2 comprising the amino acid sequence set forth in SEQ ID NO:22. In some embodiments, the uric acid transporter comprises a SLC34A2 comprising the amino acid sequence set forth in SEQ ID NO:23. In some embodiments, the uric acid transporter comprises a MRP4 comprising the amino acid sequence set forth in SEQ ID NO:24. In some embodiments, the uric acid transporter comprises a OAT2 comprising the amino acid sequence set forth in SEQ ID NO:44. In some embodiments, the uric acid transporter comprises a NPT4 comprising the amino acid sequence set forth in SEQ ID NO:45. In some embodiments, the uric acid transporter comprises a NPT1 comprising the amino acid sequence set forth in SEQ ID NO:46. In some embodiments, the uric acid transporter comprises a MCT9 comprising the amino acid sequence set forth in SEQ ID NO:47.

In some embodiments, the uric acid transporter comprises a variant of a wild-type uric acid transporter having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 44, SEQ ID NO: 45, SEQ ID NO: 46, or SEQ ID NO: 47. In some embodiment the variant of the uric acid transporter possesses a function of the wild-type uric acid transporter from which it was derived (e.g., the ability to import uric acid).

In a particular embodiment, the uric acid transporter comprises an amino acid sequence that is at least about 95% identical (e.g., 96%, 97%, 98%, 99% or 100% identical) to an amino acid sequence set forth in any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 44, SEQ ID NO: 45, SEQ ID NO: 46, or SEQ ID NO: 47.

In a particular embodiment, the uric acid transporter consists of the amino acid sequence of any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 44, SEQ ID NO: 45, SEQ ID NO: 46, or SEQ ID NO: 47

In some embodiments, the uric acid transporter comprises a fragment of a URAT1, a GLUT9, a OAT4, a OAT1, a OAT3, a Gal-9, an ABCG2, a SLC34A2, a MRP4, an OAT2, a NPT4, a NPT1, or a MCT9. In some embodiments, the fragment of the uric acid transporter comprises at least 10, at least 20, at least 30, at least 40, at least 50, at least 60, at least 70, at least 80, at least 90, at least 100, at least 110, at least 120, at least 130, at least 140, at least 150 or at least 160 amino acid residues (e.g., contiguous amino acid residues) of any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 44, SEQ ID NO: 45, SEQ ID NO: 46, or SEQ ID NO: 47, or a variant thereof. In some embodiments, the fragment of the uric acid transporter comprises fewer than 20, fewer than 30, fewer than 40, fewer than 50, fewer than 60, fewer than 70, fewer than 80, fewer than 90, fewer than 100, fewer than 110, fewer than 120, fewer than 130, fewer than 140, fewer than 150 or fewer than 160 amino acid residues (e.g., contiguous amino acid residues) of any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO: 44, SEQ ID NO: 45, SEQ ID NO: 46, or SEQ ID NO: 47, or a variant thereof. In some embodiments, fragments or variants of the uric acid transporter retain at least 50%, at least 55%, at least 60%, at least 65%, at least 70%, at least 75%, at least 80%, at least 85%, at least 90%, at least 95%, at least 98% or at least 99% of the function (e.g., the ability to import uric acid) as compared to the uric acid transporter from which they were derived.

In general, a variant uric acid transporter, may be produced, for example, to enhance production of the protein in an engineered cell, to improve turnover/half-life of the protein or mRNA encoding the protein, and/or to modulate (enhance or reduce) the activity of the uric acid transporter. The uric acid transporter may also be in a form that is truncated, either at the amino terminal, or at the carboxyl terminal, or at both terminals.

In some embodiments, the invention provides an engineered erythroid cell (e.g. an engineered erythroid precursor cell) comprising a nucleic acid sequence encoding a uric acid transporter as described herein. In some embodiments, the invention provides an engineered erythroid cell prepared by using a nucleic acid sequence encoding a uric acid transporter as described herein. In some embodiments, the nucleic acid sequence encodes a uric acid transporter (e.g. a URAT1, a GLUT9, a OAT4, a OAT1, a OAT3, a Gal-9, an ABCG2, a SLC34A2, a MRP4, an OAT2, a NPT4, a NPT1, or a MCT9) as described herein.

In some embodiments, the uric acid transporter is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%. at least 97%, at least 98%, at least 99%, or 100% identical to the corresponding uric acid transporter nucleic acid that encodes a protein that possesses a function of a uric acid transporter described herein, e.g., the regulation of uric acid transport or the import of uric acid.

In some embodiments, the nucleic acid sequence encoding the uric acid transporter or a component thereof, is codon optimized (e.g., codon optimized for expression in a human cell). For example, in some embodiments, the nucleic acid sequence encoding the uric acid transporter (e.g. a URAT1, a GLUT9, a OAT4, a OAT1, a OAT3, a Gal-9, an ABCG2, a SLC34A2, a MRP4, an OAT2, a NPT4, a NPT1, or a MCT9) is codon optimized. In other embodiments, the nucleic acid sequence encoding the uric acid transporter or a component thereof is not codon optimized. For example, in some embodiments, the nucleic acid sequence encoding the uric acid transporter (e.g. URAT1, GLUT9, OAT4, OAT1, OAT3, Gal-9, ABCG2, SLC34A2, and MRP4) is not codon optimized.

In some embodiments, an engineered erythroid cell or an enucleated cell comprises an exogenous polypeptide comprising a uric acid transporter that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP)). The polypeptide of interest may be disposed in any configuration of the exogenous polypeptide (e.g., the polypeptide of interest may be fused to the N-terminus or C-terminus of the uric acid transporter).

In some embodiments, the exogenous polypeptide may include a linker (e.g., a linker described herein) disposed between the uric acid transporter and the at least one polypeptide of interest. In some embodiments, the linker comprises or consists of a poly-glycine poly-serine linker with one or more amino acid substitutions, deletions, and/or additions and which lacks the amino acid sequence GSG.

In some embodiments, the exogenous polypeptide comprises a transmembrane domain or a transmembrane polypeptide (e.g., SMIM1, GPA, or Kell) and a uric acid transporter. In some embodiments, the transmembrane domain is derived from SMIM1. In some embodiments, the transmembrane domain is derived from GPA.

In some embodiments, the exogenous polypeptide does not include a transmembrane domain or a transmembrane polypeptide. In some embodiments, the exogenous polypeptide does not include a polypeptide that is endogenous to the cell. In some embodiments, a linker (e.g., any linker provided herein) is disposed between the transmembrane domain or transmembrane polypeptide and the uric acid transporter.

In some embodiments the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA.

### Catalases

According to some embodiments, the engineered erythroid cell of the present invention comprises an enzyme that degrades or otherwise utilizes hydrogen peroxide, such that the hydrogen peroxide level in the cell is reduced, e.g., a catalase. According to some embodiments, the engineered erythroid cell of the present invention comprises an exogenous polypeptide comprising a catalase. In these embodiments, the presence of an exogenous polypeptide comprising a catalase in an engineered enucleated cell aids in counteracting the production of hydrogen peroxide resulting from the breakdown of uric acid by an exogenous polypeptide comprising a uric acid degrading polypeptide (e.g., a uricase).

Accordingly, in one aspect, the disclosure provides an engineered erythroid cell comprising a first exogenous polypeptide comprising a catalase, or a variant thereof. In another aspect, the disclosure provides an erythroid cell engineered to degrade uric acid, wherein the cell comprises a first exogenous polypeptide comprising a uric acid degrading polypeptide, e.g., uricase, or a variant thereof, and a second exogenous polypeptide comprising a catalase. In another aspect, the disclosure provides an erythroid cell engineered to degrade uric acid, wherein the cell comprises a first exogenous polypeptide comprising a uric acid degrading polypeptide, e.g., uricase, or a variant thereof, a second exogenous polypeptide comprising a uric acid transporter, or a variant thereof, and further comprises a third exogenous polypeptide comprising a catalase.

In some embodiments, the engineered erythroid cell comprises at least one (e.g., one, two, three, four, or more) exogenous polypeptide comprising a catalase. In some embodiments, the engineered enucleated cell comprises more than one (e.g., two, three, four, or more) exogenous polypeptides each comprising a catalase. In some embodiments, the engineered erythroid cell comprises at least one exogenous polypeptide comprising both a uric acid degrading polypeptide and a catalase. In some embodiments, the engineered erythroid cell comprises at least one exogenous polypeptide comprising a uric acid transporter and a catalase.

Catalase is a common enzyme found in living organisms. Catalases catalyze the decomposition of hydrogen peroxide to water and oxygen (see, e.g., Chelikani et al. (2004) Cellular and Molecular Life Sciences 61 (2): 192-208, the entire contents of which is incorporated by reference in its entirety herein). The activity of a catalase is influenced by several factors including, but not limited to, the concentration of hydrogen peroxide, temperature, pH, and the presence of inhibitors or activators.

In some embodiments, an erythroid cell of the disclosure comprises an exogenous polypeptide comprising a catalase selected from the group consisting of: P83657, CAT1_COMTR; Q9C168, CAT1_NEUCR; P81138, CAT1_PENJA; Q9C169, CAT3_NEUCR; Q96528, CATA1_ARATH; Q27487, CATA1_CAEEL; P48350, CATA1_CUCPE; P17598, CATA1_GOSHI; P55307, CATA1_HORVU; P18122, CATA1_MAIZE; P49315, CATA1_NICPL; P00549, CATA1_ORYSI; Q0E4K1, CATA1_ORYSJ; Q01297, CATA1_RICCO; P30264, CATA1_SOLLC; P49284, CATA1_SOLTU; P29756, CATA1_SOYBN; P49319, CATA1_TOBAC; Q43206, CATA1_WHEAT; P25819, CATA2 _ARATH; O61235, CATA2_CAEEL; P48351, CATA2_CUCPE; P30567, CATA2_GOSHI; P55308, CATA2_HORVU; P12365, CATA2_MAIZE; P49316, CATA2_NICPL; A2YH64, CATA2_ORYSI; Q0D9C4, CATA2_ORYSJ; P49318, CATA2_RICCO; Q9XHH3, CATA2_SOLLC; P55312, CATA2_SOLTU; P55313, CATA2_WHEAT; Q42547, CATA3_ARATH; _P48352, CATA3_CUCPE; P18123, CATA3_MAIZE; P49317, CATA3_NICPL; O48560, CATA3_SOYBN; O48561, CATA4_SOYBN; P90682, CATA_ASCSU; P78574, CATA_ASPFU; Q9AXH0, CATA_AVIMR; P45737, CATA_BACFR; P26901, CATA_BACSU; Q8X1P0, CATA_BLUGH; P0A324, CATA_BORBR; P0A325, CATA_BORPA; P0A323, CATA_BORPE; P55304, CATA_BOTFU; P00432, CATA_BOVIN; P0A327, CATA_BRUAB; P0A326, CATA_BRUME; Q8FWU0, CATA_BRUSU; Q2I6W4, CATA_CALJA; Q59296, CATA_CAMJE; O13289, CATA_CANAL; Q96VB8, CATA_CANBO; O97492, CATA_CANLF; P07820, CATA_CANTR; Q9M5L6, CATA_CAPAN; Q64405, CATA_CAVPO; Q9PT92, CATA_DANRE; Q59337, CATA_DEIRA; Q9ZN99, CATA_DESVM; O77229, CATA_DICDI; P17336, CATA_DROME; P55305, CATA_EMENI; P44390, CATA_HAEIN; P45739, CATA_HELAN; Q9ZKX5, CATA_HELPJ; P77872, CATA_HELPY; P04040, CATA_HUMAN; P07145, CATA_IPOBA; P30265, CATA_LACSK; Q926X0, CATA_LISIN; Q8Y3P9, CATA_LISMO; P24168, CATA_LISSE; O93662, CATA_METBF; P29422, CATA_MICLU; P24270, CATA_MOUSE; Q59602, CATA_NEIGO; Q27710, CATA_ONCVE; P25890, CATA_PEA; P11934, CATA_PENJA; P30263, CATA_PICAN; O62839, CATA_PIG; Q5RF10, CATA_PONAB; P42321, CATA_PROMI; O52762, CATA_PSEAE; Q59714, CATA_PSEPU; P04762, CATA_RAT; P95631, CATA_RHIME; Q9PWF7, CATA_RUGRU; P55306, CATA_SCHPO; P55310, CATA_SECCE; O24339, CATA_SOLAP; P55311, CATA_SOLME; Q2FH99, CATA_STAA3; Q2FYU7, CATA_STAA8; Q2YXT2, CATA_STAAB; Q5HG86, CATA_STAAC; Q99UE2, CATA_STAAM; Q7A5T2, CATA_STAAN; Q6GH72, CATA_STAAR; Q6G9M4, CATA_STAAS; Q9L4S1, CATA_STAAU; Q8NWV5, CATA_STAAW; Q2PUJ9, CATA_STAEP; Q5HPK8, CATA_STAEQ; Q8CPDO, CATA_STAES; Q4L643, CATA_STAHJ; Q49XC1, CATA_STAS1; Q9KW19, CATA_STAWA; Q9EV50, CATA_STAXY; Q9Z598, CATA_STRCO; Q9XZD5, CATA_TOXGO; Q9KRQ1, CATA_VIBCH; O68146, CATA_VIBF1; Q87JE8, CATA_VIBPA; Q8D452, CATA_VIBVU; Q7MFM6, CATA_VIBVY; P32290, CATA_VIGRR; P15202, CATA_YEAST; Q9Y7C2, CATB_AJECA; Q92405, CATB_ASPFU; Q877A8, CATB_ASPOR; Q55DH8, CATB_DICDI; P78619, CATB_EMENI; Q59635, CATB_PSEAE; P46206, CATB_PSESY; Q66V81, CATB_STAXY; P30266, CATE_BACPE; P42234, CATE_BACSU; P21179, CATE_ECOLI; P50979, CATE_MYCAV; Q9I1W8, CATE_PSEAE; P95539, CATE_PSEPU; Q9X576, CATE_RHIME; P55303, CATR_ASPNG; A6ZV70, CATT_YEAS7; P06115, CATT_YEAST; P94377, CATX_BACSU, P80878, MCAT_BACSU; Q97FE0, MCAT_CLOAB; P60355, MCAT_LACPN.

In some embodiments, the catalase is a human catalase, comprising or consisting of the amino acid sequence of SEQ ID NO: 25 (below), or a variant thereof.

SEQ ID NO: 25:
MADSRDPASDQMQHWKEQRAAQKADVLTTGAGNPVGDKLNVITVGPRGPLL
VQDVVFTDEMAHFDRERIPERVVHAKGAGAFGYFEVTHDITKYSKAKVFEH
IGKKTPIAVRFSTVAGESGSADTVRDPRGFAVKFYTEDGNWDLVGNNTPIF
FIRDPILFPSFIHSQKRNPQTHLKDPDMVWDFWSLRPESLHQVSFLFSDRG
IPDGHRHMNGYGSHTFKLVNANGEAVYCKFHYKTDQGIKNLSVEDAARLSQ
EDPDYGIRDLFNAIATGKYPSWTFYIQVMTFNQAETFPFNPFDLTKVWPHK
DYPLIPVGKLVLNRNPVNYFAEVEQIAFDPSNMPPGIEASPDKMLQGRLFA
YPDTHRHRLGPNYLHIPVNCPYRARVANYQRDGPMCMQDNQGGAPNYYPNS
FGAPEQQPSALEHSIQYSGEVRRFNTANDDNVTQVRAFYVNVLNEEQRKRL
CENIAGHLKDAQIFIQKKAVKNFTEVHPDYGSHIQALLDKYNAEKPKNAIH
TFVQSGSHLAAREKANL

In some embodiments, the catalase comprises or consists of the amino acid sequence of SEQ ID NO: 43 (below), or a variant thereof.

SEQ ID NO: 43:
MADSRDPASDQMQHWKEQRAAQKADVLTTGAGNPVGDKLNVITVGPRGPLL
VQDVVFTDEMAHFDRERIPERVVHAKGAGAFGYFEVTHDITKYSKAKVFEH
IGKKTPIAVRFSTVAGESGSADTVRDPRGFAVKFYTEDGNWDLVGNNTPIF
FIRDPILFPSFIHSQKRNPQTHLKDPDMVWDFWSLRPESLHQVSFLFSDRG
IPDGHRHMNGYGSHTFKLVNANGEAVYCKFHYKTDQGIKNLSVEDAARLSQ
EDPDYGIRDLFNAIATGKYPSWTFYIQVMTFNQAETFPFNPFDLTKVWPHK
DYPLIPVGKLVLNRNPVNYFAEVEQIAFDPSNMPPGIEASPDKMLQGRLFA
YPDTHRHRLGPNYLHIPVNCPYRARVANYQRDGPMCMQDNQGGAPNYYPNS
FGAPEQQPSALEHSIQYSGEVRRFNTANDDNVTQVRAFYVNVLNEEQRKRL
CENIAGHLKDAQIFIQKKAVKNFTEVHPDYGSHIQALLDKYNAEKPKNAIH
TFVQSGSHLAARE

In some embodiments, the catalase comprises a variant of a human catalase having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of either SEQ ID NO: 25 or SEQ ID NO: 43. In a particular embodiment, the catalase consists of the amino acid sequence of SEQ ID NO: 25. In another particular embodiment, the catalase consists of the amino acid sequence of SEQ ID NO: 43.

In some embodiments, the catalase is not a human catalase. In some embodiments, the catalase is not a human catalase comprising an amino acid sequence at least 80% identical (e.g., at least 80%, 85%, 90%, 95%, 96%, 97%, 98%, or 99%) to SEQ ID NO: 25. In some embodiments, the catalase is not a human catalase consisting of SEQ ID NO: 25.

In some embodiments, the catalase is engineered to delete a peroxisome targeting signal (e.g., a native peroxisome targeting signal). In some embodiments, the catalase, or variant thereof, lacks a peroxisome targeting signal.

In some embodiments, the catalase comprises a fragment of the amino acid sequence set forth in either SEQ ID NO: 25 or SEQ ID NO: 43. In some embodiments, the fragment of the catalase comprises at least 10, at least 20, at least 30, at least 40, at least 50, at least 60, at least 70, at least 80, at least 90, at least 100, at least 110, at least 120, at least 130, at least 140, at least 150 or at least 160 amino acid residues (e.g., contiguous amino acid residues) of either SEQ ID NO: 25 or SEQ ID NO: 43. In some embodiments, the fragment of the catalase comprises fewer than 20, fewer than 30, fewer than 40, fewer than 50, fewer than 60, fewer than 70, fewer than 80, fewer than 90, fewer than 100, fewer than 110, fewer than 120, fewer than 130, fewer than 140, fewer than 150 or fewer than 160 amino acid residues (e.g., contiguous amino acid residues) of either SEQ ID NO: 25 or SEQ ID NO: 43. In some embodiments, fragments or variants of the catalase retain at least 50%, at least 55%, at least 60%, at least 65%, at least 70%, at least 75%, at least 80%, at least 85%, at least 90%, at least 95%, at least 98% or at least 99% of the function (e.g., the ability to catalyze the decomposition of hydrogen peroxide to water and oxygen) of the catalase from which it was derived.

In general, a variant of a catalase, may be produced, for example, to enhance production of the protein in an engineered cell, to improve turnover/half-life of the protein or mRNA encoding the protein, and/or to modulate (enhance or reduce) the activity of the catalase. The catalase may also be in a form that is truncated, either at the amino terminal, or at the carboxyl terminal, or at both terminals.

In some embodiments, the invention provides an engineered erythroid cell (e.g. an engineered erythroid precursor cell) comprising a nucleic acid sequence encoding a catalase as described herein. In some embodiments, the invention provides an engineered erythroid cell prepared by using a nucleic acid sequence encoding a catalase as described herein. In some embodiments, the nucleic acid sequence encodes a catalase (e.g. a catalase comprising SEQ ID NO: 25 or SEQ ID NO: 43) as described herein.

In some embodiments, the catalase is encoded by a nucleic acid that comprises a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%. at least 97%, at least 98%, at least 99%, or 100% identical to the corresponding catalase nucleic acid that encodes a protein that possesses a function of a catalase described herein, e.g., catalyze the decomposition of hydrogen peroxide to water and oxygen.

In some embodiments, the nucleic acid sequence encoding the catalase or a component thereof is codon optimized (e.g., codon optimized for expression in a human cell). In other embodiments, the nucleic acid sequence encoding the catalase or a component thereof is not codon optimized.

In some embodiments, an engineered erythroid cell or an enucleated cell comprises an exogenous polypeptide comprising a catalase that is fused to at least one (e.g., one, two, three, four, or five) polypeptide(s) of interest (e.g., an endogenous polypeptide, a signal sequence, a tag (e.g., a GST tag, a myc-tag, a HA tag, or a poly-His tag), a tracking moiety (e.g., a fluorescent polypeptide such as green fluorescent protein (GFP)). The polypeptide of interest may be disposed in any configuration of the exogenous polypeptide (e.g., the polypeptide of interest may be fused to the N-terminus or C-terminus of the catalase).

In some embodiments, the exogenous polypeptide may include a linker (e.g., a linker described herein) disposed between the catalase and the at least one polypeptide of interest. In some embodiments, the linker comprises or consists of a poly-glycine poly-serine linker with one or more amino acid substitutions, deletions, and/or additions and which lacks the amino acid sequence GSG.

In some embodiments, the exogenous polypeptide comprises a transmembrane domain or a transmembrane polypeptide (e.g., SMIM1, GPA, or Kell) and a catalase. In some embodiments, the transmembrane domain is derived from SMIM1. In some embodiments, the transmembrane domain is derived from GPA.

In some embodiments, the transmembrane domain or transmembrane polypeptide is disposed in the exogenous polypeptide such that the catalase present in the exogenous polypeptide locates to the cytosol of the cell (e.g., proximate to the inner leaflet of the plasma membrane). In some embodiments, the transmembrane domain or transmembrane polypeptide is disposed in the exogenous polypeptide such that the catalase present in the exogenous polypeptide locates in the outer surface of the cell (e.g., facing the extracellular milieu of the cell). In some embodiments, the exogenous polypeptide does not include a transmembrane domain or a transmembrane polypeptide. In some embodiments, the exogenous polypeptide does not include a polypeptide that is endogenous to the cell. In some embodiments, a linker (e.g., any linker provided herein) is disposed between the transmembrane domain or transmembrane polypeptide and the catalase.

In some embodiments the exogenous polypeptide comprises a leader or signal sequence at the N-terminal of the polypeptide. Said leader sequence may be processed and cleaved from by a peptidase (e.g., during translocation). Thus, in some embodiments, the exogenous polypeptide does not comprise a leader or signal sequence. In some embodiments, the leader or signal sequence is derived from GPA.

In some embodiments, the nucleic acid sequence encoding any of the exogenous polypeptide(s) described herein, or a component thereof, is codon-optimized (e.g., for expression in a mammalian cell). For example, in some embodiments, a nucleic acid sequence encoding an exogenous polypeptide comprising a uricase, a uric acid transporter, and/or a catalase is codon optimized. In other embodiments, the nucleic acid sequence encoding the exogenous polypeptide, or a component thereof, is not codon optimized. For example, in some embodiments, the nucleic acid sequence encoding an exogenous polypeptide comprising a uricase, a uric acid transporter, and/or a catalase is not codon optimized.

Various methods and software programs can be used to determine the homology between two or more peptides or nucleic acids, such as NCBI BLAST, Clustal W, MAFFT, Clustal Omega, AlignMe, Praline, or another suitable method or algorithm. In some embodiments, percent identity is calculated by FastDB based upon the following parameters: mismatch penalty of 1; gap penalty of 1; gap size penalty of 0.33; and joining penalty of 30.

An example of a useful algorithm is PILEUP. PILEUP creates a multiple sequence alignment from a group of related sequences using progressive, pairwise alignments. It can also plot a tree showing the clustering relationships used to create the alignment. Useful PILEUP parameters including a default gap weight of 3.00, a default gap length weight of 0.10, and weighted end gaps.

Another example of a useful algorithm is the BLAST algorithm. A useful example of a BLAST program is the WU-BLAST-2 program. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

An additional useful tool is Clustal, a series of commonly used computer programs tor multiple sequence alignment. Recent versions of Clustal include ClustalW, ClustalX and Clustal Omega. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

### Polypeptides and Nucleic Acids

In one aspect, the disclosure provides isolated uric acid degrading polypeptides (e.g., uricase) and uric acid transporters described herein. In some embodiments, the uric acid degrading polypeptides comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to the amino acid sequences of a uric acid degrading polypeptide described herein. In some embodiments, the uric acid transporters comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to the amino acid sequences of a uric acid transporter described herein. In some embodiments, the uric acid degrading polypeptides and uric acid transporters are recombinantly produced. Methods for producing recombinant proteins are known in the art and described herein.

In another aspect, the disclosure provides nucleic acids (e.g., DNA or RNA (e.g., mRNA)) encoding a uric acid degrading polypeptide described herein. In another aspect, the disclosure provides nucleic acids (e.g., DNA or RNA (e.g., mRNA)) encoding a uric acid transporter described herein. In some embodiments, the nucleic acids are codon-optimized for expression in a desired cell type (e.g., a bacterial or mammalian cell).

### Specific Activity of Uric Acid Degrading Enzymes

Specific activity can be defined as the number of enzyme units per milligram of protein, where one unit of activity is defined as degradation of 1 μmol of uric acid per minute.

In one aspect, the disclosure provides an erythroid cell engineered to degrade uric acid, comprising a first exogenous polypeptide comprising a uric acid degrading polypeptide (e.g, uricase), or a variant thereof, wherein the specific activity of the uric acid degrading polypeptide is greater than about 5 mol/min/mg at a neutral pH, for example greater than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 7, 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 5 μmol/min/mg or more.

In some embodiments, the specific activity of the uric acid degrading polypeptide (e.g, uricase) is greater than about 10 mol/min/mg at a neutral pH. In some embodiments, the specific activity of the uric acid degrading polypeptide (e.g, uricase) is greater than about 20 mol/min/mg at a neutral pH. In some embodiments, the specific activity of the uric acid degrading polypeptide (e.g, uricase) is greater than about 30 μmol/min/mg at a neutral pH. In some embodiments, the specific activity of the uric acid degrading polypeptide (e.g, uricase) is greater than about 40 mol/min/mg at a neutral pH. In some embodiments, the specific activity of the uric acid degrading polypeptide (e.g, uricase) is greater than about 50 μmol/min/mg at a neutral pH. In some embodiments, the specific activity of the uric acid degrading polypeptide (e.g, uricase) is greater than about 60 μmol/min/mg at a neutral pH. In some embodiments, the specific activity of the uric acid degrading polypeptide (e.g, uricase) is greater than about 70 μmol/min/mg at a neutral pH. In some embodiments, the specific activity of the uric acid degrading polypeptide (e.g, uricase) is greater than about 80 μmol/min/mg at a neutral pH. In some embodiments, the specific activity of the uric acid degrading polypeptide (e.g, uricase) is greater than about 90 μmol/min/mg at a neutral pH. In some embodiments, the specific activity of the uric acid degrading polypeptide (e.g, uricase) is greater than about 100 μmol/min/mg at a neutral pH.

In some embodiments, the specific activity of the uric acid degrading polypeptide (e.g, uricase) is greater than about 10 μmol/min/mg at a neutral pH, for example greater than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 7, 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 5 μmol/min/mg or more.

In one aspect, the disclosure provides an erythroid cell engineered to degrade uric acid, comprising a first exogenous polypeptide comprising a uric acid degrading polypeptide (e.g, uricase), or a variant thereof, wherein the specific activity of the uric acid degrading polypeptide is greater than about 50 μmol/min/mg at a neutral pH, for example greater than 50, 51, 52, 53, 54, 55, 56, 57, 58, 59, 60, 61, 62, 63, 64, 65, 66, 67, 68, 69, 70, 71, 7, 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 μmol/min/mg or more.

In some embodiments, the specific activity of the uric acid degrading polypeptide (e.g, uricase) is greater than about 100 μmol/min/mg at a neutral pH, for example greater than 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 μmol/min/mg or more.

In one aspect, the disclosure provides an erythroid cell engineered to degrade uric acid, comprising a first exogenous polypeptide comprising a uricase, or a variant thereof, wherein the specific activity of the uricase is greater than about 5 μmol/min/mg at a neutral pH, for example greater than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 7, 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 μmol/min/mg or more.

In some embodiments, the specific activity of the uricase is greater than about 10 μmol/min/mg at a neutral pH, for example greater than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 7, 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 μmol/min/mg or more.

In one aspect, the disclosure provides an erythroid cell engineered to degrade uric acid, comprising a first exogenous polypeptide comprising a uricase, or a variant thereof, wherein the specific activity of the uricase is greater than about 50 μmol/min/mg at a neutral pH, for example greater than 50, 51, 52, 53, 54, 55, 56, 57, 58, 59, 60, 61, 62, 63, 64, 65, 66, 67, 68, 69, 70, 71, 7, 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 μmol/min/mg or more.

In some embodiments, the specific activity of the uricase is greater than about 100 μmol/min/mg at a neutral pH, for example greater than 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 μmol/min/mg or more.

### Copy Number

In one aspect, the disclosure provides an erythroid cell engineered to degrade uric acid, comprising a first exogenous polypeptide comprising a uric acid degrading polypeptide, or a variant thereof. In some embodiments, the engineered erythroid cell is an enucleated cell, e.g. an erythrocyte or a reticulocyte. In some embodiments, the engineered erythroid cell is a nucleated cell. In some embodiments, the first exogenous polypeptide is expressed inside the erythroid cell. In some embodiments, the erythroid cell comprises at least 10,000, 20,000, 30,000, 40,000, 50,000, 60,000, 70,000, 80,000, 90,000, 100,000, 120,000, 130,000, 140,000, 150,000, 160,000, 170,000, 180,000, 190,000, 200,000, 210,000, 230,000, 240,000, 250,000, 260,000, 270,000, 280,000, 290,000, or 300,000 or more copies of the first exogenous polypeptide. In some embodiments, the erythroid cell comprises at least 50,000 copies of the first exogenous polypeptide. In some embodiments, the engineered erythroid cell comprises at least about 100,000 copies of the first exogenous polypeptide. In some embodiments, the engineered erythroid cell comprises at least about 150,000 copies of the first exogenous polypeptide. In some embodiments, the engineered erythroid cell comprises at least about 170,000 copies of the first exogenous polypeptide. In some embodiments, the engineered erythroid cell comprises about 50,000-300,000 copies of the first exogenous polypeptide, for example about 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000 or 300,000 copies of the first exogenous polypeptide.

In some embodiments, the first exogenous polypeptide comprises a uric acid degrading polypeptide which is a uricase, or a variant thereof, and the engineered erythroid cell comprises at least 10,000, 20,000, 30,000, 40,000, 50,000, 60,000, 70,000, 80,000, 90,000, 100,000, 120,000, 130,000, 140,000, 150,000, 160,000, 170,000, 180,000, 190,000, 200,000, 210,000, 230,000, 240,000, 250,000, 260,000, 270,000, 280,000, 290,000, or 300,000 or more copies of the first exogenous polypeptide. In some embodiments, the engineered erythroid cell comprises at least 50,000 copies of the first exogenous polypeptide. In some embodiments, the engineered erythroid cell comprises at least about 100,000 copies of the first exogenous polypeptide. In some embodiments, the engineered erythroid cell comprises at least about 150,000 copies of the first exogenous polypeptide. In some embodiments, the engineered erythroid cell comprises at least about 170,000 copies of the first exogenous polypeptide. In some embodiments, the engineered erythroid cell comprises about 50,000-300,000 copies of the first exogenous polypeptide, for example about 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 200,000, 205,000, 210,000, 215,000, 220,000, 225,000, 230,000, 235,000, 240,000, 245,000, 255,000, 260,000, 265,000, 270,000, 275,000, 280,000, 285,000, 290,000, 295,000 or 300,000 copies of the first exogenous polypeptide.

In some embodiments, the first exogenous polypeptide comprises a uric acid degrading polypeptide which is an allantoinase, or a variant thereof, and the engineered erythroid cell comprises at least 10,000, 20,000, 30,000, 40,000, 50,000, 60,000, 70,000, 80,000, 90,000, 100,000, 120,000, 130,000, 140,000, 150,000, 160,000, 170,000, 180,000, 190,000, 200,000, 210,000, 230,000, 240,000, 250,000, 260,000, 270,000, 280,000, 290,000, or 300,000 or more copies of the first exogenous polypeptide. In some embodiments, the erythroid cell comprises at least 50,000 copies of the first exogenous polypeptide. In some embodiments, the engineered erythroid cell comprises at least about 100,000 copies of the first exogenous polypeptide. In some embodiments, the engineered erythroid cell comprises at least about 150,000 copies of the first exogenous polypeptide. In some embodiments, the engineered erythroid cell comprises at least about 170,000 copies of the first exogenous polypeptide. In some embodiments, the engineered erythroid cell comprises about 50,000-300,000 copies of the first exogenous polypeptide, for example about 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 200,000, 205,000, 210,000, 215,000, 220,000, 225,000, 230,000, 235,000, 240,000, 245,000, 255,000, 260,000, 265,000, 270,000, 275,000, 280,000, 285,000, 290,000, 295,000 or 300,000 copies of the first exogenous polypeptide.

In another aspect, the disclosure provides an erythroid cell engineered to include a first exogenous polypeptide, wherein the first exogenous polypeptide comprises a catalase, or a variant thereof, and the engineered erythroid cell comprises at least 10,000, 20,000, 30,000, 40,000, 50,000, 60,000, 70,000, 80,000, 90,000, 100,000, 120,000, 130,000, 140,000, 150,000, 160,000, 170,000, 180,000, 190,000, 200,000, 210,000, 230,000, 240,000, 250,000, 260,000, 270,000, 280,000, 290,000, or 300,000 or more copies of the first exogenous polypeptide. In some embodiments, the engineered erythroid cell comprises about 50,000-300,000 copies of the first exogenous polypeptide, for example about 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 200,000, 205,000, 210,000, 215,000, 220,000, 225,000, 230,000, 235,000, 240,000, 245,000, 255,000, 260,000, 265,000, 270,000, 275,000, 280,000, 285,000, 290,000, 295,000 or 300,000 copies of the first exogenous polypeptide.

In another aspect, the disclosure provides an erythroid cell engineered to degrade uric acid, comprising a first exogenous polypeptide comprising a uric acid degrading polypeptide, or a variant thereof and further comprising a second exogenous polypeptide comprising a uric acid transporter, or a variant thereof. In some embodiments, the engineered erythroid cell is an enucleated cell, e.g. an erythrocyte or a reticulocyte. In some embodiments, the engineered erythroid cell is a nucleated cell. In some embodiments, the first exogenous polypeptide is expressed inside the erythroid cell. In some embodiments, the second exogenous polypeptide is presented at the surface of the erythroid cell. In some embodiments, the engineered erythroid cell comprises at least 10,000, 20,000, 30,000, 40,000, 50,000, 60,000, 70,000, 80,000, 90,000, 100,000, 120,000, 130,000, 140,000, 150,000, 160,000, 170,000, 180,000, 190,000, 200,000, 210,000, 230,000, 240,000, 250,000, 260,000, 270,000, 280,000, 290,000, or 300,000 or more copies of the first exogenous polypeptide. In some embodiments, the engineered erythroid cell comprises at least 50,000 copies of the first exogenous polypeptide. In some embodiments, the engineered erythroid cell comprises at least about 100,000 copies of the first exogenous polypeptide. In some embodiments, the engineered erythroid cell comprises at least about 150,000 copies of the first exogenous polypeptide. In some embodiments, the engineered erythroid cell comprises at least about 170,000 copies of the first exogenous polypeptide. In some embodiments, the engineered erythroid cell comprises about 50,000-300,000 copies of the first exogenous polypeptide, for example about 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 200,000, 205,000, 210,000, 215,000, 220,000, 225,000, 230,000, 235,000, 240,000, 245,000, 255,000, 260,000, 265,000, 270,000, 275,000, 280,000, 285,000, 290,000, 295,000 or 300,000 copies of the first exogenous polypeptide. In some embodiments, the erythroid cell comprises at least 10,000 copies of the second exogenous polypeptide. In some embodiments, the erythroid cell comprises at least 20,000 copies of the second exogenous polypeptide. In some embodiments, the erythroid cell comprises at least 30,000 copies of the second exogenous polypeptide. In some embodiments, the erythroid cell comprises at least 10,000, 20,000, 30,000, 40,000, 50,000, 60,000, 70,000, 80,000, 90,000 or 100,000 copies of the second exogenous polypeptide. In some embodiments, the erythroid cell comprises about 10,000-100,000 copies of the second exogenous polypeptide, for example about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000 or 100,000 copies of the second exogenous polypeptide.

In some embodiments, the first exogenous polypeptide is present at a copy number of no more than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% greater, or no more than 2, 3, 4, 5, 6, 7, 8, 9, 10, 20, 25, 30, 35, 40, 45, 50, 60, 70, 80, 90, 100, 150, 200, 300, 400, 500, or 1000 times greater than the copy number of the second exogenous polypeptide. In some embodiments, the second exogenous polypeptide is present at a copy number of no more than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% greater, or no more than 2, 3, 4, 5, 6, 7, 8, 9, 10, 20, 25, 30, 35, 40, 45, 50, 60, 70, 80, 90, 100, 150, 200, 300, 400, 500, or 1000 times greater than the copy number of the first exogenous polypeptide.

In some embodiments, the second exogenous polypeptide comprising a uric acid transporter is URAT1, or a variant thereof. In some embodiments, the erythroid cell comprises at least 10,000 copies of the second exogenous polypeptide. In some embodiments, the erythroid cell comprises at least 20,000 copies of the second exogenous polypeptide. In some embodiments, the erythroid cell comprises at least 30,000 copies of the second exogenous polypeptide. In some embodiments, the erythroid cell comprises at least 10,000, 20,000, 30,000, 40,000, 50,000, 60,000, 70,000, 80,000, 90,000 or 100,000 copies of the second exogenous polypeptide. In some embodiments, the erythroid cell comprises about 10,000-100,000 copies of the second exogenous polypeptide, for example about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000 or 100,000 copies of the second exogenous polypeptide.

In some embodiments, the second exogenous polypeptide comprising a uric acid transporter is GLUT9, or a variant thereof. In some embodiments, the erythroid cell comprises at least 10,000 copies of the second exogenous polypeptide. In some embodiments, the erythroid cell comprises at least 20,000 copies of the second exogenous polypeptide. In some embodiments, the erythroid cell comprises at least 30,000 copies of the second exogenous polypeptide. In some embodiments, the erythroid cell comprises at least 10,000, 20,000, 30,000, 40,000, 50,000, 60,000, 70,000, 80,000, 90,000 or 100,000 copies of the second exogenous polypeptide. In some embodiments, the erythroid cell comprises about 10,000-100,000 copies of the second exogenous polypeptide, for example about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000 or 100,000 copies of the second exogenous polypeptide.

In some embodiments, the first exogenous polypeptide and the second exogenous polypeptide have an abundance ratio of about 1:1, from about 2:1 to 1:2, from about 3:1 to 1:3, from about 1:4 to 4:1, from about 5:1 to 1:5, from about 6:1 to 1:6, from 7:1 to 1:7, from about 8:1 to 1:8, from about 9:1 to 1:9, from about 10:1 to 1:10, from about 20:1 to 1:20, from about 50:1 to 1:50, or from about 100:1 to 1:100 by weight or by copy number.

In some embodiments, the engineered erythroid cell comprises a first exogenous polypeptide comprising a uric acid degrading polypeptide, or a variant thereof, and further comprises a second exogenous polypeptide comprising a uric acid transporter, or a variant thereof, wherein the first and second exogenous polypeptides are present in an amount or copy number sufficient to reside in circulation for 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or longer.

In some embodiments, the engineered erythroid cell comprises a first exogenous polypeptide comprising a uricase, or a variant thereof, and further comprises a second exogenous polypeptide comprising a uric acid transporter, or a variant thereof, wherein the first and second exogenous polypeptides are present in an amount or copy number sufficient to reside in circulation for 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or longer.

In some embodiments, the engineered erythroid cell comprises a first exogenous polypeptide comprising Candida utilis uricase, or a variant thereof, and further comprises a second exogenous polypeptide comprising human URAT1, or a variant thereof, wherein the first and second exogenous polypeptides are present in an amount or copy number sufficient to reside in circulation for 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or longer.

In some embodiments of any of the foregoing aspects, the erythroid cell engineered to degrade uric acid comprising a first exogenous polypeptide comprising a uric acid degrading polypeptide, or a variant thereof, and a second exogenous polypeptide comprising a uric acid transporter, or a variant thereof, additionally comprises a third exogenous polypeptide comprising a catalase, or a variant thereof, and the engineered erythroid cell comprises at least 10,000, 20,000, 30,000, 40,000, 50,000, 60,000, 70,000, 80,000, 90,000, 100,000, 120,000, 130,000, 140,000, 150,000, 160,000, 170,000, 180,000, 190,000, 200,000, 210,000, 230,000, 240,000, 250,000, 260,000, 270,000, 280,000, 290,000, or 300,000 or more copies of the third exogenous polypeptide. In some embodiments, the engineered erythroid cell comprises about 50,000-300,000 copies of the third exogenous polypeptide, for example about 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 200,000, 205,000, 210,000, 215,000, 220,000, 225,000, 230,000, 235,000, 240,000, 245,000, 255,000, 260,000, 265,000, 270,000, 275,000, 280,000, 285,000, 290,000, 295,000 or 300,000 copies of the third exogenous polypeptide.

In some embodiments of the above aspects, the engineered erythroid cell is an enucleated cell, e.g., a reticulocyte or an erythrocyte. In some embodiments of the above aspects, the engineered erythroid cell is a nucleated cell.

### In Vivo Half-Life

In some embodiments, the first exogenous polypeptide, the second exogenous polypeptide, or both the first and second exogenous polypeptides (e.g., the uric acid degrading polypeptide, uric acid transporter, or both the uric acid degrading polypeptide and the uric acid transporter) are expressed in an engineered erythroid cell have a prolonged in vivo half-life as compared to a corresponding exogenous polypeptide (e.g., the uric acid degrading polypeptide, uric acid transporter, or both the uric acid degrading polypeptide and the uric acid transporter) that is administered by itself (i.e., not on or in a cell described herein). In some embodiments, any one, two or all three of the first exogenous polypeptide, the second exogenous polypeptide, and the third exogenous polypeptide (e.g., the uric acid degrading polypeptide, uric acid transporter, or catalase, or any two of the uric acid degrading polypeptide, the uric acid transporter and the catalase, or all three of the uric acid degrading polypeptide, the uric acid transporter and the catalase) expressed in an engineered erythroid cell have a prolonged in vivo half-life as compared to a corresponding exogenous polypeptide (e.g., the uric acid degrading polypeptide, uric acid transporter, or catalase, or any two of the uric acid degrading polypeptide, the uric acid transporter and the catalase, or all three of the uric acid degrading polypeptide, the uric acid transporter and the catalase) that is administered by itself (i.e., not on or in a cell described herein). In some embodiments, the first exogenous polypeptide, the second exogenous polypeptide, or both the first and second exogenous polypeptides have an in vivo half-life that is longer than the half-life of the first exogenous polypeptide, the second exogenous polypeptide, or both the first and second exogenous polypeptides, or a pegylated version of the first exogenous polypeptide, the second exogenous polypeptide, or both the first and second exogenous polypeptides, which are not comprised in an engineered erythroid cell. In some embodiments, the first exogenous polypeptide, the second exogenous polypeptide, the third exogenous polypeptide, or any two or all three of the first, second and third exogenous polypeptides have an in vivo half-life that is longer than the half-life of the first exogenous polypeptide, the second exogenous polypeptide, the third exogenous polypeptide, or any two or all three of the first, second and third exogenous polypeptides, or a pegylated version of the first exogenous polypeptide, the second exogenous polypeptide, the third exogenous polypeptide, or any two or all three of the first, second and third exogenous polypeptides, which are not copmrised in an engineered erythroid cell.

In some embodiments, the first exogenous polypeptide, the second exogenous polypeptide, or both the first and second exogenous polypeptides have an in vivo half-life of between about 24 hours and 60 days. In some embodiments, the first exogenous polypeptide, the second exogenous polypeptide, the third exogenous polypeptide, or any two or all three of the first, second and third exogenous polypeptides have an in vivo half-life of between about 24 hours and 60 days. In some embodiments, the first exogenous polypeptide, the second exogenous polypeptide, or both the first and second exogenous polypeptides have an in vivo half-life of at least 24 hours. In some embodiments, the first exogenous polypeptide, the second exogenous polypeptide, the third exogenous polypeptide, or any two or all three of the first, second and third exogenous polypeptides have an in vivo half-life of at least 24 hours. In some embodiments, the first exogenous polypeptide, the second exogenous polypeptide, or both the first and second exogenous polypeptides have an in vivo half-life of greater than 36 hours. In some embodiments, the first exogenous polypeptide, the second exogenous polypeptide, the third exogenous polypeptide, or any two or all three of the first, second and third exogenous polypeptides have an in vivo half-life of greater than 36 hours. In some embodiments, the first exogenous polypeptide, the second exogenous polypeptide, or both the first and second exogenous polypeptides have an in vivo half-life of greater than 48 hours. In some embodiments, the first exogenous polypeptide, the second exogenous polypeptide, the third exogenous polypeptide, or any two or all three of the first, second and third exogenous polypeptides have an in vivo half-life of greater than 48 hours. In some embodiments, the first exogenous polypeptide, the second exogenous polypeptide, or both the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first exogenous polypeptide, the second exogenous polypeptide, or both the first and second exogenous polypeptides have an in vivo half-life of 1 week, 2 weeks, 3 weeks, 1 month, 2 months, 3 months, 4 months or longer. In some embodiments, the first exogenous polypeptide, the second exogenous polypeptide, the third exogenous polypeptide, or all of the first, second and third exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first exogenous polypeptide, the second exogenous polypeptide, the third exogenous polypeptide, or all of the first, second and third exogenous polypeptides have an in vivo half-life of 1 week, 2 weeks, 3 weeks, 1 month, 2 months, 3 months, 4 months or longer.

In some embodiments, the engineered erythroid cell comprises a first exogenous polypeptide comprising a uric acid degrading polypeptide, or a variant thereof, and further comprises a second exogenous polypeptide comprising a uric acid transporter, or a variant thereof, wherein the first and second exogenous polypeptides have an in vivo half-life of at least 24 hours. In some embodiments, the first and second exogenous polypeptides have an in vivo half-life of greater than 36 hours. In some embodiments, the first and second exogenous polypeptides have an in vivo half-life of greater than 48 hours. In some embodiments, the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first and second exogenous polypeptides have an in vivo half-life of 1 week, 2 weeks, 3 weeks, 1 month, 2 months, 3 months, 4 months or longer.

In some embodiments, the engineered erythroid cell comprises a first exogenous polypeptide comprising a uricase, or a variant thereof, and further comprises a second exogenous polypeptide comprising a uric acid transporter, or a variant thereof, wherein the first and second exogenous polypeptides have an in vivo half-life of at least 24 hours. In some embodiments, the first and second exogenous polypeptides have an in vivo half-life of greater than 36 hours. In some embodiments, the first and second exogenous polypeptides have an in vivo half-life of greater than 48 hours. In some embodiments, the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first and second exogenous polypeptides have an in vivo half-life of 1 week, 2 weeks, 3 weeks, 1 month, 2 months, 3 months, 4 months or longer.

In some embodiments, the engineered erythroid cell comprises a first exogenous polypeptide comprising a uricase, or a variant thereof, a second exogenous polypeptide comprising a uric acid transporter, or a variant thereof, and a third exogenous polypeptide comprising a catalase, or a variant thereof, wherein the first, second and third exogenous polypeptides have an in vivo half-life of at least 24 hours. In some embodiments, the first, second and third exogenous polypeptides have an in vivo half-life of greater than 36 hours. In some embodiments, the first, second and third exogenous polypeptides have an in vivo half-life of greater than 48 hours. In some embodiments, the first, second and third exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first, second and third exogenous polypeptides have an in vivo half-life of 1 week, 2 weeks, 3 weeks, 1 month, 2 months, 3 months, 4 months or longer.

In some embodiments, the engineered erythroid cell comprises a first exogenous polypeptide comprising Candida utilis uricase, or a variant thereof, and further comprises a second exogenous polypeptide comprising human URAT1, or a variant thereof, wherein the first and second exogenous polypeptides have an in vivo half-life of at least 24 hours. In some embodiments, the first and second exogenous polypeptides have an in vivo half-life of greater than 36 hours. In some embodiments, the first and second exogenous polypeptides have an in vivo half-life of greater than 48 hours. In some embodiments, the first and second exogenous polypeptides have an in vivo half-life of about 24 hours, 36 hours, 48 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32, days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, or longer. In some embodiments, the first and second exogenous polypeptides have an in vivo half-life of 1 week, 2 weeks, 3 weeks, 1 month, 2 months, 3 months, 4 months or longer.

### Modifications

One or more of the exogenous proteins may have post-translational modifications characteristic of eukaryotic cells, e.g., mammalian cells, e.g., human cells. In some embodiments, one or more (e.g., 2, 3, 4, 5, 6, 7, 8, 9, 10, or more) of the exogenous proteins are glycosylated, phosphorylated, or both. In vitro detection of glycoproteins can be accomplished on SDS-PAGE gels and Western Blots using a modification of Periodic acid-Schiff (PAS) methods. Cellular localization of glycoproteins can be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., flavin mononucleotide (FMN) or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments of the above aspects and embodiments, the engineered erythroid cell is an enucleated cell, e.g. an erythrocyte or a reticulocyte. In some embodiments of the above aspects and embodiments, the engineered erythroid cell is a nucleated cell.

### Populations of Engineered Erythroid Cells

In one aspect, the invention features cell populations comprising the engineered erythroid cells of the invention, e.g., a plurality or population of the engineered erythroid cells. In various embodiments, the engineered erythroid cell population comprises predominantly enucleated cells, predominantly nucleated cells, or a mixture of enucleated and nucleated cells. In such cell populations, the enucleated cells can comprise reticulocytes, erythrocytes, or a mixture of reticulocytes and erythrocytes. In some embodiments, the enucleated cells are reticulocytes. In some embodiments, the enucleated cells are erythrocytes.

In some embodiments, the engineered erythroid cell population consists essentially of enucleated cells. In some embodiments, the engineered erythroid cell population comprises predominantly or substantially enucleated cells. For example, In some embodiments, the population of engineered erythroid cells comprises at least about 80% or more enucleated cells. In some embodiments, the population provided herein comprises at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99, or about 100% enucleated cells. In some embodiments, the population provided herein comprises greater than about 80% enucleated cells. In some embodiments, the population of engineered erythroid cells comprises greater than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% enucleated cells. In some embodiments, the population of engineered erythroid cells comprises between about 80% and about 100% enucleated cells, for example between about 80% and about 95%, about 80% and about 90%, about 80% and about 85%, about 85% and about 100%, about 85% and about 95%, about 85% and about 90%, about 90% and about 100%, about 90% and about 95%, or about 95% and about 100% of enucleated cells.

In some embodiments, the population of engineered erythroid cells comprises less than about 20% nucleated cells. For example, in embodiments, the population of engineered erythroid cells comprises less than about 1%, about 2%, about 3%, about 5%, about 6%, about 7%, about 8%, about 9%, about 10%, about 11%, about 12%, about 13%, about 14%, about 15%, about 16%, about 17%, about 18%, about 19%, or less than about 20% nucleated cells. In some embodiments, the population of engineered erythroid cells comprises less than about 1% nucleated cells. In some embodiments, the population of engineered erythroid cells comprises less than about 2% nucleated cells. In some embodiments, the population of engineered erythroid cells comprises less than about 3% nucleated cells. In some embodiments, the population of engineered erythroid cells comprises less than about 4% nucleated cells. In some embodiments, the population of engineered erythroid cells comprises less than about 5% nucleated cells. In some embodiments, the population of engineered erythroid cells comprises less than about 10% nucleated cells. In some embodiments, the population of engineered erythroid cells comprises less than about 15% nucleated cells. In some embodiments, the population of engineered erythroid cells comprises between 0% and 20% nucleated cells. In some embodiments, the populations of engineered erythroid cells comprise between about 0% and 20% nucleated cells, for example between about 0% and 19%, between about 0% and 15%, between about 0% and 10%, between about 0% and 5%, between about 0% and 4%, between about 0% and 3%, between about 0% and 2% nucleated cells, or between about 5% and 20%, between about 10% and 20%, or between about 15% and 20% nucleated cells.

In some embodiments, the disclosure features a population of the engineered erythroid cells of the invention, wherein the population of engineered erythroid cells comprises less than 20% nucleated cells and at least 80% enucleated cells, or comprises less than 15% nucleated cells and at least 85% nucleated cells, or comprises less than 10% nucleated cells and at least 90% enucleated cells, or comprises less than 5% nucleated cells and at least 95% enucleated cells. In some embodiments, the disclosure features populations of the engineered erythroid cells of the invention, wherein the population of engineered erythroid cells comprises about 0% nucleated cells and about 100% enucleated cells, about 1% nucleated cells and about 99% enucleated cells, about 2% nucleated cells and about 98% enucleated cells, about 3% nucleated cells and about 97% enucleated cells, about 4% nucleated cells and about 96% enucleated cells, about 5% nucleated cells and about 95% enucleated cells, about 6% nucleated cells and about 94% enucleated cells, about 7% nucleated cells and about 93% enucleated cells, about 8% nucleated cells and about 92% enucleated cells, about 9% nucleated cells and about 91% enucleated cells, about 10% nucleated cells and about 90% enucleated cells, about 11% nucleated cells and about 89% enucleated cells, about 12% nucleated cells and about 88% enucleated cells, about 13% nucleated cells and about 87% enucleated cells, about 14% nucleated cells and about 86% enucleated cells, about 85% nucleated cells and about 85% enucleated cells, about 16% nucleated cells and about 84% enucleated cells, about 17% nucleated cells and about 83% enucleated cells, about 18% nucleated cells and about 82% enucleated cells, about 19% nucleated cells and about 81% enucleated cells, or about 20% nucleated cells and about 80% enucleated cells.

In some embodiments, the engineered erythroid cell population comprises predominantly or substantially nucleated cells. In some embodiments, the engineered erythroid cell population consists essentially of nucleated cells. In various embodiments, the nucleated cells in the engineered erythroid cell population are erythrocyte (or fully mature red blood cell) precursor cells. In embodiments, the erythroid precursor cells are selected from the group consisting of pluripotent hematopoietic stem cells (HSCs), multipotent myeloid progenitor cells, CFU-S cells, BFU-E cells, CFU-E cells, pronormoblasts, basophilic normoblasts, polychromatophilic normoblasts and orthochromatophilic normoblasts.

In certain embodiments, the population of engineered erythroid cells comprises at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, at least about 99% or 100% nucleated cells.

It will be understood that during the preparation of the engineered erythroid cells of the invention, some fraction of cells may not become conjugated with an exogenous polypeptide or transduced to express an exogenous polypeptide. Accordingly, in some embodiments, a population of engineered erythroid cells provided herein comprises a mixture of engineered erythroid cells and unmodified erythroid cells, i.e., some fraction of cells in the population will not comprise, present, or express an exogenous polypeptide. For example, a population of engineered erythroid cells can comprise, in various embodiments, at least about 10%, 20%, 30%, 40%, 50%, 60%, 70%, 80%, 90%, 91%, 92%, 93%, 94%, 95%, 96%, 97%, 98% or 99% engineered erythroid cells, wherein the remaining erythroid cells in the population are not engineered. In embodiments, a single unit dose of engineered erythroid cells can comprise, in various embodiments, at least about 10%, 20%, 30%, 40%, 50%, 60%, 70%, 80%, 90%, 91%, 92%, 93%, 94%, 95%, 96%, 97%, 98% or 99% engineered erythroid cells, wherein the remaining erythroid cells in the dose are not engineered.

### III. Methods of Making Engineered Erythroid Cells

Various methods of making engineered erythroid cells, e.g., enucleated erythroid cells, or enucleated cells, are contemplated by the present disclosure.

Methods of manufacturing enucleated erythroid cells comprising an exogenous agent (e.g., a polypeptide) are described, e.g., in International Application Publication Nos. WO2015/073587 and WO2015/153102, each of which is incorporated by reference in its entirety.

In some embodiments, hematopoietic progenitor cells, e.g., CD34⁺ hematopoietic progenitor cells (e.g., human (e.g., adult human) or mouse cells), are contacted with a nucleic acid or nucleic acids encoding one or more exogenous polypeptides, and the cells are allowed to expand and differentiate in culture. In some embodiments, the CD34⁺ cells are immortalized, e.g., comprise a human papilloma virus (HPV; e.g., HPV type 16) E6 and/or E7 genes. In some embodiments, the immortalized CD34⁺ hematopoietic progenitor cell is a BEL-A cell line cell (see Trakarnasanga et al. (2017) Nat. Commun. 8: 14750). Additional immortalized CD34⁺ hematopoietic progenitor cells are described in U.S. Pat. Nos. 9,951,350, and 8,975,072. In some embodiments, an immortalized CD34⁺ hematopoietic progenitor cell is contacted with a nucleic acid or nucleic acids encoding one or more exogenous polypeptides, and the cells are allowed to expand and differentiate in culture.

In one aspect, the disclosure features an engineered enucleated cell (e.g., engineered enucleated erythroid cell), comprising a first exogenous polypeptide comprising a uric acid degrading polypeptide, or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure features an engineered enucleated cell (e.g., engineered enucleated erythroid cell), comprising a first exogenous polypeptide comprising a uric acid transporter, or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure features an engineered enucleated cell (e.g., engineered enucleated erythroid cell), comprising a first exogenous polypeptide comprising a catalase or a variant thereof, produced by a process comprising introducing an exogenous nucleic acid encoding the first exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide.

In another aspect, the disclosure features an engineered enucleated cell (e.g., engineered enucleated erythroid cell), comprising a first exogenous polypeptide comprising a uric acid degrading polypeptide, or a variant thereof, and a second exogenous polypeptide comprising a uric acid transporter, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide and the second exogenous polypeptide.

In another aspect, the disclosure features an engineered enucleated cell (e.g., engineered enucleated erythroid cell), comprising a first exogenous polypeptide comprising a uric acid degrading polypeptide, or a variant thereof, a second exogenous polypeptide comprising a uric acid transporter, or a variant thereof, and a third exogenous polypeptide comprising a catalase, or a variant thereof, produced by a process comprising introducing an exogenous nucleic acid encoding the first exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); introducing an exogenous nucleic acid encoding the second exogenous polypeptide into a nucleated erythroid cell (e.g., erythroid precursor cell); and culturing the nucleated erythroid cell under conditions suitable for enucleation of the nucleated erythroid cell and for production of the first exogenous polypeptide, the second exogenous polypeptide and the third exogenous polypeptide.

In some embodiments, the uric acid degrading polypeptide is a uricase, or a variant thereof. In some embodiments, the uric acid degrading polypeptide is an HIU hydrolase, or a variant thereof. In some embodiments, the uric acid degrading polypeptide is an OHCU decarboxylase, or a variant thereof. In some embodiments, the uric acid degrading polypeptide is an allantoinase, or variant thereof. In some embodiments, the uric acid degrading polypeptide is an allantoicase, or a variant thereof. In some embodiments, the uric acid degrading polypeptide is a myeloperoxidase, or variant thereof. In some embodiments, the uric acid degrading polypeptide is a FAD-dependent urate hydroxylase, or variant thereof. In some embodiments, the uric acid degrading polypeptide is a xanthine dehydrogenase, or variant thereof. In some embodiments, the uric acid degrading polypeptide is a nucleoside deoxyribosyltransferase, or variant thereof. In some embodiments, the uric acid degrading polypeptide is a dioxotetrahydropyrimidine phosphoribosyltransferase, or variant thereof. In some embodiments, the uric acid degrading polypeptide is a dihydropyrimidinase, or variant thereof. In some embodiments, the uric acid degrading polypeptide is a guanine deaminase, or variant thereof. In some embodiments, more than one uric acid degradation polypeptide, or variant thereof, may be combined in one or more erythroid cells, as described herein.

The processes of making the engineered erythroid cells are described in more detail below.

### Physical Characteristics of Engineered Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an engineered erythroid cell, e.g., an enucleated erythroid cell, that includes an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the engineered erythroid cell e.g., enucleated erythroid cell, comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the engineered erythroid cell e.g., enucleated erythroid cell, exhibits substantially the same osmotic membrane fragility as an isolated, uncultured erythroid cell that does not comprise an exogenous polypeptide. In some embodiments, the population of engineered erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the engineered erythroid cell, e.g., enucleated erythroid cell, has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In some embodiments the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In some embodiments the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the engineered erythroid cell, e.g., enucleated cell, has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the engineered erythroid cell, e.g., enucleated cell, has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587, which is herein incorporated by reference in its entirety.

Other Characteristics

In some embodiments, an engineered erythroid cell (e.g., engineered enucleated erythroid cell) or an engineered enucleated cell, or a population of engineered erythroid cells or engineered enucleated cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of erythroid cells or enucleated cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA⁺ (i.e., CD235a⁺) cells. In some embodiments, the population of engineered erythroid cells (engineered enucleated erythroid cells) or engineered enucleated cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁻ (CD36-negative) cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36⁻ (CD36-negative) cells. The presence of CD36 is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34⁻ (CD34-negative) cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺ cells.

In some embodiments, the population of engineered erythroid cells (engineered enucleated erythroid cells) or engineered enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺/CD34⁻/CD36⁻ cells. In some embodiments, the population of engineered erythroid cells (engineered enucleated erythroid cells) or engineered enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD34⁻/CD36⁻ cells.

In some embodiments, a population of engineered erythroid cells (engineered enucleated erythroid cells) or engineered enucleated cells comprising erythroid cells comprises less than about 10%, 9%, 8%, 7%, 6%, 5%, 4%, 3%, 2%, or 1% echinocytes.

In some embodiments, a population of engineered erythroid cells (e.g. artificial antigen presenting cells as described herein) comprising erythroid cells comprises less than about 10%, 9%, 8%, 7%, 6%, 5%, 4%, 3%, 2%, or 1% echinocytes.

In some embodiments, a population of engineered erythroid cells (engineered enucleated erythroid cells) or engineered enucleated cells comprises less than about 10%, 9%, 8%, 7%, 6%, 5%, 4%, 3%, 2%, or 1% pyrenocytes.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated. In some embodiments, the engineered erythroid cell is an enucleated cell. In some embodiments, the engineered erythroid cell is a nucleated cell.

### Isolating Erythrocytes

Mature erythrocytes may be isolated using various methods such as, for example, a cell washer, a continuous flow cell separator, density gradient separation, fluorescence-activated cell sorting (FACS), Miltenyi immunomagnetic depletion (MACS), or a combination of these methods (See, e.g., van der Berg et al., Clin. Chem. 33:1081-1082 (1987); Bar-Zvi et al., J. Biol. Chem. 262:17719-17723 (1987); Goodman et al., Exp. Biol. Med. 232:1470-1476 (2007)).

Erythrocytes may be isolated from whole blood by simple centrifugation (See, e.g., van der Berg et al., Clin. Chem. 33:1081-1082 (1987)). For example, EDTA-anticoagulated whole blood may be centrifuged at 800×g for 10 min at 4° C. The platelet-rich plasma and buffy coat are removed and the red blood cells are washed three times with isotonic saline solution (NaCl, 9 g/L).

Alternatively, erythrocytes may be isolated using density gradient centrifugation with various separation mediums such as, for example, Ficoll, Hypaque, Histopaque, Percoll,

Sigmacell, or combinations thereof. For example, a volume of Histopaque-1077 is layered on top of an equal volume of Histopaque-1119. EDTA-anticoagulated whole blood diluted 1:1 in an equal volume of isotonic saline solution (NaCl, 9 g/L) is layered on top of the Histopaque and the sample is centrifuged at 700×g for 30 min at room temperature. Under these conditions, granulocytes migrate to the 1077/1119 interface, lymphocytes, other mononuclear cells and platelets remain at the plasma/1077 interface, and the red blood cells are pelleted. The red blood cells are washed twice with isotonic saline solution.

Alternatively, erythrocytes may be isolated by centrifugation using a Percoll step gradient (See, e.g., Bar-Zvi et al., J. Biol. Chem. 262:17719-17723 (1987)). For example, fresh blood is mixed with an anticoagulant solution containing 75 mM sodium citrate and 38 mM citric acid and the cells washed briefly in Hepes-buffered saline. Leukocytes and platelets are removed by adsorption with a mixture of α-cellulose and Sigmacell (1:1). The erythrocytes are further isolated from reticulocytes and residual white blood cells by centrifugation through a 45/75% Percoll step gradient for 10 min at 2500 rpm in a Sorvall SS34 rotor. The erythrocytes are recovered in the pellet while reticulocytes band at the 45/75% interface and the remaining white blood cells band at the 0/45% interface. The Percoll is removed from the erythrocytes by several washes in Hepes-buffered saline. Other materials that may be used to generate density gradients for isolation of erythrocytes include OPTIPREP, a 60% solution of iodixanol in water (from Axis-Shield, Dundee, Scotland).

Erythrocytes may be separated from reticulocytes, for example, using flow cytometry (See, e.g., Goodman el al., Exp. Biol. Med. 232:1470-1476 (2007)). In this instance, whole blood is centrifuged (550×g, 20 min, 25° C.) to separate cells from plasma. The cell pellet is resuspended in phosphate buffered saline solution and further fractionated on Ficoll-Paque (1.077 density), for example, by centrifugation (400×g, 30 min, 25° C.) to separate the erythrocytes from the white blood cells. The resulting cell pellet is resuspended in RPMI supplemented with 10% serum and sorted on a FACS instrument such as, for example, a Becton Dickinson FACSCalibur (BD Biosciences, Franklin Lakes, N.J., USA) based on size and granularity.

Erythrocytes may be isolated by immunomagnetic depletion (See, e.g., Goodman, el al., (2007) Exp. Biol. Med. 232:1470-1476). In this instance, magnetic beads with cell-type specific antibodies are used to eliminate non-erythrocytes. For example, erythrocytes are isolated from the majority of other blood components using a density gradient as described herein followed by immunomagnetic depletion of any residual reticulocytes. The cells are pre-treated with human antibody serum for 20 min at 25° C. and then treated with antibodies against reticulocyte specific antigens such as, for example, CD71 and CD36. The antibodies may be directly attached to magnetic beads or conjugated to PE, for example, to which magnetic beads with anti-PE antibody will react. The antibody-magnetic bead complex is able to selectively extract residual reticulocytes, for example, from the erythrocyte population.

Erythrocytes may also be isolated using apheresis. The process of apheresis involves removal of whole blood from a patient or donor, separation of blood components using centrifugation or cell sorting, withdrawal of one or more of the separated portions, and transfusion of remaining components back into the patient or donor. A number of instruments are currently in use for this purpose such as for example the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods may be necessary to achieve the appropriate degree of cell purity.

Reticulocytes are immature red blood cells and compose approximately 1% of the red blood cells in the human body. Reticulocytes develop and mature in the bone marrow. Once released into circulation, reticulocytes rapidly undergo terminal differentiation to mature erythrocytes. Like mature erythrocytes, reticulocytes do not have a cell nucleus.

Reticulocytes of varying age may be isolated from peripheral blood based on the differences in cell density as the reticulocytes mature. Reticulocytes may be isolated from peripheral blood using differential centrifugation through various density gradients. For example, Percoll gradients may be used to isolate reticulocytes (See, e.g., Noble el al., Blood 74:475-481 (1989)). Sterile isotonic Percoll solutions of density 1.096 and 1.058 g/ml are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). These solutions have an osmolarity between 295 and 310 mOsm. Five milliliters, for example, of the first Percoll solution (density 1.096) is added to a sterile 15 ml conical centrifuge tube. Two milliliters, for example, of the second Percoll solution (density 1.058) is layered over the higher density first Percoll solution. Two to four milliliters of whole blood are layered on top of the tube. The tube is centrifuged at 250×g for 30 min in a refrigerated centrifuge with swing-out tube holders. Reticulocytes and some white cells migrate to the interface between the two Percoll layers. The cells at the interface are transferred to a new tube and washed twice with phosphate buffered saline (PBS) with 5 mM glucose, 0.03 mM sodium azide and 1 mg/ml BSA. Residual white blood cells are removed by chromatography in PBS over a size exclusion column.

Alternatively, reticulocytes may be isolated by positive selection using an immunomagnetic separation approach (See, e.g., Brun et al., Blood 76:2397-2403 (1990)). This approach takes advantage of the large number of transferrin receptors that are expressed on the surface of reticulocytes relative to erythrocytes prior to maturation. Magnetic beads coated with an antibody to the transferrin receptor may be used to selectively isolate reticulocytes from a mixed blood cell population. Antibodies to the transferrin receptor of a variety of mammalian species, including human, are available from commercial sources (e.g., Affinity BioReagents, Golden, Colo., USA; Sigma-Aldrich, Saint Louis, Mo., USA). The transferrin antibody may be directly linked to the magnetic beads. Alternatively, the transferrin antibody may be indirectly linked to the magnetic beads via a secondary antibody. For example, mouse monoclonal antibody 10D2 (Affinity BioReagents, Golden, Colo., USA) against human transferrin may be mixed with immunomagnetic beads coated with a sheep anti-mouse immunoglobulin G (Dynal/Invitrogen, Carlsbad, Calif., USA). The immunomagnetic beads are then incubated with a leukocyte-depleted red blood cell fraction. The beads and red blood cells are incubated at 22° C. with gentle mixing for 60-90 min followed by isolation of the beads with attached reticulocytes using a magnetic field. The isolated reticulocytes may be removed from the magnetic beads using, for example, DETACHaBEAD solution (from Invitrogen, Carlsbad, Calif., USA). Alternatively, reticulocytes may be isolated from in vitro growth and maturation of CD34+ hematopoietic stem cells using the methods described herein.

Terminally-differentiated enucleated erythrocytes can be separated from other cells based on their DNA content. In a non-limiting example, cells are first labeled with a vital DNA dye, such as Hoechst 33342 (Invitrogen Corp.). Hoechst 33342 is a cell-permeant nuclear counterstain that emits blue fluorescence when bound to double-stranded DNA. Undifferentiated precursor cells, macrophages or other nucleated cells in the culture are stained by Hoechst 33342, while enucleated erythrocytes are Hoechst-negative. The Hoechst-positive cells can be separated from enucleated erythrocytes by using fluorescence activated cell sorters or other cell sorting techniques. The Hoechst dye can be removed from the isolated erythrocytes by dialysis or other suitable methods.

### Vehicles for Polypeptides Described Herein

While in many embodiments herein, the one or more (e.g., two or more) exogenous polypeptides (e.g. a uric acid degrading polypeptide, a uric acid transporter, a catalase) are situated on or in an enucleated erythroid cell, it is understood that any polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more exogenous polypeptides described herein. In some embodiments, the vehicle comprises two or more exogenous polypeptides described herein, e.g., any pair of exogenous polypeptides described herein.

In one aspect, one or more polypeptides described herein are loaded onto, attached (e.g., immobilized or conjugated) to the surface of, and/or enclosed in a non-cellular delivery vehicle. The non-cellular delivery vehicle can be, for example, a nanolipidgel, a polymeric particle, an agarose particle, a latex particle, a silica particle, a liposome, or a multilamellar vesicles. In some embodiments, the non-cellular delivery vehicle comprises or consists of a nanoparticle of from about 1 nm to about 900 nm in diameter. In some embodiments, the non-cellular delivery vehicle comprises an average diameter of from about 0.1 to about 20 microns (such as from about 0.5 microns to about 10 microns, e.g., about 5 microns or less (e.g., about 2.5 to about 5 microns)). In some embodiments, the non-cellular delivery vehicle comprises an average diameter of from about 1 μm to about 10 μm. In some embodiments, the non-cellular delivery vehicle comprises a biodegradable polymer. In some embodiments, the non-cellular delivery vehicle comprises a natural polymer. In some embodiments, the non-cellular delivery vehicle comprises a synthetic polymer. Representative polymers include, but are not limited to, a poly(hydroxy acid), a polyhydroxyalkanoate, a polycaprolactone, a polycarbonate, a polyamide, a polyesteramide, poly(acrylamide), poly(ester), poly(alkylcyanoacrylates), poly(lactic acid) (PLA), poly(glycolic acids) (PGA), and poly(D,L-lactic-co-glycolic acid) (PLGA), and combinations thereof. In some embodiments, the non-cellular delivery vehicle comprises agarose, latex, or polystyrene. One or more of the polypeptides described herein can be conjugated to a non-cellular delivery vehicle using standard methods known in the art (see, e.g., Ulbrich et al. (2016) Chem Rev. 116(9): 5338-431). Conjugation can be either covalent or non-covalent. For example, in embodiments in which the non-cellular delivery vehicle is a liposome, a polypeptide described herein may be attached to the liposome via a polyethylene glycol (PEG) chain. Conjugation of a polypeptide to a liposome can also involve thioester bonds, for example by reaction of thiols and maleimide groups. Cross-linking agents can be used to create sulfhydryl groups for attachment of polypeptides to non-cellular delivery vehicles (see, e.g., Paszko and Senge (2012) Curr. Med. Chem. 19(31): 5239-77). In some embodiments, the non-cellular delivery vehicles comprising one or more of the polypeptides described herein may be used in any of therapeutic methods provided herein.

### Heterogeneous Populations of Cells

While in many embodiments herein, the one or more (e.g., two or more) exogenous polypeptides (e.g. a uric acid degrading polypeptide, a uric acid transporter, a catalase) are situated on or in a single cell, it is understood that any polypeptide or combination of polypeptides described herein can also be situated on a plurality of cells. For instance, in some aspects, the disclosure provides a plurality of erythroid cells, wherein a first cell of the plurality comprises a first exogenous polypeptide and a second cell of the plurality comprises a second exogenous polypeptide. In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

### Erythroid Precursor Cells

Provided herein are engineered erythroid precursor cells, and methods of making the engineered erythroid precursor cells, reticulocytes and erythrocytes.

Pluripotent stem cells give rise to erythrocytes by the process of erythropoiesis. The stem cell looks like a small lymphocyte and lacks the functional capabilities of the erythrocyte. The stem cells have the capacity of infinite division, something the mature cells lack. Some of the daughter cells arising from the stem cell acquire erythroid characters over generations and time. Most of the erythroid cells in the bone marrow have a distinct morphology but commitment to erythroid maturation is seen even in cells that have not acquired morphological features distinctive of the erythroid lineage. These cells are recognized by the type of colonies they form in vitro. Two such cells are recognized. Burst-forming unit erythroid (BFU-E) arise from the stem cell and gives rise to colony-forming unit erythroid (CFU-E). CFU-E gives rise to pronormoblast, the most immature of erythroid cells with a distinct morphology. BFU-E and CFU-E form a very small fraction of bone marrow cells. Morphologically five erythroid precursors are identifiable in the bone marrow stained with Romanovsky stains. The five stages from the most immature to the most mature are the proerythroblast, the basophilic normoblast (early erythroblast), polychromatophilic normoblast (intermediate erythroblast), orthochromatophilic normoblast (late erythroblast) and reticulocyte. BFU-E (burst forming unit-erythroid), CFU-E (erythroid colony-forming unit), pronormoblast (proerythroblast), basophilic normoblast, polychromatophilic normoblast and orthochromatophilic normoblast are lineage restricted.

The Table 3 below summarizes the morphological features of erythroid precursor cells and erythrocytes.

TABLE 3
Morphological features of erythroid precursor and erythroid cells

| Cell | Nucleus |
|---|---|
| Hematopoietic stem cell (HSC) | Yes |
| CMP (Common myeloid progenitor) | Yes |
| CFU-S (spleen colony forming cell; | Yes; Can differentiate into |
| myeloid precursor cell) | erythrocytes, platelets, |
| | macrophages. |
| BFU-E (burst forming unit-erythroid) | Yes |
| CFU-E (erythroid colony-forming | Yes |
| unit) | |
| Pronormoblast (proerythroblast) | Yes; fine chromatin, many nucleoli |
| Basophilic Normoblast | Yes; granular chromatin, |
| | nonucleoli |
| Polychromatophilic Normoblast | Yes; chromatin is visibly clumped |
| | with dark staining areas |
| Orthochromatophilic normoblast | Yes; featureless nucleus with dense |
| | chromatin |
| Reticulocyte | No Nucleus |
| Erythrocyte (fully matured RBC) | No Nucleus |

Normal human erythrocytes express CD36, an adhesion molecule of monocytes, platelets, and endothelial cells (van Schravendijk MR et al., Blood. 1992 Oct. 15; 80(8):2105-14). Accordingly, in some embodiments, an anti-CD36 antibody can be used to identify human erythrocytes.

Any type of cell known in the art that is capable of differentiating into an erythrocyte, i.e., any erythroid precursor cell, can be modified in accordance with the methods described herein to produce engineered erythroid precursor cells. In certain embodiments, the erythroid precursor cells modified in accordance with the methods described herein are cells that are in the process of differentiating into an erythrocyte, i.e., the cells are of a type known to exist during mammalian erythropoiesis. For example, the cells may be pluripotent hematopoietic stem cells (HSCs) or CD34+ cells, multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The modified erythroid precursor cells provided herein can be differentiated into engineered reticulocytes or erythrocytes in vitro using methods known in the art, i.e., using molecules known to promote erythropoiesis, e.g., SCF, Erythropoietin, IL-3, and/or GM-CSF, described herein below. Alternatively, the modified erythroid precursor cells are provided in a composition of the invention, and are capable of differentiating into erythrocytes upon administration to a subject in vivo.

In some embodiments, the erythroid precursor cells, e.g., hematopoietic stem cells, are from an O-negative donor. In some embodiments, the erythroid precursor cells lack (e.g., do not express or encode) A and/or B antigen.

### Culturing

Sources for generating engineered erythroid cells described herein include circulating erythroid cells. A suitable cell source may be isolated from a subject as described herein from patient-derived hematopoietic or erythroid progenitor cells, derived from immortalized erythroid cell lines, or derived from induced pluripotent stem cells, optionally cultured and differentiated. Methods for generating erythrocytes using cell culture techniques are well known in the art, e.g., Giarratana et al., Blood 2011, 118:5071, Huang et al., Mol Ther 2013, epub ahead of print September 3, or Kurita et al., PLOS One 2013, 8:e59890. Protocols vary according to growth factors, starting cell lines, culture period, and morphological traits by which the resulting cells are characterized. Culture systems have also been established for blood production that may substitute for donor transfusions (Fibach et al. 1989 Blood 73:100). Recently, CD34+ cells were differentiated to the reticulocyte stage, followed by successful transfusion into a human subject (Giarratana et al., Blood 2011, 118:5071).

Provided herein are culturing methods for erythroid cells and engineered erythroid cells. Erythroid cells can be cultured from hematopoietic progenitor cells, including, for example, CD34+ hematopoietic progenitor cells (Giarratana et al., Blood 2011, 118:5071), induced pluripotent stem cells (Kurita et al., PLOS One 2013, 8:e59890), and embryonic stem cells (Hirose et al. 2013 Stem Cell Reports 1:499). Cocktails of growth and differentiation factors that are suitable to expand and differentiate progenitor cells are known in the art. Examples of suitable expansion and differentiation factors include, but are not limited to, stem cell factor (SCF), an interleukin (IL) such as IL-1, IL-2, IL-3, IL-4, IL-5, IL-6, IL-7, IL-8, IL-9, IL-11, IL-12, CSF, G-CSF, thrombopoietin (TPO), GM-CSF, erythropoietin (EPO), Flt3, Flt2, PIXY 321, and leukemia inhibitory factor (LIF).

Erythroid cells can be cultured from hematopoietic progenitors, such as CD34+ cells, by contacting the progenitor cells with defined factors in a multi-step culture process. For example, erythroid cells can be cultured from hematopoietic progenitors in a three-step process.

The first step may comprise contacting the cells in culture with stem cell factor (SCF) at 1-1000 ng/mL, erythropoietin (EPO) at 1-100 U/mL, and interleukin-3 (IL-3) at 0.1-100 ng/mL. The first step optionally comprises contacting the cells in culture with a ligand that binds and activates a nuclear hormone receptor, such as e.g., the glucocorticoid receptor, the estrogen receptor, the progesterone receptor, the androgen receptor, or the pregnane x receptor. The ligands for these receptors include, for example, a corticosteroid, such as, e.g., dexamethasone at 10 nM-100 μM or hydrocortisone at 10 nM-100 μM; an estrogen, such as, e.g., beta-estradiol at 10 nM-100 μM; a progestogen, such as, e.g., progesterone at 10 nM-100 μM, hydroxyprogesterone at 10 nM-100 μM, 5a-dihydroprogesterone at 10 nM-100 μM, 11-deoxycorticosterone at 10 nM-100 μM, or a synthetic progestin, such as, e.g., chlormadinone acetate at 10 nM-100 μM; an androgen, such as, e.g., testosterone at 10 nM-100 μM, dihydrotestosterone at 10 nM-100 μM or androstenedione at 10 nM-100 μM; or a pregnane x receptor ligand, such as, e.g., rifampicin at 10 nM-100 μM, hyperforin at 10 nM-100 St. John's Wort (hypericin) at 10 nM-100 μM, or vitamin E-like molecules, such as, e.g., tocopherol at 10 nM-100 The first step may also optionally comprise contacting the cells in culture with an insulin-like molecule, such as, e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The first step further may optionally comprise contacting the cells in culture with transferrin at 0.1-5 mg/mL.

The first step may optionally comprise contacting the cells in culture with one or more interleukins (IL) or growth factors such as, e.g., IL-1, IL-2, IL-4, IL-5, IL-6, IL-7, IL-8, IL-9, IL-11, IL-12, granulocyte colony-stimulating factor (G-CSF), macrophage colony-stimulating factor (M-CSF), granulocyte-macrophage colony-stimulating factor (GM-CSF), thrombopoietin, fibroblast growth factor (FGF), platelet-derived growth factor (PDGF), transforming growth factor beta (TGF-B), tumor necrosis factor alpha (TNF-A), megakaryocyte growth and development factor (MGDF), leukemia inhibitory factor (LIF), and Flt3 ligand. Each interleukin or growth factor may typically be supplied at a concentration of 0.1-100 ng/mL. The first step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The second step may comprise contacting the cells in culture with stem cell factor (SCF) at 1-1000 ng/mL and erythropoietin (EPO) at 1-100 U/mL. The second step may also optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The second step may further optionally comprise contacting the cells in culture with transferrin at 0.1-5 mg/mL. The second may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The third step may comprise contacting the cells in culture with erythropoietin (EPO) at 1-100 U/mL. The third step may optionally comprise contacting the cells in culture with stem cell factor (SCF) at 1-1000 ng/mL. The third step may further optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The third step may also optionally comprise contacting the cells in culture with transferrin at 0.1-5 mg/mL. The third step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

In some embodiments, methods of expansion and differentiation of the engineered erythroid cells presenting one or more exogenous polypeptides, do not include culturing the engineered erythroid cells in a medium comprising a myeloproliferative receptor (mpl) ligand.

The culture process may optionally comprise contacting cells by a method known in the art with a molecule, e.g., a DNA molecule, an RNA molecule, a mRNA, an siRNA, a microRNA, a lncRNA, a shRNA, a hormone, or a small molecule, that activates or knocks down one or more genes. Target genes can include, for example, genes that encode a transcription factor, a growth factor, or a growth factor receptor, including but not limited to, e.g., GATA1, GATA2, CMyc, hTERT, p53, EPO, SCF, insulin, EPO-R, SCF-R, transferrin-R, insulin-R.

In some embodiments, CD34+ cells are placed in a culture containing varying amounts of IMDM, FBS, glutamine, BSA, holotransferrin, insulin, dexamethasone, .beta.-estradiol, IL-3, SCF, and erythropoietin, in three separate differentiation stages for a total of 22 days.

In some embodiments, CD34+ cells are placed in a culture containing varying amounts of IMDM, FBS, glutamine, BSA, holotransferrin, insulin, dexamethasone, .beta.-estradiol, IL-3, SCF, and thrombopoietin, in three separate differentiation stages for a total of 14 days.

In some embodiments, CD34+ cells are placed in a culture containing varying amounts of IMDM, FBS, glutamine, BSA, holotransferrin, insulin, dexamethasone, .beta.-estradiol, IL-3, SCF, and GCSF, in three separate differentiation stages for a total of 15 days.

In some embodiments, the erythroid cells are expanded at least 100, 1000, 2000, 5000, 10,000, 20,000, 50,000, or 100,000 fold (and optionally up to 100,000, 200,000, or 500,000 fold). Number of cells is measured, in some embodiments, using an automated cell counter. In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated erythroid cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, it may be desirable during culturing to only partially differentiate the erythroid progenitor cells, e.g., hematopoietic stem cells, in vitro, allowing further differentiation, e.g., differentiation into reticulocytes or fully mature erythrocytes, to occur upon introduction to a subject in vivo (See, e.g., Neildez-Nguyen et al., Nature Biotech. 20:467-472 (2002)). It will be understood that, in various embodiments of the invention, maturation and/or differentiation in vitro may be arrested at any stage desired. For example, isolated CD34+ hematopoietic stem cells may be expanded in vitro as described elsewhere herein, e.g., in medium containing various factors, including, for example, interleukin 3, Flt3 ligand, stem cell factor, thrombopoietin, erythropoietin, transferrin, and insulin growth factor, to reach a desired stage of differentiation. The resulting engineered erythroid cells may be characterized by the surface expression of CD36 and GPA, and other characteristics specific to the particular desired cell type, and may be transfused into a subject where terminal differentiation to mature erythrocytes is allowed to occur.

In some embodiments, engineered erythroid cells are partially expanded from erythroid progenitor cells to any stage of maturation prior to but not including enucleation, and thus remain nucleated cells, e.g., erythroid precursor cells. In certain embodiments, the resulting cells are nucleated and erythroid lineage restricted. In certain embodiments, the resulting cells are selected from multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The final differentiation steps, including enucleation, occur only after administration of the engineered erythroid cell to a subject, that is, in such embodiments, the enucleation step occurs in vivo. In some embodiments, engineered erythroid cells are expanded and differentiated in vitro through the stage of enucleation to become, e.g., reticulocytes. In such embodiments where the engineered erythroid cells are differentiated to the stage of reticuloyctes, the final differentiation step to become erythrocytes occurs only after administration of the engineered erythroid cell to a subject, that is, the terminal differentiation step occurs in vivo. In some embodiments, engineered erythroid cells are expanded and differentiated in vitro through the terminal differentiation stage to become erythrocytes.

It will be further recognized that in some embodiments, the engineered erythroid cells may be expanded and differentiated from erythroid progenitor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. Such engineered platelets including exogenous polypeptides as described herein are considered to be encompassed by the present invention.

It will be further recognized that in some embodiments, the engineered erythroid cells may be expanded and differentiated from erythroid progenitor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. In some embodiments, such engineered platelets including exogenous polypeptides as described herein are considered to be encompassed by the present invention.

In some embodiments of the above aspects and embodiments, the engineered erythroid cell is an enucleated cell, e.g. an erythrocyte or a reticulocyte. In some embodiments of the above aspects and embodiments, the engineered erythroid cell is a nucleated cell.

In some embodiments, an enucleated cell provided herein is a platelet. Methods of manufacturing platelets in vitro are known in the art (see, e.g., Wang and Zheng (2016) Springerplus 5(1): 787, and U.S. Pat. No. 9,574,178). Methods of manufacturing platelets including an exogenous polypeptide are described, e.g., in International Patent Application Publication Nos. WO2015/073587 and WO2015/153102, each of which is incorporated by reference in its entirety. Platelet production is in part regulated by signaling mechanisms induced by interaction between thrombopoietin (TPO) and its cellular receptor TPOR/MPUc-MPL. In addition, multiple cytokines (e.g., stem cell factor (SCF), IL-1, IL-3, IL-6, IL-11, leukemia inhibiting factor (LIF), G-CSF, GM-CSF, M-CSF, erythropoietin (EPO), kit ligand, and interferon) have been shown to possess thrombocytopoietic activity.

In some embodiments, platelets are generated from hematopoietic progenitor cells, such as CD34⁺ hematopoietic stem cells, induced pluripotent stem cells or embryonic stem cells. In some embodiments, platelets are produced by contacting the progenitor cells with defined factors in a multi-step culture process. In some embodiments, the multi-step culture process comprises: culturing a population of hematopoietic progenitor cells under conditions suitable to produce a population of megakaryocyte progenitor cells, and culturing the population of megakaryocyte progenitor cells under conditions suitable to produce platelets. Cocktails of growth and differentiation factors that are suitable to expand and differentiate progenitor cells and produce platelets are known in the art. Examples of suitable expansion and differentiation factors include, but are not limited to, stem cell factor (SCF), Flt-3/Flk-2 ligand (FL), TPO, IL-11, IL-3, IL-6, and IL-9. For instance, in some embodiments, platelets may be produced by seeding CD34⁺ HSCs in a serum-free medium at 2-4×10⁴ cells/mL, and refreshing the medium on culture day 4 by adding an equal volume of media. On culture day 6, cells are counted and analyzed: 1.5×10⁵ cells are washed and placed in 1 mL of the same medium supplemented with a cytokine cocktail comprising TPO (30 ng/mL), SCF (1 ng/mL), IL-6 (7.5 ng/mL), and IL-9 (13.5 ng/mL) to induce megakaryocyte differentiation. At culture day 10, from about one quarter to about half of the suspension culture is replaced with fresh media. The cells are cultured in a humidified atmosphere (10% CO₂) at 39° C. for the first 6 culture days, and at 37° C. for the last 8 culture days. Viable nucleated cells are counted with a hemocytometer following trypan blue staining. The differentiation state of platelets in culture can be assessed by flow cytometry or quantitative PCR as described in Examples 44 and 45 of in International Patent Application Publication No. WO2015/073587, incorporated herein by reference.

### Expression of Exogenous Polypeptides

In some embodiments, the engineered erythroid cells described herein are generated by contacting a suitable isolated cell, e.g., an erythroid cell, a reticulocyte, an erythroid precursor cell, a platelet, or a platelet precursor, with an exogenous nucleic acid encoding a polypeptide of the disclosure (e.g. a uricase and/or a uric acid transporter and/or a catalase).

In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleate erythroid cell, a nucleated platelet precursor cell, or a reticulocyte. In some embodiments, the exogenous polypeptide is contacted with a primary platelet, a nucleated erythroid cell (e.g., erythroid precursor cell), a nucleated platelet precursor cell, a reticulocyte, or an erythrocyte.

An exogenous polypeptide may be expressed from a transgene introduced into an erythroid cell by electroporation, chemical or polymeric transfection, viral transduction, mechanical membrane disruption, or other method; an exogenous polypeptide that is expressed from mRNA that is introduced into a cell by electroporation, chemical or polymeric transfection, viral transduction, mechanical membrane disruption, or other method; an exogenous polypeptide that is over-expressed from the native locus by the introduction of an external factor, e.g., a transcriptional activator, transcriptional repressor, or secretory pathway enhancer; and/or a polypeptide that is synthesized, extracted, or produced from a production cell or other external system and incorporated into the erythroid cell.

In certain embodiments, the introducing step comprises viral transduction. In some embodiments, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector.

In some embodiments, the introducing step comprises introducing the first exogenous nucleic acid encoding the first exogenous polypeptide by transfection of a lentiviral vector.

Exogenous polypeptides (e.g. a uricase or a uric acid transporter or a catalase) can be introduced by transfection of single or multiple copies of genes, transduction with a virus, or electroporation in the presence of DNA or RNA. Methods for expression of exogenous proteins in mammalian cells are well known in the art. For example, expression of exogenous factor IX in hematopoietic cells is induced by viral transduction of CD34+ progenitor cells, see Chang et al., Nat Biotechnol 2006, 24:1017.

In some embodiments, when there are more than one polypeptides (e.g. two or more) the polypeptides may be encoded in a single nucleic acid, e.g. a single vector. When both uricase and uric acid transporter are encoded in the same vector, there are multiple possible sub-strategies useful for this method of co-expression. In some embodiments, the single vector has a separate promoter for each gene, has two proteins that are initially transcribed into a single polypeptide having a protease cleavage site in the middle (e.g. a T2A site), so that subsequent proteolytic processing yields two proteins, or any other suitable configuration. In some embodiments, the recombinant nucleic acid comprises a gene encoding a first exogenous polypeptide, wherein the first exogenous polypeptide is uricase, or a variant thereof, and a gene encoding a second exogenous polypeptide, wherein the second exogenous polypeptide is a uric acid transporter, or a variant thereof, wherein the second gene is separated from the gene encoding the exogenous polypeptide by a viral-derived T2A sequence (gagggcagaggaagtcttctaacatgcggtgacgtggaggsgsstcccggccct (SEQ ID NO: 55)) that is post-translationally cleaved into two mature proteins.

For dual expression via 2 promoters, the MSCV promoter may be used as promoter #1 and the EF1 promoter as promoter #2, although the disclosure is not to be limited by these two exemplary promoters. Another strategy is to express both uricase and uric acid transporter proteins by inserting an internal ribosome entry site (IRES) between the two genes. Still another strategy is to express uricase and uric acid transporter as direct peptide fusions separated by a linker.

In some embodiments, the two or more polypeptides are encoded in two or more nucleic acids, e.g., each vector encodes one of the polypeptides.

In certain embodiments, the lentiviral vector comprises a promoter selected from the group consisting of beta-globin promoter, murine stem cell virus (MSCV) promoter, Gibbon ape leukemia virus (GALV) promoter, human elongation factor lalpha (EFlalpha) promoter, CAG CMV immediate early enhancer and the chicken beta-actin (CAG), and human phosphoglycerate kinase 1 (PGK) promoter.

Nucleic acids such as DNA expression vectors or mRNA for producing the exogenous polypeptides may be introduced into progenitor cells (e.g., an erythroid cell progenitor or a platelet progenitor and the like) that are suitable to produce the exogenous polypeptides described herein. The progenitor cells can be isolated from an original source or obtained from expanded progenitor cell population via routine recombinant technology as provided herein. In some instances, the expression vectors can be designed such that they can incorporate into the genome of cells by homologous or non-homologous recombination by methods known in the art.

In some embodiments, hematopoietic progenitor cells, e.g., CD34+ hematopoietic progenitor cells, are contacted with a nucleic acid or nucleic acids encoding one or more exogenous polypeptides, and the cells are allowed to expand and differentiate in culture.

According to some embodiments, one or more exogenous polypeptides may be cloned into plasmid constructs for transfection. Methods for transferring expression vectors into cells that are suitable to produce the engineered erythroid cells described herein include, but are not limited to, viral mediated gene transfer, liposome mediated transfer, transformation, gene guns, transfection and transduction, e.g., viral mediated gene transfer such as the use of vectors based on DNA viruses such as adenovirus, adenoassociated virus and herpes virus, as well as retroviral based vectors. Examples of modes of gene transfer include e.g., naked DNA, CaPO₄ precipitation, DEAE dextran, electroporation, protoplast fusion, lipofection, and cell microinjection.

According to some embodiments, recombinant DNA encoding each exogenous polypeptide may be cloned into a lentiviral vector plasmid for integration into erythroid cells. In some embodiments, the lentiviral vector comprises DNA encoding a single exogenous polypeptide for integration into erythroid cells. For example, in some embodiments, the lentiviral vector comprises DNA encoding a uricase polypeptide for integration into erythroid cells. In some embodiments, the lentiviral vector comprises DNA encoding a uric acid transporter for integration into erythroid cells. In other embodiments, the lentiviral vector comprises two, three, four or more exogenous polypeptides as described herein for integration into erythroid cells. For example, in some embodiments, the lentiviral vector comprises DNA encoding a uricase polypeptide and a uric acid transporter polypeptide for integration into erythroid cells. According to some embodiments, recombinant DNA encoding the one or more exogenous polypeptides may be cloned into a plasmid DNA construct encoding a selectable trait, such as an antibiotic resistance gene. According to some embodiments, recombinant DNA encoding the exogenous polypeptides may be cloned into a plasmid construct that is adapted to stably express each recombinant protein in the erythroid cells.

According to some embodiments, the lentiviral system may be employed where the transfer vector with exogenous polypeptides sequences (e.g., one, two, three, four or more exogenous polypeptide sequences), an envelope vector, and a packaging vector are each transfected into host cells for virus production. According to some embodiments, the lentiviral vectors may be transfected into host cells by any of calcium phosphate precipitation transfection, lipid based transfection, or electroporation, and incubated overnight. For embodiments where the exogenous polypeptide sequence may be accompanied by a fluorescence reporter, inspection of the host cells for florescence may be checked after overnight incubation. The culture medium of the host cells comprising virus particles may be harvested 2 or 3 times every 8-12 hours and centrifuged to sediment detached cells and debris. The culture medium may then be used directly, frozen or concentrated as needed.

A progenitor cell subject to transfer of an exogenous nucleic acid that encodes an exogenous polypeptide can be cultured under suitable conditions allowing for differentiation into mature red blood cells, e.g., the in vitro culturing process described herein. The resulting red blood cells display proteins associated with mature erythrocytes, e.g., hemoglobin, glycophorin A, and exogenous polypeptides which can be validated and quantified by standard methods (e.g., Western blotting or FACS analysis). Isolated mature red blood cells comprising a first exogenous polypeptide, a second exogenous polypeptide, or both a first and a second exogenous polypeptide are non-limiting examples of engineered erythroid cells of the disclosure.

In some embodiments, the engineered erythroid cell is generated by contacting an erythroid precursor cell with an exogenous nucleic acid encoding an exogenous polypeptide. In some embodiments, the exogenous polypeptide is encoded by an RNA which is contacted with an erythroid precursor cell.

Isolated erythroid precursor cells may be transfected with mRNA encoding one or more exogenous polypeptides to generate an engineered erythroid cell. Messenger RNA may be derived from in vitro transcription of a cDNA plasmid construct containing the coding sequence corresponding to the one or more exogenous polypeptides. For example, the cDNA sequence corresponding to the exogenous polypeptide may be inserted into a cloning vector containing a promoter sequence compatible with specific RNA polymerases. For example, the cloning vector ZAP EXPRESS pBK-CMV (Stratagene, La Jolla, Calif., USA) contains T3 and T7 promoter sequence compatible with T3 and T7 RNA polymerase, respectively. For in vitro transcription of sense mRNA, the plasmid is linearized at a restriction site downstream of the stop codon(s) corresponding to the end of the coding sequence of the exogenous polypeptide. The mRNA is transcribed from the linear DNA template using a commercially available kit such as, for example, the RNAMAXX High Yield Transcription Kit (from Stratagene, La Jolla, Calif., USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase I to eliminate the linearized DNA template followed by precipitation in 70% ethanol in the presence of lithium chloride, sodium acetate or ammonium acetate. The integrity of the transcribed mRNA may be assessed using electrophoresis with an agarose-formaldehyde gel or commercially available Novex pre-cast TBE gels (e.g., Novex, Invitrogen, Carlsbad, Calif., USA).

Messenger RNA encoding the one or more exogenous polypeptides may be introduced into reticulocytes using a variety of approaches including, for example, lipofection and electroporation (van Tandeloo et al., Blood 98:49-56 (2001)). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C(Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891 (2001)). The resulting mRNA/lipid complexes are incubated with cells (1-2×10⁶ cells/ml) for 2 h at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, and Easyject Plus device (EquiBio, Kent, United Kingdom). In some instances, it may be necessary to test various voltages, capacitances and electroporation volumes to determine the useful conditions for transfection of a particular mRNA into a reticulocyte. In general, the electroporation parameters required to efficiently transfect cells with mRNA appear to be less detrimental to cells than those required for electroporation of DNA (van Tandeloo et al., Blood 98:49-56 (2001)).

Alternatively, mRNA may be transfected into an erythroid precursor cell using a peptide-mediated RNA delivery strategy (see, e.g., Bettinger et al., Nucleic Acids Res. 29:3882-3891 (2001)). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment and then removed and the transfected cells allowed to continue growing in culture.

In some embodiments, the engineered erythroid cell is generated by contacting a suitable isolated erythroid precursor cell or a platelet precursor cell with an exogenous nucleic acid encoding one or more exogenous polypeptides. In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleate erythroid cell, or a nucleated platelet precursor cell.

The one or more exogenous polypeptides may be genetically introduced into an erythroid precursor cell, a platelet precursor cell, or a nucleated erythroid cell (e.g., erythroid precursor cell), prior to terminal differentiation using a variety of DNA techniques, including transient or stable transfections and gene therapy approaches. The exogenous polypeptides may be expressed on the surface and/or in the cytoplasm of mature red blood cell or platelet.

Viral gene transfer may be used to transfect the cells with DNA encoding one or more exogenous polypeptides. A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example (See, e.g., Osten et al., HEP 178:177-202 (2007)). Retroviruses, for example, efficiently transduce mammalian cells including human cells and integrate into chromosomes, conferring stable gene transfer.

One or more exogenous polypeptides may be transfected into an erythroid precursor cell, a platelet precursor cell, or a nucleated erythroid cell (e.g., erythroid precursor cell), expressed and subsequently retained and exhibited in a mature red blood cell or platelet. A suitable vector is the Moloney murine leukemia virus (MMLV) vector backbone (Malik et al., Blood 91:2664-2671 (1998)). Vectors based on MMLV, an oncogenic retrovirus, are currently used in gene therapy clinical trials (Hossle et al., News Physiol. Sci. 17:87-92 (2002)). For example, a DNA construct containing the cDNA encoding an exogenous polypeptide can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid precursor cell, a platelet precursor cell, or a nucleated erythroid cell (e.g., erythroid precursor cell) that has been isolated and cultured or has been freshly isolated as described herein. The expression of the exogenous polypeptide may be monitored using FACS analysis (fluorescence-activated cell sorting), for example, with a fluorescently labeled antibody directed against the exogenous polypeptide, if it is located on the surface of the engineered erythroid cell Similar methods may be used to express an exogenous polypeptide that is located in the inside of the engineered erythroid cell.

Optionally, a fluorescent tracking molecule such as, for example, green fluorescent protein (GFP) may be transfected using a viral-based approach (Tao et al., Stem Cells 25:670-678 (2007)). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce e.g., an erythroid precursor cell, a platelet precursor cell, or a nucleated erythroid cell (e.g., erythroid precursor cell). In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of cells expressing EGFP or DsRed-Express may be assessed by FACS. Other reporter genes that may be used to assess transduction efficiency include, for example, beta-galactosidase, chloramphenicol acetyltransferase, and luciferase as well as low-affinity nerve growth factor receptor (LNGFR), and the human cell surface CD24 antigen (Bierhuizen et al., Leukemia 13:605-613 (1999)).

Nonviral vectors may be used to introduce genetic material into suitable erythroid cells, platelets or precursors thereof to generate the engineered erythroid cells described herein. Nonviral-mediated gene transfer differs from viral-mediated gene transfer in that the plasmid vectors contain no proteins, are less toxic and easier to scale up, and have no host cell preferences. The “naked DNA” of plasmid vectors is by itself inefficient in delivering genetic material encoding a polypeptide to a cell and therefore is combined with a gene delivery method that enables entry into cells. A number of delivery methods may be used to transfer nonviral vectors into suitable erythroid cells, platelets or precursors thereof including chemical and physical methods.

A nonviral vector encoding one or more exogenous polypeptides may be introduced into suitable erythroid cells, platelets or precursors thereof using synthetic macromolecules such as cationic lipids and polymers (Papapetrou et al., Gene Therapy 12:S118-S130 (2005)). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (See, e.g., Keller et al., Gene Therapy 6:931-938 (1999)). For erythroid cells, platelets or precursors thereof the plasmid DNA (approximately 0.5 μg in 25-100 μL of a serum free medium, such as, for example, OptiMEM (Invitrogen, Carlsbad, Calif.)) is mixed with a cationic liposome (approximately 4 μ.g in 25 μ.L of serum free medium) such as the commercially available transfection reagent Lipofectamine.™. (Invitrogen, Carlsbad, Calif.) and allowed to incubate for at least 20 min to form complexes. The DNA/liposome complex is added to suitable erythroid cells, platelets or precursors thereof and allowed to incubate for 5-24 hours, after which time transgene expression of the polypeptide may be assayed. Alternatively, other commercially available liposome transfection agents may be used (e.g., In vivo GeneSHUTTLE., Qbiogene, Carlsbad, Calif.).

Optionally, a cationic polymer such as, for example, polyethylenimine (PEI) may be used to efficiently transfect erythroid cell progenitor cells, for example hematopoietic and umbilical cord blood-derived CD34+ cells (See, e.g., Shin et al., Biochim. Biophys. Acta 1725:377-384 (2005)). Human CD34+ cells are isolated from human umbilical cord blood and cultured in Iscove's modified Dulbecco's medium supplemented with 200 ng/ml stem cell factor and 20% heat-inactivated fetal bovine serum. Plasmid DNA encoding the exogenous polypeptide is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/ml distilled water and slightly acidified to pH 5.0 using HCl. The DNA may be combined with the PEI for 30 min at room temperature at various nitrogen/phosphate ratios based on the calculation that 1μg of DNA contains 3 nmol phosphate and 1 μl of PEI stock solution contains 10 nmol amine nitrogen. The isolated CD34+ cells are seeded with the DNA/cationic complex, centrifuged at 280×g for 5 min and incubated in culture medium for 4 or more h until gene expression of the polypeptide is assessed.

A plasmid vector may be introduced into suitable erythroid cells, platelets or precursors thereof using a physical method such as particle-mediated transfection, “gene gun”, biolistics, or particle bombardment technology (Papapetrou, et al., (2005) Gene Therapy 12:S118-S130). In this instance, DNA encoding the polypeptide is absorbed onto gold particles and administered to cells by a particle gun. This approach may be used, for example, to transfect erythroid progenitor cells, e.g., hematopoietic stem cells derived from umbilical cord blood (See, e.g., Verma et al., Gene Therapy 5:692-699 (1998)). As such, umbilical cord blood is isolated and diluted three fold in phosphate buffered saline. CD34+ cells are purified using an anti-CD34 monoclonal antibody in combination with magnetic microbeads coated with a secondary antibody and a magnetic isolation system (e.g., Miltenyi MiniMac System, Auburn, Calif., USA). The CD34+ enriched cells may be cultured as described herein. For transfection, plasmid DNA encoding the polypeptide is precipitated onto a particle, for example gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into suitable erythroid cells, platelets or precursors thereof. Electroporation creates transient pores in the cell membrane, allowing for the introduction of various molecules into the cells including, for example, DNA and RNA as well as antibodies and drugs. As such, CD34+ cells are isolated and cultured as described herein. Immediately prior to electroporation, the cells are isolated by centrifugation for 10 min at 250×g at room temperature and resuspended at 0.2-10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μl of cell suspension. Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser XCELL, BioRad, Hercules, Calif.; Cellject Duo, Thermo Science, Milford, Mass.). Alternatively, efficient electroporation of isolated CD34+ cells may be performed using the following parameters: 4 mm cuvette, 1600 μF, 550 V/cm, and 10 μg of DNA per 500 μl of cells at 1×10⁵ cells/ml (Oldak et al., Acta Biochimica Polonica 49:625-632 (2002)).

Nucleofection, a form of electroporation, may also be used to transfect suitable erythroid cells, platelets or precursors thereof. In this instance, transfection is performed using electrical parameters in cell-type specific solutions that enable DNA (or other reagents) to be directly transported to the nucleus thus reducing the risk of possible degradation in the cytoplasm. For example, a Human CD34 CELL NYCLEOFECTOR Kit (from Amaxa Inc.) may be used to transfect suitable erythroid cells, platelets or precursors thereof. In this instance, 1-5×10⁶ cells in Human CD34 Cell NUCLEOFECTOR Solution are mixed with 1-5 μg of DNA and transfected in the NUCLEOFECTOR instrument using preprogrammed settings as determined by the manufacturer.

Erythroid cells, platelets or precursors thereof may be non-virally transfected with a conventional expression vector which is unable to self-replicate in mammalian cells unless it is integrated in the genome. Alternatively, erythroid cells, platelets or precursors thereof may be transfected with an episomal vector which may persist in the host nucleus as autonomously replicating genetic units without integration into chromosomes (Papapetrou et al., Gene Therapy 12:S118-S130 (2005)). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV) and Simian virus 40 (SV40). Mammalian artificial chromosomes may also be used for nonviral gene transfer (Vanderbyl et al., Exp. Hematol. 33:1470-1476 (2005)).

Exogenous nucleic acids encoding one or more exogenous polypeptides may be assembled into expression vectors by standard molecular biology methods known in the art, e.g., restriction digestion, overlap-extension PCR, and Gibson assembly.

Exogenous nucleic acids may comprise a gene encoding one or more exogenous polypeptides that are not normally expressed on the cell surface, e.g., of an erythroid cell, fused to a gene that encodes an endogenous or native membrane protein, such that the exogenous polypeptide is expressed on the cell surface. For example, an exogenous gene encoding an exogenous polypeptide can be cloned at the N terminus following the leader sequence of a type 1 membrane protein, at the C terminus of a type 2 membrane protein, or upstream of the GPI attachment site of a GPI-linked membrane protein.

Standard cloning methods can be used to introduce flexible amino acid linkers between two fused genes. For example, the flexible linker is a poly-glycine poly-serine linker such as [Gly4Ser]3 (SEQ ID NO: 26) commonly used in generating single-chain antibody fragments from full-length antibodies (Antibody Engineering: Methods & Protocols, Lo 2004), or ala-gly-ser-thr polypeptides such as those used to generate single-chain Arc repressors (Robinson & Sauer, PNAS 1998). In some embodiments, the flexible linker provides the polypeptide with more flexibility and steric freedom than the equivalent construct without the flexible linker. In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO: 48), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Exemplary linkers include, but are not limited to, GGGGSGGGG (SEQ ID NO: 33), GGGGSGGGGS (SEQ ID NO: 49), GSGSGSGSGS (SEQ ID NO: 50), PSTSTST (SEQ ID NO: 51), and EIDKPSQ (SEQ ID NO: 52), and multimers thereof.

An epitope tag may be placed between two fused genes, such as, e.g., a nucleic acid sequence encoding an HA epitope tag—amino acids YPYDVPDYA (SEQ ID NO: 27), a CMyc tag—amino acids EQKLISEEDL (SEQ ID NO: 28), or a Flag tag—amino acids DYKDDDDK (SEQ ID NO: 29). The epitope tag may be used for the facile detection and quantification of expression using antibodies against the epitope tag by flow cytometry, western blot, or immunoprecipitation.

In some embodiments, the engineered erythroid cell comprises one or more exogenous polypeptides and at least one other heterologous polypeptide. The at least one other heterologous polypeptide can be a fluorescent protein. The fluorescent protein can be used as a reporter to assess transduction efficiency. In some embodiments, the fluorescent protein is used as a reporter to assess expression levels of the exogenous polypeptide if both are made from the same transcript. In some embodiments, the at least one other polypeptide is heterologous and provides a function, such as, e.g., multiple antigens, multiple capture targets, enzyme cascade.

In certain embodiments, the engineered erythroid cell is an erythroid cell that presents a first exogenous polypeptide that is conjugated with a second exogenous polypeptide. Conjugation may be achieved chemically or enzymatically. Chemical conjugation may be accomplished by covalent bonding of the exogenous antigen-presenting polypeptide to one or more exogenous polypeptides, with or without the use of a linker. Chemical conjugation may be accomplished by the covalent bonding of a costimulatory polypeptide and a binding pair member, with or without the use of a linker. Chemical conjugation may be accomplished by the covalent bonding of a coinhibitory polypeptide and a binding pair member, with or without the use of a linker. The formation of such conjugates is within the skill of artisans and various techniques are known for accomplishing the conjugation, with the choice of the particular technique being guided by the materials to be conjugated. The addition of amino acids to the polypeptide (C- or N-terminal) which contain ionizable side chains, e.g., aspartic acid, glutamic acid, lysine, arginine, cysteine, histidine, or tyrosine, and are not contained in the active portion of the polypeptide sequence, serve in their unprotonated state as a potent nucleophile to engage in various bioconjugation reactions with reactive groups attached to polymers, e.g., homo- or hetero-bi-functional PEG (e.g., Lutolf and Hubbell, Biomacromolecules 2003; 4:713-22, Hermanson, Bioconjugate Techniques, London. Academic Press Ltd; 1996).

Other molecular fusions may be formed between exogenous polypeptides and include direct or indirect conjugation. The exogenous polypeptides may be directly conjugated to each other or indirectly through a linker. The linker may be a peptide, a polymer, an aptamer, or a nucleic acid. The polymer may be, e.g., natural, synthetic, linear, or branched. Exogenous polypeptides can comprise a heterologous fusion protein that comprises a first polypeptide and a second polypeptide with the fusion protein comprising the polypeptides directly joined to each other or with intervening linker sequences and/or further sequences at one or both ends. The conjugation to the linker may be through covalent bonds or ionic bonds.

Erythroid cells described herein can also be produced using coupling reagents to link an exogenous polypeptide to a cell. For instance, click chemistry can be used. Thus, in some embodiments, any one or combination of exogenous polypeptides described herein may be conjugated onto the surface of an erythroid cell (e.g., an enucleated erythroid cell) using click chemistry. Coupling reagents can be used to couple an exogenous polypeptide to a cell, for example, when the exogenous polypeptide is a complex or difficult to express polypeptide, e.g., a polypeptide, e.g., a multimeric polypeptide; large polypeptide; polypeptide derivatized in vitro; an exogenous polypeptide that may have toxicity to, or which is not expressed efficiently in, the erythroid cells. Click chemistry and other conjugation methods for functionalizing erythroid cells is described in International Application No. PCT/US2018/000042, which claims priority to U.S. Provisional Application No. 62/460589, filed Feb. 17, 2017 and U.S. Provisional Application No. 62/542142, filed Jul. 8, 2017, incorporated by reference in their entireties herein.

Thus, in some embodiments, an erythroid cell described herein comprises many as, at least, more than, or about 5,000, 10,000, 50,000, 100,000, 200,000, 300,000, 400,000, 500,000 coupling reagents per cell. In some embodiments, the erythroid cells are made by a method comprising a) coupling a first coupling reagent to an erythroid cell, thereby making a pharmaceutical preparation, product, or intermediate. In an embodiment, the method further comprises: b) contacting the cell with an exogenous polypeptide coupled to a second coupling reagent e.g., under conditions suitable for reaction of the first coupling reagent with the second coupling reagent. In embodiments, two or more exogenous polypeptides are coupled to the cell (e.g., using click chemistry). In embodiments, a first exogenous polypeptide is coupled to the cell (e.g., using click chemistry) and a second exogenous polypeptide comprises a polypeptide expressed from an exogenous nucleic acid.

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (https://clickchemistrytools.com/) or Lahann, J (ed) (2009) Click Chemistry for Biotechnology and Materials Science, each of which is incorporated herein by reference in its entirety.

In some embodiments, the exogenous polypeptide is attached to an erythroid cell via a covalent attachment to generate an engineered erythroid cell comprising a cell presenting, e.g., comprising on the cell surface, one or more exogenous polypeptides (e.g. a first exogenous polypeptide and a second exogenous polypeptide). For example, the exogenous polypeptide may be derivatized and bound to the erythroid cell or platelet using a coupling compound containing an electrophilic group that will react with nucleophiles on the erythroid cell or platelet to form the interbonded relationship. Representative of these electrophilic groups are αβ unsaturated carbonyls, alkyl halides and thiol reagents such as substituted maleimides. In addition, the coupling compound can be coupled to an exogenous polypeptide via one or more of the functional groups in the polypeptide such as amino, carboxyl and tyrosine groups. For this purpose, coupling compounds should contain free carboxyl groups, free amino groups, aromatic amino groups, and other groups capable of reaction with enzyme functional groups. Highly charged exogenous polypeptides can also be prepared for immobilization on, e.g., erythroid cells or platelets through electrostatic bonding to generate an engineered erythroid cell. Examples of these derivatives would include polylysyl and polyglutamyl enzymes.

The choice of the reactive group embodied in the derivative depends on the reactive conditions employed to couple the electrophile with the nucleophilic groups on the erythroid cell or platelet for immobilization. A controlling factor is the desire not to inactivate the coupling agent prior to coupling of the exogenous polypeptide immobilized by the attachment to the erythroid cell or platelet. Such coupling immobilization reactions can proceed in a number of ways. Typically, a coupling agent can be used to form a bridge between the exogenous polypeptide and the erythroid cell or platelet. In this case, the coupling agent should possess a functional group such as a carboxyl group which can be caused to react with the exogenous polypeptide. One way of preparing the exogenous polypeptide for conjugation includes the utilization of carboxyl groups in the coupling agent to form mixed anhydrides which react with the exogenous polypeptide, in which use is made of an activator which is capable of forming the mixed anhydride. Representative of such activators are isobutylchloroformate or other chloroformates which give a mixed anhydride with coupling agents such as 5,5′-(dithiobis(2-nitrobenzoic acid) (DTNB), p-chloromercuribenzoate (CMB), or m-maleimidobenzoic acid (MBA). The mixed anhydride of the coupling agent reacts with the exogenous polypeptide to yield the reactive derivative which in turn can react with nucleophilic groups on the erythroid cell or platelet to immobilize the exogenous polypeptide.

Functional groups on an exogenous polypeptide, such as carboxyl groups can be activated with carbodiimides and the like activators. Subsequently, functional groups on the bridging reagent, such as amino groups, will react with the activated group on the exogenous polypeptide to form the reactive derivative. In addition, the coupling agent should possess a second reactive group which will react with appropriate nucleophilic groups on the erythroid cell or platelet to form the bridge. Typical of such reactive groups are alkylating agents such as iodoacetic acid, αβ unsaturated carbonyl compounds, such as acrylic acid and the like, thiol reagents, such as mercurials, substituted maleimides and the like.

Alternatively, functional groups on the exogenous polypeptide can be activated so as to react directly with nucleophiles on, e.g., erythroid cells or platelets to obviate the need for a bridge-forming compound. For this purpose, use is made of an activator such as Woodward's Reagent K or the like reagent which brings about the formation of carboxyl groups in the exogenous polypeptide into enol esters, as distinguished from mixed anhydrides. The enol ester derivatives of exogenous polypeptides subsequently react with nucleophilic groups on, e.g., an erythroid cell or platelet to effect immobilization of the exogenous polypeptide, thereby creating an engineered erythroid cell.

In some embodiments, the engineered erythroid cell comprising an exogenous polypeptide (e.g. a first and/or a second exogenous polypeptide) is generated by contacting an erythroid cell with an exogenous polypeptide and optionally a payload, wherein contacting does not include conjugating the exogenous polypeptide to the erythroid cell using an attachment site comprising Band 3 (CD233), aquaporin-1, Glut-1, Kidd antigen, RhAg/R1i50 (CD241), Rli (CD240), Rh30CE (CD240CE), Rh30D (CD240D), Kx, glycophorin B (CD235b), glycophorin C (CD235c), glycophorin D (CD235d), Kell (CD238), Duffy/DARCi (CD234), CR1 (CD35), DAF (CD55), Globoside, CD44, ICAM-4 (CD242), Lu/B-CAM (CD239), XG1/XG2 (CD99), EMMPRIN/neurothelin (CD147), JMH, Glycosyltransferase, Cartwright, Dombrock, C4A/CAB, Scianna, MER2, stomatin, BA-1 (CD24), GPIV (CD36), CD108, CD139, or H antigen (CD173).

In some embodiments, the engineered erythroid cell comprises an erythroid cell presenting, e.g. comprising on the cell surface, one or more exogenous polypeptides, wherein the one or more exogenous polypeptides are enzymatically conjugated onto the cell.

In specific embodiments, the exogenous polypeptide can be conjugated to the surface of, e.g., an erythroid cell or platelet by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme to connect one polypeptide containing the acceptor sequence LPXTG (SEQ ID NO: 30) or LPXTA (SEQ ID NO: 31) with a polypeptide containing the N-terminal donor sequence GGG, see e.g., Swee et al., PNAS 2013. The methods also include combination methods, such as e.g., sortase-mediated conjugation of Click Chemistry handles (an azide and an alkyne) on the antigen and the cell, respectively, followed by a cyclo-addition reaction to chemically bond the antigen to the cell, see e.g., Neves et al., Bioconjugate Chemistry, 2013. Sortase-mediated modification of proteins is described in International Application No. PCT/US2014/037545 and International Application No. PCT/US2014/037554, both of which are incorporated by reference in their entireties herein.

In some embodiments, a protein is modified by the conjugation of a sortase substrate comprising an amino acid, a peptide, a protein, a polynucleotide, a carbohydrate, a tag, a metal atom, a contrast agent, a catalyst, a non-polypeptide polymer, a recognition element, a small molecule, a lipid, a linker, a label, an epitope, an antigen, a therapeutic agent, a toxin, a radioisotope, a particle, or moiety comprising a reactive chemical group, e.g., a click chemistry handle.

If desired, a catalytic bond-forming polypeptide domain can be expressed on or in e.g., an erythroid cell or platelet, either intracellularly or extracellularly. Many catalytic bond-forming polypeptides exist, including transpeptidases, sortases, and isopeptidases, including those derived from Spy0128, a protein isolated from Streptococcus pyogenes.

In some embodiments, any of the polypeptides described herein are not conjugated to the cell using a sortase.

It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Asp117 on SpyTag and Lys31 on SpyCatcher (Zakeri and Howarth, JACS 2010, 132:4526). The reaction is compatible with the cellular environment and highly specific for protein/peptide conjugation (Zakeri, B.; Fierer, J. O.; Celik, E.; Chittock, E. C.; Schwarz-Linek, U.; Moy, V. T.; Howarth, M. Proc. Natl. Acad. Sci. U.S.A. 2012, 109, E690-E697). SpyTag and SpyCatcher has been shown to direct post-translational topological modification in elastin-like protein. For example, placement of SpyTag at the N-terminus and SpyCatcher at the C-terminus directs formation of circular elastin-like proteins (Zhang et al, Journal of the American Chemical Society, 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this document, when SpyTag and SpyCatcher are used, it is to be understood that the complementary molecule could be substituted in its place.

A catalytic bond-forming polypeptide, such as a SpyTag/SpyCatcher system, can be used to attach the exogenous

polypeptide to the surface of, e.g., an erythroid cell, to generate an engineered erythroid cell. The SpyTag polypeptide sequence can be expressed on the extracellular surface of the erythroid cell. The SpyTag polypeptide can be, for example, fused to the N terminus of a type-1 or type-3 transmembrane protein, e.g., glycophorin A, fused to the C terminus of a type-2 transmembrane protein, e.g., Kell, inserted in-frame at the extracellular terminus or in an extracellular loop of a multi-pass transmembrane protein, e.g., Band 3, fused to a GPI-acceptor polypeptide, e.g., CD55 or CD59, fused to a lipid-chain-anchored polypeptide, or fused to a peripheral membrane protein. The nucleic acid sequence encoding the SpyTag fusion can be expressed within an engineered erythroid cell. An exogenous polypeptide can be fused to SpyCatcher. The nucleic acid sequence encoding the SpyCatcher fusion can be expressed and secreted from the same erythroid cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the exogenous polypeptide to the surface of the erythroid cell to form an engineered erythroid cell.

In some embodiments, the SpyTag polypeptide may be expressed as a fusion to the N terminus of glycophorin A under the control of the Gatal promoter in an erythroid cell. An exogenous polypeptide, fused to the SpyCatcher polypeptide sequence can be expressed under the control of the Gatal promoter in the same erythroid cell. Upon expression of both fusion polypeptides, an isopeptide bond will be formed between the SpyTag and SpyCatcher polypeptides, forming a covalent bond between the erythroid cell surface and the exogenous polypeptide.

In some embodiments, the SpyTag polypeptide may be expressed as a fusion to the N terminus of glycophorin A under the control of the Gatal promoter in an erythroid cell. An exogenous polypeptide fused to the SpyCatcher polypeptide sequence can be expressed in a suitable mammalian cell expression system, for example HEK293 cells. Upon expression of the SpyTag fusion polypeptide on the erythroid cell, the SpyCatcher fusion polypeptide can be brought in contact with the cell. Under suitable reaction conditions, an isopeptide bond will be formed between the SpyTag and SpyCatcher polypeptides, forming a covalent bond between the erythroid cell surface and the exogenous polypeptide. Exogenous polypeptides can be detected on the engineered erythroid cells. The presence of the exogenous polypeptide can be validated and quantified using standard molecular biology methods, e.g., Western blotting or FACS analysis. Exogenous polypeptides present in the intracellular environment may be quantified upon cell lysis or using fluorescent detection.

In some embodiments of the above aspects and embodiments, the engineered erythroid cell is an enucleated cell, e.g. an erythrocyte or a reticulocyte. In some embodiments of the above aspects and embodiments, the engineered erythroid cell is a nucleated cell.

### IV. Methods of Use

The present disclosure provides methods of treating or preventing hyperuricemia in a subject, comprising administering to the subject the engineered erythroid cell as described herein, in an amount effective to treat or prevent hyperuricemia in the subject.

Treatment of Conditions that would Benefit from Degradation of Uric Acid

Methods of administering engineered erythroid cells comprising (e.g., presenting) exogenous agent (e.g., polypeptides) are described, e.g., in WO2015/073587 and WO2015/153102, each of which is incorporated by reference in its entirety.

In embodiments, the engineered erythroid cells described herein (e.g., engineered enucleated cells) are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In one aspect, the present disclosure provides a method of treating or preventing hyperuricemia in a subject, comprising administering to the subject an engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), in an amount effective to treat or prevent hyperuricemia in the subject.

The normal range of uric acid in blood is between 3.4 mg/dL and 7.0 mg/dL in men, between 2.4 mg/dL and 6.0 mg/dL in premenopausal women, and from 2.5 mg/dL to 5.5 mg/dL in children. Urate crystal formation/precipitation typically occurs in men at levels of 6.6 mg/dL or higher and in women at levels of 6.0 mg/dL or higher. Also, what may be in the normal range for the population as a whole may be elevated for the individual. Cardiovascular and other consequences of elevated uric acid can occur with blood levels well within these “normal” ranges. Therefore, a diagnosis of hyperuricemia is not necessarily a prerequisite for the beneficial effects of the engineered erythroid cells of the invention.

In some embodiments, the subject has a serum urate level greater than about 6.8 mg/dl, for example 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0 mg/dL or more, prior to administering the engineered erythroid cell. In some embodiments, the subject has a serum urate level greater than about 8.0 mg/dL, for example 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0 mg/dL or more, prior to administering the engineered erythroid cell.

In some embodiments, the methods described herein comprise selecting a subject having a serum urate level greater than about 6.8 mg/dl, for example 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0 mg/dL or more, and administering an engineered erythroid cell described herein.

In some embodiments, the subject has a serum urate level less than about 6.8 mg/dl, for example 6.7, 6.6, 6.5, 6.4, 6.3, 6.2, 6.1, 6.0, 5.9, 5.8, 5.7, 5.6, 5.5, 5.4, 5.3, 5.2, 5.1, 5.1 mg/dL or less, after administering the engineered erythroid cell. In some embodiments, the subject has a serum urate level of about 6.0 mg/dl after administering the engineered erythroid cell, for example 6.0, 5.9, 5.8, 5.7, 5.6, 5.5, 5.4, 5.3, 5.2, 5.1, 5.1 mg/dL or less, after administering the engineered erythroid cell.

### Hyperuricemia

Hyperuricemia is the presence of high levels of uric acid in the blood. Hyperuricemia may occur because of decreased excretion. Hyperuricemia may also occur from increased production, or a combination of the two mechanisms. Underexcretion accounts for the majority of cases of hyperuricemia. Overproduction accounts for only a minority of patients presenting with hyperuricemia. Consumption of purine-rich diets is one of the main causes of hyperuricemia. Other dietary causes are ingestion of high protein and fat, and starvation. Starvation results in the body metabolizing its own muscle mass for energy, in the process releasing purines into the bloodstream. Purine bases composition of foods varies. Foods with higher content of purine bases adenine and hypoxanthine are suggested to be more potent in exacerbating hyperuricemia.

Humans lack uricase, an enzyme which degrades uric acid. Increased levels predispose for gout and, if very high, renal failure. Apart from normal variation (with a genetic component), tumor lysis syndrome produces extreme levels of uric acid, mainly leading to renal failure. The Lesch-Nyhan syndrome is also associated with extremely high levels of uric acid. The Metabolic syndrome often presents with hyperuricemia, while a hyperuricemic syndrome is also common in Dalmatian dogs. A uric acid degrading polypeptide, e.g., uricase, described herein and a pH increasing agent, alone or in combination with another agent, e.g., another agent described herein, can be used to treat hyperuricemia.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat hyperuricemia.

### Asymptomatic Hyperuricemia

Asymptomatic hyperuricemia is the term for an abnormally high serum urate level, without gouty arthritis or nephrolithiasis. Hyperuricemia is defined as a serum urate concentration greater than about 6.8 mg per dL, the approximate level at which urate is supersaturated in plasma. Serum uric acid levels above 360 uM are considered pathogenic.

Although gouty arthritis characteristically occurs in patients with hyperuricemia, hyperuricemia is not necessarily associated with clinical gout. Researchers from the Normative Aging Study followed 2,046 initially healthy men for 15 years by taking serial measurements of serum urate levels. The five-year cumulative incidence rates of gouty arthritis were 2.0 percent for a serum urate level of 8.0 mg per dL (475 μmol per L) or lower, 19.8 percent for urate levels from 9.0 to 10.0 mg per dL (535 to 595 μmol per L) and 30 percent for a serum urate level higher than 10 mg per dL (595 μmol per L). Hyperuricemia predisposes patients to both gout and nephrolithiasis, but therapy is occasionally not warranted in the asymptomatic patient. Recognizing hyperuricemia in the asymptomatic patient, however, provides the physician with an opportunity to modify or correct underlying acquired causes of hyperuricemia.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat asymptomatic hyperuricemia.

### Hyperuricosuria

Hyperuricosuria is defined as urinary excretion of uric acid greater than 800 mg/d in men and greater than 750 mg/d in women. This may be due to either excess dietary intake of purine-rich foods or endogenous uric acid overproduction. Hyperuricosuria may be associated with hyperuricemia.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat hyperuricosuria.

### Gout

Gout is a condition that results from crystals of uric acid depositing in tissues of the body. Gout is characterized by an overload of uric acid in the body and recurring attacks of joint inflammation (arthritis). Chronic gout can lead to deposits of hard lumps of uric acid in and around the joints, decreased kidney function, and kidney stones.

Gout is generally divided into four categories based upon progressively more severe symptoms:

1) Asymptomatic. Elevated uric acid levels in the blood, but no overt symptoms.

2) Acute gouty arthritis: Sudden onset of symptoms, often in a single joint (commonly a big toe), and then involving other joints. Symptoms include pain, swelling, redness and fever.

3) Intercritical gout: Asymptomatic phases between gout attacks.

4) Chronic tophaceous gout: A chronic condition that may include frequent attacks, constant mild pain and inflammation of joints, destruction of cartilage and bone, development of uric acid crystal deposits, kidney dysfunction and kidney stones.

Excess serum accumulation of uric acid can lead to a type of arthritis known as gout (gouty arthritis). Gouty arthritis is usually an extremely painful attack with a rapid onset of joint inflammation. The joint inflammation is precipitated by deposits of uric acid crystals in the joint fluid (synovial fluid) and joint lining (synovial lining). Intense joint inflammation occurs as white blood cells engulf the uric acid crystals and release chemicals of inflammation, causing pain, heat, and redness of the joint tissues.

The small joint at the base of the big toe is the most common site of an acute gout attack. Other joints that can be affected include the ankles, knees, wrists, fingers, and elbows. Acute gout attacks are characterized by a rapid onset of pain in the affected joint followed by warmth, swelling, reddish discoloration, and marked tenderness.

Hyperuricemia and gout are particularly significant issues in organ transplant recipients (Stamp, L., et al, Drugs (2005) 65(18): 2593-2611). Uric acid is often elevated in patients with renal transplants, and common immunosupressive drugs such as cyclosporine can cause particularly severe hyperuricemia. In transplant patients, allopurinol is contra-indicated due to interactions with some immunosupressants such as azathioprine, and due to bone marrow failure caused by the combination. Furthermore, elevated uric acid may contribute to graft failure (Armstrong, K. A. et al., Transplantation (2005) 80(11): 1565-1571).

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat gout.

In a particular embodiment, an engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat refractory gout. In some embodiments, a subject has refractory gout if they have demonstrated contraindication to allopurinol, or have a medical history of failure to normalize uric acid (e.g., to less than 6 mg/dL) with at least 3 months of allopurinol treatment at the maximum medically appropriate dose.

### Lesch-Nyhan Syndrome

Lesch-Nyhan syndrome (LNS), also known as Nyhan's syndrome, is a rare, inherited disorder caused by a deficiency of the enzyme hypoxanthine-guanine phosphoribosyltransferase (HGPRT). LNS is an X-linked recessive disease: the gene is carried by the mother and passed on to her son. LNS is present at birth in baby boys. Patients have severe mental and physical problems throughout life. The lack of HGPRT causes a build-up of uric acid in all body fluids, and leads to problems such as severe gout, poor muscle control, and moderate mental retardation, which appear in the first year of life. Abnormally high uric acid levels can cause sodium uric acid crystals to form in the joints, kidneys, central nervous system and other tissues of the body, leading to gout-like swelling in the joints and severe kidney problems. Neurological symptoms include facial grimacing, involuntary writhing, and repetitive movements of the arms and legs similar to those seen in Huntington's disease. The direct cause of the neurological abnormalities remains unknown. Because a lack of HGPRT causes the body to poorly utilize vitamin B 12, some boys may develop a rare disorder called megaloblastic anemia.

The symptoms caused by the buildup of uric acid (arthritis and renal symptoms) respond well to treatment with drugs such as allopurinol that reduce the levels of uric acid in the blood. There is no cure, but many patients live to adulthood. LNS is rare, affecting about one in 380,000 live births.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat Lesch-Nyhan syndrome.

### Uric Acid Nephrolothiasis

Uric acid stones account for about 5 to 10% of all kidneys stones in Western countries and Japan. The stones can be composed of uric acid alone or admixed with calcium oxalate. Sex distribution indicates a male to female ratio of more than one, which tends to diminish in the post-menopausal age. Kidney stones, also called renal calculi, are solid concretions (crystal aggregations) of dissolved minerals in urine; calculi typically form inside the kidneys or 5 bladder. The terms nephrolithiasis and urolithiasis refer to the presence of calculi in the kidneys and urinary tract, respectively.

The formation of uric acid stones is associated with conditions that cause high blood uric acid levels, such as gout, leukemias/lymphomas treated by chemotherapy (secondary gout from the death of leukemic cells), and acid/base metabolism disorders where the urine is excessively acid resulting in uric acid precipitation.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat kidney stones and nephrolithiasis, e.g., kidney stones and/or nephrolithiasis caused by, or associated with, elevated uric acid concentrations.

### Vascular Conditions

Diseases related to elevated soluble uric acid often involve vascular problems: hypertension (Sundstrom et al., Relations of serum uric acid to longitudinal blood pressure tracking and hypertension incidence. Hypertension. 45(1):28-33, 2005), prehypertension (Syamela, S. et al., Association between serum uric acid and prehypertension among US adults. J Hypertens. 25 (8) 1583-1589, (2007), atherosclerosis (Ishizaka et al., Association between serum uric acid, metabolic syndrome, and carotid atherosclerosis in Japanese individuals. Arterioscler Thromb Vasc Biol. (5):1038-44, 2005), peripheral artery disease (Shankar, A. et al., Association between serum uric acid level and peripheral artery disease. Atherosclerosis doi 10: 1016, 2007), vascular inflammation (Zoccali et al., Uric acid and endothelial dysfunction in essential hypertension. J Am Soc Nephrol. 17(5):1466-71, 2006), heart failure (Strasak, A. M. et al., Serum uric acid and risk of cardiovascular mortality: A prospective, long-term study of 83,683 Austrian men, Clin Chem. 54 (2) 273-284, 2008; Pascual-Figal, Hyperuricaemia and long-term outcome after hospital discharge in acute heart failure patients. Eur J Heart Fail. 2006 Oct. 23; [Epub ahead of print]; Cengel, A., et al., “Serum uric Acid Levels as a Predictor of In-hospital Death in Patients Hospitalized for Decompensated Heart Failure.” Acta Cardiol. (October 2005) 60(5): 489-492), myocardial infarctions (Strasak, A. M. et al.; Bos et al., Uric acid is a risk factor for myocardial infarction and stroke: the Rotterdam study. Stroke. 2006 June; 37(6):1503-7), renal dysfunction (Cirillo et al., Uric Acid, the metabolic syndrome, and renal disease. J Am Soc Nephrol. 17(12 Suppl 3):S165-8, 2006), and strokes (Bos et al., 2006). Uric acid directly causes endothelial dysfunction (Kanellis, et al., Uric acid as a mediator of endothelial dysfunction, inflammation, and vascular disease. Semin Nephrol. 25(1):39-42, 2005; Khosla et al, Hyperuricemia induces endothelial dysfunction. Kidney Int. 67(5):1739-42, 2005). In children, early-onset essential hypertension is associated with elevated serum uric acid, and reduction of uric acid with allopurinol reduced blood pressure in a small cohort of patients (Feig and Johnson, The role of uric acid in pediatric hypertension. J Ren Nutrition 17(1): 79-83, 2007). Hyperuricemia is an independent risk factor in all of these conditions.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat cardiovascular disease caused by, or associated with, elevated uric acid concentrations.

### Diabetes

Elevated levels of uric acid are associated with prediabetes, insulin resistance, the development of Type 2 diabetes, and an increased probability of a variety of undesirable conditions in people with diabetes, such as peripheral artery disease, strokes, and increased mortality risk. Studies have shown that high serum uric acid is associated with higher risk of type 2 diabetes independent of obesity, dyslipidemia, and hypertension. Serum uric acid is a strong predictor of stroke in patients with non-insulin dependent diabetes mellitus.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat diabetes caused by, or associated with, elevated uric acid concentrations.

### Metabolic Syndrome

Metabolic syndrome is a cluster of conditions that occur together, increasing the risk of heart disease, stroke and diabetes. Metabolic syndrome involves having several disorders related to metabolism at the same time, including: obesity; elevated blood pressure; an elevated level of triglycerides; a low level of high-density lipoprotein (HDL) cholesterol; high blood pressure and/or high insulin levels. Hyperuricemia is associated with components of metabolic syndrome, and may play a pathogenic role in the metabolic syndrome.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat metabolic syndrome caused by, or associated with, elevated uric acid concentrations.

### Inflammatory Responses

Elevated soluble uric acid is also associated with or directly induces inflammatory responses. For example, uric acid is transported into vascular smooth muscle cells via organic acid transporters, especially the uric acid transporter URAT1, and then stimulates vascular smooth muscle cells to produce C-reactive protein, MCP-1 and other cytokines, thereby stimulating proliferation and other changes associated with atherosclerosis (Price et al., Human vascular smooth muscle cells express a uric acid transporter. J Am Soc Nephrol. 17(7):1791-5, 2006; Kang et al., Uric acid causes vascular smooth muscle cell proliferation by entering cells via a functional uric acid transporter. Am J Nephrol. 2005 25(5):425-33 (2005); Yamamoto et al., Allopurinol reduces neointimal hyperplasia in the carotid artery ligation model in spontaneously hypertensive rats. Hypertens. Res. 29 (11) 915-921, 2006), stimulates human mononuclear cells to produce IL-1β, IL-6 and TNF-α, causes marked increases in TNF-α when infused into mice, activates endothelial cells and platelets, and increases platelet adhesiveness (Coutinho et al., “Associations of Serum Uric Acid with Markers of Inflammation, Metabolic Syndrome, and Subclinical Coronary Atherosclerosis”, Amer. J. Hypertens. (2007) 20: 83-89; Levya, F., et al., “Uric Acid in Chronic Heart Failure: A Marker of Chronic Inflammation”, Eur. Heart J. (1998) 19(12): 1814-1822). Uric acid has also been shown to inhibit bioavailability of endothelial nitric oxide and activate the renin-angiotensin system.

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase and a uric acid transporter, or an engineered erythroid cell comprising a uric acid degrading polypeptide, e.g., uricase, a uric acid transporter and a catalase), alone or in combination with another agent, e.g., another agent described herein, can be used to treat inflammatory responses caused by, or associated with, elevated uric acid concentrations.

### Cognitive Impairment

Hyperuricemia is also associated with cognitive impairment and other forms of central nervous system dysfunction. (Schretlen, D. J. et al., “Serum Uric Acid and Cognitive Function in Community-Dwelling Older Adults”, Neuropsychology (January 2007) 21(1): 136-140; Watanabe, S., et al., “Cerebral Oxidative Stress and Mitochondrial Dysfunction in Oxonate-Induced Hyperuricemic Mice”, J. Health Science (2006) 52: 730-737).

An engineered erythroid cell as described herein (e.g. an engineered erythroid cell comprising a uric acid degrading polypeptide, e.g., uricase, an engineered erythroid cell comprising a uric acid transporter, an engineered erythroid cell comprising a catalase, an engineered erythroid cell comprising a uric acid degrading polypeptide, e.g., uricase, and a uric acid transporter or an engineered erythroid cell comprising a uric acid degrading polypeptide, e.g., uricase, a uric acid transporter and a catalase), alone or in combination with another agent, e.g., another agent described herein, can be used to treat cognitive impairment caused by, or associated with, elevated uric acid concentrations.

### Dosing

The present disclosure is based, at least in part, on a determination of the target uricase activity of an engineered erythroid cell required to achieve clinical efficacy, and to establish clinical dosing feasibility. Taking into account the target uric acid degradation activity needed to achieve clinical efficacy and the uric acid uptake activity by a uric acid transporter expressed on the uricase-expressing engineered erythroid cell, levels of expression of the uric acid degrading polypeptide by the engineered erythroid cell were optimized to meet the target activity.

In one embodiment, a dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In some embodiments, the engineered erythroid cell comprises a first exogenous polypeptide comprising a uric acid degrading polypeptide, or a variant thereof, a second exogenous polypeptide comprising a uric acid transporter, or variant thereof, and a third exogenous polypeptide comprising a catalase, or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In some embodiments, the engineered erythroid cell comprises a first exogenous polypeptide comprising a uricase, or a variant thereof, and a second exogenous polypeptide comprising a uric acid transporter, or variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹⁰-5×10¹¹, 5×10¹¹-1×10¹² cells/dose. In some embodiments, the engineered erythroid cell comprises a first exogenous polypeptide comprising a Candida utilis uricase, or a variant thereof, and a second exogenous polypeptide comprising a uric acid transporter, or variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹⁰-1×10¹² cells/dose. In some embodiments, the engineered erythroid cell comprises a first exogenous polypeptide comprising a Candida utilis uricase, or a variant thereof, and a second exogenous polypeptide comprising human URAT1, or variant thereof, where the dose of engineered erythroid cells as described herein comprises about 1×10¹⁰-1×10¹² engineered erythroid cells per dose, for example 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹² cells/dose.

Exemplary doses of engineered erythroid cells as described herein can be about 1×10¹⁰ cells/dose, 2×10¹⁰ cells/dose, 3×10¹⁰ cells/dose, 4×10¹⁰ cells/dose, 5×10¹⁰ cells/dose, 6×10¹⁰ cells/dose, 7×10¹⁰ cells/dose, 8×10¹⁰ cells/dose, 9×10¹⁰ cells/dose, 1×10¹¹ cells/dose, 2×10¹¹ cells/dose, 3×10¹¹ cells/dose, 4×10¹¹ cells/dose, 5×10¹¹ cells/dose, 6×10¹¹ cells/dose, 7×10¹¹ cells/dose, 8×10¹¹ cells/dose, 9×10¹¹ cells/dose, 1×10¹²cells/dose or more. In some embodiments, a dose of engineered erythroid cells as described herein comprises about 3×10¹⁰ engineered erythroid cells per dose.

In some embodiments, the dose of engineered erythroid cells is determined based on the target uricolytic activity per engineered erythroid cell. For example, In some embodiments, the uricolytic activity is based on the transport rate of uric acid into the engineered erythroid cells by the uric acid transporter, and the subsequent break down of uric acid by uricase inside the engineered erythroid cells. In some embodiments, the engineered erythroid cell has between 1e-9 and 1e-11 units of uricolytic activity per cell, for example between 1e-9 and 1e-10, or between 1e-10 and 1e-11, or between 5e-9 and 5e-10, or between 5e-10 and 1e-11 units of uricoloytic activity per cell. In some embodiments, the engineered erythroid cell has at least 3-6×10⁻¹⁰ units of uricolytic activity per cell, for example at least 3e-10, 3.1 e-10, 3.2e-10, 3.3e-10, 3.4e-10, 3.5e-10, 3.6e-10, 3.7e-10, 3.8e-10, 3.9e-10, 4e-10, 4.1 e-10, 4.2e-10, 4.3e-10, 4.4e-10, 4.5e-10, 4.6e-10, 4.7e-10, 4.8e-10, 4.9e-10, 5e-10, 5.1e-10, 5.2e-10, 5.3e-10, 5.4e-10, 5.5e-10, 5.6e-10, 5.7e-10, 5.8e-10, 5.9e-10, or 6e-10 units of uricoytic activity per cell. In some embodiments, the engineered erythroid cell has at least 1-2×10⁻¹⁰ units of uricolytic activity per cell, for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cell has between about 0.1-10×10⁻¹⁰ , or between about 0.5-5×10⁻¹⁰, or between about 1-2×10⁻¹⁰ units of uricolytic activity per cell, for example about 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell.

In some embodiments, the engineered erythroid cell comprises a first exogenous polypeptide comprising a uric acid degrading polypeptide, or a variant thereof, and a second exogenous polypeptide comprising a uric acid transporter, or variant thereof, where the engineered erythroid cell has between 1e-9 and 1e-11 units of uricolytic activity per cell, for example between 1e-9 and 1e-10, or between 1e-10 and 1e-11, or between 5e-9 and 5e-10, or between 5e-10 and 1e-11 units of uricoloytic activity per cell. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the engineered erythroid cell has between 3e-10 and 6e-10 units of uricolytic activity per cell, for example between 3e-10 and 4e-10, or between 3e-10 and 5e-10, or between 4e-10 and 5e-10, or between 5e-10 and 6e-10 units of uricoloytic activity per cell. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the engineered erythroid cell has at least 1-2×10⁻¹⁰ units of uricolytic activity per cell, for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the engineered erythroid cell has between about 3-6×10⁻¹⁰, or between about 3.5-6×10⁻¹⁰, or between about 4-6×10⁻¹⁰, or between about 4.5-6×10⁻¹⁰, or between about 5-6×10⁻¹⁰, or between about 5.5-6×10⁻¹⁰ units of uricolytic activity per cell, for example about 3e-10, 3.1 e-10, 3.2e-10, 3.3e-10, 3.4e-10, 3.5e-10, 3.6e-10, 3.7e-10, 3.8e-10, 3.9e-10, 4e-10, 4.1 e-10, 4.2e-10, 4.3e-10, 4.4e-10, 4.5e-10, 4.6e-10, 4.7e-10, 4.8e-10, 4.9e-10, 5e-10, 5.1 e-10, 5.2e-10, 5.3e-10, 5.4e-10, 5.5e-10, 5.6e-10, 5.7e-10, 5.8e-10, 5.9e-10, 6e-10 units of uricoytic activity per cell. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the engineered erythroid cell has between about 0.1-10×10⁻¹⁰, or between about 0.5-5×10⁻¹⁰, or between about 1-2×10⁻¹⁰ units of uricolytic activity per cell, for example about 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell.

In some embodiments, the engineered erythroid cell comprises a first exogenous polypeptide comprising a uricase, or a variant thereof, and a second exogenous polypeptide comprising a uric acid transporter, or variant thereof, where the engineered erythroid cell has between 1e-9 and 1e-11 units of uricolytic activity per cell, for example between 1e-9 and 1e-10, or between 1e-10 and 1e-11, or between 5e-9 and 5e-10, or between 5e-10 and 1e-11 units of uricoloytic activity per cell. In some embodiments, the engineered erythroid cell comprises a first exogenous polypeptide comprising a uricase, or a variant thereof, and a second exogenous polypeptide comprising a uric acid transporter, or variant thereof, where the engineered erythroid cell has between 3e-10 and 6e-10 units of uricolytic activity per cell, for example between 3e-10 and 4e-10, or between 3e-10 and 5e-10, or between 4e-10 and 5e-10, or between 5e-10 and 6e-10 units of uricoloytic activity per cell. In some embodiments, the engineered erythroid cell comprises a first exogenous polypeptide comprising a uricase, or a variant thereof, and a second exogenous polypeptide comprising a uric acid transporter, or variant thereof, where the engineered erythroid cell has at least 1-2×10-¹⁰ units of uricolytic activity per cell, for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cell comprises a first exogenous polypeptide comprising a uricase, or a variant thereof, and a second exogenous polypeptide comprising a uric acid transporter, or variant thereof, where the engineered erythroid cell has between about 3-6×10-¹⁰ , or between about 3.5-6×10-¹⁰, or between about 4-6×10-¹⁰, or between about 4.5-6×10-¹⁰, or between about 5-6×10-¹⁰, or between about 5.5-6×10-¹⁰ units of uricolytic activity per cell, for example about 3e-10, 3.1 e-10, 3.2e-10, 3.3e-10, 3.4e-10, 3.5e-10, 3.6e-10, 3.7e-10, 3.8e-10, 3.9e-10, 4e-10, 4.1 e-10, 4.2e-10, 4.3e-10, 4.4e-10, 4.5e-10, 4.6e-10, 4.7e-10, 4.8e-10, 4.9e-10, 5e-10, 5.1 e-10, 5.2e-10, 5.3e-10, 5.4e-10, 5.5e-10, 5.6e-10, 5.7e-10, 5.8e-10, 5.9e-10, 6e-10 units of uricoytic activity per cell. In some embodiments, the engineered erythroid cell comprises a first exogenous polypeptide comprising a uricase, or a variant thereof, and a second exogenous polypeptide comprising a uric acid transporter, or variant thereof, where the engineered erythroid cell has between about 0.1-10×10⁻¹⁰ , or between about 0.5-5×10⁻¹⁰, or between about 1-2×10⁻¹⁰ units of uricolytic activity per cell, for example about 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell.

In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the engineered erythroid cell has between 1e-9 and 1e-11 units of uricolytic activity per cell, for example between 1e-9 and 1e-10, or between le-10 and 1e-11, or between 5e-9 and 5e-10, or between 5e-10 and 1e-11 units of uricoloytic activity per cell. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the engineered erythroid cell has between 3e-10 and 6e-10 units of uricolytic activity per cell, for example between 3e-10 and 4e-10, or between 3e-10 and 5e-10, or between 4e-10 and 5e-10, or between 5e-10 and 6e-10 units of uricoloytic activity per cell. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the engineered erythroid cell has at least 1-2×10-¹⁰ units of uricolytic activity per cell, for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the engineered erythroid cell has between about 3-6×10⁻¹⁰, or between about 3.5-6×10⁻¹⁰, or between about 4-6×10⁻¹⁰, or between about 4.5-6×10⁻¹⁰, or between about 5-6×10⁻¹⁰, or between about 5.5-6×10⁻¹⁰ units of uricolytic activity per cell, for example about 3e-10, 3.1 e-10, 3.2e-10, 3.3e-10, 3.4e-10, 3.5e-10, 3.6e-10, 3.7e-10, 3.8e-10, 3.9e-10, 4e-10, 4.1 e-10, 4.2e-10, 4.3e-10, 4.4e-10, 4.5e-10, 4.6e-10, 4.7e-10, 4.8e-10, 4.9e-10, 5e-10, 5.1 e-10, 5.2e-10, 5.3e-10, 5.4e-10, 5.5e-10, 5.6e-10, 5.7e-10, 5.8e-10, 5.9e-10, 6e-10 units of uricoytic activity per cell. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the engineered erythroid cell has between about 0.1-10×10⁻¹⁰, or between about 0.5-5×10⁻¹⁰, or between about 1-2×10⁻¹⁰ units of uricolytic activity per cell, for example about 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell.

In some embodiments, the engineered erythroid cell comprises a first exogenous polypeptide comprising a Candida utilis uricase, or a variant thereof, and a second exogenous polypeptide comprising a uric acid transporter, or variant thereof, where the engineered erythroid cell has between 1e-9 and 1e-11 units of uricolytic activity per cell, for example between 1e-9 and 1e-10, or between 1e-10 and 1e-11, or between 5e-9 and 5e-10, or between 5e-10 and 1e-11 units of uricoloytic activity per cell. In some embodiments, the engineered erythroid cell comprises a first exogenous polypeptide comprising a Candida utilis uricase, or a variant thereof, and a second exogenous polypeptide comprising a uric acid transporter, or variant thereof, where the engineered erythroid cell has at least 1-2×10-¹⁰ units of uricolytic activity per cell, for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cell comprises a first exogenous polypeptide comprising a Candida utilis uricase, or a variant thereof, and a second exogenous polypeptide comprising a uric acid transporter, or variant thereof, where the engineered erythroid cell has between about 3-6×10⁻¹⁰ , or between about 3.5-6×10⁻¹⁰, or between about 4-6×10⁻¹⁰ , or between about 4.5-6×10⁻¹⁰, or between about 5-6×10⁻¹⁰, or between about 5.5-6×10⁻¹⁰ units of uricolytic activity per cell, for example about 3e-10, 3.1 e-10, 3.2e-10, 3.3e-10, 3.4e-10, 3.5e-10, 3.6e-10, 3.7e-10, 3.8e-10, 3.9e-10, 4e-10, 4.1 e-10, 4.2e-10, 4.3e-10, 4.4e-10, 4.5e-10, 4.6e-10, 4.7e-10, 4.8e-10, 4.9e-10, 5e-10, 5.1 e-10, 5.2e-10, 5.3e-10, 5.4e-10, 5.5e-10, 5.6e-10, 5.7e-10, 5.8e-10, 5.9e-10, 6e-10 units of uricoytic activity per cell. In some embodiments, the engineered erythroid cell comprises a first exogenous polypeptide comprising a Candida utilis uricase, or a variant thereof, and a second exogenous polypeptide comprising a uric acid transporter, or variant thereof, where the engineered erythroid cell has between about 0.1-10×10-¹⁰, or between about 0.5-5×10-¹⁰, or between about 1-2×10-¹⁰ units of uricolytic activity per cell, for example about 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell.

In some embodiments, the engineered erythroid cell comprises a first exogenous polypeptide comprising a Candida utilis uricase, or a variant thereof, and a second exogenous polypeptide comprising human URAT1, or variant thereof, where the engineered erythroid cell has between 1e-9 and 1e-11 units of uricolytic activity per cell, for example between 1e-9 and le-10, or between 1e-10 and 1e-11, or between 5e-9 and 5e-10, or between 5e-10 and 1e-11 units of uricoloytic activity per cell. In some embodiments, the engineered erythroid cell comprises a first exogenous polypeptide comprising a Candida utilis uricase, or a variant thereof, and a second exogenous polypeptide comprising human URAT1, or variant thereof, where the engineered erythroid cell has at least 1-2×10-¹⁰ units of uricolytic activity per cell, for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cell comprises a first exogenous polypeptide comprising a Candida utilis uricase, or a variant thereof, and a second exogenous polypeptide comprising URAT1, or variant thereof, where the engineered erythroid cell has between about 3-6×10⁻¹⁰, or between about 3.5-6×10⁻¹⁰, or between about 4-6×10⁻¹⁰, or between about 4.5-6×10⁻¹⁰, or between about 5-6×10⁻¹⁰, or between about 5.5-6×10⁻¹⁰ units of uricolytic activity per cell, for example about 3e-10, 3.1 e-10, 3.2e-10, 3.3e-10, 3.4e-10, 3.5e-10, 3.6e-10, 3.7e-10, 3.8e-10, 3.9e-10, 4e-10, 4.1 e-10, 4.2e-10, 4.3e-10, 4.4e-10, 4.5e-10, 4.6e-10, 4.7e-10, 4.8e-10, 4.9e-10, 5e-10, 5.1 e-10, 5.2e-10, 5.3e-10, 5.4e-10, 5.5e-10, 5.6e-10, 5.7e-10, 5.8e-10, 5.9e-10, 6e-10 units of uricoytic activity per cell. In some embodiments, the engineered erythroid cell comprises a first exogenous polypeptide comprising a Candida utilis uricase, or a variant thereof, and a second exogenous polypeptide comprising human URAT1, or variant thereof, where the engineered erythroid cell has between about 0.1-10×10-¹⁰, or between about 0.5-5×10-¹⁰, or between about 1-2×10-¹⁰ units of uricolytic activity per cell, for example about 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell.

In some embodiments, the effective amount of engineered erythroid cells is based on units of uricolytic activity per dose. In some embodiments, the effective amount of engineered erythroid cells comprises between 5-50 units of uricolytic activity per dose, for example between 10-50, between 10-40, between 10-30, or between 10-20 units of uricolytic activity per dose. In some embodiments, the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a uric acid degrading polypeptide, or a variant thereof, and a second exogenous polypeptide comprising a uric acid transporter, or variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a uric acid degrading polypeptide, or a variant thereof, and a second exogenous polypeptide comprising a uric acid transporter, or variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a uric acid degrading polypeptide, or a variant thereof, a second exogenous polypeptide comprising a uric acid transporter, or variant thereof, and a third exogenous polypeptide comprising a catalase, or a variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a uric acid degrading polypeptide, or a variant thereof, a second exogenous polypeptide comprising a uric acid transporter, or variant thereof, and a third exogenous polypeptide comprising a catalase, or a variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a uric acid degrading polypeptide, or a variant thereof, a second exogenous polypeptide comprising a uric acid transporter, or variant thereof, and a third exogenous polypeptide comprising a catalase, or a variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a uricase, or a variant thereof, and a second exogenous polypeptide comprising a uric acid transporter, or variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a uricase, or a variant thereof, and a second exogenous polypeptide comprising a uric acid transporter, or variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a uricase, or a variant thereof, and a second exogenous polypeptide comprising a uric acid transporter, or variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a uricase, or a variant thereof, a second exogenous polypeptide comprising a uric acid transporter, or variant thereof, and a third exogenous polypeptide comprising a catalase, or a variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a Candida utilis uricase, or a variant thereof, and a second exogenous polypeptide comprising a uric acid transporter, or variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a Candida utilis uricase, or a variant thereof, and a second exogenous polypeptide comprising a uric acid transporter, or variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a Candida utilis uricase, or a variant thereof, and a second exogenous polypeptide comprising a uric acid transporter, or variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a Candida utilis uricase, or a variant thereof, a second exogenous polypeptide comprising a uric acid transporter, or variant thereof, and a third exogenous polypeptide comprising a catalase, or a variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a Candida utilis uricase, or a variant thereof, a second exogenous polypeptide comprising a uric acid transporter, or variant thereof, and a third exogenous polypeptide comprising a catalase, or a variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a Candida utilis uricase, or a variant thereof, a second exogenous polypeptide comprising a uric acid transporter, or variant thereof, and a third exogenous polypeptide comprising a catalase, or a variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a Candida utilis uricase, or a variant thereof, and a second exogenous polypeptide comprising human URAT1, or variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a Candida utilis uricase, or a variant thereof, and a second exogenous polypeptide comprising human URAT1, or variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a Candida utilis uricase, or a variant thereof, and a second exogenous polypeptide comprising human URAT1, or variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In some embodiments, the engineered erythroid cell comprises a first exogenous polypeptide comprising a Candida utilis uricase, or a variant thereof, a second exogenous polypeptide comprising human URAT1, or variant thereof, and a third exogenous polypeptide comprising a catalase, or a variant thereof, where the effective amount of engineered erythroid cells comprises between 5-50 units of uricolytic activity per dose, for example between 10-50, between 10-40, between 10-30, or between 10-20 units of uricolytic activity per dose. In some embodiments, the engineered erythroid cell comprises a first exogenous polypeptide comprising a Candida utilis uricase, or a variant thereof, a second exogenous polypeptide comprising human URAT1, or variant thereof, and a third exogenous polypeptide comprising a catalase, or a variant thereof, where the effective amount of engineered erythroid cells comprises at least 10-20 units of uricolytic activity per dose, for example at least 10, 11, 12, 13, 14, 15, 16, 17, 18, 19 or 20 units of uricolytic activity per dose. In some embodiments, the engineered erythroid cell comprises a first exogenous polypeptide comprising a Candida utilis uricase, or a variant thereof, a second exogenous polypeptide comprising human URAT1, or variant thereof, and a third exogenous polypeptide comprising a catalase, or a variant thereof, where the effective amount of engineered erythroid cells comprises about 10-20 units of uricolytic activity per dose, for example about 10, 11, 12, 13, 14, 15, 16, 17, 18, 19 or 20 units of uricolytic activity per dose.

In one example, administration of the engineered erythroid cell is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that may appear.

Any one of the doses provided herein for an engineered erythroid cell as described herein can be used in any one of the methods or kits provided herein. Generally, when referring to a dose to be administered to a subject the dose is a label dose. Thus, in any one of the methods provided herein the dose(s) are label dose(s).

Also provided herein are a number of possible dosing schedules. Accordingly, any one of the subjects provided herein may be treated according to any one of the dosing schedules provided herein. As an example, any one of the subject provided herein may be treated with an engineered erythroid cell as described herein. In certain embodiments, the engineered erythroid cell comprises a first exogenous polypeptide comprising uricase, or a variant thereof, and a second exogenous polypeptide comprising a uric acid transporter, or a variant thereof. In certain embodiments, the engineered erythroid cell comprises a first exogenous polypeptide comprising uricase, or a variant thereof, a second exogenous polypeptide comprising a uric acid transporter, or a variant thereof, and a third exogenous polypeptide comprising a catalase, or a variant thereof.

Each dose of synthetic membrane-receiver polypeptide complexes can be administered at intervals such as once daily, once weekly, twice weekly, once monthly, or twice monthly.

In some embodiments, a subject is dosed on a monthly dosing schedule.

In some embodiments, the engineered erythroid cell is administered to the subject about once every four weeks.

The mode of administration for the composition(s) of any one of the treatment methods provided may be by intravenous administration, such as an intravenous infusion that, for example, may take place over about 1 hour. Additionally, any one of the methods of treatment provided herein may also include administration of an additional therapeutic, as described in more detail below. The administration of the additional therapeutic may be according to any one of the applicable treatment regimens provided herein.

In some embodiments of any one of the methods provided herein, the level of uric acid is measured in the subject at one or more time points before, during and/or after the treatment period.

### Subjects

The methods described herein are intended for use with any subject that may experience the benefits of these methods. Thus, “subjects,” “patients,” and “individuals” (used interchangeably) include humans as well as non-human subjects, particularly domesticated animals. Subjects provided herein can be in need of treatment according to any one of the methods or compositions or kits provided herein. Such subjects include those with elevated serum uric acid levels or uric acid deposits. Such subjects include those with hyperuricemia. It is within the skill of a clinician to be able to determine subjects in need of a treatment as provided herein.

In some embodiments, the subject and/or animal is a mammal, e g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. In some embodiments, the subject and/or animal is a human. In some embodiments, the human is a pediatric human In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal In certain embodiments, the subject is a human cancer patient that cannot receive chemotherapy, e.g. the patient is unresponsive to chemotherapy or too ill to have a suitable therapeutic window for chemotherapy (e.g. experiencing too many dose- or regimen-limiting side effects). In certain embodiments, the subject is a human subject having gout or another disease or condition associated with hyperuricemia. In other certain embodiments, the subject is a human subject having chronic refractory gout.

In some embodiments, any one of the subjects for treatment as provided in any one of the methods provided has gout or a condition associated with gout or another condition as provided herein. In some embodiments, any one of the subjects for treatment as provided in any one of the methods provided has been diagnosed with a disease selected from the group consisting of gout, rheumatoid arthritis, osteoarthritis, cerebral stroke, ischemic heart disease, arrhythmia, and chronic renal disease. In some embodiments, any one of the subjects for treatment as provided in any one of the methods provided has chronic refractory gout. In some embodiments, any one of the subjects for treatment as provided in any one of the methods provided the subject has had or is expected to have gout flare. In some embodiments, any one of the subjects for treatment as provided in any one of the methods provided has one or more risk factors for hyperurecemia selected from the group consisting of insulin resistance, obesity, a purine rich diet and advanced age. In some embodiments, any one of the subjects for treatment as provided in any one of the methods provided has been diagnosed with symptomatic gout with at least 3 gout flares in the previous 18 months. In some embodiments, any one of the subjects for treatment as provided in any one of the methods provided has been diagnosed with at least 1 gout tophus or gouty arthritis. In some embodiments, any one of the subjects for treatment as provided in any one of the methods provided has a contraindication to allopurinol. Contraindications to allopurinol include extreme loss of body water, chronic heart failure, allergic reaction causing inflammation of blood vessels, liver problems and moderate to severe kidney impairment. In some embodiments, any one of the subjects for treatment as provided in any one of the methods provided has a failure to normalize uric acid to less than 6 mg/dL after at least 3 months of allopurinol treatment.

In some embodiments, the subject has or is at risk of having an elevated uric acid level, e.g., an elevated plasma or serum uric acid level. When blood levels of uric acid may exceed the physiologic limit of solubility, the uric acid may crystallize in the tissues, including the joints, and may cause gout and gout-associated conditions.

In some embodiments, serum uric acid levels >5 mg/dL, >6 mg/dL, or >7 mg/dL are indicative that a subject may be a candidate for treatment with any one of the methods or compositions or kits described herein. In some embodiments, such a subject has a serum level of uric acid greater than 6 mg/dL, for example, between 6.1 mg/dL-15 mg/dL, between 6.1 mg/dL-10 mg/dL, 7 mg/dL-15 mg/dL, 7 mg/dL-10 mg/dL, 8 mg/dL-15 mg/dL, 8 mg/dL-10 mg/dL, 9 mg/dL-15 mg/dL, 9 mg/dL-10 mg/dL, 10 mg/dL-15 mg/dL, or 11 mg/dL-14 mg/dL. In some embodiments, the subject has serum level of uric acid of about 6.1 mg/dL, 6.2 mg/dL, 6.3 mg/dL, 6.4 mg/dL, 6.5 mg/dL, 6.7 mg/dL, 6.8 mg/dL, 6.9 mg/dL, 7.0 mg/dL, 7.1 mg/dL, 7.2 mg/dL, 7.3 mg/dL, 7.4 mg/dL, 7.5 mg/dL, 7.6 mg/dL 7.7 mg/dL, 7.8 mg/dL, 7.9 mg/dL, 8.0 mg/dL, 8.1 mg/dL, 8.2 mg/dL, 8.3 mg/dL, 8.4 mg/dL, 8.5 mg/dL, 8.6 mg/dL, 8.7 mg/dL, 8.8 mg/dL, 8.9 mg/dL, 9.0 mg/dL, 9.1 mg/dL, 9.2 mg/dL, 9.3 mg/dL, 9.4 mg/dL, 9.5 mg/dL, 9.6 mg/dL, 9.7 mg/dL, 9.8 mg/dL, 9.9 mg/dL, 10.0 mg/dL, 10.1 mg/dL, 10.2 mg/dL, 10.3 mg/dL, 10.4 mg/dL, 10.5 mg/dL, 10.6 mg/dL, 10.7 mg/dL, 10.8 mg/dL, 10.9 mg/dL, 11.0 mg/dL, 11.1 mg/dL, 11.2 mg/dL, 11.3 mg/dL, 11.4 mg/dL, 11.5 mg/dL, 11.6 mg/dL, 11.7 mg/dL, 11.8 mg/dL, 11.9 mg/dL, 12.0 mg/dL, 12.1 mg/dL, 12.2 mg/dL, 12.3 mg/dL, 12.4 mg/dL, 12.5 mg/dL, 12.6 mg/dL, 12.7 mg/dL, 12.8 mg/dL, 12.9 mg/dL, 13.0 mg/dL, 13.1 mg/dL, 13.2 mg/dL, 13.3 mg/dL, 13.4 mg/dL, 13.5 mg/dL, 13.6 mg/dL, 13.7 mg/dL, 13.8 mg/dL, 13.9 mg/dL, 14.0 mg/dL, 14.1 mg/dL, 14.2 mg/dL, 14.3 mg/dL, 14.4 mg/dL, 14.5 mg/dL, 14.6 mg/dL, 14.7 mg/dL, 14.8 mg/dL, 14.9 mg/dL, 15.0 mg/dL or higher. In some embodiments, the subject has a plasma or serum uric acid level of 5.0 mg/dL, 5.1 mg/dL, 5.2 mg/dL, 5.3 mg/dL, 5.4 mg/dL, 5.5 mg/dL, 5.6 mg/dL, 5.7 mg/dL, 5.8 mg/dL, 5.9 mg/dL, 6.0 mg/dL, 6.1 mg/dL, 6.2 mg/dL, 6.3 mg/dL, 6.4 mg/dL, 6.5 mg/dL, 6.6 mg/dL, 6.7 mg/dL, 6.8 mg/dL, 6.9 mg/dL, or 7.0 mg/dL. In some embodiments, the subject has a plasma or serum uric acid level of greater than or equal to 5.0 mg/dL, 5.1 mg/dL, 5.2 mg/dL, 5.3 mg/dL, 5.4 mg/dL, 5.5 mg/dL, 5.6 mg/dL, 5.7 mg/dL, 5.8 mg/dL, 5.9 mg/dL, 6.0 mg/dL, 6.1 mg/dL, 6.2 mg/dL, 6.3 mg/dL, 6.4 mg/dL, 6.5 mg/dL, 6.6 mg/dL, 6.7 mg/dL, 6.8 mg/dL, 6.9 mg/dL, or 7.0 mg/dL.

In some embodiments, the subject has, or is at risk of having, hyperuricemia. In some embodiments, the subject has, or is at risk of having, gout, acute gout, acute intermittent gout, gouty arthritis, acute gouty arthritis, acute gouty arthropathy, acute polyarticular gout, recurrent gouty arthritis, chronic gout (with our without tophi), tophaceous gout, chronic tophaceous gout, chronic advanced gout (with our without tophi), chronic polyarticular gout (with our without tophi), chronic gouty arthropathy (with our without tophi), idiopathic gout, idiopathic chronic gout (with or without tophi), primary gout, chronic primary gout (with or without tophi), refractory gout, such as chronic refractory gout, axial gouty arthropathy, a gout attack, a gout flare, podagra (i.e., monarticular arthritis of the great toe), chiragra (i.e., monarticular arthritis of the hand), gonagra (i.e., monarticular arthritis of the knee), gouty bursitis, gouty spondylitis, gouty synovitis, gouty tenosynovitis, gout that affects tendons and ligaments, lead-induced gout (i.e., saturnine gout), drug induced gout, gout due to renal impairment, gout due to kidney disease, chronic gout due to renal impairment (with or without tophi), chronic gout due to kidney disease (with or without tophi), erosive bone disease associated with gout, stroke associated with gout, vascular plaque associated with gout, cirrhosis or steatohepatitis associated with gout, liver-associated gout, incident and recurrent gout, diabetes associated with damage to pancreas in gout, general inflammatory diseases exacerbated by gout, other secondary gout, or unspecified gout.

In some embodiments, the subject has, or is at risk of having, a condition associated with the renal system, for example, calculus of urinary tract due to gout, uric acid urolithiasis, uric acid nephrolithiasis, uric acid kidney stones, gouty nephropathy, acute gouty nephropathy, chronic gouty nephropathy, urate nephropathy, uric acid nephropathy, and gouty interstitial nephropathy.

In some embodiments, the subject has, or is at risk of having, a condition associated with the nervous system, for example, peripheral autonomic neuropathy due to gout, gouty neuropathy, gouty peripheral neuropathy, gouty entrapment neuropathy, or gouty neuritis.

In some embodiments, the subject has, or is at risk of having, a condition associated with the cardiovascular system, for example, metabolic syndrome, hypertension, obesity, diabetes, myocardial infarction, stroke, dyslipidemia, hypertriglyceridemia, insulin resistance/hyperglycemia, coronary artery disease/coronary heart disease, coronary artery disease or blockage associated with gout or hyperuricemia, heart failure, peripheral arterial disease, stroke/cerebrovascular disease, peripheral vascular disease, and cardiomyopathy due to gout.

In some embodiments, the subject has, or is at risk of having, a condition associated with the ocular system including, for example, gouty iritis, inflammatory disease in the eye caused by gout, dry eye syndrome, red eye, uveitis, intraocular hypertension, glaucoma, and cataracts.

In some embodiments, the subject has, or is at risk of having, a condition associated with the skin including, for example, gout of the external ear, gouty dermatitis, gouty eczema, gouty panniculitis, and miliarial gout.

In some embodiments, the subject is selected for treatment with an erythroid cell engineered to degrade uric acid of the present disclosure. In some embodiments, the subject is selected for treatment of hyperuricemia with an engineered erythroid cell of the present disclosure. In some embodiments, the subject is selected for treatment of gout with an engineered erythroid cell of the present disclosure. In some embodiments, the subject is selected for treatment of chronic refractory gout with an engineered erythroid cell of the present disclosure.

In certain embodiments, the methods of the present disclosure provide treatment of gout and diseases or disorders associated with hyperuricemia to human patients suffering therefrom. The treatment population is thus human subjects diagnosed as suffering from gout or hyperuricemia. The invention encompasses the treatment of a human subject at risk of suffering from a recurrent gout episode or for developing hyperuricemia or gout.

The present disclosure also encompasses treating a population of patients with drug-induced gout flares, including flares induced by gout therapeutics such as xanthine oxidase inhibitors, such as allopurinol and febuxostat; flares induced by urate oxidase, for example, uricase, rasburicase and pegylated uricase; and flares induced by uricosuric agents, such as probenecid, sulfinpyrazone, benzbromarone, and fenofibrate. By “drug-induced” gout flare is meant occurrence of or increased incidence of a gout flare associated with initiation of therapy to treat gout and/or administration of a therapeutic agent for the treatment of gout, for example, initiation of therapy with a xanthine oxidase inhibitor, urate oxidase, or a uricosuric agent. A gout flare is “associated” with initiation of gout therapy when the flare occurs contemporaneously or following at least a first administration of a therapeutic agent for the treatment of gout.

### V. Pharmaceutical Compositions

The present disclosure encompasses the preparation and use of pharmaceutical compositions comprising an engineered erythroid cell (e.g., engineered enucleated cells) of the disclosure as an active ingredient. Such a pharmaceutical composition may consist of the active ingredient alone, as a combination of at least one active ingredient (e.g., an effective dose of an engineered erythroid cell) in a form suitable for administration to a subject, or the pharmaceutical composition may comprise the active ingredient and one or more pharmaceutically acceptable carriers, one or more additional (active and/or inactive) ingredients, or some combination of these.

In some embodiments, a pharmaceutical composition comprises a plurality of the engineered erythroid cells described herein, and a pharmaceutically acceptable carrier. In further embodiments, the pharmaceutical composition comprises a therapeutically effective dose of the engineered erythroid cells.

In some embodiments, the pharmaceutical composition comprises between 1e10 and 1e12 engineered erythroid cells. In some embodiments, the pharmaceutical composition comprises at least 1e10, 2e10, 3e10, 4e10, 5e10, 6 e10, 7e10, 8e10, 9e10, or 1e11 engineered erythroid cells. In some embodiments, the pharmaceutical composition comprises at least 3e10 engineered erythroid cells. In some embodiments, the engineered erythroid cells have about 1-2×10-¹⁰ units of uricolytic activity per cell, for example for example at least 1e-10, 1.1 e-10, 1.2e-10, 1.3e-10, 1.4e-10, 1.5e-10, 1.6e-10, 1.7e-10, 1.8e-10, 1.9e-10, 2e-10 units of uricoytic activity per cell. In some embodiments, the engineered erythroid cells comprise 10-20 units of uricolytic activity per dose, for example 10, 11, 12, 13, 14, 15, 16, 17, 18 19 or 20 units of uricolytic activity per cell.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the pharmaceutical compositions may be carried out in any convenient manner, including by aerosol inhalation, injection, ingestion, transfusion, implantation or transplantation. The compositions of the present disclosure may be administered to a patient subcutaneously, intradermally, intramuscularly, by intravenous (i.v.) injection, or intraperitoneally. The pharmaceutical compositions may be injected directly into a tumor or lymph node.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the disclosure may be prepared, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing the active ingredient, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The engineered erythroid cell of the disclosure can be administered to an animal, preferably a human. Where the engineered erythroid cell are administered, they can be administered in an amount ranging from about 100,000 to about one billion cells wherein the cells are infused into the animal, preferably, a human patient in need thereof. While the precise dosage administered will vary depending upon any number of factors, including but not limited to, the type of animal and type of disease state being treated, the age of the animal and the route of administration.

The engineered erythroid cell may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An engineered erythroid cell may be co-administered with the various other compounds (e.g. other therapeutic agents). Alternatively, the compound(s) may be administered an hour, a day, a week, a month, or even more, in advance of the engineered erythroid cell, or any permutation thereof. Further, the compound(s) may be administered an hour, a day, a week, or even more, after administration of the engineered erythroid cell, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the engineered erythroid cell (or T cells or NK cells expanded thereby), and the like.

Further, it would be appreciated by one skilled in the art, based upon the disclosure provided herein, that where the engineered erythroid cell is to be administered to a mammal, the cells are treated so that they are in a “state of no growth”; that is, the cells are incapable of dividing when administered to a mammal. As disclosed elsewhere herein, the cells can be irradiated to render them incapable of growth or division once administered into a mammal. Other methods, including haptenization (e.g., using dinitrophenyl and other compounds), are known in the art for rendering cells to be administered, especially to a human, incapable of growth, and these methods are not discussed further herein. Moreover, the safety of administration of engineered erythroid cells that have been rendered incapable of dividing in vivo has been established in Phase I clinical trials using engineered erythroid cell transfected with plasmid vectors encoding some of the molecules discussed herein.

In some embodiments, the disclosure features a pharmaceutical composition comprising a plurality of the engineered erythroid cells described herein, and a pharmaceutical carrier. In other embodiments, the disclosure features a pharmaceutical composition comprising a population of engineered erythroid cells as described herein, and a pharmaceutical carrier. It will be understood that any single engineered erythroid cell, plurality of engineered erythroid cells, or population of engineered erythroid cells as described elsewhere herein may be present in a pharmaceutical composition of the invention.

In some embodiments, the pharmaceutical compositions provided herein comprise engineered (i.e. modified) erythroid cells and unmodified erythroid cells. For example, a single unit dose of erythroid cells (e.g., modified and unmodified erythroid cells) can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise engineered enucleated erythroid cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

In some embodiments of the above aspects and embodiments, the engineered erythroid cell is an enucleated cell, e.g. an erythrocyte or a reticulocyte. In some embodiments of the above aspects and embodiments, the engineered erythroid cell is a nucleated cell.

### Combination Therapies

According to some embodiments, the disclosure provides methods that further comprise administering an additional agent (e.g. an additional therapeutic) to a subject. In some embodiments, the disclosure pertains to co-administration and/or co-formulation.

Additional therapeutics for elevated uric acid levels, gout, gout flare, or conditions associated with gout, may be administered to any one of the subjects provided herein, such as for the reduction of uric acid levels and/or gout treatment and/or gout flare prevention. Any one of the methods provided herein may include the administration of one or more of these additional therapeutics. In some embodiments, any one of the methods provided herein do not comprise the concomitant administration of an additional therapeutic. Examples of additional therapeutics include, but are not limited to, the following. Other examples will be known to those of skill in the art.

Additional therapeutics include anti-inflammatory therapeutics (i.e., any therapeutic that can act to reduce inflammation). Anti-inflammatory therapeutics include, but are not limited to, corticosteroids or derivatives of cortisol (hydrocortisone). Corticosteroids include, but are not limited to, glucocorticoids and mineralocorticoids. Still other examples of corticosteroids include, but are not limited to, those that are natural and those that are synthetic. Corticosteroids, particularly glycocorticoids, have anti-inflammatory and immunosuppressive effects that may be effective in managing symptoms, including pain and inflammation associated with gout, gout flare, and/or conditions associated with gout. Administration of corticosteroids may also aid in reducing hypersensitivity reactions associated with one or more additional therapies, for example uricase replacement therapy. Still other non-limiting examples of corticosteroids, include prednisone, prednisolone, Medrol, and methylprednisolone.

Additional therapeutics include short term therapies for gout flare or pain and inflammation associated with any of the symptoms associated with gout or a condition associated with gout include nonsteroidal anti-inflammatory drugs (NSAIDS), colchicine, oral corticosteroids. Non-limiting examples of NSAIDS include both over-the-counter NSAIDS, such as ibuprofen, aspirin, and naproxen, as well as prescription NSAIDS, such as celecoxib, diclofenac, diflunisal, etodolac, indomethacin, ketoprofen, ketorolac, nabumetrone, oxaprozin, piroxiam salsalate, sulindac, and tolmetin.

Colchicine is an anti-inflammatory agent that is generally considered as an alternative for NSAIDs for managing the symptoms, including pain and inflammation associated with gout, gout flare, and/or conditions associated with gout.

Further examples of additional therapeutics include xanthine oxidase inhibitors, which are molecules that inhibit xanithine oxidase, reducing or preventing the oxidation of xanthine to uric acid, thereby reducing the production of uric acid. Xanthine oxidase inhibitors are generally classified as either purine analogues and other types of xanthine oxidase inhibitors. Examples of xanthine oxidase inhibitors include allopurinol, oxypurinol, tisopurine, febuxostat, topiroxostat, inositols (e.g., phytic acid and myo-inositol), flavonoids (e.g., kaempferol, myricetin, quercetin), caffeic acid, and 3,4-dihydrox-5-nitrobenzaldehyde (DHNB).

Still other examples of additional therapeutics include uricosuric agents. Uricosuric agents aim to increase excretion of uric acid in order to reduce serum levels of uric acid by modulating renal tubule reabsorption. For example, some uricosuric agents modulate activity of renal transporters of uric acid (e.g., URAT1/SLC22A12 inhibitors). Non-limiting examples of uricosuric agents include probenecid, benzbromarone, lesinurad, sulfinpyrazone. Other additional therapeutics may also have uricosuric activity, such as aspirin.

Additional therapeutics also include other uricase-based therapies, which include pegylated uricase. Such therapies, such as when infused into humans, have been shown to reduce blood uric acid levels and improve gout symptoms. Rasburicase (ELITEK), an unpegylated recombinant uricase cloned from Aspergillus flavus, is approved for management of uric acid levels in patients with tumor lysis syndrome. KRYSTEXXA (pegloticase) is a recombinant uricase (primarily porcine with a carboxyl-terminus sequence from baboon) bound by multiple 10 kDa PEG molecules approved for the treatment of chronic refractory gout.

The treatments provided herein may allow patients to switch to oral gout therapy, such as with xanthine oxidase inhibitors, unless and until such patients experience a subsequent manifestation of uric acid deposits at which time a new course of treatment as provided herein according to any one of the methods provided is then undertaken. Any one of the methods provided herein, thus, can include the subsequent administration of an oral gout therapeutic as an additional therapeutic after the treatment regimen according to any one of the methods provided is performed. It is believed that oral therapy may not completely prevent the buildup over time of uric acid crystals in patients with a history of chronic tophaceous gout. As a result, it is anticipated that treatment as provided herein is likely to be required intermittently in such patients. Thus, in such subjects, the subject is also further administered one or more compositions according to any one of the methods provided herein.

The treatments provided herein may allow patients to subsequently be treated with a uric acid lowering therapeutic, such as a uricase. In some embodiments, without an immunosuppressant. In some embodiments, without synthetic nanocarriers comprising an immunosuppressant.

Treatment according to any one of the methods provided herein may also include a pre-treatment with an anti-gout flare therapeutic, such as with colchicine or NSAIDS. Accordingly, any one of the methods provided herein may further comprise such an anti-gout flare therapeutic whereby the anti-gout flare therapeutic is concomitantly administered with the composition comprising uricase and the composition comprising synthetic nanocarriers comprising an immunosuppressant.

Monitoring of a subject, such as the measurement of serum uric acid levels and/or ADAs, may be an additional step further comprised in any one of the methods provided herein. In some embodiments, should such subject develop an undesired immune response, the subject is further administered one or more compositions according to any one of the methods provided herein. In some embodiments of any one of the methods provided herein, the subject is monitored with dual energy computed tomography (DECT), that can be used to visualize uric acid deposits in joints and tissues. Imaging, such as with DECT, can be used to assess the efficacy of treatment with any one of the methods or compositions provided herein. As a result, any one of the methods provided herein can further include a step of imaging, such as with DECT. In some embodiments of any one of the methods provided herein, the subject is one in which the gout, such as chronic tophaceous gout, or condition associated with gout has been diagnosed with such imaging, such as with DECT.

### VI. Kits

The disclosure includes various kits which comprise an engineered erythroid cell of the disclosure, and optionally further include nucleic acids encoding the exogenous polypeptides. Although exemplary kits are described below, the contents of other useful kits will be apparent to the skilled artisan in light of the present disclosure. Each of these kits is included within the disclosure.

In some embodiments, the kit optionally includes a device suitable for administration of the composition (or one or more agents of a combination therapy), e.g., a syringe or other suitable delivery device. Moreover, in embodiments the kit further comprises an instructional material which describe the use of the kit to perform the methods described herein. These instructions simply embody the disclosure provided herein.

In some embodiments, the kit includes a pharmaceutically-acceptable carrier. The composition is provided in an appropriate amount as set forth elsewhere herein. Further, the route of administration and the frequency of administration are as previously set forth elsewhere herein.

The kit encompasses an engineered erythroid cell comprising a wide plethora of molecules, such as, but not limited to, the exogenous polypeptides set forth herein. However, the skilled artisan armed with the teachings provided herein, would readily appreciate that the disclosure is in no way limited to these, or any other, combination of molecules. Rather, the combinations set forth herein are for illustrative purposes and they in no way limit the combinations encompassed by the present disclosure.

Further, the kit comprises a kit where each molecule to be transduced into the engineered erythroid cell is provided as an isolated nucleic acid encoding a molecule, a vector comprising a nucleic acid encoding a molecule, and any combination thereof, including where at least two molecules are encoded by a contiguous nucleic acid and/or are encoded by the same vector.

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

### Example 1

### Assessment of Uricase Activity in HEK-293T Cells

Uricases from different species have different pH requirements for optimal activity. The goal of this study was to identify a uricase that has as high activity as possible at neutral pH when expressed in HEK-293T cells. GFP-fused uricases from Candida utilis, Aspergillus flavus, and Arthrobacter globiformis were expressed in human embryonic kidney cells (HEK-293T). Uricase expression was quantified by determining the mean fluorescence intensity of GFP fluorescence in uricase expressing cells and comparing the fluorescence with that of GFP-conjugated beads. The activity of the expressed uricase was determined by incubating HEK-293T cell lysate with 500 uM uric acid in pH 7.4 buffer and tracking the degradation of uric acid over time.

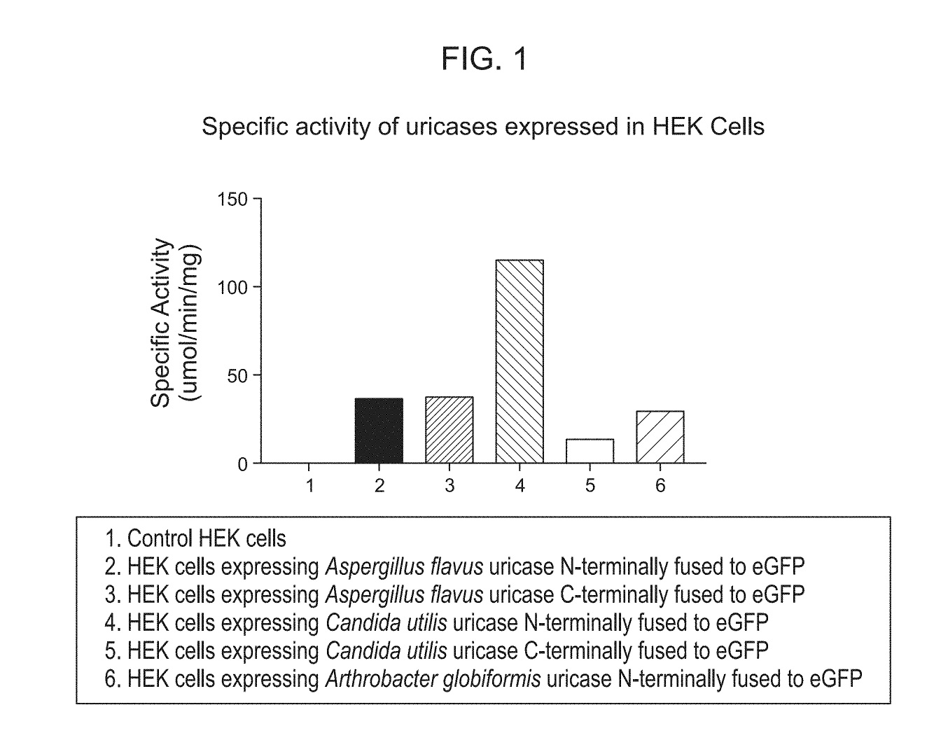

Combining activity data with quantified expression, the specific activity of uricases expressed in the HEK-293T cells was determined, as shown in FIG. 1. From the 3 uricases tested, uricase from Candida utilis yielded the highest specific activity at neutral pH (see FIG. 1).

### Example 2

### Generation of Erythroid Cells Genetically Engineered to Comprise Uricase

### Production of Lentiviral Vector

A lentiviral vector is constructed with a gene encoding uricase under the control of the MSCV promoter. Lentivirus is produced in HEK-293T cells by transfecting the cells with pPACKH1 (System Biosciences) or in-house made packaging vector mix and the lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant is collected, filtered through 0.45 um filter, and frozen in aliquots in −80° C.

### Expansion and Differentiation of Erythroid Cells

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C.

### Production of Uricase

The presence of uricase is assessed via intracellular staining using antibody specific for the uricase expressed and analysis is via flow cytometry or western blot analysis following SDS-PAGE separation.

### Example 3

### Generation of Erythroid Cells Genetically Engineered to Comprise Uric Acid Transporter

### Production of Lentiviral Vector

A lentiviral vector is constructed with a gene encoding uric acid transporter under the control of the MSCV promoter. Lentivirus is produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant is collected, filtered through 0.45 um filter, and frozen in aliquots in −80° C.

### Expansion and Differentiation of Erythroid Cells

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C.

### Production of Uric Acid Transporter

The presence of uric acid transporter is assessed either by intracellular or extracellular staining using antibody specific for the uric acid transporter produced and analysis is via flow cytometry.

### Example 4

### Generation of Erythroid Cells Genetically Engineered to Comprise Uricase and Uric Acid Transporter by Expression in Separate Lentiviral Vectors

Production of uricase and uric acid transporter in engineered erythroid cells can be accomplished in two alternate ways. In this example, the uricase and uric acid transporter are present on two separate lentivirus vectors, and the two different lentiviruses are mixed at transduction.

### Production of Lentiviral Vector

Lentiviral vector is constructed with a gene encoding uricase under the control of the MSCV promoter, as described above in Example 2. Further, another lentiviral vector is constructed with gene encoding uric acid transporter under the control of the MSCV promoter, as described in Example 3. Each lentivirus is produced separately in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant is collected, filtered through 0.45 um filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this instance, where the uricase and uric acid transporter are expressed from two separate vectors, the two lentiviruses produced from the corresponding vectors are combined together for the transduction step.

### Production of Uricase and Uric Acid Transporter:

The presence of uricase is assessed via intracellular staining using antibody specific for the uricase produced and analysis via flow cytometry or western blot analysis following SDS-PAGE separation. The presence of uric acid transporter is assessed either by intracellular or extracellular staining using antibody specific for the uric acid transporter produced and analysis via flow cytometry.

### Example 5

### Generation of Erythroid Cells Genetically Engineered to Comprise Uricase and Uric Acid Transporter by Expression in the Same Lentiviral Vector

In this example, production of uricase and uric acid transporter in erythroid cells is accomplished by including both uricase and uric acid transporter in the same lentiviral vector.

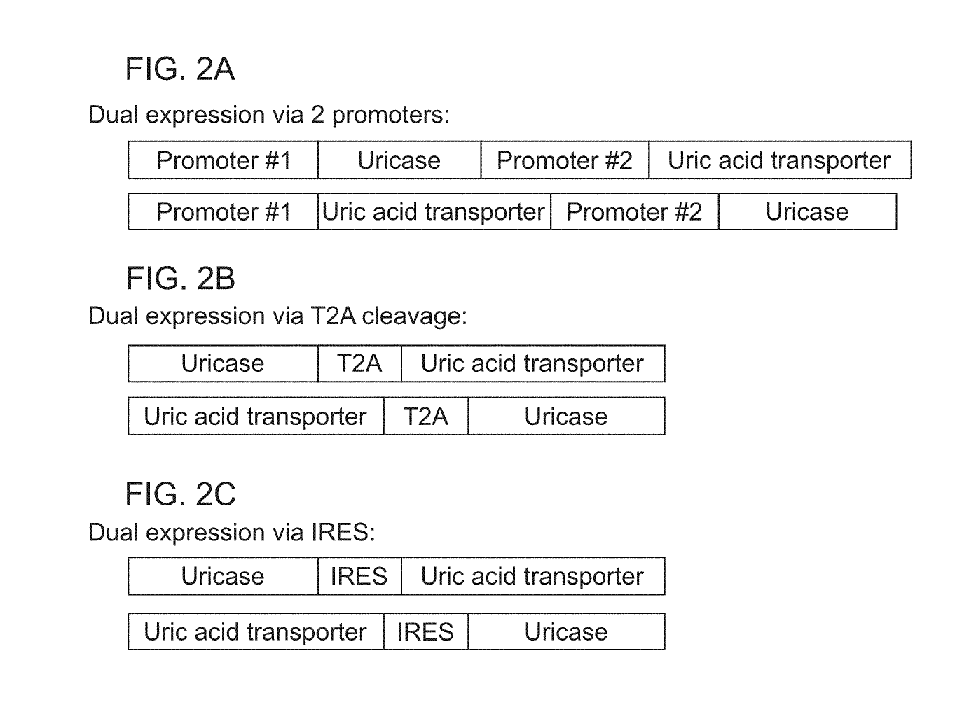

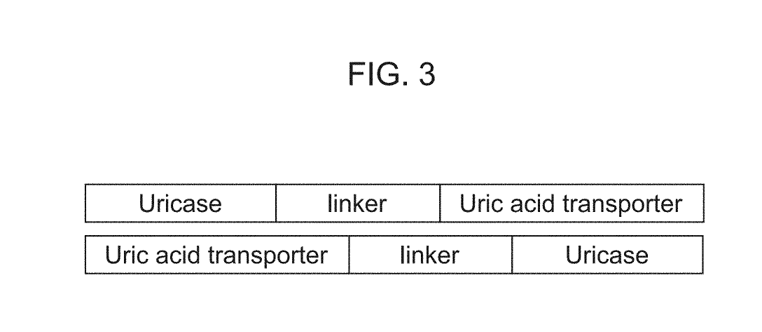

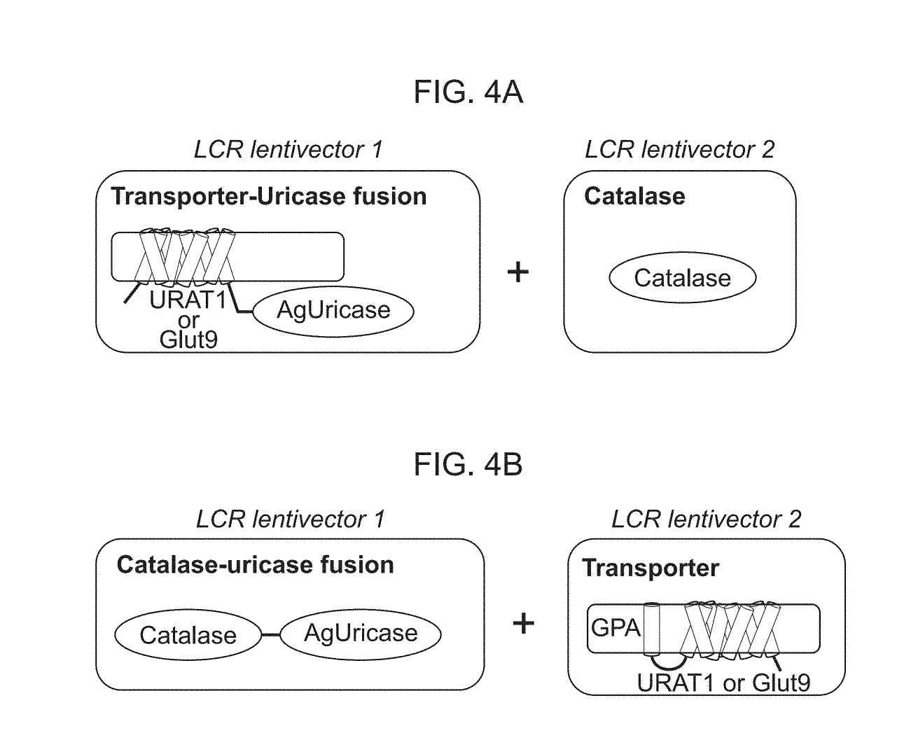

Three strategies for co-expression of uricase and uric acid transporter from a single vector are outlined in FIG. 2A, FIG. 2B and FIG. 2C, all of which lead to the translation of two separate polypeptide chains, corresponding to the uricase and uric acid transporter proteins. The first strategy involves expression of uricase and uric acid transporter from two different promoters (FIG. 2A). The second approach involves dual expression of uricase and uric acid transporter via a T2A cleavage (FIG. 2B). The third strategy involves dual expression of uricase and uric acid transporter via an IRES (FIG. 2C). Alternatively, uricase and uric acid transporter can be expressed as direct polypeptide fusions separated by a linker, as shown in FIG. 3.

### Production of Lentiviral Vector:

Lentiviral vector is constructed with a gene encoding uricase and a gene encoding uric acid transporter directly fused via a linker (FIG. 3), or a gene encoding uricase and a gene encoding uric acid transporter separated by the T2A sequence (FIG. 2B) or IRES (FIG. 2C) under the control of the MSCV promoter. Alternatively, another lentiviral vector is constructed containing a uricase and a uric acid transporter under the control of two different promoters (FIG. 2A). Lentivirus is produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant is collected, filtered through 0.45 um filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this example, since the uricase and uric acid transporter are both expressed from the same lentiviral vector, only one lentivirus produced from that vector is used for the transduction step.

### Production of Uricase and Uric Acid Transporter:

The presence of uricase is assessed via intracellular staining using antibody specific for the uricase produced and analysis via flow cytometry or western blot analysis following SDS-PAGE separation. The presence of uric acid transporter is assessed either by intracellular or extracellular staining using antibody specific for the uric acid transporter produced and analysis is via flow cytometry.

### Example 6

### Generation of Erythroid Cells Genetically Engineered to Comprise Candida Utilis Uricase and Human URAT1

### Production of Lentiviral Vector:

Two lentiviral vectors were constructed containing genes for the expression of Candida utilis uricase N-terminally fused to eGFP via a Gly₄-Ser-Gly₄ linker (SEQ ID NO: 32) (eGFP-CuUricase) or Homo sapiens URAT1 N-terminally fused to (extracellular) HA epitope tag-Glycophorin A (HA-GPA) via a (Gly₄-Ser)₄-Gly₄ linker (SEQ ID NO: 33) (HA-GPA-URAT1).

Each lentivirus was produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells were placed in fresh culturing medium. The virus supernatant was collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant was collected, filtered through 0.45 um filter, and frozen in aliquots in −80° C.

### Expansion and Differentiation of Erythroid Cells:

Human CD34+ cells derived from mobilized peripheral blood cells from normal human donors were purchased frozen from Fred Hutchinson Cancer Research Center. The expansion/differentiation procedure comprises 3 stages. In the first stage, thawed CD34+ erythroid precursors were cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 5 days. In the second stage, erythroid cells were cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 9 days. In the third stage, erythroid cells were cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 5E5 cells/mL for 10-12 days. Fresh differentiation medium was added to the cultures on various days. The cultures were maintained at 37° C. in 5% CO2 incubator.

### Transduction of Erythroid Precursor Cells:

Erythroid precursor cells were transduced on day 7 of the culture process described above. Erythroid cells in culturing medium were combined with a mixture of the lentivirus for uricase and the lentivirus for URAT1, together with 1 mg/mL poloxamer 338 and incubated overnight at 37° C. The following day, erythroid cells were gently spun down at 2000 rpm for 5 minutes, supernatant removed, cells were re-suspended in fresh erythroid differentiation medium and cultured further at 37° C.

### Example 7

### Generation of Erythroid Cells Genetically Engineered to Comprise Uricase, Uric Acid Transporter and Catalase by Expression of a Uricase-Transporter Fusion in a First Lentiviral Vector and Catalase in a Second Lentiviral Vector

Production of uricase, uric acid transporter and catalase in engineered erythroid cells can be accomplished in two alternate ways. In this example, the uricase and uric acid transporter are expressed as a fusion polypeptide in a first lentiviral vector, and catalase is expressed in a second lentiviral vector, and the two different lentiviruses are mixed at transduction.

### Production of Lentiviral Vector—Uricase-Uric Acid Transporter Fusion

Lentiviral vector is constructed as described in Example 5. Uricase and uric acid transporter are expressed as direct polypeptide fusions separated by a linker, as shown in FIG. 3.

### Production of Lentiviral Vector—Catalase

Another lentiviral vector is constructed with gene encoding catalase under the control of the MSCV promoter. Lentivirus is produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant is collected, filtered through 0.45 um filter, and frozen in aliquots in −80° C.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this instance, where the uricase and uric acid transporter are expressed from two separate vectors, the two lentiviruses produced from the corresponding vectors are combined together for the transduction step.

### Production of Uricase, Uric Acid Transporter and Catalase:

The presence of uricase is assessed via intracellular staining using antibody specific for the uricase produced and analysis via flow cytometry or western blot analysis following SDS-PAGE separation. The presence of catalase is assessed via intracellular staining using antibody specific for the catalase produced and analysis is via flow cytometry or western blot analysis following SDS-PAGE separation. The presence of uric acid transporter is assessed either by intracellular or extracellular staining using antibody specific for the uric acid transporter produced and analysis via flow cytometry.

### Example 8

### Generation of Erythroid Cells Genetically Engineered to Comprise Uricase, Uric Acid Transporter and Catalase by Expression of a Uricase-Catalase Fusion in a First Lentiviral Vector and Uric Acid Transporter in a Second Lentiviral Vector

Production of uricase, uric acid transporter and catalase in engineered erythroid cells can be accomplished in two alternate ways. In this example, the uricase and catalase are expressed as a fusion in a first lentiviral vector, and uric acid transporter is expressed in a second lentiviral vector, and the two different lentiviruses are mixed at transduction.

### Production of Lentiviral Vector—Uricase-Catalase Fusion

Catalase and uricase are expressed as direct polypeptide fusions separated by a linker, for example in a configuration as catalase-linker-uricase or in a configuration of uricase-linker-catalase (expressed as N-terminus to C-terminus). Without being bound by theory, it is thought that the catalase-linker-uricase configuration may be preferable because uricase activity decreases when fused via the C-terminus. For example, Uricase and uric acid transporter are expressed as direct polypeptide fusions separated by a linker, as shown in FIG. 3. Lentivirus is produced in HEK-293T cells by transfecting the cells with pPACKH1 (System Biosciences) or in-house made packaging vector mix and the constructed lentiviral vector using TransIT-LT1 transfection reagent (Mirus). After 12-14 hour incubation, cells are placed in fresh culturing medium. The virus supernatant is collected 48 hours post-medium change by centrifugation at 1,500 rpm for 5 minutes. The supernatant is collected, filtered through 0.45 um filter, and frozen in aliquots in −80° C.

### Production of Lentiviral Vector—Uric Acid Transporter

Lentiviral vector is constructed with gene encoding uric acid transporter under the control of the MSCV promoter, as described in Example 3.

### Expansion and Differentiation of Erythroid Cells:

Human CD34+ cells derived from mobilized peripheral blood cells from normal human donors are purchased frozen from Fred Hutchinson Cancer Research Center. The expansion/differentiation procedure comprises 3 stages. In the first stage, thawed CD34+ erythroid precursors are cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells are cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells are cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium is added to the cultures on various days. The cultures are maintained at 37° C. in 5% CO2 incubator.

### Transduction of Erythroid Precursor Cells:

Erythroid precursor cells are transduced on day 7 of the culture process described above. Erythroid cells in culturing medium are combined with lentivirus and 1 mg/mL poloxamer 338 and incubated overnight at 37° C. The following day, erythroid cells are gently spun down at 2000 rpm for 5 minutes, supernatant removed, cells are re-suspended in fresh erythroid differentiation medium and cultured further at 37° C. In this instance, where the uricase and uric acid transporter are expressed from two separate vectors, the two lentiviruses produced from the corresponding vectors are combined together for the transduction step.

### Production of Uricase, Uric Acid Transporter and Catalase:

The presence of uricase is assessed via intracellular staining using antibody specific for the uricase produced and analysis via flow cytometry or western blot analysis following SDS-PAGE separation. The presence of catalase is assessed via intracellular staining using antibody specific for the catalase produced and analysis is via flow cytometry or western blot analysis following SDS-PAGE separation. The presence of uric acid transporter is assessed either by intracellular or extracellular staining using antibody specific for the uric acid transporter produced and analysis via flow cytometry.

### Example 9

### Activity of Engineered Erythroid Cells Comprising Uricase

Engineered erythroid cells comprising uricase are prepared as described in Example 2.

The activity of the uricase itself is assessed by performing uricase activity assay using lysed engineered erythroid cells expressing uricase. In this assay, the engineered erythroid cells comprising uricase are lysed and lysate is incubated with 500 uM uric acid at 37° C. At various time points the enzymatic reaction is stopped via incubation at high temperature, acid or organic solvent-mediated protein precipitation, or another method that quenches uricase enzymatic activity. Uricase activity is assessed either by degradation of uric acid over time or by the appearance of either the direct product or the product of the spontaneous degradation of the direct product (5-Hydroxyisourate, 2-oxo-4-hydroxy-5-ureidoimidazoline, (S)-Allantoin) over time. One unit of activity is defined as degradation of 1 umole of uric acid per minute.

### Example 10

### Activity of Engineered Erythroid Cells Comprising Uric Acid Transporter

Engineered erythroid cells comprising uric acid transporter are prepared as described in Example 3.

The activity of the uric acid transporter itself is assessed by performing a uric acid transport assay. In this assay, cells are incubated with uric acid or heavy uric acid at 37° C. At various time points, cells are quickly washed with ice-cold buffer, lysed, and the amount of uric acid in the cells is measured by mass spectrometry.

### Example 11

### Activity of Engineered Erythroid Cells Comprising Uricase and Uric Acid Transporter

Engineered erythroid cells comprising uricase and uric acid transporter are prepared as described in Example 4 or 5.

The combined activity of uricase and uric acid transporter is assessed by performing a uricase activity assay using intact erythroid cells engineered to comprise uricase and uric acid transporter. In this assay, intact engineered erythroid cells are incubated with 500 uM uric acid at 37° C. in a biological neutral-pH buffer or human serum. At various time points the cells are lysed and the enzymatic reaction is stopped via protein-precipitating addition of acid, ethanol, organic solvent, or a similar agent. Uricase activity is assessed either by degradation of uric acid over time, or by the appearance of either the direct product or the product of the spontaneous degradation of the direct product (5-Hydroxyisourate, 2-oxo-4-hydroxy-5-ureidoimidazoline, (S)-Allantoin) over time. One unit of activity is defined as degradation of 1 umole of uric acid per minute.

### Example 12

### Activity of Engineered Erythroid Cells Comprising Candida utilis Uricase and Human URA T1

Engineered erythroid cells comprising uricase and uric acid transporter were prepared as described in Example 6.

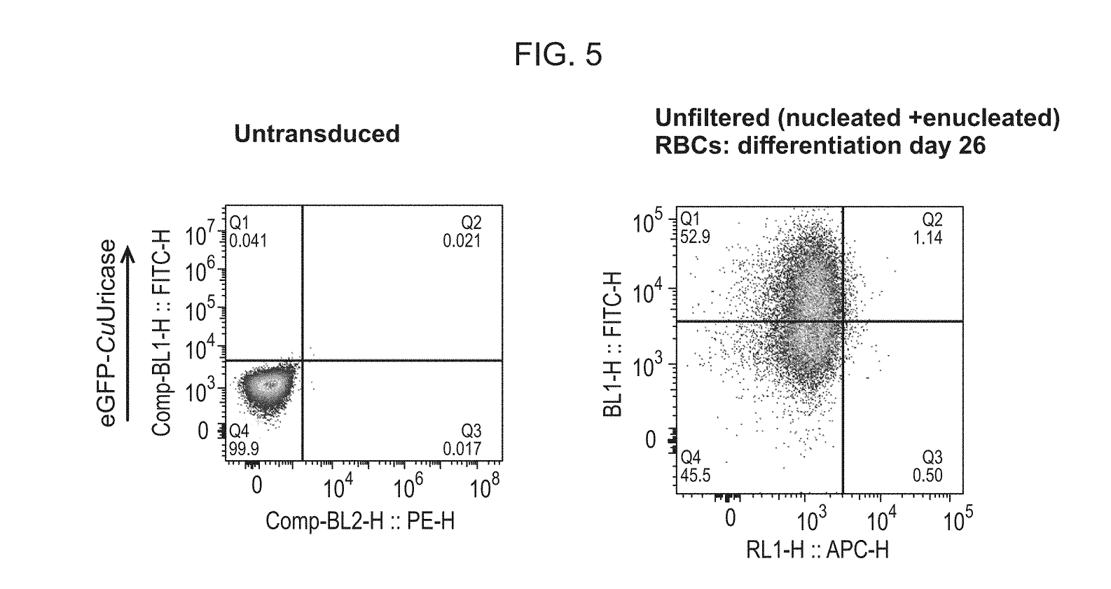

eGFP-CuUricase was tracked via eGFP fluorescence and flow cytometry. FIG. 5 shows the presence of eGFP-CuUricase in a mixture of nucleated and enucleated engineered erythroid cells on differentiation day 26. At differentiation day 26, the cells are generally at the end of the differentiation process. The amount of eGFP-CuUricase in the cells was quantified using eGFP-conjugated bead standards. It was calculated that engineered erythroid cells expressing eGFP-CuUricase contained about 170,000 eGFP-CuUricase molecules per cell. Since, typically, the same percentage of cells are GFP positive for nucleated and enucleated cells, it is expected that the enucleated cells (as well as nucleated cells) contained about 170,000 eGFP-CuUricase molecules per cell.

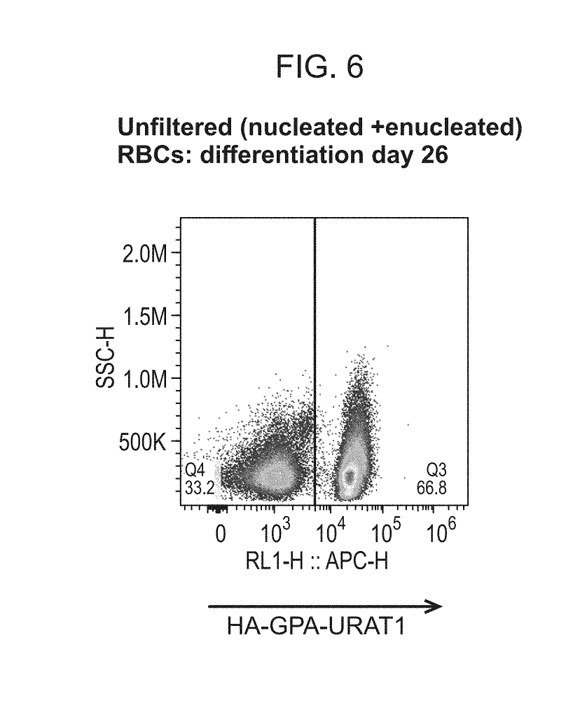

The production of HA-GPA-URAT1 was tracked via staining for extracellular HA epitope tag using fluorescently labeled anti-HA antibody and analysis by flow cytometry. FIG. 6 shows the presence of HA-GPA-URAT1 on a mixture of nucleated and enucleated engineered erythroid cells on differentiation day 21. At differentiation day 21, the cells are generally close to the end of the differentiation process. The amount of HA-GPA-URAT1 on the cells was quantified using fluorescently labeled anti-HA epitope tag antibody bound to QUANTUM Simply Cellular beads (Bangs Laboratories, Inc.). It was calculated that engineered erythroid cells comprising HA-GPA-URAT1 contained about 30,000 HA-GPA-URAT1 molecules per cell. Since, typically, the same percentage of cells are GFP positive for nucleated and enucleated cells, it is expected that the enucleated cells (as well as nucleated cells) contained about 30,000 HA-GPA-URAT1 molecules per cell.

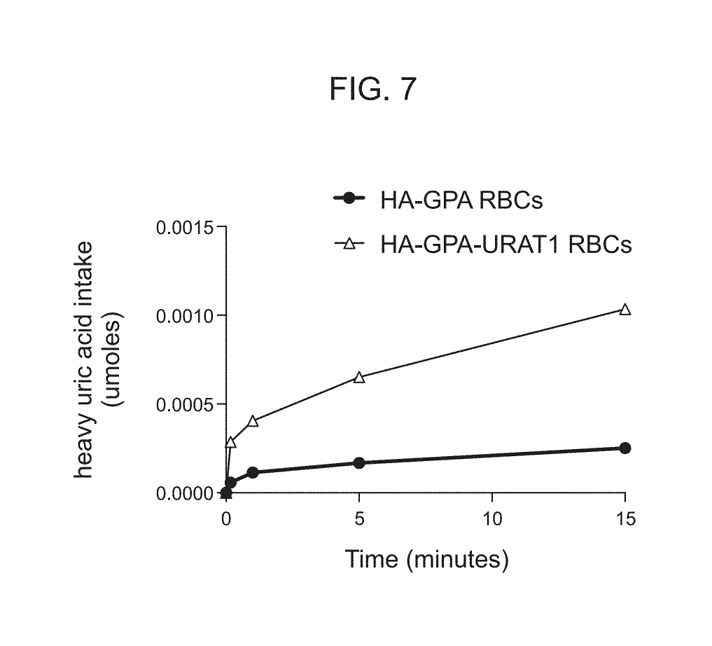

The activity of the HA-GPA-URAT1 was assessed by a uric acid transport assay (FIG. 7). Cells were incubated with heavy uric acid at 37° C. At various time points, cells were quickly washed with ice-cold buffer, lysed, and the amount of heavy uric acid in the cells was measured by mass spectrometry. The transport rate of uric acid for control red cells comprising HA-GPA was measured to be 1.8e-11 umole/minute/expressing cell. For red cells comprising HA-GPA-URAT, the uric acid transport rate was measured to be 1.3e-10 umole/minute/cell, which was at least 10 fold higher than that of the control red cells comprising HA-GPA.

### Example 13

### Dose Estimation for Engineered Erythroid Cells Comprising Uricase and Uricase Acid Transporter

Engineered erythroid cells comprising uricase and uric acid transporter are prepared as described in Example 6.

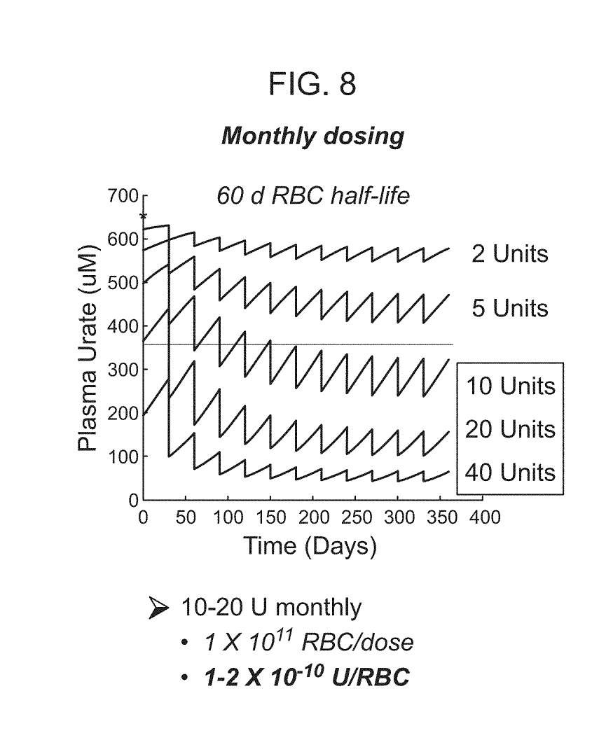

The dose estimation graph, presented in FIG. 8, shows that the target activity per dose of engineered erythroid cells comprising a uric acid degrading polypeptide (e.g., uricase), optionally together with a uric acid transporter, is approximately 10-20 units. This dose modeling calculation assumes (i) a 60 day half-life of the engineered erythroid cells, and (ii) the dose being administered monthly. Serum uric acid levels above 360 uM are considered pathogenic. If a dose is taken to be approximately 1e11 engineered erythroid cells, then for clinical application, the target activity, i.e., the transport rate of uric acid into the engineered erythroid cells and subsequent break down of uric acid by uricase inside the cell, must be at least about 1-2e⁻¹⁰ units/engineered erythroid cell (RCT), where one unit of activity is defined as degradation of 1 μmol of uric acid per minute. Alternatively, if a dose were taken to be a lower number of engineered erythroid cells, e.g., approximately 3e10 engineered erythroid cells, then for clinical application, the target activity, i.e., the transport rate of uric acid into the engineered erythroid cells and subsequent break down of uric acid by uricase inside the cell, would need to be at least about 3-6e⁻¹⁰ units/engineered erythroid cell (RCT), where one unit of activity is defined as degradation of 1 μmol of uric acid per minute.

Next, experiments were carried out to determine whether sufficient enzyme levels in the erythroid cell could be achieved in order to obtain the target activity for when a dose is taken as 1e11 engineered erythroid cells. As shown in FIG. 7, when HA-GPA-URAT1 was comprised on the surface of erythroid cells, uric acid import into the RCTs was measured to be 1.3e-10 units/RCT, which is within the needed range. As shown in FIG. 5, CuUricase was present at a level of about 170,000 molecules/cell. Therefore, given the measured specific activity for Candida utilis uricase at the desired pH, between 5e4 and 1.8e5 uricase molecules per RBC should yield at least the target activity of 1-2e-10 units/RCT.

### Example 14

### Effects of Erythroid Cell Genetically Engineered to Comprise Uricase and Uric Acid Transporter in In Vivo Studies Using Uricase Knockout Mice

The objective of this study is to assess the effect of erythroid cells engineered to comprise a uricase and uric acid transporter in a uricase knockout mouse model.

Jackson Laboratories carries a mouse model for gout in which the animals are engineered to have inactive endogenous uricase via homologous recombination (uricase knockout mice) (B6;129S7-Uox^{tm1Bay}IJ; 002223). This mouse model was first described in Wu et al. 1994 Proc Natl Acad Sci USA, 91: 742 publication, which is incorporated by reference in its entirety herein. Mice homozygous for inactive uricase have significantly decreased viability unless given allopurinol and display pronounced hyperuricemia and related nephropathy. When not treated with allopurinol, uricase knockout mice have elevated serum uric acid levels: ˜11 mg/100 ml in homozygous uricase knockout mice compared to ˜1 mg/100 ml in wild type mice. It is expected that introduction of erythroid cells genetically engineered to express uricase and uric acid transporter into the circulation of uricase knockout mice will decrease serum uric acid levels.

A group of 10 animals for each condition are tested (group 1: unmodified erythroid cells; group 2: positive control—recombinant uricase; groups 3-5: different doses of erythroid cells expressing a uricase and uric acid transporter. Homozygous knockout uricase mice are first taken off of allopurinol to induce increase in serum uric acid levels. Mice are also treated with clodronate liposomes as well as cobra venom factor prior to and around the time of human erythroid cell infusion. When uric acid levels in mice reach ˜10 mg/100 ml, control unmodified erythroid cell, recombinant control uricase, as well erythroid cells engineered to express uricase and uric acid transporter are introduced into mice via intravenous injection. Multiple doses of treatment material is injected into mice. Blood from dosed mice is periodically collected and concentration of uric acid in serum is determined. Infusion of erythroid cell expressing uricase and uric acid transporter is expected to decrease serum uric acid levels.

### Example 15

### Uric Acid Degradation Activity of Enucleated Erythroid Cells Genetically Engineered to Comprise Uricase and Human Uric Acid Transporter

### Production of Lentiviral Vector

Lentiviral vector was constructed with the gene encoding Arthrobacter globiformis uricase N-terminally fused to eGFP via a 9 amino acid linker (eGFP-AgUricase) under the control of the MSCV promoter. Lentiviral vector was also constructed with the gene encoding Candida utilis uricase N-terminally fused to eGFP via a 19 amino acid linker (eGFP-CuUricase) under the control of the MSCV promoter. Lentiviral vector was constructed with the gene encoding human uric acid transporter Glut9 N-terminally fused to Glycophorin A and HA epitope tag (HA-GPA-Glut9) under the control of the MSCV promoter. Lentiviral vector was constructed with the gene encoding human uric acid transporter URAT1 N-terminally fused to Glycophorin A and HA epitope tag (HA-GPA-URAT1) under the control of the MSCV promoter. Each lentivirus was produced separately in HEK-293T cells by transfecting the cells with pPACKH1 (System Biosciences) or in-house made packaging vector mix and the constructed lentiviral vector using TransIT-LT1 transfection reagent (Mirus). After a 12-14 hour incubation, cells were placed in fresh culturing medium. The virus supernatant was collected 48 hours post-medium change by centrifugation at 1,500 rpm for 5 minutes. After collection of viral supernatant, the virus is concentrated by ultracentrifugation or tangential flow filtration (TFF) and ultracentrifugation. The supernatant was collected, filtered through 0.45 um filter, and frozen in aliquots in −80° C.

### Expansion and Differentiation of Erythroid Cells:

Human CD34+ cells derived from mobilized peripheral blood cells from normal human donors were obtained. The expansion/differentiation procedure comprised 3 stages. In the first stage, thawed CD34+ erythroid precursors were cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells were cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells were cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium was added to the cultures on various days. The cultures were maintained at 37° C. in 5% CO2 incubator.

### Transduction of Erythroid Precursor Cells:

Erythroid precursor cells were transduced on day 7 of the culture process described above. Erythroid cells in culturing medium were combined with (1) lentiviral vector constructed with the gene encoding Candida utilis uricase and lentiviral vector constructed with the gene encoding human uric acid transporter Glut9; (2) lentiviral vector constructed with the gene encoding Arthrobacter globiformis uricase and lentiviral vector constructed with the gene encoding human uric acid transporter URAT1; (3) lentiviral vector constructed with the gene encoding Arthrobacter globiformis uricase and lentiviral vector constructed with the gene encoding human uric acid transporter Glut9, together with 1 mg/mL poloxamer 338 at either MOI (multiplicity of infection) of 25 for both viruses (total MOI=50) or MOI of 75 for both viruses (total MOI=150). Transduction reactions were incubated overnight at 37° C. The following day, erythroid cells were gently spun down at 2000 rpm for 5 minutes, supernatant removed, cells were re-suspended in fresh erythroid differentiation medium and cultured further at 37° C.

### Production of Uricase and Uric Acid Transporter:

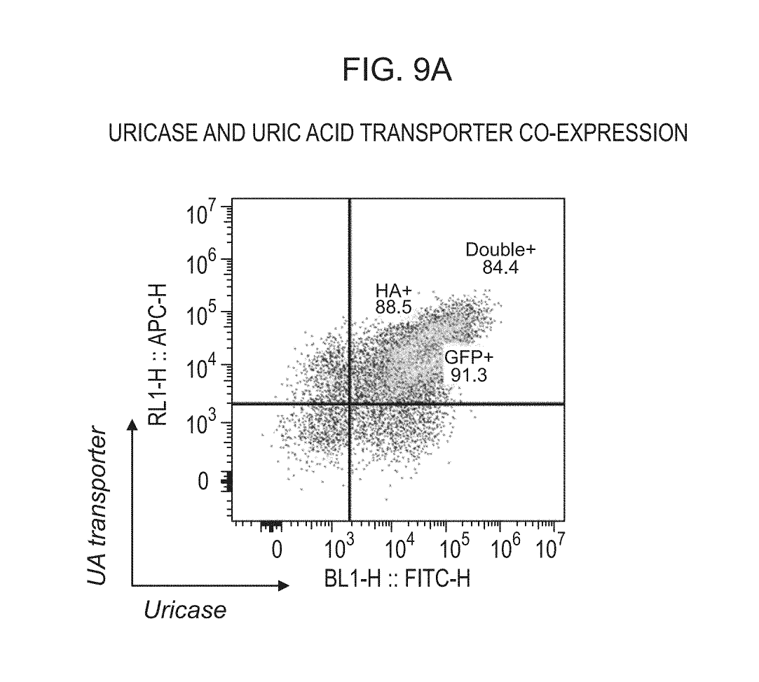

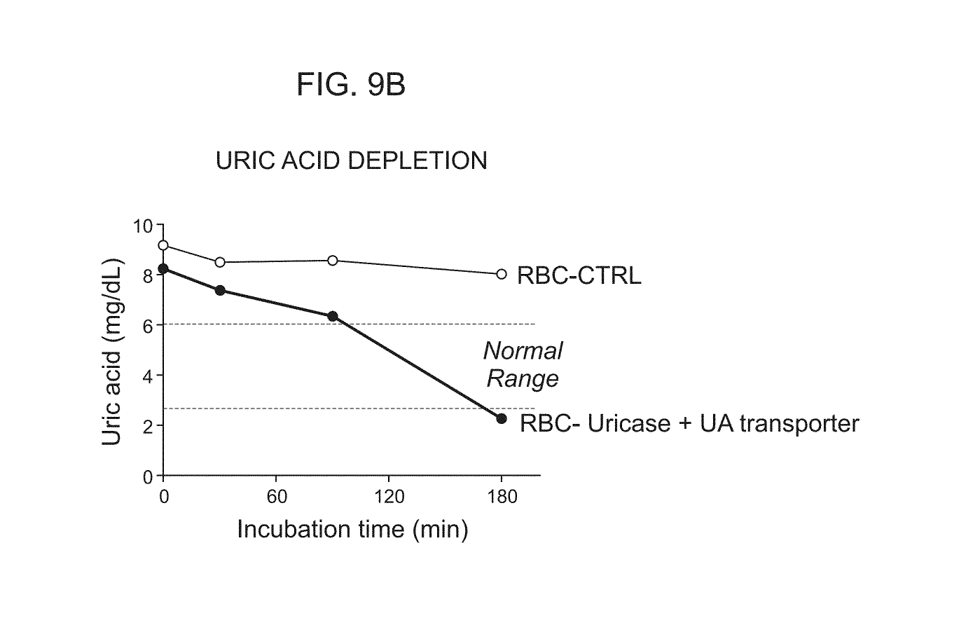

The presence of eGFP-AgUricase on day 20 enucleated erythroid cells was assessed via eGFP fluorescence detection on flow cytometer and quantified using eGFP-conjugated bead standards. The presence of HA-GPA-Glut9 on day 20 enucleated cells was assessed by extracellular staining using antibody specific for HA epitope tag and quantified using Bang's bead standards (Bangs Laboratories, Inc.) conjugated to the same anti-HA fluorescent antibody. The results, which are shown in FIG. 9A, demonstrate that a high level of co-expression of uricase and the uric acid transporter were achieved.

### Activity of Engineered Erythroid Cells Comprising Uricase and Uric Acid Transporter:

For the activity assay, intact engineered enucleated erythroid cells were incubated with 500 uM uric acid at 37° C. in human serum. At various time points, aliquots were taken, cells were spun down in a centrifuge and the concentration of uric acid in the supernatant/serum was measured via absorbance at 293 nm upon protein precipitation. Degradation of uric acid over time in erythroid cells comprising eGFP-AgUricase and HA-GPA-Glut9 is shown in FIG. 9B. As can be seen, the level of uric acid was reduced to normal range by erythroid cells co-expressing AgUricase and Glut9.

The same experiments as above were performed to determine the presence of uricase (CuUricase or AgUricase) and the presence of uric acid transporter (Glut9 or URAT1) on day 23 erythroid cells for the various combinations of uricase and uric acid transporter. The results are shown in Table 4 as % double positive enucleated erythroid cells. Additionally, the same experiments as above were performed to determine the activity of the engineered erythroid cells comprising the various combinations of uricase (CuUricase or AgUricase) and uric acid transporter (Glut9 or URAT1) for day 23 enucleated erythroid cells. One unit of activity is defined as degradation of 1 umole of uric acid per minute. The results are shown in Table 4 as activity per cell (units/cell).

As shown below, Table 4 outlines the combinations of uricase and uric acid transporter that were present in the erythroid cells, the % of enucleated erythroid cells on day 23 of differentiation containing both uricase and uric acid transporter, the number of proteins/enucleated erythroid cells on average cross the entire cell population, as well as the measured activity of the total cell population. As shown in Table 4, enucleated erythroid cells genetically engineered to comprise Arthrobacter globiformis uricase (Ag Uricase) and human uric acid transporter Glut9 or URAT1, and enucleated erythroid cells genetically engineered to comprise Candida utilis uricase (Cu Uricase) and human uric acid transporter Glut9 all showed degradation of uric acid activity, where enucleated erythroid cells genetically engineered to comprise Arthrobacter globiformis uricase (Ag Uricase) and human uric acid transporter Glut9 show the highest level of activity.

TABLE 4

| | | % double | Number of | Number of | |
|---|---|---|---|---|---|
| | | positive | uricase | transporter | Activity per |
| | | enucleated | molecules/ | molecules/ | cell in total |
| | Transduction | erythroid | cell in total | cell in total | population |
| | MOI | cells | population | population | (units/cell) |
| eGFP-CuUricase + | 25 | 51.5 | 54,308 | 7,929 | 2.69e−11 |
| HA-GPA-Glut9 | | | | | |
| eGFP-CuUricase + | 75 | 61.2 | 53,178 | 10,381 | 3.96e−11 |
| HA-GPA-Glut9 | | | | | |
| eGFP-AgUricase + | 25 | 72.2 | 103,023 | 13,558 | 2.95e−11 |
| HA-GPA-URAT1 | | | | | |
| eGFP-AgUricase + | 75 | 70.2 | 42,454 | 34,884 | 2.44e−11 |
| HA-GPA-URAT1 | | | | | |
| eGFP-AgUricase + | 25 | 53.8 | 103,570 | 7,290 | 5.56e−11 |
| HA-GPA-Glut9 | | | | | |
| eGFP-gUricase + | 75 | 67.6 | 66,908 | 13,233 | 5.3e−11 |
| HA-GPA-Glut9 | | | | | |

1. An enucleated cell engineered to degrade uric acid, comprising a first exogenous polypeptide comprising a uric acid degrading polypeptide, or a variant thereof, wherein the uric acid degrading polypeptide comprises a polypeptide selected from the group consisting of: a uricase, or a variant thereof, a 5-hydroxyisourate (HIU) hydrolase, or a variant thereof, an 2-oxo-4-hydroxy-4-carboxy-5-ureidoimidazoline (OHCU) decarboxylase, or a variant thereof, an allantoinase, or variant thereof, an allantoicase, or a variant thereof, a myeloperoxidase, or variant thereof, a flavin adenine dinucleotide (FAD)-dependent urate hydroxylase, or variant thereof, an xanthine dehydrogenase, or variant thereof, a nucleoside deoxyribosyltransferase, or variant thereof, a dioxotetrahydropyrimidine phosphoribosyltransferase, or variant thereof, a dihydropyrimidinase, or variant thereof, and a guanine deaminase, or a variant thereof.

2.-10. (canceled)

11. An enucleated cell engineered to degrade uric acid, comprising a first exogenous polypeptide comprising a uric acid degrading polypeptide, wherein the engineered enucleated cell has uricolytic activity of at least about 1e-10 units/cell.

12. The engineered enucleated cell of claim 11, wherein the engineered enucleated cell has uricolytic activity of at least about 3e-10 to about 6e-10 units/cell.

13.-17. (canceled)

18. The engineered enucleated cell of claim 11, wherein the uric acid degrading polypeptide comprises a polypeptide selected from the group consisting of: a uricase, or a variant thereof, a 5-hydroxyisourate (HIU) hydrolase, or a variant thereof, an 2-oxo-4-hydroxy-4-carboxy-5-ureidoimidazoline (OHCU) decarboxylase, or a variant thereof, an allantoinase, or variant thereof, an allantoicase, or a variant thereof, a myeloperoxidase, or variant thereof, a flavin adenine dinucleotide (FAD)-dependent urate hydroxylase, or variant thereof, an xanthine dehydrogenase, or variant thereof, a nucleoside deoxyribosyltransferase, or variant thereof, a dioxotetrahydropyrimidine phosphoribosyltransferase, or variant thereof, a dihydropyrimidinase, or variant thereof, and a guanine deaminase, or variant thereof.

19.-32. (canceled)

33. The engineered enucleated cell of claim 1, wherein the uric acid degrading polypeptide comprises a uricase, or a variant thereof, and wherein the uricase comprises a fungal uricase.

34. The engineered enucleated cell of claim 33, wherein the fungal uricase is derived from Candida utilis, Aspergillus flavus, Aspegillus niger or Penicillium freii.

35.-41. (canceled)

42. The engineered enucleated cell of claim 1, wherein the uric acid degrading polypeptide comprises a uricase, or a variant thereof, and wherein the uricase comprises a yeast uricase.

43. The engineered enucleated cell of claim 42, wherein the yeast uricase is derived from Schizosaccharomyces pombe.

44. (canceled)

45. The engineered enucleated cell of claim 1, wherein the uric acid degrading polypeptide comprises a uricase, or a variant thereof, and wherein the uricase comprises a bacterial uricase.

46. The engineered enucleated cell of claim 45, wherein the bacterial uricase is derived from Arthrobacter globiformi, Bacillus subtilis or Cellulomonas flavigena.

47.-51. (canceled)

52. The engineered enucleated cell of claim 1, 6wherein the uric acid degrading polypeptide comprises uricase, or a variant thereof, and wherein the uricase comprises a mammalian uricase.

53. The engineered enucleated cell of claim 52, wherein the mammalian uricase is derived from Mus musculus, Danio rerio, or Macaca mulatta.

54.-60. (canceled)

61. The engineered enucleated cell of claim 1, wherein the uric acid degrading polypeptide comprises uricase, or a variant thereof, and wherein the uricase comprises a plant uricase.

62. The engineered enucleated cell of claim 61, wherein the plant uricase is derived from Glycine max or Oryza sativa subsp. Japonica.

63.-65. (canceled)

66. The engineered enucleated cell of claim 1, wherein the uric acid degrading polypeptide comprises uricase, or a variant thereof, and wherein the uricase is derived from Drosophila melanogaster.

67.-71. (canceled)

72. The engineered enucleated cell of claim 1, wherein the first exogenous polypeptide is inside the enucleated cell.

73. The engineered enucleated cell of claim 1, further comprising a second exogenous polypeptide comprising a uric acid transporter, or a variant thereof.

74. The engineered enucleated cell of claim 73, wherein the uric acid transporter is selected from the group consisting of: URAT1, GLUT9, OAT4, OAT1, OAT3, Gal-9, ABCG2, SLC34A2, MRP4, OAT2, NPT4, NPT1, and MCT9.

75. The engineered enucleated cell of claim 74, wherein URAT1 comprises an amino acid sequence that is at least 95% identical to the amino acid sequence set forth in SEQ ID NO: 16 ; wherein GLUT9 comprises an amino acid sequence that is at least 95% identical to the amino acid sequence set forth in SEQ ID NO: 17; wherein OAT4 comprises an amino acid sequence that is at least 95% identical to the amino acid sequence set forth in SEQ ID NO: 18; wherein OAT1 comprises an amino acid sequence that is at least 95% identical to the amino acid sequence set forth in SEQ ID NO: 19; wherein OAT3 comprises an amino acid sequence that is at least 95% identical to the amino acid sequence set forth in SEQ ID NO: 20; wherein Gal-9 comprises an amino acid sequence that is at least 95% identical to the amino acid sequence set forth in SEQ ID NO: 21; wherein ABCG2 comprises an amino acid sequence that is at least 95% identical to the amino acid sequence set forth in SEQ ID NO: 22; wherein SLC34A2 comprises an amino acid sequence that is at least 95% identical to the amino acid sequence set forth in SEQ ID NO: 23; wherein MRP4 comprises an amino acid sequence that is at least 95% identical to the amino acid sequence set forth in SEQ ID NO: 24; wherein OAT2 comprises an amino acid sequence that is at least 95% identical to the amino acid sequence set forth in SEQ ID NO:44; wherein NPT4 comprises an amino acid sequence that is at least 95% identical to the amino acid sequence set forth in SEQ ID NO:45; wherein NPT1 comprises an amino acid sequence that is at least 95% identical to the amino acid sequence set forth in SEQ ID NO:46; or wherein MCT9 comprises an amino acid sequence that is at least 95% identical to the amino acid sequence set forth in SEQ ID NO:47.

76.-82. (canceled)

83. The engineered enucleated cell of claim 73, wherein the uric acid transporter transports uric acid from outside the enucleated cell to the inside of the enucleated cell at a rate of at least about 1.0e-10, or at least about 1.3e-10 umol uric acid per minute per cell (μmol/min/cell).

84. The engineered enucleated cell of claim 73, wherein the enucleated cell further comprises a third exogenous polypeptide comprising a catalase, or a variant thereof.

85.-87. (canceled)

88. The engineered enucleated cell of claim 1, which is an erythroid cell or a platelet.

89. (canceled)

90. An engineered enucleated cell comprising a first exogenous polypeptide comprising a uric acid transporter, or a variant thereof, wherein the uric acid transporter is selected from the group consisting of: URAT1, GLUT9, OAT4, OAT1, OAT3, Gal-9, ABCG2, SLC34A2, MRP4, OAT2, NPT4, NPT1, and MCT9.

91.-97. (canceled)

98. The engineered enucleated cell of claim 90, wherein the uric acid transporter transports uric acid from outside the enucleated cell to the inside of the enucleated cell at a rate of at least about 1.0e-10, or at least about 1.3e-10 μmol uric acid per minute per cell (μmol/min/cell).

99. The engineered enucleated cell of claim 90, which is an erythroid cell or a platelet.

100.-105. (canceled)

106. A pharmaceutical composition comprising a plurality of the engineered enucleated cells of claim 1, and a pharmaceutically acceptable carrier.

107.-113. (canceled)

114. A method of treating or preventing hyperuricemia in a subject, comprising administering to the subject the engineered enucleated cell of claim 1, or the pharmaceutical composition of claim 106, in an amount effective to treat or prevent hyperuricemia in the subject.

115.-118. (canceled)

119. The method of claim 114, wherein the subject has a disease selected from the group consisting of: gout, rheumatoid arthritis, osteoarthritis, cerebral stroke, ischemic heart disease, arrhythmia, and chronic renal disease.

120.-132. (canceled)

133. The method of claim 114, wherein the method further comprises administration of a second agent.

134. (canceled)

135. A method of making the engineered enucleated cell of claim 1, the method comprising:
introducing an exogenous nucleic acid encoding the first exogenous polypeptide into a nucleated erythroid cell; and
culturing the nucleated erythroid cell under conditions suitable for enucleation of the nucleated erythroid cell and for production of the first exogenous polypeptide,
thereby making the engineered enucleated cell of claim 1.

136. A method of making the engineered enucleated cell of claim 90, the method comprising:
introducing an exogenous nucleic acid encoding the first exogenous polypeptide into a nucleated erythroid cell; and
culturing the nucleated erythroid cell under conditions suitable for enucleation of the nucleated erythroid cell and for production of the first exogenous polypeptide,
thereby making the engineered enucleated cell of claim 90.

137.-162. (canceled)
